SEQUENCE LISTING (9 sequences)

SEQ ID NO: 1 - DNA, 29903 bp, Severe acute respiratory syndrome coronavirus 2
  source 1..29903: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 21282 bp, Severe acute respiratory syndrome coronavirus 2
  source 1..21282: mol_type = genomic DNA
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtctgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactattattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctataaaatgggcagataacaactgttatcttgccactgcattgttaacactccaacaaatagagttgaagtttaatccacctgctctacaagatgcttattacagagcaagggctggtgaagctgataacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttattttacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattacaatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaaccgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacctggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcatcttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacctcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccatccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataggcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 3 - DNA, 21291 bp, Severe acute respiratory syndrome coronavirus 2
  source 1..21291: mol_type = genomic DNA
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaaccgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggatgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 4 - DNA, 21291 bp, Severe acute respiratory syndrome coronavirus 2
  source 1..21291: mol_type = genomic DNA
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaaccgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 5 - DNA, 25 bp, synthetic construct
  misc_feature 1..25: note = Description of Artificial Sequence: Synthetic oligonucleotide
  source 1..25: mol_type = other DNA
accctgaaagtacgtgcattacatg

SEQ ID NO: 6 - DNA, 25 bp, synthetic construct
  misc_feature 1..25: note = Description of Artificial Sequence: Synthetic oligonucleotide
  source 1..25: mol_type = other DNA
atgtctattacgtcacactattatg

SEQ ID NO: 7 - DNA, 106 bp, synthetic construct
  misc_feature 1..106: note = Description of Artificial Sequence: Synthetic polynucleotide
  source 1..106: mol_type = other DNA
aataacccctttaccgaaaactgggatcatgcttcttcggtgcccattacgaggtgcggatagctaccgcgcagacacgatagtctatagaatttggacaatcatg

SEQ ID NO: 8 - DNA, 33 bp, synthetic construct
  misc_feature 1..33: note = Description of Artificial Sequence: Synthetic oligonucleotide
  source 1..33: mol_type = other DNA
ttaactactccagttttcggtaaaggggttatt

SEQ ID NO: 9 - DNA, 27 bp, synthetic construct
  misc_feature 1..27: note = Description of Artificial Sequence: Synthetic oligonucleotide
  source 1..27: mol_type = other DNA
catgattgtccaaattctagattaatg

(19) United States
(12) Patent Application Publication
(10) Pub. No.: US 2023/0295692 A1
(43) Pub. Date: Sep. 21, 2023

(54) MULTIPLEXED COVID-19 PADLOCK ASSAY

(71) Applicant: ELEMENT BIOSCIENCES, INC., San Diego, CA (US)

(72) Inventors: Lorenzo BERTI, San Diego, CA (US); Semyon KRUGLYAK, San Diego, CA (US); Matthew KELLINGER, San Diego, CA (US); Molly HE, San Diego, CA (US); Sinan ARSLAN, San Diego, CA (US); Junhua ZHAO, San Diego, CA (US); Michael PREVITE, San Diego, CA (US)

(21) Appl. No.: 18/161,581

(22) Filed: Jan. 30, 2023

Related U.S. Application Data
(63) Continuation of application No. PCT/US2021/044002, filed on Jul. 30, 2021.
(60) Provisional application No. 63/059,723, filed on Jul. 31, 2020.

(51) Int. Cl. C12Q 1/6816; C12Q 1/6855

(52) U.S. Cl. CPC C12Q 1/6816; C12Q 1/6855

(57) ABSTRACT

Methods and systems for detecting the presence of a target nucleic acid sequence in one or more samples of a plurality of samples are described. The methods may comprise the use of linear barcoded nucleic acid probes that, upon hybridization to a target nucleic acid sequence, may be ligated to circularize the probe molecule, amplified, and sequenced. The use of a probe-specific barcode integrated into the nucleic acid probe molecule, and sample-specific barcodes that may be incorporated into the nucleic acid probe molecule or added during the amplification step, enable large-scale multiplexed assay and sample processing.

### CROSS-REFERENCE

This application is a continuation of International Application No. PCT/US2021/044002, filed Jul. 30, 2021, which claims the benefit of U.S. Provisional Application No. 63/059,723, filed Jul. 31, 2020, which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 11, 2023, is named “52933-732_301SL.xml” and is 104,834 bytes in size.

### BACKGROUND

The COVID-19 pandemic has highlighted the shortcomings of current PCR-based molecular diagnostic testing capability. Besides a lack of precision and unacceptable false-positive/false-negative rates for many PCR-based tests, a critical flaw of existing assays is the inability to rapidly and cost-effectively scale testing to population-level monitoring of infectious disease. These shortcomings are primarily due to the limited amount of information generated by a single assay, which is usually limited to testing only a few sample replicates and is therefore insufficient for generating high precision test results. Additionally, the molecular diagnostic techniques used (primarily PCR-based) are not currently amenable for large-scale sample multiplexing. Thus, there remains an unmet need for a cost-effective method to increase the number of sample replicate tests per assay that is also compatible with a large-scale multiplexing strategy.

This disclosure relates to compositions, methods, and systems for addressing the shortcomings of current molecular diagnostic testing capabilities as they relate to population-scale monitoring of infectious disease.

### SUMMARY

Aspects disclosed herein provide methods for nucleic acid detection, said method comprising: (a) contacting a nucleic acid sequence obtained from a sample with a nucleic acid probe molecule comprising a distal end and a proximal end under conditions sufficient to couple said distal end of said nucleic acid probe molecule and said proximal end of said nucleic acid probe molecule to said nucleic acid sequence, thereby forming a circular nucleic acid probe molecule; and (b) detecting a presence of said nucleic acid sequence by identifying a sequence of said circular nucleic acid probe molecule, wherein said detecting comprises performing a nucleotide binding reaction in the presence of a polymerizing enzyme between (i) said circular nucleic acid probe molecule or a derivative thereof and (ii) a nucleotide moiety comprising a detectable label, wherein said nucleotide binding reaction is performed in the absence of incorporation of said nucleotide moiety into said circular nucleic acid probe molecule or derivative thereof. In some embodiments, said circular nucleic acid probe molecule comprises a gap in a sequence thereof. In some embodiments, said method further comprises contacting said nucleic acid probe molecule with a polymerizing enzyme under conditions sufficient to perform an extension reaction, thereby filling said gap with a copy of a portion of said nucleic acid sequence. In some embodiments, said sequence of said circular nucleic acid probe molecule that is identified in (b) comprises said portion of said nucleic acid sequence. In some embodiments, said method further comprises contacting said nucleic acid probe molecule with a ligating enzyme under conditions sufficient to ligate said distal end of said nucleic acid probe molecule to said proximal end of said nucleic acid probe molecule following said extension reaction. In some embodiments, said gap comprises between 1 and 200 contiguous nucleotides in length. In some embodiments, said method further comprises contacting said nucleic acid probe molecule with a ligating enzyme under conditions sufficient to ligate said distal end of said nucleic acid probe molecule to said proximal end of said nucleic acid probe molecule, thereby forming said circular nucleic acid probe molecule. In some embodiments, said nucleic acid probe molecule is linear when unhybridized. In some embodiments, said nucleic acid sequence of said circular nucleic acid probe molecule that is identified in (b) comprises a barcode sequence that uniquely identifies said presence of said nucleic acid sequence when it is identified. In some embodiments, said method further comprises: (c) repeating (a) to (b) to identify a plurality of said nucleic acid sequence of a plurality of said circular nucleic acid probe molecule in a sample; and (d) counting a number of times each said nucleic acid sequence of said plurality of said nucleic acid sequence is identified in (c). In some embodiments, said method further comprises determining a copy number of said nucleic acid sequence in said sample, wherein said copy number of said nucleic acid sequence in said sample is proportional to said number of said times said each said nucleic acid sequence is counted in (d). In some embodiments, said method further comprises multiplexing said method comprising: (c) repeating (a) to (b) to identify a plurality of said nucleic acid sequence of a plurality of said circular nucleic acid probe molecule in said sample, wherein a first subset of said plurality of said circular nucleic acid probe molecule is different from a second subset of said plurality of said circular nucleic acid molecule; and (d) counting a number of times a first nucleic acid sequence of said first subset and a second nucleic acid sequence of said second subset are identified in (c). In some embodiments, said first subset of said plurality of said circular nucleic acid probe molecule is different from said second subset of said plurality of said circular nucleic acid molecule in that: (i) said first subset comprises a different barcode sequence from said second subset; (ii) said first subset comprises a different distal end or proximal end from said second subset; or (iii) a combination of (i) and (ii). In some embodiments, said method further comprises detecting a presence of a second nucleic acid sequence in said sample, comprising: (c) contacting said second nucleic acid sequence in said sample with a second nucleic acid probe molecule under conditions sufficient to couple said second nucleic acid sequence with said second nucleic acid probe molecule, thereby forming a second circular nucleic acid probe molecule; and (d) bringing said second circular nucleic acid probe molecule or derivative thereof in contact with (i) a second polymerizing enzyme and (ii) a second nucleotide moiety comprising a second detectable label under conditions sufficient to cause a second nucleotide binding reaction to occur between said second circular nucleic acid probe molecule or derivative thereof and said second nucleotide moiety in the absence of incorporation of said second nucleotide moiety into said second circular nucleic acid probe molecule or derivative thereof, wherein said second nucleic acid sequence is different from said nucleic acid sequence detected in (b). In some embodiments, said method further comprises amplifying said circular nucleic acid probe molecule to produce said derivative thereof. In some embodiments, said amplifying comprises performing rolling circle amplification. In some embodiments, said nucleotide moiety is coupled to a polymer core in a polymer-nucleotide composition, forming a polymer-nucleotide conjugate. In some embodiments, said detectable label is coupled to said polymer core of said polymer-nucleotide composition. In some embodiments, said nucleotide binding reaction comprises two or more binding events between two or more of said nucleotide moiety and two or more copies of said nucleic acid sequence. In some embodiments, said detectable label comprises a fluorescent label. In some embodiments, said method further comprises detecting a presence of a second nucleic acid sequence derived from a second sample, comprising: (c) contacting said second nucleic acid sequence in said second sample with a second nucleic acid probe molecule under conditions sufficient to couple said second nucleic acid sequence with said second nucleic acid probe molecule, thereby forming a second circular nucleic acid probe molecule; and (d) bringing said second circular nucleic acid probe molecule or derivative thereof in contact with (i) a second polymerizing enzyme and (ii) a second nucleotide moiety comprising a second detectable label under conditions sufficient to cause a second nucleotide binding reaction to occur between said second circular nucleic acid probe molecule or derivative thereof and said second nucleotide moiety in the absence of incorporation of said second nucleotide moiety into said second circular nucleic acid probe molecule or derivative thereof, wherein said second nucleic acid sequence is different from said nucleic acid sequence detected in (b), thereby detecting said presence of said second nucleic acid sequence in said second sample. In some embodiments, said second sample is obtained from a different source from said sample. In some embodiments, said method further comprises tracing a pathogenic infection by a pathogenic source of said nucleic acid sequence and said second nucleic acid sequence, wherein said tracing comprises comparing a first location or a first time of collection of said sample with a second location or a second time of collection of said second sample. The method of any one of claims 1-23, wherein said sample is obtained from a source comprising: (i) soil; (ii) sewage; (iii) biological tissue; (iv) food; (v) a surface of an object in contact with one or more of (i) to (iv); or (vi) any combination of (i) to (v).

Aspects disclosed herein provide systems for nucleic acid detection, said system comprising: one or more computer processors that are individually or collectively programmed to implement a method comprising: (a) contacting a nucleic acid sequence with a nucleic acid probe molecule under conditions sufficient to cause (i) a proximal end of said nucleic acid probe molecule to couple with a first portion of said nucleic acid sequence, and (ii) a distal end of said nucleic acid probe molecule to couple with a second portion of said nucleic acid sequence, thereby forming a circular nucleic acid probe molecule; and (b) bringing said circular nucleic acid probe molecule or a derivative thereof in contact with (i) a polymerizing enzyme and (ii) a nucleotide moiety comprising a detectable label under conditions sufficient to cause a nucleotide binding reaction to occur between said circular nucleic acid probe molecule or derivative thereof and said nucleotide moiety in the absence of incorporation of said nucleotide moiety into said circular nucleic acid probe molecule or derivative thereof. In some embodiments, said system further comprises said nucleic acid probe molecule, wherein said nucleic acid probe molecule comprises (i) said proximal end comprising a first nucleic acid sequence that is complementary to said first portion of said nucleic acid sequence, and (ii) said distal end comprising a second nucleic acid sequence that is complementary to said second portion of said nucleic acid sequence. In some embodiments, said system further comprises a substrate having a surface comprising a polymer layer coupled thereto, wherein said circular nucleic acid probe molecule is coupled to said polymer layer. In some embodiments, said polymer layer comprises a hydrophilic polymer. In some embodiments, said hydrophilic polymer comprises poly(ethylene glycol) (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, dextran, or any combination thereof. In some embodiments, said surface comprises two or more interior surfaces of a flow cell. In some embodiments, said system further comprises a ligating enzyme or catalytically-active fragment thereof configured to ligate said proximal end of said nucleic acid probe molecule and said distal end of said nucleic acid probe molecule to form said circular nucleic acid probe molecule. In some embodiments, said circular nucleic acid probe molecule comprises a gap in a nucleic acid sequence thereof. In some embodiments, said system further comprises a polymerizing enzyme configured to perform an extension reaction of said circular nucleic acid probe molecule, thereby filling said gap. In some embodiments, said gap is filled with a copy of a third portion of said nucleic acid sequence. In some embodiments, said gap comprises between 1 and 200 contiguous nucleotides in length. In some embodiments, said nucleic acid probe molecule is linear when unhybridized. In some embodiments, said method further comprises repeating (a) and (b) to identify a sequence of said circular nucleic acid probe molecule or derivative thereof, wherein said sequence comprises a barcode sequence that uniquely identifies said sequence. In some embodiments, said method further comprises: (c) repeating (a) to (b) to identify a plurality of said nucleic acid sequence of a plurality of said circular nucleic acid probe molecule in said sample; and (d) counting a number of times each sequence of said plurality of said sequence of said plurality of said circular nucleic acid probe molecule is identified in (c). In some embodiments, said system further comprises a plurality of said circular nucleic acid probe molecule comprising a first subset of said plurality of said circular nucleic acid probe molecule and a second subset of said plurality of said circular nucleic acid probe molecule, wherein said first subset is different from said second subset. In some embodiments, said method further comprises: (c) repeating (a) to (b) to identify a plurality of said nucleic acid sequence of a plurality of said circular nucleic acid probe molecule in said sample; and (d) counting a number of times a first sequence of said first subset and a second sequence of said second subset are identified in (c). The system of claim 39 or claim 40, said first subset of said plurality of said circular nucleic acid probe molecule is different from said second subset of said plurality of said circular nucleic acid probe molecule in that: (i) said first subset comprises a different barcode sequence from said second subset; (ii) said first subset comprises a different distal end or proximal end from said second subset; or (iii) a combination of (i) and (ii). In some embodiments, said system further comprises a second nucleic acid probe molecule, wherein said second nucleic acid probe molecule is configured to couple to a second nucleic acid sequence that is different from said nucleic acid sequence. In some embodiments, said method further comprises detecting a presence of said second nucleic acid in said sample, comprising: (c) contacting said second nucleic acid sequence in said sample with said second nucleic acid probe molecule under conditions sufficient to couple said second nucleic acid sequence with said second nucleic acid probe molecule, thereby forming a second circular nucleic acid probe molecule; and (b) bringing said second circular nucleic acid probe molecule or derivative thereof in contact with (i) a second polymerizing enzyme and (ii) a second nucleotide moiety comprising a second detectable label under conditions sufficient to cause a second nucleotide binding reaction to occur between said second circular nucleic acid probe molecule or derivative thereof and said second nucleotide moiety in the absence of incorporation of said second nucleotide moiety into said second circular nucleic acid probe molecule or derivative thereof. In some embodiments, said nucleotide moiety is coupled to a polymer core in a polymer-nucleotide composition. In some embodiments, said detectable label is coupled to said polymer core in said polymer-nucleotide composition, forming a polymer-nucleotide conjugate. In some embodiments, said nucleotide binding reaction comprises two or more binding events between two or more of said nucleotide moiety and two or more copies of said nucleic acid sequence. In some embodiments, said detectable label comprises a fluorescent label. In some embodiments, said nucleic acid sequence is obtained from a sample comprising: (i) soil; (ii) sewage; (iii) biological tissue; (iv) food; (v) a surface of an object in contact with one or more of (i) to (iv); or (vi) any combination of (i) to (v).

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides, according to some embodiments herein, a schematic illustration of a conventional padlock probe and its use for detection of single nucleotide polymorphisms (SNPs) (from New England Biolabs).

FIG. 2 provides a non-limiting example of a barcoded padlock probe of the present disclosure.

FIG. 2 discloses SEQ ID NOS 7-9, respectively, in order of appearance.

FIG. 3 provides, according to some embodiments herein, a schematic illustration of the SARS-CoV-2 (COVID-19) genome (from Johns Hopkins Center for Health Security, “Comparison of National RT-PCR Primers, Probes, and Protocols for SARS-CoV-2 Diagnostics”, Apr. 13, 2020).

FIG. 4 provides, according to some embodiments herein, a non-limiting example of a workflow for a barcoded molecular inversion probe (MIP) assay.

FIG. 5 provides, according to some embodiments herein, a schematic illustration of a workflow for performing a multiplexed padlock assay of the present disclosure that indicates the approximate times required for different steps of the assay.

FIG. 6 provides, according to some embodiments herein, a schematic illustration of a multivalent binding complex formed using the multivalent binding compositions described herein.

FIG. 7 shows, according to some embodiments herein, a generalized graphical depiction of the increase in signal intensity that has been observed during binding, persistence, and washing and removal of multivalent substrates.

FIG. 8 provides, according to some embodiments herein, a schematic illustration of a workflow for performing a multiplexed padlock assay followed by sequencing to detect barcode sequences and demultiplex the assay data.

FIGS. 9A-9C provides, according to some embodiments herein, examples of simulated data output for a multiplexed COVID-19 assay. FIG. 9A: Positive, high titer sample. FIG. 9B: Positive sample, low titer. FIG. 9C: Negative sample.

FIG. 10 schematically depicts, according to some embodiments herein, an example computer control system.

FIG. 11 provides, according to some embodiments herein, an example of image data from a study to determine the relative levels of non-specific binding of a green fluorescent dye to glass substrate surfaces treated according to different surface modification protocols.

FIG. 12 provides, according to some embodiments herein, an example of image data from a study to determine the relative levels of non-specific binding of a red fluorescent dye to glass substrate surfaces treated according to different surface modification protocols.

FIG. 13 provides, according to some embodiments herein, an example of oligonucleotide primer grafting data for substrate surfaces treated according to different surface modification protocols.

FIG. 14 provides, according to some embodiments herein, an example of images and data demonstrating “tunable” nucleic acid amplification on a low binding solid support by varying the oligonucleotide primer density on the substrate. Blue histogram: low primer density. Red histogram: high primer density. The combination of low non-specific binding and tunable nucleic acid amplification efficiency through adjustment of oligonucleotide primer density yields high CNRs and subsequent improvements in nucleic acid sequencing performance.

FIG. 15 provides, according to some embodiments herein, an example of images and data for non-specific binding of green and red fluorescent dyes to substrate surfaces treated according to different surface modification protocols. For comparison purposes, the fluorescence intensity of a clonally amplified template colony measured under the same set of experimental conditions after coupling a single Cy3-labeled nucleotide base is about 1,500 counts.

FIG. 16 provides, according to some embodiments herein, examples of fluorescence images of the low binding solid supports of the present disclosure on which tethered oligonucleotides have been amplified using different primer densities, isothermal amplification methods, and amplification buffer additives.

FIG. 17 provides, according to some embodiments herein, an example of fluorescence image and intensity data for a low-binding support of the present disclosure on which solid-phase nucleic acid amplification was performed to create clonally-amplified clusters of a template oligonucleotide sequence.

FIG. 18 provides, according to some embodiments herein, a second example of fluorescence image and intensity data for a low-binding support of the present disclosure on which solid-phase nucleic acid amplification was performed to create clonally-amplified clusters of a template oligonucleotide sequence.

FIG. 19 provides, according to some embodiments herein, an example of fluorescence image and intensity data for a low-binding support of the present disclosure on which solid-phase nucleic acid amplification was performed to create clonally-amplified clusters of a template oligonucleotide sequence.

FIGS. 20A-20B provide non-limiting examples of image data that demonstrate the improvements in hybridization stringency, speed, and efficacy that may be achieved through the reformulation of the hybridization buffer used for solid-phase nucleic acid amplification, as described herein. FIG. 20A provides examples of image data for two different hybridization buffer formulations and protocols. FIG. 20B provides an example of the corresponding image data obtained using a standard hybridization buffer and protocol.

FIGS. 21A-21J show fluorescence images of the steps in a sequencing reaction using multivalent PEG-substrate compositions. FIG. 21A. Red and green fluorescent images post exposure of DNA RCA templates (G and A first base) to 500 nM base labeled nucleotides (A-Cy3 and G-Cy5) in exposure buffer containing 20 nM Klenow polymerase and 2.5 mM Sr⁺². Images were collected after washing with imaging buffer with the same composition as the exposure buffer but containing no nucleotides or polymerase. Contrast was scaled to maximize visualization of the dimmest signals, but no signals persisted following washing with imaging buffer (FIG. 21A, inset). FIGS. 21B-21E: fluorescence images showing multivalent PEG-nucleotide (base-labeled) ligands PB1 (FIG. 21B), FIG. 21C), PB3 (FIG. 21D), and PB5 (FIG. 21E) having an effective nucleotide concentration of 500 nM after mixing in the exposure buffer and imaging in the imaging buffer as described above. FIG. 21F: fluorescence image showing multivalent PEG-nucleotide (base-labeled) ligand PB5 at 2.5 uM after mixing in the exposure buffer and imaging in the imaging buffer as above. FIGS. 21G-22I: Fluorescence images showing further base discrimination by exposure of the multivalent binding composition to inactive mutants of klenow polymerase (FIG. 21G. D882H; FIG. 21H. D882E; FIG. 21I. D882A) vs. the wild type Klenow (control) enzyme (FIG. 21J).

FIG. 22 illustrates visualization of cluster amplification in a capillary lumen according to some embodiments herein.

FIG. 23 provides a schematic illustration of a cloud-based approach to monitoring a global pandemic according to some embodiments herein.

### DETAILED DESCRIPTION

While a multitude of rapid and cost-effective assays exist on the market for the detection of COVID-19, limited sample multiplexing is a common shortcoming of all current approaches. Even in a situation where the supply chain of critical assay components is ensured, most labs struggle in dealing with the onslaught of samples, resulting in unacceptable time-to-answer. These assays are of limited value in that they can diagnose a limited number of individuals while preemptively quarantining them but are ineffective for providing a real-time geographical and environmental picture of the evolution of the pandemic. Because of the aforementioned sample-throughput limitations, the current COVID-19 testing infrastructure provides a passive, delayed view of the status of the pandemic rather than the active and pre-emptive monitoring infrastructure that is needed. The disclosed compositions, methods, and systems overcome the primary shortcoming of current molecular diagnostic testing capability—low throughput, lack of precision, unacceptable false-positive/false-negative rates, and the inability to rapidly and cost-effectively scale testing to population-level monitoring of infectious disease—by using a novel barcoded padlock probe assay or barcoded molecular inversion probe assay that leverages a proprietary sequencing platform being developed by the Applicant.

Disclosed herein are barcoded padlock assays and barcoded molecular inversion probe assays that utilize a linear nucleic acid probe molecule comprising capture sequences (e.g., target-specific capture regions or sequences) that are complementary to specific target nucleic acid sequences. In some instances, the linear nucleic acid probe molecule comprises at padlock probe. In some instances, for example, the capture sequences may be complementary to specific COVID-19 sequences or other infectious disease pathogen sequences. In some instances, the linear nucleic acid probe molecule may comprise a probe-specific barcode sequence (located in the non-target-specific regions of the probe sequence) which is adjacent to a universal priming site, e.g., an amplification primer binding site or sequencing primer binding site, where the probe-specific barcode (or simply “probe barcode”) is unique for a given pair of target-specific capture sequences. In some instances, the linear nucleic acid probe molecule may comprise a sample-specific barcode sequence (also located in the non-target-specific regions of the probe sequence) which is adjacent to a probe-specific barcode sequence and to the universal priming site, e.g., an amplification primer binding site, where the sample-specific barcode (or simply “sample barcode”) is unique for a given sample within a plurality of samples to be analyzed within one or more experimental runs. If the target nucleic acid sequence of interest (e.g., a COVID-19 sequence) is present in the sample, the padlock probe will hybridize specifically to the target sequence (or regions thereof) thereby promoting a circularization event that may be completed by ligation. Upon ligation, the circularized nucleic acid probe molecules (e.g., the positive padlock probes), including the barcode sequence(s) contained therein, may be amplified using, for example, isothermal rolling-circle amplification (RCA). In some instances, e.g., where the padlock probe does not include a sample barcode, each sample tested may be amplified using a sample-indexed amplification primer, e.g., an amplification primer that comprises a sample-specific barcode. In the instances that rolling circle amplification is utilized, this rapidly generates concatemers which include multiple copies of the probe barcode and sample barcode sequences. The concatemers will be generated if the target nucleic acid molecules (e.g., a COVID-19 target sequence) is present in a given sample, and the number of concatemers generated will be proportional to the number of target nucleic acid sequence copies originally present in the sample. Upon completion of the padlock/amplification assay (e.g., a padlock/RCA assay requiring 1 hour to perform), a plurality of barcoded samples may be pooled, tethered to a surface within a sequencing flow cell, and loaded into a sequencer that has been configured to function as a DNA-barcode reader. After priming the concatemers at the universal priming sites, the sequence/barcode reader may be used to sequence through the probe barcode (target locus ID) and sample barcode (or sample index) for each concatemer. The sample barcodes allow for demultiplexing of the concatemer sequence data, which may then be further segregated by probe barcode(s). The detection of the sample barcode in the sequence dataset indicates the presence of the target nucleic acid sequence in a given sample, the presence of a given probe barcode sequence indicates the presence of specific target sequences (e.g., COVID-19 sequence(s) or controls), and the total number of amplified concatemers for each sample (or the copy number for a given individual probe barcode for each sample) provides the titer.

The use of sequencing to read an oligonucleotide barcode sequence provides the opportunity to implement large-scale barcode-based multiplexing. While a variety of commercially available sequencing platforms exist, most have been designed primarily for genomic applications and are not easily adaptable to low-end, short-read applications such as barcode reading.

Disclosed herein, in some embodiments, are sequencing platforms designed to provide high quality, high-throughput, low-cost sequencing data of short-read sequences. In some embodiments, the sequencing platforms disclose herein have a modular format that can be reconfigured to perform high-throughput DNA barcode reading and are for high-throughput molecular diagnostic assays that require sample and probe multiplexing.

The advantages of the disclosed methods and systems for large-scale molecular testing include, but are not limited to:

1. Unprecedented assay sensitivity and precision due to the amplification of nucleic acid target sequences at very low copy number to generate hundreds of thousands of concatemers. Each concatemer is essentially a sample replicate and can be individual addressed by sequencing a DNA barcode. The large amount of concatemer data thus accessible will ensure unprecedented assay precision and provide titer information.

2. Flexible sample and probe multiplexing strategies enabled through the use of oligonucleotide barcode sequences. The use of sequenceable oligonucleotide barcodes offers the possibility of simultaneously demultiplexing the assay (e.g., by using two or more barcoded padlock probes, each directed to a different target nucleic acid sequence or control) as well as demultiplexing virtually any number of samples to be processed in parallel. The method is expected to be very economical at modest sample batch sizes (e.g., 384-1,536 samples per experimental run), making the disclosed methods and systems particularly attractive for a decentralized model of molecular diagnostic testing.

3. Enables cloud-based analysis systems for real-time collection and consolidation of the data generated by a distributed network of sequencing instruments to facilitate population-scale testing and monitoring. The ability to deploy a decentralized network of instruments that generate millions of data points per day offers the unprecedented opportunity to monitor in real-time the evolution of infectious disease such as COVID-19 as well as other potential pandemics.

### Decentralized Molecular Diagnostic Assay Platform

PCR-based assays are the method of choice for rapid and cost-effective detection of COVID-19 and other viral infections. However, a major shortcoming of these assays is their insufficient throughput, especially when considering the large volume of samples that may be assayed on a regular basis for the purpose of monitoring the spread of infectious disease through a population. The primary reason for the low throughput of these methods is the lack of practical methods for high sample multiplexing. Current multiplexing strategies rely on either color discrimination or spatial separation in the wells of a microwell plate or microarray. These approaches do not scale well above a small number of multiplexed samples (about 48/samples per run) or are very expensive to implement (1,536 samples per day using a Roche COBAS system).

The compositions, methods, and systems disclosed herein address the throughput limitations of existing molecular diagnostic testing methods by providing a scalable approach to sample multiplexing and a testing platform that allows for decentralization of molecular testing, with each testing facility able to process millions of samples per instrument per year using manageable sample batch sizes of, for example, 384-1536 samples per run. Decentralization and high sample throughput, optionally paired with cloud-based analysis, will also provide the opportunity to deploy a global and real-time monitoring network for detection of infectious disease such as COVID-19. This same sample multiplexing approach may be adapted to a variety of molecular diagnostic assays, thus providing the disclosed molecular diagnostics platform with tremendous flexibility in terms of testing applications.

### Barcoded Padlock Probe Assays

The disclosed compositions, methods, and systems provide a flexible and scalable approach to both simultaneous detection of multiple target analytes in a given sample and highly multiplexed sample processing. The disclosed DNA sequencing platform is configured to read short DNA barcodes in a barcoded padlock probe assay. In this assay, a probe barcode (or probe index) is used for demultiplexing the test results for one or more target nucleic acids, and a sample barcode (or sample index) is used to demultiplex the test results for two or more samples in a single test run.

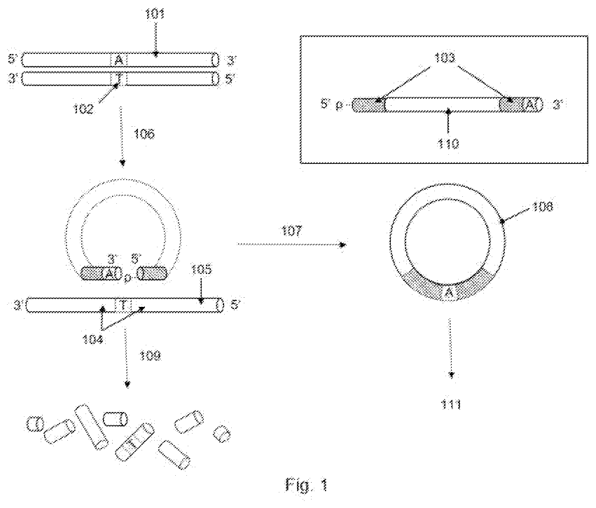

There are many existing padlock probe assays for performing molecular diagnostics testing. These assays are highly sensitive and accurate, for example, to detect of RNA viruses. Padlock assays recognize, bind, and amplify an RNA target isothermally and without RNA transcription into cDNA, thereby providing a very rapid and efficient diagnostic method. FIG. 1 provides an illustration, according to some embodiments disclosed herein, of a padlock probe 101 designed to detect the presence of a single nucleotide polymorphism (SNP) 102. A linear nucleic acid probe molecule 101 (e.g., a padlock probe molecule) comprising 5′-end and 3′-end sequences 103 that are complementary to contiguous regions 104 of the target nucleic acid molecule 105 (e.g., regions spanning the SNP of interest) is hybridized 106 to the target and ligated 107 to form a circularized nucleic acid molecule 108. The circularized nucleic acid probe molecule is formed if the target is present in the sample being tested. Following an optional treatment 109 of the sample with an exonuclease to digest any remaining target nucleic acid molecules, an amplification primer binding site included in the non-complementary region 110 of the padlock probe sequence is used to amplify and detect the circularized molecule using, e.g., PCR or rolling circle amplification (RCA) 111.

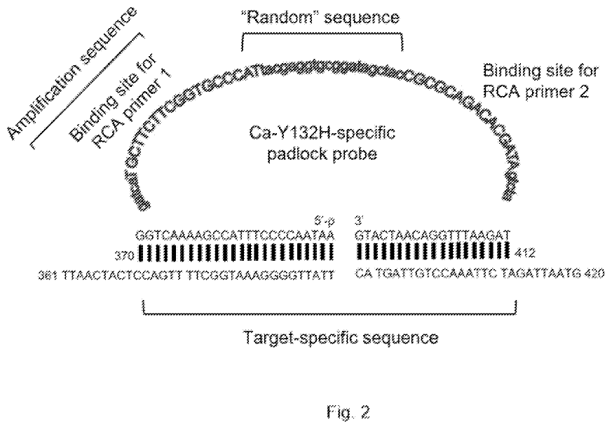

FIG. 2 illustrates, according to various embodiments disclosed herein, the architecture of a barcoded padlock probe of the present disclosure. The target-specific sequence regions recognize a target locus, bringing the 5′- and 3′-ends in close proximity upon hybridization to the target when the target nucleic acid molecule is present. In addition to the target-specific sequence regions, the non-limiting example of a barcoded padlock probe molecule shown in FIG. 2 comprises two primer binding sites for use in RCA amplification and a “random” sequence that may comprise one or more barcode sequences, e.g., a probe barcode sequence that is unique for each pair of target-specific sequence regions, a sample barcode sequence that is unique for a specific sample, or any combination thereof. In the example shown in FIG. 2, the target-specific sequence regions of the probe are designed to target the Ca-Y132H sequence of the COVID-19 genome. Ligation circularizes the probe, which can be then amplified, e.g., using RCA to generate concatemer molecules comprising multiple copies of the probe sequence including the barcode sequences. These concatemers can then be pooled and loaded on a sequencing platform that has been configured to function as a highly multiplexed barcode reader. A short locus-specific probe barcode can be quickly sequenced and decoded, and the number of probe barcodes identified for a given sample will provide both improved assay accuracy as well as viral titer information.

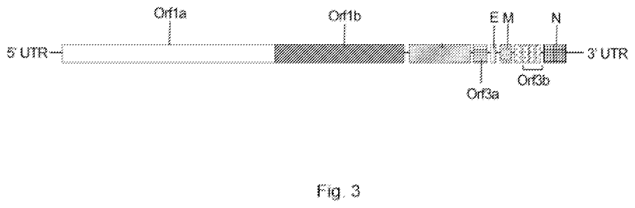

Barcoded padlock probes targeting specific nucleic acid molecules, e.g., COVID-19 specific nucleic acid sequences, as shown in FIG. 3, may be designed to include probe barcode sequences (also referred to as “probe index” or “probe ID” sequences) in the non-targeting padlock regions to facilitate assay multiplexing and expedite the identification of multiple target sequences.

In some instances, the barcoded padlock probe molecule may also comprise a sample barcode sequence. In some instances, circularization of the padlock probe will be followed by primer-indexed RCA amplification, resulting in the generation of sample-barcoded (or sample-indexed) concatemers if the target nucleic acid molecule(s) were present in the sample. These concatemers may then be loaded into a sequencing flow cell, sequenced, demultiplexed, and binned based on sample or probe barcodes. The sequencing of the sample and probe barcodes (as opposed to actual amplicons of viral genome loci) enables the use of short read lengths that drive fast time to answer and low assay cost. In some instances, the disclosed compositions, methods, and systems may enable sample-to-answer turnaround times of 2.5 hours or less, assay costs of $10 per sample or less, and sample processing throughputs of up to millions of samples per instrument per year depending on the degree of sample multiplexing implemented.

### Barcoded Padlock Probe or Molecular Inversion Probe (MP) Design

In some instances, the barcoded padlock probe or molecular inversion probe molecules of the present disclosure may comprise a target-specific 5′-end region (or sequence), one or more primer binding regions (or sequences), one or more barcode regions (or sequences), and a target-specific 3′-end region (or sequence).

In some instances, e.g., for barcoded padlock probe molecules, the 5′-end and 3′end target specific sequences may be designed to target two adjacent (contiguous) sequences within the target nucleic acid sequence, e.g., where a ligation reaction cleaves a 5-terminal phosphate group from the padlock probe and generates a circularized molecule by catalyzing the formation of a covalent linkage between the 5-terminal nucleotide moiety of the padlock probe and the Y-terminal nucleotide moiety of the padlock probe.

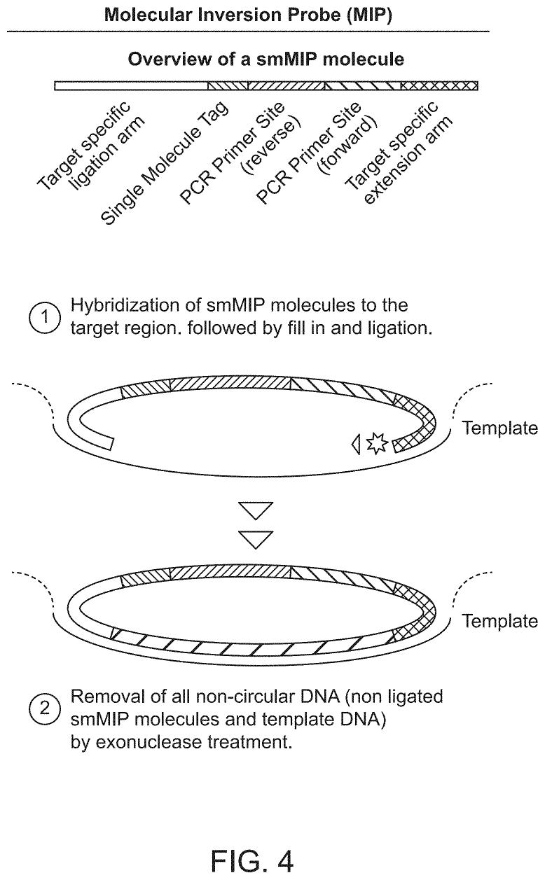

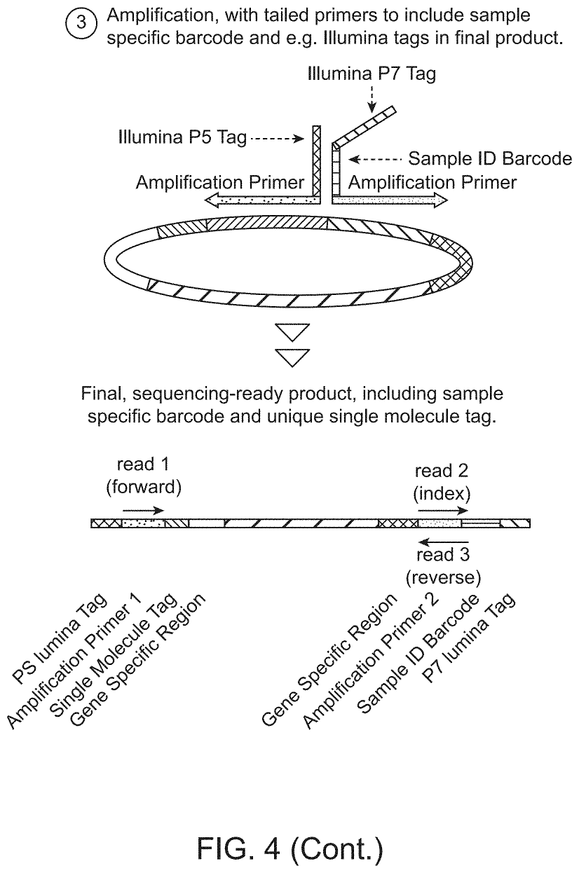

In some instances, e.g., for barcoded molecular inversion probe molecules, the 5′-end and 3′end target specific sequences may be designed to target two adjacent but not contiguous sequences within the target nucleic acid sequence that are separated by up to, e.g., 100 nucleotides, where a primer extension/fill-in reaction initiated at one end of the probe sequence is used in conjunction with a ligation reaction to complete the formation of the circularized molecule. In some instances, the two adjacent target nucleic acid sequences may be separated by up to 1, 2, 3, 4, 5, 6, 7, 8, 9, 10, 20, 30, 40, 50, 60, 70, 80, 90, 100, or more than 100 nucleotides (or any number of nucleotides within this range). FIG. 4 provides a schematic illustration of a barcoded molecular inversion probe assay.

In some instances, the 5′-end and 3′end target specific sequences of the disclosed barcoded padlock probes and barcoded molecular inversion probes may be designed to target any of a variety of target nucleic acid sequences. In some instances, for example, they may be designed to target viral nucleic acids. In some instances, they be designed to target COVID-19 nucleic acid sequences. FIG. 3 provides an illustration of the COVID-19 genome, which comprises open reading frame (Orf) sequences, the spike gene (S) sequence; the envelope gene (E) sequence; the membrane gene (M) sequence; and the nucleocapsid gene (N) sequence. Any of these open reading frame or gene sequences, or fragments thereof, may be used in designing the barcoded padlock probe molecules of the present disclosure. In some instances, the barcoded padlock probes may be designed to target the Ca-Y132H sequence of the COVID-19 genome.

In some instances, the 5′-end and 3′end target specific sequences of the disclosed barcoded padlock probes and barcoded molecular inversion probes may be the same length. In some instances, they may be different lengths. In some instances, the 5′-end and 3′end target specific sequences of the disclosed barcoded padlock probes and barcoded molecular inversion probes may range in length from about 10 nucleotides to about 30 nucleotides. In some instances, the length of the 5′-end or 3′end target specific sequences may be at least 10, at least 11, at least 12, at least at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some instances, the length of the 5′-end or 3′end target specific sequences may be at most 30, at most 29, at most 28, at most 27, at most 26, at most 25, at most 24, at most 23, at most 22, at most 21, at most 20, at most 19, at most 18, at most 17, at most 16, at most 15, at most at most 13, at most 12, at most 11, or at most 10 nucleotides. Any of the lower and upper values described in this paragraph may be combined to form a range included within the present disclosure, for example, in some instances the length of the 5′-end or 3′end target specific sequences may range from about 14 to about 26 nucleotides. It is possible that the length of the 5′-end or 3′end target specific sequences may have any value within this range, e.g., about 23 nucleotides.

In some instances, the disclosed barcoded padlock probe or molecular inversion probe molecules may comprise one, two, three, four, five, or more than five primer binding regions (or primer binding sequences or sites). In some instances, the primer binding sequences may comprise amplification primer binding sequences, sequencing primer binding sequences, universal primer binding sequences, or any combination thereof. In some instances, the one or more primer binding sequences of the disclosed padlock probe or molecular inversion probe molecules may range in length from about 10 nucleotides to about 30 nucleotides. In some instances, the length of the one or more primer binding sequences may be at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some instances, the length of the one or more primer binding sequences may be at most 30, at most 29, at most 28, at most 27, at most 26, at most 25, at most 24, at most 23, at most 22, at most 21, at most 20, at most 19, at most 18, at most 17, at most 16, at most 15, at most 14, at most 13, at most 12, at most 11, or at most 10 nucleotides. Any of the lower and upper values described in this paragraph may be combined to form a range included within the present disclosure, for example, in some instances the length of the one or more primer binding sequences may range from about 18 to about 22 nucleotides. It is possible that the length of the one or more primer binding sequences may have any value within this range, e.g., about 21 nucleotides.

In some instances, the disclosed barcoded padlock probe or molecular inversion probe molecules may comprise a probe barcode (or probe index), a sample barcode (or sample index), or both. In some instances, the disclosed barcoded padlock probe or molecular inversion probe molecules may comprise a probe barcode, and a sample barcode may be added using an indexed primer during amplification, e.g., rolling circle amplification of the circularized probe molecules.

One key advantage of the disclosed compositions, methods, and systems is the ability to attain very high target-specific probe and sample demultiplexing precision by using barcodes as proxies for sequencing. Both probe barcodes and sample index sequences may be designed according to rules that minimize the occurrence of misassignment errors or other sequencing issues. As a non-limiting example of sample barcode design, assume a conservative value of 40,000 sequencing reads per sample and 200,000,000 sequencing reads (barcodes) per sequencing run, it may be possible to process up to 5,000 samples in parallel in the same sequencing run.

The number of unique probe or sample index sequences of length L nucleotides is given by 4^{L}, but additional constraints are imposed on barcode design to avoid runs of nucleotides that can hinder synthesis or sequencing. It is also important to maintain a Hamming distance (e.g., the number of nucleotide positions in two barcode sequences of equal length for which the two nucleotides are different) of greater than 1 so that a single sequencing error does not lead to an incorrect barcode identification. For example, there are 16,384 unique sequences of length 7 nucleotides, but a subset of 4,096 sequences can be selected that have a minimum Hamming distance of 2, which may fit well with the pooling of 5,000 samples in the example above and provide significant error correction capability. By extending the barcode length further (e.g. to 10-12 bases), one can generate many more unique sequences and impose a larger Hamming distance between any pair of barcode sequences. A larger Hamming distance enables both error detection and error correction. Specifically, a minimum distance of 2d+1 may enable the correction of up to d sequencing errors. Given the high base-calling accuracy of Applicant's proprietary sequencing platform (to be discussed below) and the short barcode length required to be sequenced, it may be practical to enable the correction of at least 1, at least 2, or at least 3 errors. The process to design the probe or sample barcodes proceeds by first identifying a surplus of sequences meeting the specified Hamming distance requirement and other requirements followed by synthesis and empirical evaluation of quality.

In some instances, two smaller sets of indices (e.g., sets A and B) may be designed and then used to barcode samples using a pair of indices (where the total number of unique indices=|A|*|B|. Such a barcode design strategy may facilitate manufacturing of a large number of unique index sequences. There are many options for the design of the probe barcode including, for example, the use of a sequence of 3 nucleotides that differ from each other in every position. The design strategy may again be to design more than the required number of unique probes, and then test performance empirically. Since, in some instances, sample barcodes are not integrated into the padlock or molecular inversion probe but are added during an amplification step (performed by the customer), a padlock probe pool (or molecular inversion probe pool) can be generated without requiring physical probe separation at the synthesis stage. From a production standpoint, this essentially means that massively parallel synthetic approaches such as those offered by Twist Bioscience (San Francisco, CA) or Genscript (Piscataway, NJ) can be adopted for rapid and cost-effective customization of the probe pool.

In some instances, the probe barcode or sample barcode sequences of the disclosed barcoded padlock probe or molecular inversion probe molecules may range in length from about 3 nucleotides to about 20 nucleotides. In some instances, the probe barcode or sample barcode sequences of the disclosed barcoded padlock probe or molecular inversion probe molecules may be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some instances, the length of the probe barcode or sample barcode sequences may be at most 30, at most 29, at most 28, at most 27, at most 26, at most 25, at most 24, at most 23, at most 22, at most 21, at most 20, at most 19, at most 18, at most 17, at most 16, at most 15, at most 14, at most 13, at most 12, at most 11, at most 10, at most 9, at most 8, at most 7, at most 6, at most 5, at most 4, or at most 3 nucleotides. Any of the lower and upper values described in this paragraph may be combined to form a range included within the present disclosure, for example, in some instances the length of the probe barcode or sample barcode sequences may range from about 6 to about 10 nucleotides. It is possible that the length of the probe barcode or sample barcode sequences may have any value within this range, e.g., about 7 nucleotides.

In some instances, the total length of the disclosed barcoded padlock probe or molecular inversion probe molecules may range from about 50 nucleotides to about 200 nucleotides. In some instances, the total length of the disclosed probe molecules may be at least 50, at least 60, at least 70, at least 80, at least 90, at least 100, at least 110, at least 120, at least 130, at least 140, at least 150, at least 160, at least 170, at least 180, at least 190, or at least 200 nucleotides. In some instances, the total length of the disclosed probe molecules may be at most 200, at most 190, at most 180, at most 170, at most 160, at most 150, at most 140, at most 130, at most 120, at most 110, at most 100, at most 90, at most 80, at most 70, at most 60, or at most 50 nucleotides. Any of the lower and upper values described in this paragraph may be combined to form a range included within the present disclosure, for example, in some instances the total length of the disclosed probe molecules may range from about 80 to about 160 nucleotides. It is possible that the total length of the disclosed probe molecules may have any value within this range, e.g., about 126 nucleotides.

### Pathogens

The nucleic acids described herein comprise nucleic acid portions of pathogens from human, animal, or plant, such as fungi, bacteria, archaea, eukaryotic parasites, protozoa, or viruses, including but not limited to, filoviruses, coronaviruses, adenoviruses, retroviruses, toxin, and the like. In some embodiments, such pathogens occur naturally. In some embodiments, such pathogens may be synthesized.

In some embodiments, such viruses having nucleic acid components contemplated in this disclosure include, but are not limited to, Ebola virus, Marburg virus other filoviruses, alpha coronaviruses (such as 229E and NL63), beta coronaviruses (such as OC43 and HKU1), other coronaviruses, such as MERS-CoV, SARS-COV, 2019-nCoV, a severe respiratory syndrome 2 (SARS-CoV-2), and mild respiratory illnesses (HCoV-NL63, HCoV-229E, HCoV-OC43, and HKU1), retroviruses (such as the Human Immunodeficiency Virus and Feline Immunodeficiency Virus), adenoviruses, influenza viruses (including H1N1 and H5N1 subtypes, but contemplating all subtypes and combinations of influenza viruses), poxviruses, herpesviruses, and the like.

In some embodiments, the virus comprises a coronavirus. In some embodiments, the coronavirus may be an alpha coronavirus or a beta coronavirus. In some embodiments, such alpha coronavirus is a member of the first of the four genera (alpha, beta, gamma, or delta) of coronaviruses comprising 229E and NL63. In some embodiments, such beta coronavirus is a member of the four genera (alpha, beta, gamma, and delta) of coronaviruses comprising OC43, HKU1, severe acute respiratory syndrome (SARS) coronavirus, or Middle East Respiratory Syndrome (MERS) coronavirus. In some embodiments, said SARS coronavirus is SARS-CoV, SARS-CoV-2, or a variant thereof. In some embodiments, the MERS coronavirus is MERS-CoV or a variant thereof. In some embodiments, the SARS coronavirus causes a disease or a condition, such as coronavirus disease 2019 (COVID-19) or variants.

In some embodiments, the coronavirus can be selected from the group comprising: alphacoronavirus, beta coronavirus, delta coronavirus, and gamma coronavirus. Examples of alphacoronavirus can include, but are not limited to, bat coronavirus CDPHE15, bat coronavirus HKU10, human coronavirus 229E, human coronavirus NL63, miniopterus bat coronavirus 1, miniopterus bat coronavirus HKU8, mink coronavirus 1, porcine epidemic diarrhea virus, rhinolophus bat coronavirus HKU2, and scotophilus bat coronavirus 512. Examples of beta coronavirus can include, but are not limited to, beta coronavirus 1, hedgehog coronavirus 1, human coronavirus HKU1, middle east respiratory syndrome-related coronavirus, murine coronavirus, pipistrellus bat coronavirus HKU5, rousettus bat coronavirus HKU9, severe acute respiratory syndrome-related coronavirus, tylonycteris bat coronavirus HKU4. Examples of delta coronavirus can include, but are not limited to, bulbul coronavirus HKU11, common moorhen coronavirus HKU21, coronavirus HKU15, munia coronavirus HKU13, night heron coronavirus HKU19, thrush coronavirus HKU12, white-eye coronavirus HKU16, wigeon coronavirus HKU20. Examples of gamma coronavirus can include, but are not limited to, avian coronavirus, beluga whale coronavirus SW1. Additional examples of coronavirus can include MERS-CoV, SARS-CoV, and SARS-CoV-2. In some embodiments, the coronavirus can be SARS-CoV-2.

In some embodiments, said coronavirus 2019 (COVID-19) is caused by SARS-CoV-2 virus or a variant thereof. In some embodiments, said SARS-CoV-2 virus or a variant is encoded by a nucleic acid sequence—provided in any one of SEQ ID NOs: 1-4. In some embodiments, the coronavirus (or variant thereof) is encoded by a nucleic acid sequence that is at least about 70%, 75%, 804, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1-4. In some embodiments, the coronavirus (or variant thereof) is encoded by a nucleic acid sequence that is at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In some embodiments, the coronavirus (or variant thereof) is encoded by a nucleic acid sequence that is at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2. In some embodiments, the coronavirus (or variant thereof) is encoded by a nucleic acid sequence that is at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3. In some embodiments, the coronavirus (or variant thereof) is encoded by a nucleic acid sequence that is at least about 70%, 75, 80%, 81%, 824, 83%, 84%, 850, 86%, 87%, 88%, 89%, 90%, 91%, 92%, 93%, 94%, 95%, 96%, 97%, 98%, or 99% identical to SEQ ID NO: 4.

TABLE 1
Pathogen Sequences

| SEQ | Antibody | |
|---|---|---|
| ID | Region | Sequence |
| 1 | >NC_0455 | ATTAAAGGTTTATACCTTCCCAGGTAACAAACCAACCAACTTTCGATCTCTTGTAGATCTGTTCTCTAAA |
| | 12.2 | CGAACTTTAAAATCTGTGTGGCTGTCACTCGGCTGCATGCTTAGTGCACTCACGCAGTATAATTAATAAC |
| | Severe | TAATTACTGTCGTTGACAGGACACGAGTAACTCGTCTATCTTCTGCAGGCTGCTTACGGTTTCGTCCGTG |
| | acute | TTGCAGCCGATCATCAGCACATCTAGGTTTCGTCCGGGTGTGACCGAAAGGTAAGATGGAGAGCCTTGTC |
| | respiratory | CCTGGTTTCAACGAGAAAACACACGTCCAACTCAGTTTGCCTGTTTTACAGGTTCGCGACGTGCTCGTAC |
| | syndrome | GTGGCTTTGGAGACTCCGTGGAGGAGGTCTTATCAGAGGCACGTCAACATCTTAAAGATGGCACTTGTGG |
| | coronavirus 2 | CTTAGTAGAAGTTGAAAAAGGCGTTTTGCCTCAACTTGAACAGCCCTATGTGTTCATCAAACGTTCGGAT |
| | isolate | GCTCGAACTGCACCTCATGGTCATGTTATGGTTGAGCTGGTAGCAGAACTCGAAGGCATTCAGTACGGTC |
| | Wuhan- | GTAGTGGTGAGACACTTGGTGTCCTTGTCCCTCATGTGGGCGAAATACCAGTGGCTTACCGCAAGGTTCT |
| | Hu-1, | TCTTCGTAAGAACGGTAATAAAGGAGCTGGTGGCCATAGTTACGGCGCCGATCTAAAGTCATTTGACTTA |
| | complete | GGCGACGAGCTTGGCACTGATCCTTATGAAGATTTTCAAGAAAACTGGAACACTAAACATAGCAGTGGTG |
| | genome | TTACCCGTGAACTCATGCGTGAGCTTAACGGAGGGGCATACACTCGCTATGTCGATAACAACTTCTGTGG |
| | | CCCTGATGGCTACCCTCTTGAGTGCATTAAAGACCTTCTAGCACGTGCTGGTAAAGCTTCATGCACTTTG |
| | | TCCGAACAACTGGACTTTATTGACACTAAGAGGGGTGTATACTGCTGCCGTGAACATGAGCATGAAATTG |
| | | CTTGGTACACGGAACGTTCTGAAAAGAGCTATGAATTGCAGACACCTTTTGAAATTAAATTGGCAAAGAA |
| | | ATTTGACACCTTCAATGGGGAATGTCCAAATTTTGTATTTCCCTTAAATTCCATAATCAAGACTATTCAA |
| | | CCAAGGGTTGAAAAGAAAAAGCTTGATGGCTTTATGGGTAGAATTCGATCTGTCTATCCAGTTGCGTCAC |
| | | CAAATGAATGCAACCAAATGTGCCTTTCAACTCTCATGAAGTGTGATCATTGTGGTGAAACTTCATGGCA |
| | | GACGGGCGATTTTGTTAAAGCCACTTGCGAATTTTGTGGCACTGAGAATTTGACTAAAGAAGGTGCCACT |
| | | ACTTGTGGTTACTTACCCCAAAATGCTGTTGTTAAAATTTATTGTCCAGCATGTCACAATTCAGAAGTAG |
| | | GACCTGAGCATAGTCTTGCCGAATACCATAATGAATCTGGCTTGAAAACCATTCTTCGTAAGGGTGGTCG |
| | | CACTATTGCCTTTGGAGGCTGTGTGTTCTCTTATGTTGGTTGCCATAACAAGTGTGCCTATTGGGTTCCA |
| | | CGTGCTAGCGCTAACATAGGTTGTAACCATACAGGTGTTGTTGGAGAAGGTTCCGAAGGTCTTAATGACA |
| | | ACCTTCTTGAAATACTCCAAAAAGAGAAAGTCAACATCAATATTGTTGGTGACTTTAAACTTAATGAAGA |
| | | GATCGCCATTATTTTGGCATCTTTTTCTGCTTCCACAAGTGCTTTTGTGGAAACTGTGAAAGGTTTGGAT |
| | | TATAAAGCATTCAAACAAATTGTTGAATCCTGTGGTAATTTTAAAGTTACAAAAGGAAAAGCTAAAAAAG |
| | | GTGCCTGGAATATTGGTGAACAGAAATCAATACTGAGTCCTCTTTATGCATTTGCATCAGAGGCTGCTCG |
| | | TGTTGTACGATCAATTTTCTCCCGCACTCTTGAAACTGCTCAAAATTCTGTGCGTGTTTTACAGAAGGCC |
| | | GCTATAACAATACTAGATGGAATTTCACAGTATTCACTGAGACTCATTGATGCTATGATGTTCACATCTG |
| | | ATTTGGCTACTAACAATCTAGTTGTAATGGCCTACATTACAGGTGGTGTTGTTCAGTTGACTTCGCAGTG |
| | | GCTAACTAACATCTTTGGCACTGTTTATGAAAAACTCAAACCCGTCCTTGATTGGCTTGAAGAGAAGTTT |
| | | AAGGAAGGTGTAGAGTTTCTTAGAGACGGTTGGGAAATTGTTAAATTTATCTCAACCTGTGCTTGTGAAA |
| | | TTGTCGGTGGACAAATTGTCACCTGTGCAAAGGAAATTAAGGAGAGTGTTCAGACATTCTTTAAGCTTGT |
| | | AAATAAATTTTTGGCTTTGTGTGCTGACTCTATCATTATTGGTGGAGCTAAACTTAAAGCCTTGAATTTA |
| | | GGTGAAACATTTGTCACGCACTCAAAGGGATTGTACAGAAAGTGTGTTAAATCCAGAGAAGAAACTGGCC |
| | | TACTCATGCCTCTAAAAGCCCCAAAAGAAATTATCTTCTTAGAGGGAGAAACACTTCCCACAGAAGTGTT |
| | | AACAGAGGAAGTTGTCTTGAAAACTGGTGATTTACAACCATTAGAACAACCTACTAGTGAAGCTGTTGAA |
| | | GCTCCATTGGTTGGTACACCAGTTTGTATTAACGGGCTTATGTTGCTCGAAATCAAAGACACAGAAAAGT |
| | | ACTGTGCCCTTGCACCTAATATGATGGTAACAAACAATACCTTCACACTCAAAGGCGGTGCACCAACAAA |
| | | GGTTACTTTTGGTGATGACACTGTGATAGAAGTGCAAGGTTACAAGAGTGTGAATATCACTTTTGAACTT |
| | | GATGAAAGGATTGATAAAGTACTTAATGAGAAGTGCTCTGCCTATACAGTTGAACTCGGTACAGAAGTAA |
| | | ATGAGTTCGCCTGTGTTGTGGCAGATGCTGTCATAAAAACTTTGCAACCAGTATCTGAATTACTTACACC |
| | | ACTGGGCATTGATTTAGATGAGTGGAGTATGGCTACATACTACTTATTTGATGAGTCTGGTGAGTTTAAA |
| | | TTGGCTTCACATATGTATTGTTCTTTCTACCCTCCAGATGAGGATGAAGAAGAAGGTGATTGTGAAGAAG |
| | | AAGAGTTTGAGCCATCAACTCAATATGAGTATGGTACTGAAGATGATTACCAAGGTAAACCTTTGGAATT |
| | | TGGTGCCACTTCTGCTGCTCTTCAACCTGAAGAAGAGCAAGAAGAAGATTGGTTAGATGATGATAGTCAA |
| | | CAAACTGTTGGTCAACAAGACGGCAGTGAGGACAATCAGACAACTACTATTCAAACAATTGTTGAGGTTC |
| | | AACCTCAATTAGAGATGGAACTTACACCAGTTGTTCAGACTATTGAAGTGAATAGTTTTAGTGGTTATTT |
| | | AAAACTTACTGACAATGTATACATTAAAAATGCAGACATTGTGGAAGAAGCTAAAAAGGTAAAACCAACA |
| | | GTGGTTGTTAATGCAGCCAATGTTTACCTTAAACATGGAGGAGGTGTTGCAGGAGCCTTAAATAAGGCTA |
| | | CTAACAATGCCATGCAAGTTGAATCTGATGATTACATAGCTACTAATGGACCACTTAAAGTGGGTGGTAG |
| | | TTGTGTTTTAAGCGGACACAATCTTGCTAAACACTGTCTTCATGTTGTCGGCCCAAATGTTAACAAAGGT |
| | | GAAGACATTCAACTTCTTAAGAGTGCTTATGAAAATTTTAATCAGCACGAAGTTCTACTTGCACCATTAT |
| | | TATCAGCTGGTATTTTTGGTGCTGACCCTATACATTCTTTAAGAGTTTGTGTAGATACTGTTCGCACAAA |
| | | TGTCTACTTAGCTGTCTTTGATAAAAATCTCTATGACAAACTTGTTTCAAGCTTTTTGGAAATGAAGAGT |
| | | GAAAAGCAAGTTGAACAAAAGATCGCTGAGATTCCTAAAGAGGAAGTTAAGCCATTTATAACTGAAAGTA |
| | | AACCTTCAGTTGAACAGAGAAAACAAGATGATAAGAAAATCAAAGCTTGTGTTGAAGAAGTTACAACAAC |
| | | TCTGGAAGAAACTAAGTTCCTCACAGAAAACTTGTTACTTTATATTGACATTAATGGCAATCTTCATCCA |
| | | GATTCTGCCACTCTTGTTAGTGACATTGACATCACTTTCTTAAAGAAAGATGCTCCATATATAGTGGGTG |
| | | ATGTTGTTCAAGAGGGTGTTTTAACTGCTGTGGTTATACCTACTAAAAAGGCTGGTGGCACTACTGAAAT |
| | | GCTAGCGAAAGCTTTGAGAAAAGTGCCAACAGACAATTATATAACCACTTACCCGGGTCAGGGTTTAAAT |
| | | GGTTACACTGTAGAGGAGGCAAAGACAGTGCTTAAAAAGTGTAAAAGTGCCTTTTACATTCTACCATCTA |
| | | TTATCTCTAATGAGAAGCAAGAAATTCTTGGAACTGTTTCTTGGAATTTGCGAGAAATGCTTGCACATGC |
| | | AGAAGAAACACGCAAATTAATGCCTGTCTGTGTGGAAACTAAAGCCATAGTTTCAACTATACAGCGTAAA |
| | | TATAAGGGTATTAAAATACAAGAGGGTGTGGTTGATTATGGTGCTAGATTTTACTTTTACACCAGTAAAA |
| | | CAACTGTAGCGTCACTTATCAACACACTTAACGATCTAAATGAAACTCTTGTTACAATGCCACTTGGCTA |
| | | TGTAACACATGGCTTAAATTTGGAAGAAGCTGCTCGGTATATGAGATCTCTCAAAGTGCCAGCTACAGTT |
| | | TCTGTTTCTTCACCTGATGCTGTTACAGCGTATAATGGTTATCTTACTTCTTCTTCTAAAACACCTGAAG |
| | | AACATTTTATTGAAACCATCTCACTTGCTGGTTCCTATAAAGATTGGTCCTATTCTGGACAATCTACACA |
| | | ACTAGGTATAGAATTTCTTAAGAGAGGTGATAAAAGTGTATATTACACTAGTAATCCTACCACATTCCAC |
| | | CTAGATGGTGAAGTTATCACCTTTGACAATCTTAAGACACTTCTTTCTTTGAGAGAAGTGAGGACTATTA |
| | | AGGTGTTTACAACAGTAGACAACATTAACCTCCACACGCAAGTTGTGGACATGTCAATGACATATGGACA |
| | | ACAGTTTGGTCCAACTTATTTGGATGGAGCTGATGTTACTAAAATAAAACCTCATAATTCACATGAAGGT |
| | | AAAACATTTTATGTTTTACCTAATGATGACACTCTACGTGTTGAGGCTTTTGAGTACTACCACACAACTG |
| | | ATCCTAGTTTTCTGGGTAGGTACATGTCAGCATTAAATCACACTAAAAAGTGGAAATACCCACAAGTTAA |
| | | TGGTTTAACTTCTATTAAATGGGCAGATAACAACTGTTATCTTGCCACTGCATTGTTAACACTCCAACAA |
| | | ATAGAGTTGAAGTTTAATCCACCTGCTCTACAAGATGCTTATTACAGAGCAAGGGCTGGTGAAGCTGCTA |
| | | ACTTTTGTGCACTTATCTTAGCCTACTGTAATAAGACAGTAGGTGAGTTAGGTGATGTTAGAGAAACAAT |
| | | GAGTTACTTGTTTCAACATGCCAATTTAGATTCTTGCAAAAGAGTCTTGAACGTGGTGTGTAAAACTTGT |
| | | GGACAACAGCAGACAACCCTTAAGGGTGTAGAAGCTGTTATGTACATGGGCACACTTTCTTATGAACAAT |
| | | TTAAGAAAGGTGTTCAGATACCTTGTACGTGTGGTAAACAAGCTACAAAATATCTAGTACAACAGGAGTC |
| | | ACCTTTTGTTATGATGTCAGCACCACCTGCTCAGTATGAACTTAAGCATGGTACATTTACTTGTGCTAGT |
| | | GAGTACACTGGTAATTACCAGTGTGGTCACTATAAACATATAACTTCTAAAGAAACTTTGTATTGCATAG |
| | | ACGGTGCTTTACTTACAAAGTCCTCAGAATACAAAGGTCCTATTACGGATGTTTTCTACAAAGAAAACAG |
| | | TTACACAACAACCATAAAACCAGTTACTTATAAATTGGATGGTGTTGTTTGTACAGAAATTGACCCTAAG |
| | | TTGGACAATTATTATAAGAAAGACAATTCTTATTTCACAGAGCAACCAATTGATCTTGTACCAAACCAAC |
| | | CATATCCAAACGCAAGCTTCGATAATTTTAAGTTTGTATGTGATAATATCAAATTTGCTGATGATTTAAA |
| | | CCAGTTAACTGGTTATAAGAAACCTGCTTCAAGAGAGCTTAAAGTTACATTTTTCCCTGACTTAAATGGT |
| | | GATGTGGTGGCTATTGATTATAAACACTACACACCCTCTTTTAAGAAAGGAGCTAAATTGTTACATAAAC |
| | | CTATTGTTTGGCATGTTAACAATGCAACTAATAAAGCCACGTATAAACCAAATACCTGGTGTATACGTTG |
| | | TCTTTGGAGCACAAAACCAGTTGAAACATCAAATTCGTTTGATGTACTGAAGTCAGAGGACGCGCAGGGA |
| | | ATGGATAATCTTGCCTGCGAAGATCTAAAACCAGTCTCTGAAGAAGTAGTGGAAAATCCTACCATACAGA |
| | | AAGACGTTCTTGAGTGTAATGTGAAAACTACCGAAGTTGTAGGAGACATTATACTTAAACCAGCAAATAA |
| | | TAGTTTAAAAATTACAGAAGAGGTTGGCCACACAGATCTAATGGCTGCTTATGTAGACAATTCTAGTCTT |
| | | ACTATTAAGAAACCTAATGAATTATCTAGAGTATTAGGTTTGAAAACCCTTGCTACTCATGGTTTAGCTG |
| | | CTGTTAATAGTGTCCCTTGGGATACTATAGCTAATTATGCTAAGCCTTTTCTTAACAAAGTTGTTAGTAC |
| | | AACTACTAACATAGTTACACGGTGTTTAAACCGTGTTTGTACTAATTATATGCCTTATTTCTTTACTTTA |
| | | TTGCTACAATTGTGTACTTTTACTAGAAGTACAAATTCTAGAATTAAAGCATCTATGCCGACTACTATAG |
| | | CAAAGAATACTGTTAAGAGTGTCGGTAAATTTTGTCTAGAGGCTTCATTTAATTATTTGAAGTCACCTAA |
| | | TTTTTCTAAACTGATAAATATTATAATTTGGTTTTTACTATTAAGTGTTTGCCTAGGTTCTTTAATCTAC |
| | | TCAACCGCTGCTTTAGGTGTTTTAATGTCTAATTTAGGCATGCCTTCTTACTGTACTGGTTACAGAGAAG |
| | | GCTATTTGAACTCTACTAATGTCACTATTGCAACCTACTGTACTGGTTCTATACCTTGTAGTGTTTGTCT |
| | | TAGTGGTTTAGATTCTTTAGACACCTATCCTTCTTTAGAAACTATACAAATTACCATTTCATCTTTTAAA |
| | | TGGGATTTAACTGCTTTTGGCTTAGTTGCAGAGTGGTTTTTGGCATATATTCTTTTCACTAGGTTTTTCT |
| | | ATGTACTTGGATTGGCTGCAATCATGCAATTGTTTTTCAGCTATTTTGCAGTACATTTTATTAGTAATTC |
| | | TTGGCTTATGTGGTTAATAATTAATCTTGTACAAATGGCCCCGATTTCAGCTATGGTTAGAATGTACATC |
| | | TTCTTTGCATCATTTTATTATGTATGGAAAAGTTATGTGCATGTTGTAGACGGTTGTAATTCATCAACTT |
| | | GTATGATGTGTTACAAACGTAATAGAGCAACAAGAGTCGAATGTACAACTATTGTTAATGGTGTTAGAAG |
| | | GTCCTTTTATGTCTATGCTAATGGAGGTAAAGGCTTTTGCAAACTACACAATTGGAATTGTGTTAATTGT |
| | | GATACATTCTGTGCTGGTAGTACATTTATTAGTGATGAAGTTGCGAGAGACTTGTCACTACAGTTTAAAA |
| | | GACCAATAAATCCTACTGACCAGTCTTCTTACATCGTTGATAGTGTTACAGTGAAGAATGGTTCCATCCA |
| | | TCTTTACTTTGATAAAGCTGGTCAAAAGACTTATGAAAGACATTCTCTCTCTCATTTTGTTAACTTAGAC |
| | | AACCTGAGAGCTAATAACACTAAAGGTTCATTGCCTATTAATGTTATAGTTTTTGATGGTAAATCAAAAT |
| | | GTGAAGAATCATCTGCAAAATCAGCGTCTGTTTACTACAGTCAGCTTATGTGTCAACCTATACTGTTACT |
| | | AGATCAGGCATTAGTGTCTGATGTTGGTGATAGTGCGGAAGTTGCAGTTAAAATGTTTGATGCTTACGTT |
| | | AATACGTTTTCATCAACTTTTAACGTACCAATGGAAAAACTCAAAACACTAGTTGCAACTGCAGAAGCTG |
| | | AACTTGCAAAGAATGTGTCCTTAGACAATGTCTTATCTACTTTTATTTCAGCAGCTCGGCAAGGGTTTGT |
| | | TGATTCAGATGTAGAAACTAAAGATGTTGTTGAATGTCTTAAATTGTCACATCAATCTGACATAGAAGTT |
| | | ACTGGCGATAGTTGTAATAACTATATGCTCACCTATAACAAAGTTGAAAACATGACACCCCGTGACCTTG |
| | | GTGCTTGTATTGACTGTAGTGCGCGTCATATTAATGCGCAGGTAGCAAAAAGTCACAACATTGCTTTGAT |
| | | ATGGAACGTTAAAGATTTCATGTCATTGTCTGAACAACTACGAAAACAAATACGTAGTGCTGCTAAAAAG |
| | | AATAACTTACCTTTTAAGTTGACATGTGCAACTACTAGACAAGTTGTTAATGTTGTAACAACAAAGATAG |
| | | CACTTAAGGGTGGTAAAATTGTTAATAATTGGTTGAAGCAGTTAATTAAAGTTACACTTGTGTTCCTTTT |
| | | TGTTGCTGCTATTTTCTATTTAATAACACCTGTTCATGTCATGTCTAAACATACTGACTTTTCAAGTGAA |
| | | ATCATAGGATACAAGGCTATTGATGGTGGTGTCACTCGTGACATAGCATCTACAGATACTTGTTTTGCTA |
| | | ACAAACATGCTGATTTTGACACATGGTTTAGCCAGCGTGGTGGTAGTTATACTAATGACAAAGCTTGCCC |
| | | ATTGATTGCTGCAGTCATAACAAGAGAAGTGGGTTTTGTCGTGCCTGGTTTGCCTGGCACGATATTACGC |
| | | ACAACTAATGGTGACTTTTTGCATTTCTTACCTAGAGTTTTTAGTGCAGTTGGTAACATCTGTTACACAC |
| | | CATCAAAACTTATAGAGTACACTGACTTTGCAACATCAGCTTGTGTTTTGGCTGCTGAATGTACAATTTT |
| | | TAAAGATGCTTCTGGTAAGCCAGTACCATATTGTTATGATACCAATGTACTAGAAGGTTCTGTTGCTTAT |
| | | GAAAGTTTACGCCCTGACACACGTTATGTGCTCATGGATGGCTCTATTATTCAATTTCCTAACACCTACC |
| | | TTGAAGGTTCTGTTAGAGTGGTAACAACTTTTGATTCTGAGTACTGTAGGCACGGCACTTGTGAAAGATC |
| | | AGAAGCTGGTGTTTGTGTATCTACTAGTGGTAGATGGGTACTTAACAATGATTATTACAGATCTTTACCA |
| | | GGAGTTTTCTGTGGTGTAGATGCTGTAAATTTACTTACTAATATGTTTACACCACTAATTCAACCTATTG |
| | | GTGCTTTGGACATATCAGCATCTATAGTAGCTGGTGGTATTGTAGCTATCGTAGTAACATGCCTTGCCTA |
| | | CTATTTTATGAGGTTTAGAAGAGCTTTTGGTGAATACAGTCATGTAGTTGCCTTTAATACTTTACTATTC |
| | | CTTATGTCATTCACTGTACTCTGTTTAACACCAGTTTACTCATTCTTACCTGGTGTTTATTCTGTTATTT |
| | | ACTTGTACTTGACATTTTATCTTACTAATGATGTTTCTTTTTTAGCACATATTCAGTGGATGGTTATGTT |
| | | CACACCTTTAGTACCTTTCTGGATAACAATTGCTTATATCATTTGTATTTCCACAAAGCATTTCTATTGG |
| | | TTCTTTAGTAATTACCTAAAGAGACGTGTAGTCTTTAATGGTGTTTCCTTTAGTACTTTTGAAGAAGCTG |
| | | CGCTGTGCACCTTTTTGTTAAATAAAGAAATGTATCTAAAGTTGCGTAGTGATGTGCTATTACCTCTTAC |
| | | GCAATATAATAGATACTTAGCTCTTTATAATAAGTACAAGTATTTTAGTGGAGCAATGGATACAACTAGC |
| | | TACAGAGAAGCTGCTTGTTGTCATCTCGCAAAGGCTCTCAATGACTTCAGTAACTCAGGTTCTGATGTTC |
| | | TTTACCAACCACCACAAACCTCTATCACCTCAGCTGTTTTGCAGAGTGGTTTTAGAAAAATGGCATTCCC |
| | | ATCTGGTAAAGTTGAGGGTTGTATGGTACAAGTAACTTGTGGTACAACTACACTTAACGGTCTTTGGCTT |
| | | GATGACGTAGTTTACTGTCCAAGACATGTGATCTGCACCTCTGAAGACATGCTTAACCCTAATTATGAAG |
| | | ATTTACTCATTCGTAAGTCTAATCATAATTTCTTGGTACAGGCTGGTAATGTTCAACTCAGGGTTATTGG |
| | | ACATTCTATGCAAAATTGTGTACTTAAGCTTAAGGTTGATACAGCCAATCCTAAGACACCTAAGTATAAG |
| | | TTTGTTCGCATTCAACCAGGACAGACTTTTTCAGTGTTAGCTTGTTACAATGGTTCACCATCTGGTGTTT |
| | | ACCAATGTGCTATGAGGCCCAATTTCACTATTAAGGGTTCATTCCTTAATGGTTCATGTGGTAGTGTTGG |
| | | TTTTAACATAGATTATGACTGTGTCTCTTTTTGTTACATGCACCATATGGAATTACCAACTGGAGTTCAT |
| | | GCTGGCACAGACTTAGAAGGTAACTTTTATGGACCTTTTGTTGACAGGCAAACAGCACAAGCAGCTGGTA |
| | | CGGACACAACTATTACAGTTAATGTTTTAGCTTGGTTGTACGCTGCTGTTATAAATGGAGACAGGTGGTT |
| | | TCTCAATCGATTTACCACAACTCTTAATGACTTTAACCTTGTGGCTATGAAGTACAATTATGAACCTCTA |
| | | ACACAAGACCATGTTGACATACTAGGACCTCTTTCTGCTCAAACTGGAATTGCCGTTTTAGATATGTGTG |
| | | CTTCATTAAAAGAATTACTGCAAAATGGTATGAATGGACGTACCATATTGGGTAGTGCTTTATTAGAAGA |
| | | TGAATTTACACCTTTTGATGTTGTTAGACAATGCTCAGGTGTTACTTTCCAAAGTGCAGTGAAAAGAACA |
| | | ATCAAGGGTACACACCACTGGTTGTTACTCACAATTTTGACTTCACTTTTAGTTTTAGTCCAGAGTACTC |
| | | AATGGTCTTTGTTCTTTTTTTTGTATGAAAATGCCTTTTTACCTTTTGCTATGGGTATTATTGCTATGTC |
| | | TGCTTTTGCAATGATGTTTGTCAAACATAAGCATGCATTTCTCTGTTTGTTTTTGTTACCTTCTCTTGCC |
| | | ACTGTAGCTTATTTTAATATGGTCTATATGCCTGCTAGTTGGGTGATGCGTATTATGACATGGTTGGATA |
| | | TGGTTGATACTAGTTTGTCTGGTTTTAAGCTAAAAGACTGTGTTATGTATGCATCAGCTGTAGTGTTACT |
| | | AATCCTTATGACAGCAAGAACTGTGTATGATGATGGTGCTAGGAGAGTGTGGACACTTATGAATGTCTTG |
| | | ACACTCGTTTATAAAGTTTATTATGGTAATGCTTTAGATCAAGCCATTTCCATGTGGGCTCTTATAATCT |
| | | CTGTTACTTCTAACTACTCAGGTGTAGTTACAACTGTCATGTTTTTGGCCAGAGGTATTGTTTTTATGTG |
| | | TGTTGAGTATTGCCCTATTTTCTTCATAACTGGTAATACACTTCAGTGTATAATGCTAGTTTATTGTTTC |
| | | TTAGGCTATTTTTGTACTTGTTACTTTGGCCTCTTTTGTTTACTCAACCGCTACTTTAGACTGACTCTTG |
| | | GTGTTTATGATTACTTAGTTTCTACACAGGAGTTTAGATATATGAATTCACAGGGACTACTCCCACCCAA |
| | | GAATAGCATAGATGCCTTCAAACTCAACATTAAATTGTTGGGTGTTGGTGGCAAACCTTGTATCAAAGTA |
| | | GCCACTGTACAGTCTAAAATGTCAGATGTAAAGTGCACATCAGTAGTCTTACTCTCAGTTTTGCAACAAC |
| | | TCAGAGTAGAATCATCATCTAAATTGTGGGCTCAATGTGTCCAGTTACACAATGACATTCTCTTAGCTAA |
| | | AGATACTACTGAAGCCTTTGAAAAAATGGTTTCACTACTTTCTGTTTTGCTTTCCATGCAGGGTGCTGTA |
| | | GACATAAACAAGCTTTGTGAAGAAATGCTGGACAACAGGGCAACCTTACAAGCTATAGCCTCAGAGTTTA |
| | | GTTCCCTTCCATCATATGCAGCTTTTGCTACTGCTCAAGAAGCTTATGAGCAGGCTGTTGCTAATGGTGA |
| | | TTCTGAAGTTGTTCTTAAAAAGTTGAAGAAGTCTTTGAATGTGGCTAAATCTGAATTTGACCGTGATGCA |
| | | GCCATGCAACGTAAGTTGGAAAAGATGGCTGATCAAGCTATGACCCAAATGTATAAACAGGCTAGATCTG |
| | | AGGACAAGAGGGCAAAAGTTACTAGTGCTATGCAGACAATGCTTTTCACTATGCTTAGAAAGTTGGATAA |
| | | TGATGCACTCAACAACATTATCAACAATGCAAGAGATGGTTGTGTTCCCTTGAACATAATACCTCTTACA |
| | | ACAGCAGCCAAACTAATGGTTGTCATACCAGACTATAACACATATAAAAATACGTGTGATGGTACAACAT |
| | | TTACTTATGCATCAGCATTGTGGGAAATCCAACAGGTTGTAGATGCAGATAGTAAAATTGTTCAACTTAG |
| | | TGAAATTAGTATGGACAATTCACCTAATTTAGCATGGCCTCTTATTGTAACAGCTTTAAGGGCCAATTCT |
| | | GCTGTCAAATTACAGAATAATGAGCTTAGTCCTGTTGCACTACGACAGATGTCTTGTGCTGCCGGTACTA |
| | | CACAAACTGCTTGCACTGATGACAATGCGTTAGCTTACTACAACACAACAAAGGGAGGTAGGTTTGTACT |
| | | TGCACTGTTATCCGATTTACAGGATTTGAAATGGGCTAGATTCCCTAAGAGTGATGGAACTGGTACTATC |
| | | TATACAGAACTGGAACCACCTTGTAGGTTTGTTACAGACACACCTAAAGGTCCTAAAGTGAAGTATTTAT |
| | | ACTTTATTAAAGGATTAAACAACCTAAATAGAGGTATGGTACTTGGTAGTTTAGCTGCCACAGTACGTCT |
| | | ACAAGCTGGTAATGCAACAGAAGTGCCTGCCAATTCAACTGTATTATCTTTCTGTGCTTTTGCTGTAGAT |
| | | GCTGCTAAAGCTTACAAAGATTATCTAGCTAGTGGGGGACAACCAATCACTAATTGTGTTAAGATGTTGT |
| | | GTACACACACTGGTACTGGTCAGGCAATAACAGTTACACCGGAAGCCAATATGGATCAAGAATCCTTTGG |
| | | TGGTGCATCGTGTTGTCTGTACTGCCGTTGCCACATAGATCATCCAAATCCTAAAGGATTTTGTGACTTA |
| | | AAAGGTAAGTATGTACAAATACCTACAACTTGTGCTAATGACCCTGTGGGTTTTACACTTAAAAACACAG |
| | | TCTGTACCGTCTGCGGTATGTGGAAAGGTTATGGCTGTAGTTGTGATCAACTCCGCGAACCCATGCTTCA |
| | | GTCAGCTGATGCACAATCGTTTTTAAACGGGTTTGCGGTGTAAGTGCAGCCCGTCTTACACCGTGCGGCA |
| | | CAGGCACTAGTACTGATGTCGTATACAGGGCTTTTGACATCTACAATGATAAAGTAGCTGGTTTTGCTAA |
| | | ATTCCTAAAAACTAATTGTTGTCGCTTCCAAGAAAAGGACGAAGATGACAATTTAATTGATTCTTACTTT |
| | | GTAGTTAAGAGACACACTTTCTCTAACTACCAACATGAAGAAACAATTTATAATTTACTTAAGGATTGTC |
| | | CAGCTGTTGCTAAACATGACTTCTTTAAGTTTAGAATAGACGGTGACATGGTACCACATATATCACGTCA |
| | | ACGTCTTACTAAATACACAATGGCAGACCTCGTCTATGCTTTAAGGCATTTTGATGAAGGTAATTGTGAC |
| | | ACATTAAAAGAAATACTTGTCACATACAATTGTTGTGATGATGATTATTTCAATAAAAAGGACTGGTATG |
| | | ATTTTGTAGAAAACCCAGATATATTACGCGTATACGCCAACTTAGGTGAACGTGTACGCCAAGCTTTGTT |
| | | AAAAACAGTACAATTCTGTGATGCCATGCGAAATGCTGGTATTGTTGGTGTACTGACATTAGATAATCAA |
| | | GATCTCAATGGTAACTGGTATGATTTCGGTGATTTCATACAAACCACGCCAGGTAGTGGAGTTCCTGTTG |
| | | TAGATTCTTATTATTCATTGTTAATGCCTATATTAACCTTGACCAGGGCTTTAACTGCAGAGTCACATGT |
| | | TGACACTGACTTAACAAAGCCTTACATTAAGTGGGATTTGTTAAAATATGACTTCACGGAAGAGAGGTTA |
| | | AAACTCTTTGACCGTTATTTTAAATATTGGGATCAGACATACCACCCAAATTGTGTTAACTGTTTGGATG |
| | | ACAGATGCATTCTGCATTGTGCAAACTTTAATGTTTTATTCTCTACAGTGTTCCCACCTACAAGTTTTGG |
| | | ACCACTAGTGAGAAAAATATTTGTTGATGGTGTTCCATTTGTAGTTTCAACTGGATACCACTTCAGAGAG |
| | | CTAGGTGTTGTACATAATCAGGATGTAAACTTACATAGCTCTAGACTTAGTTTTAAGGAATTACTTGTGT |
| | | ATGCTGCTGACCCTGCTATGCACGCTGCTTCTGGTAATCTATTACTAGATAAACGCACTACGTGCTTTTC |
| | | AGTAGCTGCACTTACTAACAATGTTGCTTTTCAAACTGTCAAACCCGGTAATTTTAACAAAGACTTCTAT |
| | | GACTTTGCTGTGTCTAAGGGTTTCTTTAAGGAAGGAAGTTCTGTTGAATTAAAACACTTCTTCTTTGCTC |
| | | AGGATGGTAATGCTGCTATCAGCGATTATGACTACTATCGTTATAATCTACCAACAATGTGTGATATCAG |
| | | ACAACTACTATTTGTAGTTGAAGTTGTTGATAAGTACTTTGATTGTTACGATGGTGGCTGTATTAATGCT |
| | | AACCAAGTCATCGTCAACAACCTAGACAAATCAGCTGGTTTTCCATTTAATAAATGGGGTAAGGCTAGAC |
| | | TTTATTATGATTCAATGAGTTATGAGGATCAAGATGCACTTTTCGCATATACAAAACGTAATGTCATCCC |
| | | TACTATAACTCAAATGAATCTTAAGTATGCCATTAGTGCAAAGAATAGAGCTCGCACCGTAGCTGGTGTC |
| | | TCTATCTGTAGTACTATGACCAATAGACAGTTTCATCAAAAATTATTGAAATCAATAGCCGCCACTAGAG |
| | | GAGCTACTGTAGTAATTGGAACAAGCAAATTCTATGGTGGTTGGCACAACATGTTAAAAACTGTTTATAG |
| | | TGATGTAGAAAACCCTCACCTTATGGGTTGGGATTATCCTAAATGTGATAGAGCCATGCCTAACATGCTT |
| | | AGAATTATGGCCTCACTTGTTCTTGCTCGCAAACATACAACGTGTTGTAGCTTGTCACACCGTTTCTATA |
| | | GATTAGCTAATGAGTGTGCTCAAGTATTGAGTGAAATGGTCATGTGTGGCGGTTCACTATATGTTAAACC |
| | | AGGTGGAACCTCATCAGGAGATGCCACAACTGCTTATGCTAATAGTGTTTTTAACATTTGTCAAGCTGTC |
| | | ACGGCCAATGTTAATGCACTTTTATCTACTGATGGTAACAAAATTGCCGATAAGTATGTCCGCAATTTAC |
| | | AACACAGACTTTATGAGTGTCTCTATAGAAATAGAGATGTTGACACAGACTTTGTGAATGAGTTTTACGC |
| | | ATATTTGCGTAAACATTTCTCAATGATGATACTCTCTGACGATGCTGTTGTGTGTTTCAATAGCACTTAT |
| | | GCATCTCAAGGTCTAGTGGCTAGCATAAAGAACTTTAAGTCAGTTCTTTATTATCAAAACAATGTTTTTA |
| | | TGTCTGAAGCAAAATGTTGGACTGAGACTGACCTTACTAAAGGACCTCATGAATTTTGCTCTCAACATAC |
| | | AATGCTAGTTAAACAGGGTGATGATTATGTGTACCTTCCTTACCCAGATCCATCAAGAATCCTAGGGGCC |
| | | GGCTGTTTTGTAGATGATATCGTAAAAACAGATGGTACACTTATGATTGAACGGTTCGTGTCTTTAGCTA |
| | | TAGATGCTTACCCACTTACTAAACATCCTAATCAGGAGTATGCTGATGTCTTTCATTTGTACTTACAATA |
| | | CATAAGAAAGCTACATGATGAGTTAACAGGACACATGTTAGACATGTATTCTGTTATGCTTACTAATGAT |
| | | AACACTTCAAGGTATTGGGAACCTGAGTTTTATGAGGCTATGTACACACCGCATACAGTCTTACAGGCTG |
| | | TTGGGGCTTGTGTTCTTTGCAATTCACAGACTTCATTAAGATGTGGTGCTTGCATACGTAGACCATTCTT |
| | | ATGTTGTAAATGCTGTTACGACCATGTCATATCAACATCACATAAATTAGTCTTGTCTGTTAATCCGTAT |
| | | GTTTGCAATGCTCCAGGTTGTGATGTCACAGATGTGACTCAACTTTACTTAGGAGGTATGAGCTATTATT |
| | | GTAAATCACATAAACCACCCATTAGTTTTCCATTGTGTGCTAATGGACAAGTTTTTGGTTTATATAAAAA |
| | | TACATGTGTTGGTAGCGATAATGTTACTGACTTTAATGCAATTGCAACATGTGACTGGACAAATGCTGGT |
| | | GATTACATTTTAGCTAACACCTGTACTGAAAGACTCAAGCTTTTTGCAGCAGAAACGCTCAAAGCTACTG |
| | | AGGAGACATTTAAACTGTCTTATGGTATTGCTACTGTACGTGAAGTGCTGTCTGACAGAGAATTACATCT |
| | | TTCATGGGAAGTTGGTAAACCTAGACCACCACTTAACCGAAATTATGTCTTTACTGGTTATCGTGTAACT |
| | | AAAAACAGTAAAGTACAAATAGGAGAGTACACCTTTGAAAAAGGTGACTATGGTGATGCTGTTGTTTACC |
| | | GAGGTACAACAACTTACAAATTAAATGTTGGTGATTATTTTGTGCTGACATCACATACAGTAATGCCATT |
| | | AAGTGCACCTACACTAGTGCCACAAGAGCACTATGTTAGAATTACTGGCTTATACCCAACACTCAATATC |
| | | TCAGATGAGTTTTCTAGCAATGTTGCAAATTATCAAAAGGTTGGTATGCAAAAGTATTCTACACTCCAGG |
| | | GACCACCTGGTACTGGTAAGAGTCATTTTGCTATTGGCCTAGCTCTCTACTACCCTTCTGCTCGCATAGT |
| | | GTATACAGCTTGCTCTCATGCCGCTGTTGATGCACTATGTGAGAAGGCATTAAAATATTTGCCTATAGAT |
| | | AAATGTAGTAGAATTATACCTGCACGTGCTCGTGTAGAGTGTTTTGATAAATTCAAAGTGAATTCAACAT |
| | | TAGAACAGTATGTCTTTTGTACTGTAAATGCATTGCCTGAGACGACAGCAGATATAGTTGTCTTTGATGA |
| | | AATTTCAATGGCCACAAATTATGATTTGAGTGTTGTCAATGCCAGATTACGTGCTAAGCACTATGTGTAC |
| | | ATTGGCGACCCTGCTCAATTACCTGCACCACGCACATTGCTAACTAAGGGCACACTAGAACCAGAATATT |
| | | TCAATTCAGTGTGTAGACTTATGAAAACTATAGGTCCAGACATGTTCCTCGGAACTTGTCGGCGTTGTCC |
| | | TGCTGAAATTGTTGACACTGTGAGTGCTTTGGTTTATGATAATAAGCTTAAAGCACATAAAGACAAATCA |
| | | GCTCAATGCTTTAAAATGTTTTATAAGGGTGTTATCACGCATGATGTTTCATCTGCAATTAACAGGCCAC |
| | | AAATAGGCGTGGTAAGAGAATTCCTTACACGTAACCCTGCTTGGAGAAAAGCTGTCTTTATTTCACCTTA |
| | | TAATTCACAGAATGCTGTAGCCTCAAAGATTTTGGGACTACCAACTCAAACTGTTGATTCATCACAGGGC |
| | | TCAGAATATGACTATGTCATATTCACTCAAACCACTGAAACAGCTCACTCTTGTAATGTAAACAGATTTA |
| | | ATGTTGCTATTACCAGAGCAAAAGTAGGCATACTTTGCATAATGTCTGATAGAGACCTTTATGACAAGTT |
| | | GCAATTTACAAGTCTTGAAATTCCACGTAGGAATGTGGCAACTTTACAAGCTGAAAATGTAACAGGACTC |
| | | TTTAAAGATTGTAGTAAGGTAATCACTGGGTTACATCCTACACAGGCACCTACACACCTCAGTGTTGACA |
| | | CTAAATTCAAAACTGAAGGTTTATGTGTTGACATACCTGGCATACCTAAGGACATGACCTATAGAAGACT |
| | | CATCTCTATGATGGGTTTTAAAATGAATTATCAAGTTAATGGTTACCCTAACATGTTTATCACCCGCGAA |
| | | GAAGCTATAAGACATGTACGTGCATGGATTGGCTTCGATGTCGAGGGGTGTCATGCTACTAGAGAAGCTG |
| | | TTGGTACCAATTTACCTTTACAGCTAGGTTTTTCTACAGGTGTTAACCTAGTTGCTGTACCTACAGGTTA |
| | | TGTTGATACACCTAATAATACAGATTTTTCCAGAGTTAGTGCTAAACCACCGCCTGGAGATCAATTTAAA |
| | | CACCTCATACCACTTATGTACAAAGGACTTCCTTGGAATGTAGTGCGTATAAAGATTGTACAAATGTTAA |
| | | GTGACACACTTAAAAATCTCTCTGACAGAGTCGTATTTGTCTTATGGGCACATGGCTTTGAGTTGACATC |
| | | TATGAAGTATTTTGTGAAAATAGGACCTGAGCGCACCTGTTGTCTATGTGATAGACGTGCCACATGCTTT |
| | | TCCACTGCTTCAGACACTTATGCCTGTTGGCATCATTCTATTGGATTTGATTACGTCTATAATCCGTTTA |
| | | TGATTGATGTTCAACAATGGGGTTTTACAGGTAACCTACAAAGCAACCATGATCTGTATTGTCAAGTCCA |
| | | TGGTAATGCACATGTAGCTAGTTGTGATGCAATCATGACTAGGTGTCTAGCTGTCCACGAGTGCTTTGTT |
| | | AAGCGTGTTGACTGGACTATTGAATATCCTATAATTGGTGATGAACTGAAGATTAATGCGGCTTGTAGAA |
| | | AGGTTCAACACATGGTTGTTAAAGCTGCATTATTAGCAGACAAATTCCCAGTTCTTCACGACATTGGTAA |
| | | CCCTAAAGCTATTAAGTGTGTACCTCAAGCTGATGTAGAATGGAAGTTCTATGATGCACAGCCTTGTAGT |
| | | GACAAAGCTTATAAAATAGAAGAATTATTCTATTCTTATGCCACACATTCTGACAAATTCACAGATGGTG |
| | | TATGCCTATTTTGGAATTGCAATGTCGATAGATATCCTGCTAATTCCATTGTTTGTAGATTTGACACTAG |
| | | AGTGCTATCTAACCTTAACTTGCCTGGTTGTGATGGTGGCAGTTTGTATGTAAATAAACATGCATTCCAC |
| | | ACACCAGCTTTTGATAAAAGTGCTTTTGTTAATTTAAAACAATTACCATTTTTCTATTACTCTGACAGTC |
| | | CATGTGAGTCTCATGGAAAACAAGTAGTGTCAGATATAGATTATGTACCACTAAAGTCTGCTACGTGTAT |
| | | AACACGTTGCAATTTAGGTGGTGCTGTCTGTAGACATCATGCTAATGAGTACAGATTGTATCTCGATGCT |
| | | TATAACATGATGATCTCAGCTGGCTTTAGCTTGTGGGTTTACAAACAATTTGATACTTATAACCTCTGGA |
| | | ACACTTTTACAAGACTTCAGAGTTTAGAAAATGTGGCTTTTAATGTTGTAAATAAGGGACACTTTGATGG |
| | | ACAACAGGGTGAAGTACCAGTTTCTATCATTAATAACACTGTTTACACAAAAGTTGATGGTGTTGATGTA |
| | | GAATTGTTTGAAAATAAAACAACATTACCTGTTAATGTAGCATTTGAGCTTTGGGCTAAGCGCAACATTA |
| | | AACCAGTACCAGAGGTGAAAATACTCAATAATTTGGGTGTGGACATTGCTGCTAATACTGTGATCTGGGA |
| | | CTACAAAAGAGATGCTCCAGCACATATATCTACTATTGGTGTTTGTTCTATGACTGACATAGCCAAGAAA |
| | | CCAACTGAAACGATTTGTGCACCACTCACTGTCTTTTTTGATGGTAGAGTTGATGGTCAAGTAGACTTAT |
| | | TTAGAAATGCCCGTAATGGTGTTCTTATTACAGAAGGTAGTGTTAAAGGTTTACAACCATCTGTAGGTCC |
| | | CAAACAAGCTAGTCTTAATGGAGTCACATTAATTGGAGAAGCCGTAAAAACACAGTTCAATTATTATAAG |
| | | AAAGTTGATGGTGTTGTCCAACAATTACCTGAAACTTACTTTACTCAGAGTAGAAATTTACAAGAATTTA |
| | | AACCCAGGAGTCAAATGGAAATTGATTTCTTAGAATTAGCTATGGATGAATTCATTGAACGGTATAAATT |
| | | AGAAGGCTATGCCTTCGAACATATCGTTTATGGAGATTTTAGTCATAGTCAGTTAGGTGGTTTACATCTA |
| | | CTGATTGGACTAGCTAAACGTTTTAAGGAATCACCTTTTGAATTAGAAGATTTTATTCCTATGGACAGTA |
| | | CAGTTAAAAACTATTTCATAACAGATGCGCAAACAGGTTCATCTAAGTGTGTGTGTTCTGTTATTGATTT |
| | | ATTACTTGATGATTTTGTTGAAATAATAAAATCCCAAGATTTATCTGTAGTTTCTAAGGTTGTCAAAGTG |
| | | ACTATTGACTATACAGAAATTTCATTTATGCTTTGGTGTAAAGATGGCCATGTAGAAACATTTTACCCAA |
| | | AATTACAATCTAGTCAAGCGTGGCAACCGGGTGTTGCTATGCCTAATCTTTACAAAATGCAAAGAATGCT |
| | | ATTAGAAAAGTGTGACCTTCAAAATTATGGTGATAGTGCAACATTACCTAAAGGCATAATGATGAATGTC |
| | | GCAAAATATACTCAACTGTGTCAATATTTAAACACATTAACATTAGCTGTACCCTATAATATGAGAGTTA |
| | | TACATTTTGGTGCTGGTTCTGATAAAGGAGTTGCACCAGGTACAGCTGTTTTAAGACAGTGGTTGCCTAC |
| | | GGGTACGCTGCTTGTCGATTCAGATCTTAATGACTTTGTCTCTGATGCAGATTCAACTTTGATTGGTGAT |
| | | TGTGCAACTGTACATACAGCTAATAAATGGGATCTCATTATTAGTGATATGTACGACCCTAAGACTAAAA |
| | | ATGTTACAAAAGAAAATGACTCTAAAGAGGGTTTTTTCACTTACATTTGTGGGTTTATACAACAAAAGCT |
| | | AGCTCTTGGAGGTTCCGTGGCTATAAAGATAACAGAACATTCTTGGAATGCTGATCTTTATAAGCTCATG |
| | | GGACACTTCGCATGGTGGACAGCCTTTGTTACTAATGTGAATGCGTCATCATCTGAAGCATTTTTAATTG |
| | | GATGTAATTATCTTGGCAAACCACGCGAACAAATAGATGGTTATGTCATGCATGCAAATTACATATTTTG |
| | | GAGGAATACAAATCCAATTCAGTTGTCTTCCTATTCTTTATTTGACATGAGTAAATTTCCCCTTAAATTA |
| | | AGGGGTACTGCTGTTATGTCTTTAAAAGAAGGTCAAATCAATGATATGATTTTATCTCTTCTTAGTAAAG |
| | | GTAGACTTATAATTAGAGAAAACAACAGAGTTGTTATTTCTAGTGATGTTCTTGTTAACAACTAAACGAA |
| | | CAATGTTTGTTTTTCTTGTTTTATTGCCACTAGTCTCTAGTCAGTGTGTTAATCTTACAACCAGAACTCA |
| | | ATTACCCCCTGCATACACTAATTCTTTCACACGTGGTGTTTATTACCCTGACAAAGTTTTCAGATCCTCA |
| | | GTTTTACATTCAACTCAGGACTTGTTCTTACCTTTCTTTTCCAATGTTACTTGGTTCCATGCTATACATG |
| | | TCTCTGGGACCAATGGTACTAAGAGGTTTGATAACCCTGTCCTACCATTTAATGATGGTGTTTATTTTGC |
| | | TTCCACTGAGAAGTCTAACATAATAAGAGGCTGGATTTTTGGTACTACTTTAGATTCGAAGACCCAGTCC |
| | | CTACTTATTGTTAATAACGCTACTAATGTTGTTATTAAAGTCTGTGAATTTCAATTTTGTAATGATCCAT |
| | | TTTTGGGTGTTTATTACCACAAAAACAACAAAAGTTGGATGGAAAGTGAGTTCAGAGTTTATTCTAGTGC |
| | | GAATAATTGCACTTTTGAATATGTCTCTCAGCCTTTTCTTATGGACCTTGAAGGAAAACAGGGTAATTTC |
| | | AAAAATCTTAGGGAATTTGTGTTTAAGAATATTGATGGTTATTTTAAAATATATTCTAAGCACACGCCTA |
| | | TTAATTTAGTGCGTGATCTCCCTCAGGGTTTTTCGGCTTTAGAACCATTGGTAGATTTGCCAATAGGTAT |
| | | TAACATCACTAGGTTTCAAACTTTACTTGCTTTACATAGAAGTTATTTGACTCCTGGTGATTCTTCTTCA |
| | | GGTTGGACAGCTGGTGCTGCAGCTTATTATGTGGGTTATCTTCAACCTAGGACTTTTCTATTAAAATATA |
| | | ATGAAAATGGAACCATTACAGATGCTGTAGACTGTGCACTTGACCCTCTCTCAGAAACAAAGTGTACGTT |
| | | GAAATCCTTCACTGTAGAAAAAGGAATCTATCAAACTTCTAACTTTAGAGTCCAACCAACAGAATCTATT |
| | | GTTAGATTTCCTAATATTACAAACTTGTGCCCTTTTGGTGAAGTTTTTAACGCCACCAGATTTGCATCTG |
| | | TTTATGCTTGGAACAGGAAGAGAATCAGCAACTGTGTTGCTGATTATTCTGTCCTATATAATTCCGCATC |
| | | ATTTTCCACTTTTAAGTGTTATGGAGTGTCTCCTACTAAATTAAATGATCTCTGCTTTACTAATGTCTAT |
| | | GCAGATTCATTTGTAATTAGAGGTGATGAAGTCAGACAAATCGCTCCAGGGCAAACTGGAAAGATTGCTG |
| | | ATTATAATTATAAATTACCAGATGATTTTACAGGCTGCGTTATAGCTTGGAATTCTAACAATCTTGATTC |
| | | TAAGGTTGGTGGTAATTATAATTACCTGTATAGATTGTTTAGGAAGTCTAATCTCAAACCTTTTGAGAGA |
| | | GATATTTCAACTGAAATCTATCAGGCCGGTAGCACACCTTGTAATGGTGTTGAAGGTTTTAATTGTTACT |
| | | TTCCTTTACAATCATATGGTTTCCAACCCACTAATGGTGTTGGTTACCAACCATACAGAGTAGTAGTACT |
| | | TTCTTTTGAACTTCTACATGCACCAGCAACTGTTTGTGGACCTAAAAAGTCTACTAATTTGGTTAAAAAC |
| | | AAATGTGTCAATTTCAACTTCAATGGTTTAACAGGCACAGGTGTTCTTACTGAGTCTAACAAAAAGTTTC |
| | | TGCCTTTCCAACAATTTGGCAGAGACATTGCTGACACTACTGATGCTGTCCGTGATCCACAGACACTTGA |
| | | GATTCTTGACATTACACCATGTTCTTTTGGTGGTGTCAGTGTTATAACACCAGGAACAAATACTTCTAAC |
| | | CAGGTTGCTGTTCTTTATCAGGATGTTAACTGCACAGAAGTCCCTGTTGCTATTCATGCAGATCAACTTA |
| | | CTCCTACTTGGCGTGTTTATTCTACAGGTTCTAATGTTTTTCAAACACGTGCAGGCTGTTTAATAGGGGC |
| | | TGAACATGTCAACAACTCATATGAGTGTGACATACCCATTGGTGCAGGTATATGCGCTAGTTATCAGACT |
| | | CAGACTAATTCTCCTCGGCGGGCACGTAGTGTAGCTAGTCAATCCATCATTGCCTACACTATGTCACTTG |
| | | GTGCAGAAAATTCAGTTGCTTACTCTAATAACTCTATTGCCATACCCACAAATTTTACTATTAGTGTTAC |
| | | CACAGAAATTCTACCAGTGTCTATGACCAAGACATCAGTAGATTGTACAATGTACATTTGTGGTGATTCA |
| | | ACTGAATGCAGCAATCTTTTGTTGCAATATGGCAGTTTTTGTACACAATTAAACCGTGCTTTAACTGGAA |
| | | TAGCTGTTGAACAAGACAAAAACACCCAAGAAGTTTTTGCACAAGTCAAACAAATTTACAAAACACCACC |
| | | AATTAAAGATTTTGGTGGTTTTAATTTTTCACAAATATTACCAGATCCATCAAAACCAAGCAAGAGGTCA |
| | | TTTATTGAAGATCTACTTTTCAACAAAGTGACACTTGCAGATGCTGGCTTCATCAAACAATATGGTGATT |
| | | GCCTTGGTGATATTGCTGCTAGAGACCTCATTTGTGCACAAAAGTTTAACGGCCTTACTGTTTTGCCACC |
| | | TTTGCTCACAGATGAAATGATTGCTCAATACACTTCTGCACTGTTAGCGGGTACAATCACTTCTGGTTGG |
| | | ACCTTTGGTGCAGGTGCTGCATTACAAATACCATTTGCTATGCAAATGGCTTATAGGTTTAATGGTATTG |
| | | GAGTTACACAGAATGTTCTCTATGAGAACCAAAAATTGATTGCCAACCAATTTAATAGTGCTATTGGCAA |
| | | AATTCAAGACTCACTTTCTTCCACAGCAAGTGCACTTGGAAAACTTCAAGATGTGGTCAACCAAAATGCA |
| | | CAAGCTTTAAACACGCTTGTTAAACAACTTAGCTCCAATTTTGGTGCAATTTCAAGTGTTTTAAATGATA |
| | | TCCTTTCACGTCTTGACAAAGTTGAGGCTGAAGTGCAAATTGATAGGTTGATCACAGGCAGACTTCAAAG |
| | | TTTGCAGACATATGTGACTCAACAATTAATTAGAGCTGCAGAAATCAGAGCTTCTGCTAATCTTGCTGCT |
| | | ACTAAAATGTCAGAGTGTGTACTTGGACAATCAAAAAGAGTTGATTTTTGTGGAAAGGGCTATCATCTTA |
| | | TGTCCTTCCCTCAGTCAGCACCTCATGGTGTAGTCTTCTTGCATGTGACTTATGTCCCTGCACAAGAAAA |
| | | GAACTTCACAACTGCTCCTGCCATTTGTCATGATGGAAAAGCACACTTTCCTCGTGAAGGTGTCTTTGTT |
| | | TCAAATGGCACACACTGGTTTGTAACACAAAGGAATTTTTATGAACCACAAATCATTACTACAGACAACA |
| | | CATTTGTGTCTGGTAACTGTGATGTTGTAATAGGAATTGTCAACAACACAGTTTATGATCCTTTGCAACC |
| | | TGAATTAGACTCATTCAAGGAGGAGTTAGATAAATATTTTAAGAATCATACATCACCAGATGTTGATTTA |
| | | GGTGACATCTCTGGCATTAATGCTTCAGTTGTAAACATTCAAAAAGAAATTGACCGCCTCAATGAGGTTG |
| | | CCAAGAATTTAAATGAATCTCTCATCGATCTCCAAGAACTTGGAAAGTATGAGCAGTATATAAAATGGCC |
| | | ATGGTACATTTGGCTAGGTTTTATAGCTGGCTTGATTGCCATAGTAATGGTGACAATTATGCTTTGCTGT |
| | | ATGACCAGTTGCTGTAGTTGTCTCAAGGGCTGTTGTTCTTGTGGATCCTGCTGCAAATTTGATGAAGACG |
| | | ACTCTGAGCCAGTGCTCAAAGGAGTCAAATTACATTACACATAAACGAACTTATGGATTTGTTTATGAGA |
| | | ATCTTCACAATTGGAACTGTAACTTTGAAGCAAGGTGAAATCAAGGATGCTACTCCTTCAGATTTTGTTC |
| | | GCGCTACTGCAACGATACCGATACAAGCCTCACTCCCTTTCGGATGGCTTATTGTTGGCGTTGCACTTCT |
| | | TGCTGTTTTTCAGAGCGCTTCCAAAATCATAACCCTCAAAAAGAGATGGCAACTAGCACTCTCCAAGGGT |
| | | GTTCACTTTGTTTGCAACTTGCTGTTGTTGTTTGTAACAGTTTACTCACACCTTTTGCTCGTTGCTGCTG |
| | | GCCTTGAAGCCCCTTTTCTCTATCTTTATGCTTTAGTCTACTTCTTGCAGAGTATAAACTTTGTAAGAAT |
| | | AATAATGAGGCTTTGGCTTTGCTGGAAATGCCGTTCCAAAAACCCATTACTTTATGATGCCAACTATTTT |
| | | CTTTGCTGGCATACTAATTGTTACGACTATTGTATACCTTACAATAGTGTAACTTCTTCAATTGTCATTA |
| | | CTTCAGGTGATGGCACAACAAGTCCTATTTCTGAACATGACTACCAGATTGGTGGTTATACTGAAAAATG |
| | | GGAATCTGGAGTAAAAGACTGTGTTGTATTACACAGTTACTTCACTTCAGACTATTACCAGCTGTACTCA |
| | | ACTCAATTGAGTACAGACACTGGTGTTGAACATGTTACCTTCTTCATCTACAATAAAATTGTTGATGAGC |
| | | CTGAAGAACATGTCCAAATTCACACAATCGACGGTTCATCCGGAGTTGTTAATCCAGTAATGGAACCAAT |
| | | TTATGATGAACCGACGACGACTACTAGCGTGCCTTTGTAAGCACAAGCTGATGAGTACGAACTTATGTAC |
| | | TCATTCGTTTCGGAAGAGACAGGTACGTTAATAGTTAATAGCGTACTTCTTTTTCTTGCTTTCGTGGTAT |
| | | TCTTGCTAGTTACACTAGCCATCCTTACTGCGCTTCGATTGTGTGCGTACTGCTGCAATATTGTTAACGT |
| | | GAGTCTTGTAAAACCTTCTTTTTACGTTTACTCTCGTGTTAAAAATCTGAATTCTTCTAGAGTTCCTGAT |
| | | CTTCTGGTCTAAACGAACTAAATATTATATTAGTTTTTCTGTTTGGAACTTTAATTTTAGCCATGGCAGA |
| | | TTCCAACGGTACTATTACCGTTGAAGAGCTTAAAAAGCTCCTTGAACAATGGAACCTAGTAATAGGTTTC |
| | | CTATTCCTTACATGGATTTGTCTTCTACAATTTGCCTATGCCAACAGGAATAGGTTTTTGTATATAATTA |
| | | AGTTAATTTTCCTCTGGCTGTTATGGCCAGTAACTTTAGCTTGTTTTGTGCTTGCTGCTGTTTACAGAAT |
| | | AAATTGGATCACCGGTGGAATTGCTATCGCAATGGCTTGTCTTGTAGGCTTGATGTGGCTCAGCTACTTC |
| | | ATTGCTTCTTTCAGACTGTTTGCGCGTACGCGTTCCATGTGGTCATTCAATCCAGAAACTAACATTCTTC |
| | | TCAACGTGCCACTCCATGGCACTATTCTGACCAGACCGCTTCTAGAAAGTGAACTCGTAATCGGAGCTGT |
| | | GATCCTTCGTGGACATCTTCGTATTGCTGGACACCATCTAGGACGCTGTGACATCAAGGACCTGCCTAAA |
| | | GAAATCACTGTTGCTACATCACGAACGCTTTCTTATTACAAATTGGGAGCTTCGCAGCGTGTAGCAGGTG |
| | | ACTCAGGTTTTGCTGCATACAGTCGCTACAGGATTGGCAACTATAAATTAAACACAGACCATTCCAGTAG |
| | | CAGTGACAATATTGCTTTGCTTGTACAGTAAGTGACAACAGATGTTTCATCTCGTTGACTTTCAGGTTAC |
| | | TATAGCAGAGATATTACTAATTATTATGAGGACTTTTAAAGTTTCCATTTGGAATCTTGATTACATCATA |
| | | AACCTCATAATTAAAAATTTATCTAAGTCACTAACTGAGAATAAATATTCTCAATTAGATGAAGAGCAAC |
| | | CAATGGAGATTGATTAAACGAACATGAAAATTATTCTTTTCTTGGCACTGATAACACTCGCTACTTGTGA |
| | | GCTTTATCACTACCAAGAGTGTGTTAGAGGTACAACAGTACTTTTAAAAGAACCTTGCTCTTCTGGAACA |
| | | TACGAGGGCAATTCACCATTTCATCCTCTAGCTGATAACAAATTTGCACTGACTTGCTTTAGCACTCAAT |
| | | TTGCTTTTGCTTGTCCTGACGGCGTAAAACACGTCTATCAGTTACGTGCCAGATCAGTTTCACCTAAACT |
| | | GTTCATCAGACAAGAGGAAGTTCAAGAACTTTACTCTCCAATTTTTCTTATTGTTGCGGCAATAGTGTTT |
| | | ATAACACTTTGCTTCACACTCAAAAGAAAGACAGAATGATTGAACTTTCATTAATTGACTTCTATTTGTG |
| | | CTTTTTAGCCTTTCTGCTATTCCTTGTTTTAATTATGCTTATTATCTTTTGGTTCTCACTTGAACTGCAA |
| | | GATCATAATGAAACTTGTCACGCCTAAACGAACATGAAATTTCTTGTTTTCTTAGGAATCATCACAACTG |
| | | TAGCTGCATTTCACCAAGAATGTAGTTTACAGTCATGTACTCAACATCAACCATATGTAGTTGATGACCC |
| | | GTGTCCTATTCACTTCTATTCTAAATGGTATATTAGAGTAGGAGCTAGAAAATCAGCACCTTTAATTGAA |
| | | TTGTGCGTGGATGAGGCTGGTTCTAAATCACCCATTCAGTACATCGATATCGGTAATTATACAGTTTCCT |
| | | GTTTACCTTTTACAATTAATTGCCAGGAACCTAAATTGGGTAGTCTTGTAGTGCGTTGTTCGTTCTATGA |
| | | AGACTTTTTAGAGTATCATGACGTTCGTGTTGTTTTAGATTTCATCTAAACGAACAAACTAAAATGTCTG |
| | | ATAATGGACCCCAAAATCAGCGAAATGCACCCCGCATTACGTTTGGTGGACCCTCAGATTCAACTGGCAG |
| | | TAACCAGAATGGAGAACGCAGTGGGGCGCGATCAAAACAACGTCGGCCCCAAGGTTTACCCAATAATACT |
| | | GCGTCTTGGTTCACCGCTCTCACTCAACATGGCAAGGAAGACCTTAAATTCCCTCGAGGACAAGGCGTTC |
| | | CAATTAACACCAATAGCAGTCCAGATGACCAAATTGGCTACTACCGAAGAGCTACCAGACGAATTCGTGG |
| | | TGGTGACGGTAAAATGAAAGATCTCAGTCCAAGATGGTATTTCTACTACCTAGGAACTGGGCCAGAAGCT |
| | | GGACTTCCCTATGGTGCTAACAAAGACGGCATCATATGGGTTGCAACTGAGGGAGCCTTGAATACACCAA |
| | | AAGATCACATTGGCACCCGCAATCCTGCTAACAATGCTGCAATCGTGCTACAACTTCCTCAAGGAACAAC |
| | | ATTGCCAAAAGGCTTCTACGCAGAAGGGAGCAGAGGCGGCAGTCAAGCCTCTTCTCGTTCCTCATCACGT |
| | | AGTCGCAACAGTTCAAGAAATTCAACTCCAGGCAGCAGTAGGGGAACTTCTCCTGCTAGAATGGCTGGCA |
| | | ATGGCGGTGATGCTGCTCTTGCTTTGCTGCTGCTTGACAGATTGAACCAGCTTGAGAGCAAAATGTCTGG |
| | | TAAAGGCCAACAACAACAAGGCCAAACTGTCACTAAGAAATCTGCTGCTGAGGCTTCTAAGAAGCCTCGG |
| | | CAAAAACGTACTGCCACTAAAGCATACAATGTAACACAAGCTTTCGGCAGACGTGGTCCAGAACAAACCC |
| | | AAGGAAATTTTGGGGACCAGGAACTAATCAGACAAGGAACTGATTACAAACATTGGCCGCAAATTGCACA |
| | | ATTTGCCCCCAGCGCTTCAGCGTTCTTCGGAATGTCGCGCATTGGCATGGAAGTCACACCTTCGGGAACG |
| | | TGGTTGACCTACACAGGTGCCATCAAATTGGATGACAAAGATCCAAATTTCAAAGATCAAGTCATTTTGC |
| | | TGAATAAGCATATTGACGCATACAAAACATTCCCACCAACAGAGCCTAAAAAGGACAAAAAGAAGAAGGC |
| | | TGATGAAACTCAAGCCTTACCGCAGAGACAGAAGAAACAGCAAACTGTGACTCTTCTTCCTGCTGCAGAT |
| | | TTGGATGATTTCTCCAAACAATTGCAACAATCCATGAGCAGTGCTGACTCAACTCAGGCCTAAACTCATG |
| | | CAGACCACACAAGGCAGATGGGCTATATAAACGTTTTCGCTTTTCCGTTTACGATATATAGTCTACTCTT |
| | | GTGCAGAATGAATTCTCGTAACTACATAGCACAAGTAGATGTAGTTAACTTTAATCTCACATAGCAATCT |
| | | TTAATCAGTGTGTAACATTAGGGAGGACTTGAAAGAGCCACCACATTTTCACCGAGGCCACGCGGAGTAC |
| | | GATCGAGTGTACAGTGAACAATGCTAGGGAGAGCTGCCTATATGGAAGAGCCCTAATGTGTAAAATTAAT |
| | | TTTAGTAGTGCTATCCCCATGTGATTTTAATAGCTTCTTAGGAGAATGACAAAAAAAAAAAAAAAAAAAA |
| | | AAAAAAAAAAAAA |
| 2 | Severe | ATGGAGAGCCTTGTCCCTGGTTTCAACGAGAAAACACACGTCCAACTCAGTTTGCCTGTTTTACAGGTTC |
| | acute | GCGACGTGCTCGTACGTGGCTTTGGAGACTCCGTGGAGGAGGTCTTATCAGAGGCACGTCAACATCTTAA |
| | respiratory | AGATGGCACTTGTGGCTTAGTAGAAGTTGAAAAAGGCGTTTTGCCTCAACTTGAACAGCCCTATGTGTTC |
| | syndrome | ATCAAACGTTCGGATGCTCGAACTGCACCTCATGGTCATGTTATGGTTGAGCTGGTAGCAGAACTCGAAG |
| | coronavirus 2 | GCATTCAGTACGGTCGTAGTGGTGAGACACTTGGTGTCCTTGTCCCTCATGTGGGCGAAATACCAGTGGC |
| | isolate | TTACCGCAAGGTTCTTCTTCGTAAGAACGGTAATAAAGGAGCTGGTGGCCATAGTTACGGCGCCGATCTA |
| | SARS- | AAGTCATTTGACTTAGGCGACGAGCTTGGCACTGATCCTTATGAAGATTTTCAAGAAAACTGGAACACTA |
| | CoV- | AACATAGCAGTGGTGTTACCCGTGAACTCATGCGTGAGCTTAACGGAGGGGCATACACTCGCTATGTCGA |
| | 2/human/ | TAACAACTTCTGTGGCCCTGATGGCTACCCTCTTGAGTGCATTAAAGACCTTCTAGCACGTGCTGGTAAA |
| | BGD/G039 | GCTTCATGCACTTTGTCTGAACAACTGGACTTTATTGACACTAAGAGGGGTGTATACTGCTGCCGTGAAC |
| | 392/2021, | ATGAGCATGAAATTGCTTGGTACACGGAACGTTCTGAAAAGAGCTATGAATTGCAGACACCTTTTGAAAT |
| | complete | TAAATTGGCAAAGAAATTTGACACCTTCAATGGGGAATGTCCAAATTTTGTATTTCCCTTAAATTCCATA |
| | genome | ATCAAGACTATTCAACCAAGGGTTGAAAAGAAAAAGCTTGATGGCTTTATGGGTAGAATTCGATCTGTCT |
| | | ATCCAGTTGCGTCACCAAATGAATGCAACCAAATGTGCCTTTCAACTCTCATGAAGTGTGATCATTGTGG |
| | | TGAAACTTCATGGCAGACGGGCGATTTTGTTAAAGCCACTTGCGAATTTTGTGGCACTGAGAATTTGACT |
| | | AAAGAAGGTGCCACTACTTGTGGTTACTTACCCCAAAATGCTGTTGTTAAAATTTATTGTCCAGCATGTC |
| | | ACAATTCAGAAGTAGGACCTGAGCATAGTCTTGCCGAATACCATAATGAATCTGGCTTGAAAACCATTCT |
| | | TCGTAAGGGTGGTCGCACTATTGCCTTTGGAGGCTGTGTGTTCTCTTATGTTGGTTGCCATAACAAGTGT |
| | | GCCTATTGGGTTCCACGTGCTAGCGCTAACATAGGTTGTAACCATACAGGTGTTGTTGGAGAAGGTTCCG |
| | | AAGGTCTTAATGACAACCTTCTTGAAATACTCCAAAAAGAGAAAGTCAACATCAATATTGTTGGTGACTT |
| | | TAAACTTAATGAAGAGATCGCCATTATTTTGGCATCTTTTTCTGCTTCCACAAGTGCTTTTGTGGAAACT |
| | | GTGAAAGGTTTGGATTATAAAGCATTCAAACAAATTGTTGAATCCTGTGGTAATTTTAAAGTTACAAAAG |
| | | GAAAAGCTAAAAAAGGTGCCTGGAATATTGGTGAACAGAAATCAATACTGAGTCCTCTTTATGCATTTGC |
| | | ATCAGAGGCTGCTCGTGTTGTACGATCAATTTTCTCCCGCACTCTTGAAACTGCTCAAAATTCTGTGCGT |
| | | GTTTTACAGAAGGCCGCTATAACAATACTAGATGGAATTTCACAGTATTCACTGAGACTCATTGATGCTA |
| | | TGATGTTCACATCTGATTTGGCTACTAACAATCTAGTTGTAATGGCCTACATTACAGGTGGTGTTGTTCA |
| | | GTTGACTTCGCAGTGGCTAACTAACATCTTTGGCACTGTTTATGAAAAACTCAAACCCGTCCTTGATTGG |
| | | CTTGAAGAGAAGTTTAAGGAAGGTGTAGAGTTTCTTAGAGACGGTTGGGAAATTGTTAAATTTATCTCAA |
| | | CCTGTGCTTGTGAAATTGTCGGTGGACAAATTGTCACCTGTGCAAAGGAAATTAAGGAGAGTGTTCAGAC |
| | | ATTCTTTAAGCTTGTAAATAAATTTTTGGCTTTGTGTGCTGACTCTATCATTATTGGTGGAGCTAAACTT |
| | | AAAGCCTTGAATTTAGGTGAAACATTTGTCACGCACTCAAAGGGATTGTACAGAAAGTGTGTTAAATCCA |
| | | GAGAAGAAACTGGCCTACTCATGCCTCTAAAAGCCCCAAAAGAAATTATCTTCTTAGAGGGAGAAACACT |
| | | TCCCACAGAAGTGTTAACAGAGGAAGTTGTCTTGAAAACTGGTGATTTACAACCATTAGAACAACCTACT |
| | | AGTGAAGCTGTTGAAGCTCCATTGGTTGGTACACCAGTTTGTATTAACGGGCTTATGTTGCTCGAAATCA |
| | | AAGACACAGAAAAGTACTGTGCCCTTGCACCTAATATGATGGTAACAAACAATACCTTCACACTCAAAGG |
| | | CGGTGCACCAACAAAGGTTACTTTTGGTGATGACACTGTGATAGAAGTGCAAGGTTACAAGAGTGTGAAT |
| | | ATCACTTTTGAACTTGATGAAAGGATTGATAAAGTACTTAATGAGAAGTGCTCTGCCTATACAGTTGAAC |
| | | TCGGTACAGAAGTAAATGAGTTCGCCTGTGTTGTGGCAGATGCTGTCATAAAAACTTTGCAACCAGTATC |
| | | TGAATTACTTACACCACTGGGCATTGATTTAGATGAGTGGAGTATGGCTACATACTACTTATTTGATGAG |
| | | TCTGGTGAGTTTAAATTGGCTTCACATATGTATTGTTCTTTTTACCCTCCAGATGAGGATGAAGAAGAAG |
| | | GTGATTGTGAAGAAGAAGAGTTTGAGCCATCAACTCAATATGAGTATGGTACTGAAGATGATTACCAAGG |
| | | TAAACCTTTGGAATTTGGTGCCACTTCTGCTGCTCTTCAACCTGAAGAAGAGCAAGAAGAAGATTGGTTA |
| | | GATGATGATAGTCAACAAACTGTTGGTCAACAAGACGGCAGTGAGGACAATCAGACAACTATTATTCAAA |
| | | CAATTGTTGAGGTTCAACCTCAATTAGAGATGGAACTTACACCAGTTGTTCAGACTATTGAAGTGAATAG |
| | | TTTTAGTGGTTATTTAAAACTTACTGACAATGTATACATTAAAAATGCAGACATTGTGGAAGAAGCTAAA |
| | | AAGGTAAAACCAACAGTGGTTGTTAATGCAGCCAATGTTTACCTTAAACATGGAGGAGGTGTTGCAGGAG |
| | | CCTTAAATAAGGCTACTAACAATGCCATGCAAGTTGAATCTGATGATTACATAGCTACTAATGGACCACT |
| | | TAAAGTGGGTGGTAGTTGTGTTTTAAGCGGACACAATCTTGCTAAACACTGTCTTCATGTTGTCGGCCCA |
| | | AATGTTAACAAAGGTGAAGACATTCAACTTCTTAAGAGTGCTTATGAAAATTTTAATCAGCACGAAGTTC |
| | | TACTTGCACCATTATTATCAGCTGGTATTTTTGGTGCTGACCCTATACATTCTTTAAGAGTTTGTGTAGA |
| | | TACTGTTCGCACAAATGTCTACTTAGCTGTCTTTGATAAAAATCTCTATGACAAACTTGTTTCAAGCTTT |
| | | TTGGAAATGAAGAGTGAAAAGCAAGTTGAACAAAAGATCGCTGAGATTCCTAAAGAGGAAGTTAAGCCAT |
| | | TTATAACTGAAAGTAAACCTTCAGTTGAACAGAGAAAACAAGATGATAAGAAAATCAAAGCTTGTGTTGA |
| | | AGAAGTTACAACAACTCTGGAAGAAACTAAGTTCCTCACAGAAAACTTGTTACTTTATATTGACATTAAT |
| | | GGCAATCTTCATCCAGATTCTGCCACTCTTGTTAGTGACATTGACATCACTTTCTTAAAGAAAGATGCTC |
| | | CATATATAGTGGGTGATGTTGTTCAAGAGGGTGTTTTAACTGCTGTGGTTATACCTACTAAAAAGGCTGG |
| | | TGGCACTACTGAAATGCTAGCGAAAGCTTTGAGAAAAGTGCCAACAGACAATTATATAACCACTTACCCG |
| | | GGTCAGGGTTTAAATGGTTACACTGTAGAGGAGGCAAAGACAGTGCTTAAAAAGTGTAAAAGTGCCTTTT |
| | | ACATTCTACCATCTATTATCTCTAATGAGAAGCAAGAAATTCTTGGAACTGTTTCTTGGAATTTGCGAGA |
| | | AATGCTTGCACATGCAGAAGAAACACGCAAATTAATGCCTGTCTGTGTGGAAACTAAAGCCATAGTTTCA |
| | | ACTATACAGCGTAAATATAAGGGTATTAAAATACAAGAGGGTGTGGTTGATTATGGTGCTAGATTTTACT |
| | | TTTACACCAGTAAAACAACTGTAGCGTCACTTATCAACACACTTAACGATCTAAATGAAACTCTTGTTAC |
| | | AATGCCACTTGGCTATGTAACACATGGCTTAAATTTGGAAGAAGCTGCTCGGTATATGAGATCTCTCAAA |
| | | GTGCCAGCTACAGTTTCTGTTTCTTCACCTGATGCTGTTACAGCGTATAATGGTTATCTTACTTCTTCTT |
| | | CTAAAACACCTGAAGAACATTTTATTGAAACCATCTCACTTGCTGGTTCCTATAAAGATTGGTCCTATTC |
| | | TGGACAATCTACACAACTAGGTATAGAATTTCTTAAGAGAGGTGATAAAAGTGTATATTACACTAGTAAT |
| | | CCTACCACATTCCACCTAGATGGTGAAGTTATCACCTTTGACAATCTTAAGACACTTCTTTCTTTGAGAG |
| | | AAGTGAGGACTATTAAGGTGTTTACAACAGTAGACAACATTAACCTCCACACGCAAGTTGTGGACATGTC |
| | | AATGACATATGGACAACAGTTTGGTCCAACTTATTTGGATGGAGCTGATGTTACTAAAATAAAACCTCAT |
| | | AATTCACATGAAGGTAAAACATTTTATGTTTTACCTAATGATGACACTCTACGTGTTGAGGCTTTTGAGT |
| | | ACTACCACACAACTGATCCTAGTTTTCTGGGTAGGTACATGTCAGCATTAAATCACACTAAAAAGTGGAA |
| | | ATACCCACAAGTTAATGGTTTAACTTCTATAAAATGGGCAGATAACAACTGTTATCTTGCCACTGCATTG |
| | | TTAACACTCCAACAAATAGAGTTGAAGTTTAATCCACCTGCTCTACAAGATGCTTATTACAGAGCAAGGG |
| | | CTGGTGAAGCTGATAACTTTTGTGCACTTATCTTAGCCTACTGTAATAAGACAGTAGGTGAGTTAGGTGA |
| | | TGTTAGAGAAACAATGAGTTACTTGTTTCAACATGCCAATTTAGATTCTTGCAAAAGAGTCTTGAACGTG |
| | | GTGTGTAAAACTTGTGGACAACAGCAGACAACCCTTAAGGGTGTAGAAGCTGTTATGTACATGGGCACAC |
| | | TTTCTTATGAACAATTTAAGAAAGGTGTTCAGATACCTTGTACGTGTGGTAAACAAGCTACAAAATATCT |
| | | AGTACAACAGGAGTCACCTTTTGTTATGATGTCAGCACCACCTGCTCAGTATGAACTTAAGCATGGTACA |
| | | TTTACTTGTGCTAGTGAGTACACTGGTAATTACCAGTGTGGTCACTATAAACATATAACTTCTAAAGAAA |
| | | CTTTGTATTGCATAGACGGTGCTTTACTTACAAAGTCCTCAGAATACAAAGGTCCTATTACGGATGTTTT |
| | | CTACAAAGAAAACAGTTACACAACAACCATAAAACCAGTTACTTATAAATTGGATGGTGTTGTTTGTACA |
| | | GAAATTGACCCTAAGTTGGACAATTATTATAAGAAAGACAATTCTTATTTTACAGAGCAACCAATTGATC |
| | | TTGTACCAAACCAACCATATCCAAACGCAAGCTTCGATAATTTTAAGTTTGTATGTGATAATATCAAATT |
| | | TGCTGATGATTTAAACCAGTTAACTGGTTATAAGAAACCTGCTTCAAGAGAGCTTAAAGTTACATTTTTC |
| | | CCTGACTTAAATGGTGATGTGGTGGCTATTGATTATAAACACTACACACCCTCTTTTAAGAAAGGAGCTA |
| | | AATTGTTACATAAACCTATTGTTTGGCATGTTAACAATGCAACTAATAAAGCCACGTATAAACCAAATAC |
| | | CTGGTGTATACGTTGTCTTTGGAGCACAAAACCAGTTGAAACATCAAATTCGTTTGATGTACTGAAGTCA |
| | | GAGGACGCGCAGGGAATGGATAATCTTGCCTGCGAAGATCTAAAACCAGTCTCTGAAGAAGTAGTGGAAA |
| | | ATCCTACCATACAGAAAGACGTTCTTGAGTGTAATGTGAAAACTACCGAAGTTGTAGGAGACATTATACT |
| | | TAAACCAGCAAATAATAGTTTAAAAATTACAGAAGAGGTTGGCCACACAGATCTAATGGCTGCTTATGTA |
| | | GACAATTCTAGTCTTACTATTAAGAAACCTAATGAATTATCTAGAGTATTAGGTTTGAAAACCCTTGCTA |
| | | CTCATGGTTTAGCTGCTGTTAATAGTGTCCCTTGGGATACTATAGCTAATTATGCTAAGCCTTTTCTTAA |
| | | CAAAGTTGTTAGTACAACTACTAACATAGTTACACGGTGTTTAAACCGTGTTTGTACTAATTATATGCCT |
| | | TATTTCTTTACTTTATTGCTACAATTGTGTACTTTTACTAGAAGTACAAATTCTAGAATTAAAGCATCTA |
| | | TGCCGACTACTATAGCAAAGAATACTGTTAAGAGTGTCGGTAAATTTTGTCTAGAGGCTTCATTTAATTA |
| | | TTTGAAGTCACCTAATTTTTCTAAACTGATAAATATTACAATTTGGTTTTTACTATTAAGTGTTTGCCTA |
| | | GGTTCTTTAATCTACTCAACCGCTGCTTTAGGTGTTTTAATGTCTAATTTAGGCATGCCTTCTTACTGTA |
| | | CTGGTTACAGAGAAGGCTATTTGAACTCTACTAATGTCACTATTGCAACCTACTGTACTGGTTCTATACC |
| | | TTGTAGTGTTTGTCTTAGTGGTTTAGATTCTTTAGACACCTATCCTTCTTTAGAAACTATACAAATTACC |
| | | ATTTCATCTTTTAAATGGGATTTAACTGCTTTTGGCTTAGTTGCAGAGTGGTTTTTGGCATATATTCTTT |
| | | TCACTAGGTTTTTCTATGTACTTGGATTGGCTGCAATCATGCAATTGTTTTTCAGCTATTTTGCAGTACA |
| | | TTTTATTAGTAATTCTTGGCTTATGTGGTTAATAATTAATCTTGTACAAATGGCCCCGATTTCAGCTATG |
| | | GTTAGAATGTACATCTTCTTTGCATCATTTTATTATGTATGGAAAAGTTATGTGCATGTTGTAGACGGTT |
| | | GTAATTCATCAACTTGTATGATGTGTTACAAACGTAATAGAGCAACAAGAGTCGAATGTACAACTATTGT |
| | | TAATGGTGTTAGAAGGTCCTTTTATGTCTATGCTAATGGAGGTAAAGGCTTTTGCAAACTACACAATTGG |
| | | AATTGTGTTAATTGTGATACATTCTGTGCTGGTAGTACATTTATTAGTGATGAAGTTGCGAGAGACTTGT |
| | | CACTACAGTTTAAAAGACCAATAAATCCTACTGACCAGTCTTCTTACATCGTTGATAGTGTTACAGTGAA |
| | | GAATGGTTCCATCCATCTTTACTTTGATAAAGCTGGTCAAAAGACTTATGAAAGACATTCTCTCTCTCAT |
| | | TTTGTTAACTTAGACAACCTGAGAGCTAATAACACTAAAGGTTCATTGCCTATTAATGTTATAGTTTTTG |
| | | ATGGTAAATCAAAATGTGAAGAATCATCTGCAAAATCAGCGTCTGTTTACTACAGTCAGCTTATGTGTCA |
| | | ACCTATACTGTTACTAGATCAGGCATTAGTGTCTGATGTTGGTGATAGTGCGGAAGTTGCAGTTAAAATG |
| | | TTTGATGCTTACGTTAATACGTTTTCATCAACTTTTAACGTACCAATGGAAAAACTCAAAACACTAGTTG |
| | | CAACTGCAGAAGCTGAACTTGCAAAGAATGTGTCCTTAGACAATGTCTTATCTACTTTTATTTCAGCAGC |
| | | TCGGCAAGGGTTTGTTGATTCAGATGTAGAAACTAAAGATGTTGTTGAATGTCTTAAATTGTCACATCAA |
| | | TCTGACATAGAAGTTACTGGCGATAGTTGTAATAACTATATGCTCACCTATAACAAAGTTGAAAACATGA |
| | | CACCCCGTGACCTTGGTGCTTGTATTGACTGTAGTGCGCGTCATATTAATGCGCAGGTAGCAAAAAGTCA |
| | | CAACATTGCTTTGATATGGAACGTTAAAGATTTCATGTCATTGTCTGAACAACTACGAAAACAAATACGT |
| | | AGTGCTGCTAAAAAGAATAACTTACCTTTTAAGTTGACATGTGCAACTACTAGACAAGTTGTTAATGTTG |
| | | TAACAACAAAGATAGCACTTAAGGGTGGTAAAATTGTTAATAATTGGTTGAAGCAGTTAATTAAAGTTAC |
| | | ACTTGTGTTCCTTTTTGTTGCTGCTATTTTCTATTTAATAACACCTGTTCATGTCATGTCTAAACATACT |
| | | GACTTTTCAAGTGAAATCATAGGATACAAGGCTATTGATGGTGGTGTCACTCGTGACATAGCATCTACAG |
| | | ATACTTGTTTTGCTAACAAACATGCTGATTTTGACACATGGTTTAGCCAGCGTGGTGGTAGTTATACTAA |
| | | TGACAAAGCTTGCCCATTGATTGCTGCAGTCATAACAAGAGAAGTGGGTTTTGTCGTGCCTGGTTTGCCT |
| | | GGCACGATATTACGCACAACTAATGGTGACTTTTTGCATTTCTTACCTAGAGTTTTTAGTGCAGTTGGTA |
| | | ACATCTGTTACACACCATCAAAACTTATAGAGTACACTGACTTTGCAACATCAGCTTGTGTTTTGGCTGC |
| | | TGAATGTACAATTTTTAAAGATGCTTCTGGTAAGCCAGTACCATATTGTTATGATACCAATGTACTAGAA |
| | | GGTTCTGTTGCTTATGAAAGTTTACGCCCTGACACACGTTATGTGCTCATGGATGGCTCTATTATTCAAT |
| | | TTCCTAACACCTACCTTGAAGGTTCTGTTAGAGTGGTAACAACTTTTGATTCTGAGTACTGTAGGCACGG |
| | | CACTTGTGAAAGATCAGAAGCTGGTGTTTGTGTATCTACTAGTGGTAGATGGGTACTTAACAATGATTAT |
| | | TACAGATCTTTACCAGGAGTTTTCTGTGGTGTAGATGCTGTAAATTTACTTACTAATATGTTTACACCAC |
| | | TAATTCAACCTATTGGTGCTTTGGACATATCAGCATCTATAGTAGCTGGTGGTATTGTAGCTATCGTAGT |
| | | AACATGCCTTGCCTACTATTTTATGAGGTTTAGAAGAGCTTTTGGTGAATACAGTCATGTAGTTGCCTTT |
| | | AATACTTTACTATTCCTTATGTCATTCACTGTACTCTGTTTAACACCAGTTTACTCATTCTTACCTGGTG |
| | | TTTATTCTGTTATTTACTTGTACTTGACATTTTATCTTACTAATGATGTTTCTTTTTTAGCACATATTCA |
| | | GTGGATGGTTATGTTCACACCTTTAGTACCTTTCTGGATAACAATTGCTTATATCATTTGTATTTCCACA |
| | | AAGCATTTCTATTGGTTCTTTAGTAATTACCTAAAGAGACGTGTAGTCTTTAATGGTGTTTCCTTTAGTA |
| | | CTTTTGAAGAAGCTGCGCTGTGCACCTTTTTGTTAAATAAAGAAATGTATCTAAAGTTGCGTAGTGATGT |
| | | GCTATTACCTCTTACGCAATATAATAGATACTTAGCTCTTTATAATAAGTACAAGTATTTTAGTGGAGCA |
| | | ATGGATACAACTAGCTACAGAGAAGCTGCTTGTTGTCATCTCGCAAAGGCTCTCAATGACTTCAGTAACT |
| | | CAGGTTCTGATGTTCTTTACCAACCACCACAAACCTCTATCACCTCAGCTGTTTTGCAGAGTGGTTTTAG |
| | | AAAAATGGCATTCCCATCTGGTAAAGTTGAGGGTTGTATGGTACAAGTAACTTGTGGTACAACTACACTT |
| | | AACGGTCTTTGGCTTGATGACGTAGTTTACTGTCCAAGACATGTGATCTGCACCTCTGAAGACATGCTTA |
| | | ACCCTAATTATGAAGATTTACTCATTCGTAAGTCTAATCATAATTTCTTGGTACAGGCTGGTAATGTTCA |
| | | ACTCAGGGTTATTGGACATTCTATGCAAAATTGTGTACTTAAGCTTAAGGTTGATACAGCCAATCCTAAG |
| | | ACACCTAAGTATAAGTTTGTTCGCATTCAACCAGGACAGACTTTTTCAGTGTTAGCTTGTTACAATGGTT |
| | | CACCATCTGGTGTTTACCAATGTGCTATGAGGCCCAATTTCACTATTAAGGGTTCATTCCTTAATGGTTC |
| | | ATGTGGTAGTGTTGGTTTTAACATAGATTATGACTGTGTCTCTTTTTGTTACATGCACCATATGGAATTA |
| | | CCAACTGGAGTTCATGCTGGCACAGACTTAGAAGGTAACTTTTATGGACCTTTTGTTGACAGGCAAACAG |
| | | CACAAGCAGCTGGTACGGACACAACTATTACAGTTAATGTTTTAGCTTGGTTGTACGCTGCTGTTATAAA |
| | | TGGAGACAGGTGGTTTCTCAATCGATTTACCACAACTCTTAATGACTTTAACCTTGTGGCTATGAAGTAC |
| | | AATTATGAACCTCTAACACAAGACCATGTTGACATACTAGGACCTCTTTCTGCTCAAACTGGAATTGCCG |
| | | TTTTAGATATGTGTGCTTCATTAAAAGAATTACTGCAAAATGGTATGAATGGACGTACCATATTGGGTAG |
| | | TGCTTTATTAGAAGATGAATTTACACCTTTTGATGTTGTTAGACAATGCTCAGGTGTTACTTTCCAAAGT |
| | | GCAGTGAAAAGAACAATCAAGGGTACACACCACTGGTTGTTACTCACAATTTTGACTTCACTTTTAGTTT |
| | | TAGTCCAGAGTACTCAATGGTCTTTGTTCTTTTTTTTGTATGAAAATGCCTTTTTACCTTTTGCTATGGG |
| | | TATTATTGCTATGTCTGCTTTTGCAATGATGTTTGTCAAACATAAGCATGCATTTCTCTGTTTGTTTTTG |
| | | TTACCTTCTCTTGCCACTGTAGCTTATTTTAATATGGTCTATATGCCTGCTAGTTGGGTGATGCGTATTA |
| | | TGACATGGTTGGATATGGTTGATACTAGTTTGAAGCTAAAAGACTGTGTTATGTATGCATCAGCTGTAGT |
| | | GTTACTAATCCTTATGACAGCAAGAACTGTGTATGATGATGGTGCTAGGAGAGTGTGGACACTTATGAAT |
| | | GTCTTGACACTCGTTTATAAAGTTTATTATGGTAATGCTTTAGATCAAGCCATTTCCATGTGGGCTCTTA |
| | | TAATCTCTGTTACTTCTAACTACTCAGGTGTAGTTACAACTGTCATGTTTTTGGCCAGAGGTATTGTTTT |
| | | TATGTGTGTTGAGTATTGCCCTATTTTCTTCATAACTGGTAATACACTTCAGTGTATAATGCTAGTTTAT |
| | | TGTTTCTTAGGCTATTTTTGTACTTGTTACTTTGGCCTCTTTTGTTTACTCAACCGCTACTTTAGACTGA |
| | | CTCTTGGTGTTTATGATTACTTAGTTTCTACACAGGAGTTTAGATATATGAATTCACAGGGACTACTCCC |
| | | ACCCAAGAATAGCATAGATGCCTTCAAACTCAACATTAAATTGTTGGGTGTTGGTGGCAAACCTTGTATC |
| | | AAAGTAGCCACTGTACAGTCTAAAATGTCAGATGTAAAGTGCACATCAGTAGTCTTACTCTCAGTTTTGC |
| | | AACAACTCAGAGTAGAATCATCATCTAAATTGTGGGCTCAATGTGTCCAGTTACACAATGACATTCTCTT |
| | | AGCTAAAGATACTACTGAAGCCTTTGAAAAAATGGTTTCACTACTTTCTGTTTTGCTTTCCATGCAGGGT |
| | | GCTGTAGACATAAACAAGCTTTGTGAAGAAATGCTGGACAACAGGGCAACCTTACAAGCTATAGCCTCAG |
| | | AGTTTAGTTCCCTTCCATCATATGCAGCTTTTGCTACTGCTCAAGAAGCTTATGAGCAGGCTGTTGCTAA |
| | | TGGTGATTCTGAAGTTGTTCTTAAAAAGTTGAAGAAGTCTTTGAATGTGGCTAAATCTGAATTTGACCGT |
| | | GATGCAGCCATGCAACGTAAGTTGGAAAAGATGGCTGATCAAGCTATGACCCAAATGTATAAACAGGCTA |
| | | GATCTGAGGACAAGAGGGCAAAAGTTACTAGTGCTATGCAGACAATGCTTTTCACTATGCTTAGAAAGTT |
| | | GGATAATGATGCACTCAACAACATTATCAACAATGCAAGAGATGGTTGTGTTCCCTTGAACATAATACCT |
| | | CTTACAACAGCAGCCAAACTAATGGTTGTCATACCAGACTATAACACATATAAAAATACGTGTGATGGTA |
| | | CAACATTTACTTATGCATCAGCATTGTGGGAAATCCAACAGGTTGTAGATGCAGATAGTAAAATTGTTCA |
| | | ACTTAGTGAAATTAGTATGGACAATTCACCTAATTTAGCATGGCCTCTTATTGTAACAGCTTTAAGGGCC |
| | | AATTCTGCTGTCAAATTACAGAATAATGAGCTTAGTCCTGTTGCACTACGACAGATGTCTTGTGCTGCCG |
| | | GTACTACACAAACTGCTTGCACTGATGACAATGCGTTAGCTTACTACAACACAACAAAGGGAGGTAGGTT |
| | | TGTACTTGCACTGTTATCCGATTTACAGGATTTGAAATGGGCTAGATTCCCTAAGAGTGATGGAACTGGT |
| | | ACTATCTATACAGAACTGGAACCACCTTGTAGGTTTGTTACAGACACACCTAAAGGTCCTAAAGTGAAGT |
| | | ATTTATACTTTATTAAAGGATTAAACAACCTAAATAGAGGTATGGTACTTGGTAGTTTAGCTGCCACAGT |
| | | ACGTCTACAAGCTGGTAATGCAACAGAAGTGCCTGCCAATTCAACTGTATTATCTTTCTGTGCTTTTGCT |
| | | GTAGATGCTGCTAAAGCTTACAAAGATTATCTAGCTAGTGGGGGACAACCAATCACTAATTGTGTTAAGA |
| | | TGTTGTGTACACACACTGGTACTGGTCAGGCAATAACAGTTACACCGGAAGCCAATATGGATCAAGAATC |
| | | CTTTGGTGGTGCATCGTGTTGTCTGTACTGCCGTTGCCACATAGATCATCCAAATCCTAAAGGATTTTGT |
| | | GACTTAAAAGGTAAGTATGTACAAATACCTACAACTTGTGCTAATGACCCTGTGGGTTTTACACTTAAAA |
| | | ACACAGTCTGTACCGTCTGCGGTATGTGGAAAGGTTATGGCTGTAGTTGTGATCAACTCCGCGAACCCAT |
| | | GCTTCAGTCAGCTGATGCACAATCGTTTTTAAACCGGGTTTGCGGTGTAAGTGCAGCCCGTCTTACACCG |
| | | TGCGGCACAGGCACTAGTACTGATGTCGTATACAGGGCTTTTGACATCTACAATGATAAAGTAGCTGGTT |
| | | TTGCTAAATTCCTAAAAACTAATTGTTGTCGCTTCCAAGAAAAGGACGAAGATGACAATTTAATTGATTC |
| | | TTACTTTGTAGTTAAGAGACACACTTTCTCTAACTACCAACATGAAGAAACAATTTATAATTTACTTAAG |
| | | GATTGTCCAGCTGTTGCTAAACATGACTTCTTTAAGTTTAGAATAGACGGTGACATGGTACCACATATAT |
| | | CACGTCAACGTCTTACTAAATACACAATGGCAGACCTCGTCTATGCTTTAAGGCATTTTGATGAAGGTAA |
| | | TTGTGACACATTAAAAGAAATACTTGTCACATACAATTGTTGTGATGATGATTATTTCAATAAAAAGGAC |
| | | TGGTATGATTTTGTAGAAAACCCAGATATATTACGCGTATACGCCAACTTAGGTGAACGTGTACGCCAAG |
| | | CTTTGTTAAAAACAGTACAATTCTGTGATGCCATGCGAAATGCTGGTATTGTTGGTGTACTGACATTAGA |
| | | TAATCAAGATCTCAATGGTAACTGGTATGATTTCGGTGATTTCATACAAACCACGCCAGGTAGTGGAGTT |
| | | CCTGTTGTAGATTCTTATTATTCATTGTTAATGCCTATATTAACCTTGACCAGGGCTTTAACTGCAGAGT |
| | | CACATGTTGACACTGACTTAACAAAGCCTTACATTAAGTGGGATTTGTTAAAATATGACTTCACGGAAGA |
| | | GAGGTTAAAACTCTTTGACCGTTATTTTAAATATTGGGATCAGACATACCACCCAAATTGTGTTAACTGT |
| | | TTGGATGACAGATGCATTCTGCATTGTGCAAACTTTAATGTTTTATTCTCTACAGTGTTCCCACTTACAA |
| | | GTTTTGGACCACTAGTGAGAAAAATATTTGTTGATGGTGTTCCATTTGTAGTTTCAACTGGATACCACTT |
| | | CAGAGAGCTAGGTGTTGTACATAATCAGGATGTAAACTTACATAGCTCTAGACTTAGTTTTAAGGAATTA |
| | | CTTGTGTATGCTGCTGACCCTGCTATGCACGCTGCTTCTGGTAATCTATTACTAGATAAACGCACTACGT |
| | | GCTTTTCAGTAGCTGCACTTACTAACAATGTTGCTTTTCAAACTGTCAAACCTGGTAATTTTAACAAAGA |
| | | CTTCTATGACTTTGCTGTGTCTAAGGGTTTCTTTAAGGAAGGAAGTTCTGTTGAATTAAAACACTTCTTC |
| | | TTTGCTCAGGATGGTAATGCTGCTATCAGCGATTATGACTACTATCGTTATAATCTACCAACAATGTGTG |
| | | ATATCAGACAACTACTATTTGTAGTTGAAGTTGTTGATAAGTACTTTGATTGTTACGATGGTGGCTGTAT |
| | | TAATGCTAACCAAGTCATCGTCAACAACCTAGACAAATCAGCTGGTTTTCCATTTAATAAATGGGGTAAG |
| | | GCTAGACTTTATTATGATTCAATGAGTTATGAGGATCAAGATGCACTTTTCGCATATACAAAACGTAATG |
| | | TCATCCCTACTATAACTCAAATGAATCTTAAGTATGCCATTAGTGCAAAGAATAGAGCTCGCACCGTAGC |
| | | TGGTGTCTCTATCTGTAGTACTATGACCAATAGACAGTTTCATCAAAAATTATTGAAATCAATAGCCGCC |
| | | ACTAGAGGAGCTACTGTAGTAATTGGAACAAGCAAATTCTATGGTGGTTGGCACAACATGTTAAAAACTG |
| | | TTTATAGTGATGTAGAAAACCCTCATCTTATGGGTTGGGATTATCCTAAATGTGATAGAGCCATGCCTAA |
| | | CATGCTTAGAATTATGGCCTCACTTGTTCTTGCTCGCAAACATACAACGTGTTGTAGCTTGTCACACCGT |
| | | TTCTATAGATTAGCTAATGAGTGTGCTCAAGTATTGAGTGAAATGGTCATGTGTGGCGGTTCACTATATG |
| | | TTAAACCAGGTGGAACCTCATCAGGAGATGCCACAACTGCTTATGCTAATAGTGTTTTTAACATTTGTCA |
| | | AGCTGTCACGGCCAATGTTAATGCACTTTTATCTACTGATGGTAACAAAATTGCCGATAAGTATGTCCGC |
| | | AATTTACAACACAGACTTTATGAGTGTCTCTATAGAAATAGAGATGTTGACACAGACTTTGTGAATGAGT |
| | | TTTACGCATATTTGCGTAAACATTTCTCAATGATGATACTCTCTGACGATGCTGTTGTGTGTTTCAATAG |
| | | CACTTATGCATCTCAAGGTCTAGTGGCTAGCATAAAGAACTTTAAGTCAGTTCTTTATTATCAAAACAAT |
| | | GTTTTTATGTCTGAAGCAAAATGTTGGACTGAGACTGACCTTACTAAAGGACCTCATGAATTTTGCTCTC |
| | | AACATACAATGCTAGTTAAACAGGGTGATGATTATGTGTACCTTCCTTACCCAGATCCATCAAGAATCCT |
| | | AGGGGCCGGCTGTTTTGTAGATGATATCGTAAAAACAGATGGTACACTTATGATTGAACGGTTCGTGTCT |
| | | TTAGCTATAGATGCTTACCCACTTACTAAACATCCTAATCAGGAGTATGCTGATGTCTTTCATTTGTACT |
| | | TACAATACATAAGAAAGCTACATGATGAGTTAACAGGACACATGTTAGACATGTATTCTGTTATGCTTAC |
| | | TAATGATAACACCTCAAGGTATTGGGAACCTGAGTTTTATGAGGCTATGTACACACCGCATACAGTCTTA |
| | | CAGGCTGTTGGGGCTTGTGTTCTTTGCAATTCACAGACTTCATTAAGATGTGGTGCTTGCATACGTAGAC |
| | | CATTCTTATGTTGTAAATGCTGTTACGACCATGTCATATCAACATCACATAAATTAGTCTTGTCTGTTAA |
| | | TCCGTATGTTTGCAATGCTCCAGGTTGTGATGTCACAGATGTGACTCAACTTTACTTAGGAGGTATGAGC |
| | | TATTATTGTAAATCACATAAACCATCCATTAGTTTTCCATTGTGTGCTAATGGACAAGTTTTTGGTTTAT |
| | | ATAAAAATACATGTGTTGGTAGCGATAATGTTACTGACTTTAATGCAATTGCAACATGTGACTGGACAAA |
| | | TGCTGGTGATTACATTTTAGCTAACACCTGTACTGAAAGACTCAAGCTTTTTGCAGCAGAAACGCTCAAA |
| | | GCTACTGAGGAGACATTTAAACTGTCTTATGGTATTGCTACTGTACGTGAAGTGCTGTCTGACAGAGAAT |
| | | TACATCTTTCATGGGAAGTTGGTAAACCTAGACCACCACTTAACCGAAATTATGTCTTTACTGGTTATCG |
| | | TGTAACTAAAAACAGTAAAGTACAAATAGGAGAGTACACCTTTGAAAAAGGTGACTATGGTGATGCTGTT |
| | | GTTTACCGAGGTACAACAACTTACAAATTAAATGTTGGTGATTATTTTGTGCTGACATCACATACAGTAA |
| | | TGCCATTAAGTGCACCTACACTAGTGCCACAAGAGCACTATGTTAGAATTACTGGCTTATACCCAACACT |
| | | CAATATCTCAGATGAGTTTTCTAGCAATGTTGCAAATTATCAAAAGGTTGGTATGCAAAAGTATTCTACA |
| | | CTCCAGGGACCACCTGGTACTGGTAAGAGTCATTTTGCTATTGGCCTAGCTCTCTACTACCCTTCTGCTC |
| | | GCATAGTGTATACAGCTTGCTCTCATGCCGCTGTTGATGCACTATGTGAGAAGGCATTAAAATATTTGCC |
| | | TATAGATAAATGTAGTAGAATTATACCTGCACGTGCTCGTGTAGAGTGTTTTGATAAATTCAAAGTGAAT |
| | | TCAACATTAGAACAGTATGTCTTTTGTACTGTAAATGCATTGCCTGAGACGACAGCAGATATAGTTGTCT |
| | | TTGATGAAATTTCAATGGCCACAAATTATGATTTGAGTGTTGTCAATGCCAGATTACGTGCTAAGCACTA |
| | | TGTGTACATTGGCGACCCTGCTCAATTACCTGCACCACGCACATTGCTAACTAAGGGCACACTAGAACCA |
| | | GAATATTTCAATTCAGTGTGTAGACTTATGAAAACTATAGGTCCAGACATGTTCCTCGGAACTTGTCGGC |
| | | GTTGTCCTGCTGAAATTGTTGACACTGTGAGTGCTTTGGTTTATGATAATAGGCTTAAAGCACATAAAGA |
| | | CAAATCAGCTCAATGCTTTAAAATGTTTTATAAGGGTGTTATCACGCATGATGTTTCATCTGCAATTAAC |
| | | AGGCCACAAATAGGCGTGGTAAGAGAATTCCTTACACGTAACCCTGCTTGGAGAAAAGCTGTCTTTATTT |
| | | CACCTTATAATTCACAGAATGCTGTAGCCTCAAAGATTTTGGGACTACCAACTCAAACTGTTGATTCATC |
| | | ACAGGGCTCAGAATATGACTATGTCATATTCACTCAAACCACTGAAACAGCTCACTCTTGTAATGTAAAC |
| | | AGATTTAATGTTGCTATTACCAGAGCAAAAGTAGGCATACTTTGCATAATGTCTGATAGAGACCTTTATG |
| | | ACAAGTTGCAATTTACAAGTCTTGAAATTCCACGTAGGAATGTGGCAACTTTACAAGCTGAAAATGTAAC |
| | | AGGACTCTTTAAAGATTGTAGTAAGGTAATCACTGGGTTACATCCTACACAGGCACCTACACACCTCAGT |
| | | GTTGACACTAAATTCAAAACTGAAGGTTTATGTGTTGACATACCTGGCATACCTAAGGACATGACCTATA |
| | | GAAGACTCATCTCTATGATGGGTTTTAAAATGAATTATCAAGTTAATGGTTACCCTAACATGTTTATCAC |
| | | CCGCGAAGAAGCTATAAGACATGTACGTGCATGGATTGGCTTCGATGTCGAGGGGTGTCATGCTACTAGA |
| | | GAAGCTGTTGGTACCAATTTACCTTTACAGCTAGGTTTTTCTACAGGTGTTAACCTAGTTGCTGTACCTA |
| | | CAGGTTATGTTGATACACCTAATAATACAGATTTTTCCAGAGTTAGTGCTAAACCACCGCCTGGAGATCA |
| | | ATTTAAACACCTCATACCACTTATGTACAAAGGACTTCCTTGGAATGTAGTGCGTATAAAGATTGTACAA |
| | | ATGTTAAGTGACACACTTAAAAATCTCTCTGACAGAGTCGTATTTGTCTTATGGGCACATGGCTTTGAGT |
| | | TGACATCTATGAAGTATTTTGTGAAAATAGGACCTGAGCGCACCTGTTGTCTATGTGATAGACGTGCCAC |
| | | ATGCTTTTCCACTGCTTCAGACACTTATGCCTGTTGGCATCATTCTATTGGATTTGATTACGTCTATAAT |
| | | CCGTTTATGATTGATGTTCAACAATGGGGTTTTACAGGTAACCTACAAAGCAACCATGATCTGTATTGTC |
| | | AAGTCCATGGTAATGCACATGTAGCTAGTTGTGATGCAATCATGACTAGGTGTCTAGCTGTCCACGAGTG |
| | | CTTTGTTAAGCGTGTTGACTGGACTATTGAATATCCTATAATTGGTGATGAACTGAAGATTAATGCGGCT |
| | | TGTAGAAAGGTTCAACACATGGTTGTTAAAGCTGCATTATTAGCAGACAAATTCCCAGTTCTTCACGACA |
| | | TTGGTAACCCTAAAGCTATTAAGTGTGTACCTCAAGCTGATGTAGAATGGAAGTTCTATGATGCACAGCC |
| | | TTGTAGTGACAAAGCTTATAAAATAGAAGAATTATTCTATTCTTATGCCACACATTCTGACAAATTCACA |
| | | GATGGTGTATGCCTATTTTGGAATTGCAATGTCGATAGATATCCTGCTAATTCCATTGTTTGTAGATTTG |
| | | ACACTAGAGTGCTATCTAACCTTAACTTGCCTGGTTGTGATGGTGGCAGTTTGTATGTAAATAAACATGC |
| | | ATTCCACACACCAGCTTTTGATAAAAGTGCTTTTGTTAATTTAAAACAATTACCATTTTTCTATTACTCT |
| | | GACAGTCCATGTGAGTCTCATGGAAAACAAGTAGTGTCAGATATAGATTATGTACCACTAAAGTCTGCTA |
| | | CGTGTATAACACGTTGCAATTTAGGTGGTGCTGTCTGTAGACATCATGCTAATGAGTACAGATTGTATCT |
| | | CGATGCTTATAACATGATGATCTCAGCTGGCTTTAGCTTGTGGGTTTACAAACAATTTGATACTTATAAC |
| | | CTCTGGAACACTTTTACAAGACTTCAGAGTTTAGAAAATGTGGCTTTTAATGTTGTAAATAAGGGACACT |
| | | TTGATGGACAACAGGGTGAAGTACCAGTTTCTATCATTAATAACACTGTTTACACAAAAGTTGATGGTGT |
| | | TGATGTAGAATTGTTTGAAAATAAAACAACATTACCTGTTAATGTAGCATTTGAGCTTTGGGCTAAGCGC |
| | | AACATTAAACCAGTACCAGAGGTGAAAATACTCAATAATTTGGGTGTGGACATTGCTGCTAATACTGTGA |
| | | TCTGGGACTACAAAAGAGATGCTCCAGCACATATATCTACTATTGGTGTTTGTTCTATGACTGACATAGC |
| | | CAAGAAACCAACTGAAACGATTTGTGCACCACTCACTGTCTTTTTTGATGGTAGAGTTGATGGTCAAGTA |
| | | GACTTATTTAGAAATGCCCGTAATGGTGTTCTTATTACAGAAGGTAGTGTTAAAGGTTTACAACCATCTG |
| | | TAGGTCCCAAACAAGCTAGTCTTAATGGAGTCACATTAATTGGAGAAGCCGTAAAAACACAGTTCAATTA |
| | | TTATAAGAAAGTTGATGGTGTTGTCCAACAATTACCTGAAACTTACTTTACTCAGAGTAGAAATTTACAA |
| | | GAATTTAAACCCAGGAGTCAAATGGAAATTGATTTCTTAGAATTAGCTATGGATGAATTCATTGAACGGT |
| | | ATAAATTAGAAGGCTATGCCTTCGAACATATCGTTTATGGAGATTTTAGTCATAGTCAGTTAGGTGGTTT |
| | | ACATCTACTGATTGGACTAGCTAAACGTTTTAAGGAATCACCTTTTGAATTAGAAGATTTTATTCCTATG |
| | | GACAGTACAGTTAAAAACTATTTCATAACAGATGCGCAAACAGGTTCATCTAAGTGTGTGTGTTCTGTTA |
| | | TTGATTTATTACTTGATGATTTTGTTGAAATAATAAAATCCCAAGATTTATCTGTAGTTTCTAAGGTTGT |
| | | CAAAGTGACTATTGACTATACAGAAATTTCATTTATGCTTTGGTGTAAAGATGGCCATGTAGAAACATTT |
| | | TACCCAAAATTACAATCTAGTCAAGCGTGGCAACCGGGTGTTGCTATGCCTAATCTTTACAAAATGCAAA |
| | | GAATGCTATTAGAAAAGTGTGACCTTCAAAATTATGGTGATAGTGCAACATTACCTAAAGGCATAATGAT |
| | | GAATGTCGCAAAATATACTCAACTGTGTCAATATTTAAACACATTAACATTAGCTGTACCCTATAATATG |
| | | AGAGTTATACATTTTGGTGCTGGTTCTGATAAAGGAGTTGCACCAGGTACAGCTGTTTTAAGACAGTGGT |
| | | TGCCTACGGGTACGCTGCTTGTCGATTCAGATCTTAATGACTTTGTCTCTGATGCAGATTCAACTTTGAT |
| | | TGGTGATTGTGCAACTGTACATACAGCTAATAAATGGGATCTCATTATTAGTGATATGTACGACCCTAAG |
| | | ACTAAAAATGTTACAAAAGAAAATGACTCTAAAGAGGGTTTTTTCACTTACATTTGTGGGTTTATACAAC |
| | | AAAAGCTAGCTCTTGGAGGTTCCGTGGCTATAAAGATAACAGAACATTCTTGGAATGCTGATCTTTATAA |
| | | GCTCATGGGACACTTCGCATGGTGGACAGCCTTTGTTACTAATGTGAATGCGTCATCATCTGAAGCATTT |
| | | TTAATTGGATGTAATTATCTTGGCAAACCACGCGAACAAATAGATGGTTATGTCATGCATGCAAATTACA |
| | | TATTTTGGAGGAATACAAATCCAATTCAGTTGTCTTCCTATTCTTTATTTGACATGAGTAAATTTCCCCT |
| | | TAAATTAAGGGGTACTGCTGTTATGTCTTTAAAAGAAGGTCAAATCAATGATATGATTTTATCTCTTCTT |
| | | AGTAAAGGTAGACTTATAATTAGAGAAAACAACAGAGTTGTTATTTCTAGTGATGTTCTTGTTAACAACT |
| | | AA |
| 3 | Severe | ATGGAGAGCCTTGTCCCTGGTTTCAACGAGAAAACACACGTCCAACTCAGTTTGCCTGTTTTACAGGTTC |
| | acute | GCGACGTGCTCGTACGTGGCTTTGGAGACTCCGTGGAGGAGGTCTTATCAGAGGCACGTCAACATCTTAA |
| | respiratory | AGATGGCACTTGTGGCTTAGTAGAAGTTGAAAAAGGCGTTTTGCCTCAACTTGAACAGCCCTATGTGTTC |
| | syndrome | ATCAAACGTTCGGATGCTCGAACTGCACCTCATGGTCATGTTATGGTTGAGCTGGTAGCAGAACTCGAAG |
| | coronavirus 2 | GCATTCAGTACGGTCGTAGTGGTGAGACACTTGGTGTCCTTGTCCCTCATGTGGGCGAAATACCAGTGGC |
| | isolate | TTACCGCAAGGTTCTTCTTCGTAAGAACGGTAATAAAGGAGCTGGTGGCCATAGTTACGGCGCCGATCTA |
| | SARS- | AAGTCATTTGACTTAGGCGACGAGCTTGGCACTGATCCTTATGAAGATTTTCAAGAAAACTGGAACACTA |
| | CoV- | AACATAGCAGTGGTGTTACCCGTGAACTCATGCGTGAGCTTAACGGAGGGGCATACACTCGCTATGTCGA |
| | 2/human/ | TAACAACTTCTGTGGCCCTGATGGCTACCCTCTTGAGTGCATTAAAGACCTTCTAGCACGTGCTGGTAAA |
| | ZAF/R030 | GCTTCATGCACTTTGTCCGAACAACTGGACTTTATTGACACTAAGAGGGGTGTATACTGCTGCCGTGAAC |
| | 06/2020, | ATGAGCATGAAATTGCTTGGTACACGGAACGTTCTGAAAAGAGCTATGAATTGCAGACACCTTTTGAAAT |
| | complete | TAAATTGGCAAAGAAATTTGACACCTTCAATGGGGAATGTCCAAATTTTGTATTTCCCTTAAATTCCATA |
| | genome | ATCAAGACTATTCAACCAAGGGTTGAAAAGAAAAAGCTTGATGGCTTTATGGGTAGAATTCGATCTGTCT |
| | | ATCCAGTTGCGTCACCAAATGAATGCAACCAAATGTGCCTTTCAACTCTCATGAAGTGTGATCATTGTGG |
| | | TGAAACTTCATGGCAGACGGGCGATTTTGTTAAAGCCACTTGCGAATTTTGTGGCACTGAGAATTTGACT |
| | | AAAGAAGGTGCCACTACTTGTGGTTACTTACCCCAAAATGCTGTTGTTAAAATTTATTGTCCAGCATGTC |
| | | ACAATTCAGAAGTAGGACCTGAGCATAGTCTTGCCGAATACCATAATGAATCTGGCTTGAAAACCATTCT |
| | | TCGTAAGGGTGGTCGCACTATTGCCTTTGGAGGCTGTGTGTTCTCTTATGTTGGTTGCCATAACAAGTGT |
| | | GCCTATTGGGTTCCACGTGCTAGCGCTAACATAGGTTGTAACCATACAGGTGTTGTTGGAGAAGGTTCCG |
| | | AAGGTCTTAATGACAACCTTCTTGAAATACTCCAAAAAGAGAAAGTCAACATCAATATTGTTGGTGACTT |
| | | TAAACTTAATGAAGAGATCGCCATTATTTTGGCATCTTTTTCTGCTTCCACAAGTGCTTTTGTGGAAACT |
| | | GTGAAAGGTTTGGATTATAAAGCATTCAAACAAATTGTTGAATCCTGTGGTAATTTTAAAGTTACAAAAG |
| | | GAAAAGCTAAAAAAGGTGCCTGGAATATTGGTGAACAGAAATCAATACTGAGTCCTCTTTATGCATTTGC |
| | | ATCAGAGGCTGCTCGTGTTGTACGATCAATTTTCTCCCGCACTCTTGAAACTGCTCAAAATTCTGTGCGT |
| | | GTTTTACAGAAGGCCGCTATAACAATACTAGATGGAATTTCACAGTATTCACTGAGACTCATTGATGCTA |
| | | TGATGTTCACATCTGATTTGGCTACTAACAATCTAGTTGTAATGGCCTACATTACAGGTGGTGTTGTTCA |
| | | GTTGACTTCGCAGTGGCTAACTAACATCTTTGGCACTGTTTATGAAAAACTCAAACCCGTCCTTGATTGG |
| | | CTTGAAGAGAAGTTTAAGGAAGGTGTAGAGTTTCTTAGAGACGGTTGGGAAATTGTTAAATTTATCTCAA |
| | | CCTGTGCTTGTGAAATTGTCGGTGGACAAATTGTCACCTGTGCAAAGGAAATTAAGGAGAGTGTTCAGAC |
| | | ATTCTTTAAGCTTGTAAATAAATTTTTGGCTTTGTGTGCTGACTCTATCATTATTGGTGGAGCTAAACTT |
| | | AAAGCCTTGAATTTAGGTGAAACATTTGTCACGCACTCAAAGGGATTGTACAGAAAGTGTGTTAAATCCA |
| | | GAGAAGAAACTGGCCTACTCATGCCTCTAAAAGCCCCAAAAGAAATTATCTTCTTAGAGGGAGAAACACT |
| | | TCCCACAGAAGTGTTAACAGAGGAAGTTGTCTTGAAAACTGGTGATTTACAACCATTAGAACAACCTACT |
| | | AGTGAAGCTGTTGAAGCTCCATTGGTTGGTACACCAGTTTGTATTAACGGGCTTATGTTGCTCGAAATCA |
| | | AAGACACAGAAAAGTACTGTGCCCTTGCACCTAATATGATGGTAACAAACAATACCTTCACACTCAAAGG |
| | | CGGTGCACCAACAAAGGTTACTTTTGGTGATGACACTGTGATAGAAGTGCAAGGTTACAAGAGTGTGAAT |
| | | ATCACTTTTGAACTTGATGAAAGGATTGATAAAGTACTTAATGAGAAGTGCTCTGCCTATACAGTTGAAC |
| | | TCGGTACAGAAGTAAATGAGTTCGCCTGTGTTGTGGCAGATGCTGTCATAAAAACTTTGCAACCAGTATC |
| | | TGAATTACTTACACCACTGGGCATTGATTTAGATGAGTGGAGTATGGCTACATACTACTTATTTGATGAG |
| | | TCTGGTGAGTTTAAATTGGCTTCACATATGTATTGTTCTTTTTACCCTCCAGATGAGGATGAAGAAGAAG |
| | | GTGATTGTGAAGAAGAAGAGTTTGAGCCATCAACTCAATATGAGTATGGTACTGAAGATGATTACCAAGG |
| | | TAAACCTTTGGAATTTGGTGCCACTTCTGCTGCTCTTCAACCTGAAGAAGAGCAAGAAGAAGATTGGTTA |
| | | GATGATGATAGTCAACAAACTGTTGGTCAACAAGACGGCAGTGAGGACAATCAGACAACTACTATTCAAA |
| | | CAATTGTTGAGGTTCAACCTCAATTAGAGATGGAACTTACACCAGTTGTTCAGACTATTGAAGTGAATAG |
| | | TTTTAGTGGTTATTTAAAACTTACTGACAATGTATACATTAAAAATGCAGACATTGTGGAAGAAGCTAAA |
| | | AAGGTAAAACCAACAGTGGTTGTTAATGCAGCCAATGTTTACCTTAAACATGGAGGAGGTGTTGCAGGAG |
| | | CCTTAAATAAGGCTACTAACAATGCCATGCAAGTTGAATCTGATGATTACATAGCTACTAATGGACCACT |
| | | TAAAGTGGGTGGTAGTTGTGTTTTAAGCGGACACAATCTTGCTAAACACTGTCTTCATGTTGTCGGCCCA |
| | | AATGTTAACAAAGGTGAAGACATTCAACTTCTTAAGAGTGCTTATGAAAATTTTAATCAGCACGAAGTTC |
| | | TACTTGCACCATTATTATCAGCTGGTATTTTTGGTGCTGACCCTATACATTCTTTAAGAGTTTGTGTAGA |
| | | TACTGTTCGCACAAATGTCTACTTAGCTGTCTTTGATAAAAATCTCTATGACAAACTTGTTTCAAGCTTT |
| | | TTGGAAATGAAGAGTGAAAAGCAAGTTGAACAAAAGATCGCTGAGATTCCTAAAGAGGAAGTTAAGCCAT |
| | | TTATAACTGAAAGTAAACCTTCAGTTGAACAGAGAAAACAAGATGATAAGAAAATCAAAGCTTGTGTTGA |
| | | AGAAGTTACAACAACTCTGGAAGAAACTAAGTTCCTCACAGAAAACTTGTTACTTTATATTGACATTAAT |
| | | GGCAATCTTCATCCAGATTCTGCCACTCTTGTTAGTGACATTGACATCACTTTCTTAAAGAAAGATGCTC |
| | | CATATATAGTGGGTGATGTTGTTCAAGAGGGTGTTTTAACTGCTGTGGTTATACCTACTAAAAAGGCTGG |
| | | TGGCACTACTGAAATGCTAGCGAAAGCTTTGAGAAAAGTGCCAACAGACAATTATATAACCACTTACCCG |
| | | GGTCAGGGTTTAAATGGTTACACTGTAGAGGAGGCAAAGACAGTGCTTAAAAAGTGTAAAAGTGCCTTTT |
| | | ACATTCTACCATCTATTATCTCTAATGAGAAGCAAGAAATTCTTGGAACTGTTTCTTGGAATTTGCGAGA |
| | | AATGCTTGCACATGCAGAAGAAACACGCAAATTAATGCCTGTCTGTGTGGAAACTAAAGCCATAGTTTCA |
| | | ACTATACAGCGTAAATATAAGGGTATTAAAATACAAGAGGGTGTGGTTGATTATGGTGCTAGATTTTACT |
| | | TTTACACCAGTAAAACAACTGTAGCGTCACTTATCAACACACTTAACGATCTAAATGAAACTCTTGTTAC |
| | | AATGCCACTTGGCTATGTAACACATGGCTTAAATTTGGAAGAAGCTGCTCGGTATATGAGATCTCTCAAA |
| | | GTGCCAGCTACAGTTTCTGTTTCTTCACCTGATGCTGTTACAGCGTATAATGGTTATCTTACTTCTTCTT |
| | | CTAAAACACCTGAAGAACATTTTATTGAAACCATCTCACTTGCTGGTTCCTATAAAGATTGGTCCTATTC |
| | | TGGACAATCTACACAACTAGGTATAGAATTTCTTAAGAGAGGTGATAAAAGTGTATATTACACTAGTAAT |
| | | CCTACCACATTCCACCTAGATGGTGAAGTTATCACCTTTGACAATCTTAAGACACTTCTTTCTTTGAGAG |
| | | AAGTGAGGACTATTAAGGTGTTTACAACAGTAGACAACATTAACCTCCACACGCAAGTTGTGGACATGTC |
| | | AATGACATATGGACAACAGTTTGGTCCAACTTATTTGGATGGAGCTGATGTTACTAAAATAAAACCTCAT |
| | | AATTCACATGAAGGTAAAACATTTTATGTTTTACCTAATGATGACACTCTACGTGTTGAGGCTTTTGAGT |
| | | ACTACCACACAACTGATCCTAGTTTTCTGGGTAGGTACATGTCAGCATTAAATCACACTAAAAAGTGGAA |
| | | ATACCCACAAGTTAATGGTTTAACTTCTATTAAATGGGCAGATAACAACTGTTATCTTGCCACTGCATTG |
| | | TTAACACTCCAACAAATAGAGTTGAAGTTTAATCCACCTGCTCTACAAGATGCTTATTACAGAGCAAGGG |
| | | CTGGTGAAGCTGCTAACTTTTGTGCACTTATCTTAGCCTACTGTAATAAGACAGTAGGTGAGTTAGGTGA |
| | | TGTTAGAGAAACAATGAGTTACTTGTTTCAACATGCCAATTTAGATTCTTGCAAAAGAGTCTTGAACGTG |
| | | GTGTGTAAAACTTGTGGACAACAGCAGACAACCCTTAAGGGTGTAGAAGCTGTTATGTACATGGGCACAC |
| | | TTTCTTATGAACAATTTAAGAAAGGTGTTCAGATACCTTGTACGTGTGGTAAACAAGCTACAAAATATCT |
| | | AGTACAACAGGAGTCACCTTTTGTTATGATGTCAGCACCACCTGCTCAGTATGAACTTAAGCATGGTACA |
| | | TTTACTTGTGCTAGTGAGTACACTGGTAATTACCAGTGTGGTCACTATAAACATATAACTTCTAAAGAAA |
| | | CTTTGTATTGCATAGACGGTGCTTTACTTACAAAGTCCTCAGAATACAAAGGTCCTATTACGGATGTTTT |
| | | CTACAAAGAAAACAGTTACACAACAACCATAAAACCAGTTACTTATAAATTGGATGGTGTTGTTTGTACA |
| | | GAAATTGACCCTAAGTTGGACAATTATTATAAGAAAGACAATTCTTATTTCACAGAGCAACCAATTGATC |
| | | TTGTACCAAACCAACCATATCCAAACGCAAGCTTCGATAATTTTAAGTTTGTATGTGATAATATCAAATT |
| | | TGCTGATGATTTAAACCAGTTAACTGGTTATAAGAAACCTGCTTCAAGAGAGCTTAAAGTTACATTTTTC |
| | | CCTGACTTAAATGGTGATGTGGTGGCTATTGATTATAAACACTACACACCCTCTTTTAAGAAAGGAGCTA |
| | | AATTGTTACATAAACCTATTGTTTGGCATGTTAACAATGCAACTAATAAAGCCACGTATAAACCAAATAC |
| | | CTGGTGTATACGTTGTCTTTGGAGCACAAAACCAGTTGAAACATCAAATTCGTTTGATGTACTGAAGTCA |
| | | GAGGACGCGCAGGGAATGGATAATCTTGCCTGCGAAGATCTAAAACCAGTCTCTGAAGAAGTAGTGGAAA |
| | | ATCCTACCATACAGAAAGACGTTCTTGAGTGTAATGTGAAAACTACCGAAGTTGTAGGAGACATTATACT |
| | | TAAACCAGCAAATAATAGTTTAAAAATTACAGAAGAGGTTGGCCACACAGATCTAATGGCTGCTTATGTA |
| | | GACAATTCTAGTCTTACTATTAAGAAACCTAATGAATTATCTAGAGTATTAGGTTTGAAAACCCTTGCTA |
| | | CTCATGGTTTAGCTGCTGTTAATAGTGTCCCTTGGGATACTATAGCTAATTATGCTAAGCCTTTTCTTAA |
| | | CAAAGTTGTTAGTACAACTACTAACATAGTTACACGGTGTTTAAACCGTGTTTGTACTAATTATATGCCT |
| | | TATTTCTTTACTTTATTGCTACAATTGTGTACTTTTACTAGAAGTACAAATTCTAGAATTAAAGCATCTA |
| | | TGCCGACTACTATAGCAAAGAATACTGTTAAGAGTGTCGGTAAATTTTGTCTAGAGGCTTCATTTAATTA |
| | | TTTGAAGTCACCTAATTTTTCTAAACTGATAAATATTATAATTTGGTTTTTACTATTAAGTGTTTGCCTA |
| | | GGTTCTTTAATCTACTCAACCGCTGCTTTAGGTGTTTTAATGTCTAATTTAGGCATGCCTTCTTACTGTA |
| | | CTGGTTACAGAGAAGGCTATTTGAACTCTACTAATGTCACTATTGCAACCTACTGTACTGGTTCTATACC |
| | | TTGTAGTGTTTGTCTTAGTGGTTTAGATTCTTTAGACACCTATCCTTCTTTAGAAACTATACAAATTACC |
| | | ATTTCATCTTTTAAATGGGATTTAACTGCTTTTGGCTTAGTTGCAGAGTGGTTTTTGGCATATATTCTTT |
| | | TCACTAGGTTTTTCTATGTACTTGGATTGGCTGCAATCATGCAATTGTTTTTCAGCTATTTTGCAGTACA |
| | | TTTTATTAGTAATTCTTGGCTTATGTGGTTAATAATTAATCTTGTACAAATGGCCCCGATTTCAGCTATG |
| | | GTTAGAATGTACATCTTCTTTGCATCATTTTATTATGTATGGAAAAGTTATGTGCATGTTGTAGACGGTT |
| | | GTAATTCATCAACTTGTATGATGTGTTACAAACGTAATAGAGCAACAAGAGTCGAATGTACAACTATTGT |
| | | TAATGGTGTTAGAAGGTCCTTTTATGTCTATGCTAATGGAGGTAAAGGCTTTTGCAAACTACACAATTGG |
| | | AATTGTGTTAATTGTGATACATTCTGTGCTGGTAGTACATTTATTAGTGATGAAGTTGCGAGAGACTTGT |
| | | CACTACAGTTTAAAAGACCAATAAATCCTACTGACCAGTCTTCTTACATCGTTGATAGTGTTACAGTGAA |
| | | GAATGGTTCCATCCATCTTTACTTTGATAAAGCTGGTCAAAAGACTTATGAAAGACATTCTCTCTCTCAT |
| | | TTTGTTAACTTAGACAACCTGAGAGCTAATAACACTAAAGGTTCATTGCCTATTAATGTTATAGTTTTTG |
| | | ATGGTAAATCAAAATGTGAAGAATCATCTGCAAAATCAGCGTCTGTTTACTACAGTCAGCTTATGTGTCA |
| | | ACCTATACTGTTACTAGATCAGGCATTAGTGTCTGATGTTGGTGATAGTGCGGAAGTTGCAGTTAAAATG |
| | | TTTGATGCTTACGTTAATACGTTTTCATCAACTTTTAACGTACCAATGGAAAAACTCAAAACACTAGTTG |
| | | CAACTGCAGAAGCTGAACTTGCAAAGAATGTGTCCTTAGACAATGTCTTATCTACTTTTATTTCAGCAGC |
| | | TCGGCAAGGGTTTGTTGATTCAGATGTAGAAACTAAAGATGTTGTTGAATGTCTTAAATTGTCACATCAA |
| | | TCTGACATAGAAGTTACTGGCGATAGTTGTAATAACTATATGCTCACCTATAACAAAGTTGAAAACATGA |
| | | CACCCCGTGACCTTGGTGCTTGTATTGACTGTAGTGCGCGTCATATTAATGCGCAGGTAGCAAAAAGTCA |
| | | CAACATTGCTTTGATATGGAACGTTAAAGATTTCATGTCATTGTCTGAACAACTACGAAAACAAATACGT |
| | | AGTGCTGCTAAAAAGAATAACTTACCTTTTAAGTTGACATGTGCAACTACTAGACAAGTTGTTAATGTTG |
| | | TAACAACAAAGATAGCACTTAAGGGTGGTAAAATTGTTAATAATTGGTTGAAGCAGTTAATTAAAGTTAC |
| | | ACTTGTGTTCCTTTTTGTTGCTGCTATTTTCTATTTAATAACACCTGTTCATGTCATGTCTAAACATACT |
| | | GACTTTTCAAGTGAAATCATAGGATACAAGGCTATTGATGGTGGTGTCACTCGTGACATAGCATCTACAG |
| | | ATACTTGTTTTGCTAACAAACATGCTGATTTTGACACATGGTTTAGCCAGCGTGGTGGTAGTTATACTAA |
| | | TGACAAAGCTTGCCCATTGATTGCTGCAGTCATAACAAGAGAAGTGGGTTTTGTCGTGCCTGGTTTGCCT |
| | | GGCACGATATTACGCACAACTAATGGTGACTTTTTGCATTTCTTACCTAGAGTTTTTAGTGCAGTTGGTA |
| | | ACATCTGTTACACACCATCAAAACTTATAGAGTACACTGACTTTGCAACATCAGCTTGTGTTTTGGCTGC |
| | | TGAATGTACAATTTTTAAAGATGCTTCTGGTAAGCCAGTACCATATTGTTATGATACCAATGTACTAGAA |
| | | GGTTCTGTTGCTTATGAAAGTTTACGCCCTGACACACGTTATGTGCTCATGGATGGCTCTATTATTCAAT |
| | | TTCCTAACACCTACCTTGAAGGTTCTGTTAGAGTGGTAACAACTTTTGATTCTGAGTACTGTAGGCACGG |
| | | CACTTGTGAAAGATCAGAAGCTGGTGTTTGTGTATCTACTAGTGGTAGATGGGTACTTAACAATGATTAT |
| | | TACAGATCTTTACCAGGAGTTTTCTGTGGTGTAGATGCTGTAAATTTACTTACTAATATGTTTACACCAC |
| | | TAATTCAACCTATTGGTGCTTTGGACATATCAGCATCTATAGTAGCTGGTGGTATTGTAGCTATCGTAGT |
| | | AACATGCCTTGCCTACTATTTTATGAGGTTTAGAAGAGCTTTTGGTGAATACAGTCATGTAGTTGCCTTT |
| | | AATACTTTACTATTCCTTATGTCATTCACTGTACTCTGTTTAACACCAGTTTACTCATTCTTACCTGGTG |
| | | TTTATTCTGTTATTTACTTGTACTTGACATTTTATCTTACTAATGATGTTTCTTTTTTAGCACATATTCA |
| | | GTGGATGGTTATGTTCACACCTTTAGTACCTTTCTGGATAACAATTGCTTATATCATTTGTATTTCCACA |
| | | AAGCATTTCTATTGGTTCTTTAGTAATTACCTAAAGAGACGTGTAGTCTTTAATGGTGTTTCCTTTAGTA |
| | | CTTTTGAAGAAGCTGCGCTGTGCACCTTTTTGTTAAATAAAGAAATGTATCTAAAGTTGCGTAGTGATGT |
| | | GCTATTACCTCTTACGCAATATAATAGATACTTAGCTCTTTATAATAAGTACAAGTATTTTAGTGGAGCA |
| | | ATGGATACAACTAGCTACAGAGAAGCTGCTTGTTGTCATCTCGCAAAGGCTCTCAATGACTTCAGTAACT |
| | | CAGGTTCTGATGTTCTTTACCAACCACCACAAACCTCTATCACCTCAGCTGTTTTGCAGAGTGGTTTTAG |
| | | AAAAATGGCATTCCCATCTGGTAAAGTTGAGGGTTGTATGGTACAAGTAACTTGTGGTACAACTACACTT |
| | | AACGGTCTTTGGCTTGATGACGTAGTTTACTGTCCAAGACATGTGATCTGCACCTCTGAAGACATGCTTA |
| | | ACCCTAATTATGAAGATTTACTCATTCGTAAGTCTAATCATAATTTCTTGGTACAGGCTGGTAATGTTCA |
| | | ACTCAGGGTTATTGGACATTCTATGCAAAATTGTGTACTTAAGCTTAAGGTTGATACAGCCAATCCTAAG |
| | | ACACCTAAGTATAAGTTTGTTCGCATTCAACCAGGACAGACTTTTTCAGTGTTAGCTTGTTACAATGGTT |
| | | CACCATCTGGTGTTTACCAATGTGCTATGAGGCCCAATTTCACTATTAAGGGTTCATTCCTTAATGGTTC |
| | | ATGTGGTAGTGTTGGTTTTAACATAGATTATGACTGTGTCTCTTTTTGTTACATGCACCATATGGAATTA |
| | | CCAACTGGAGTTCATGCTGGCACAGACTTAGAAGGTAACTTTTATGGACCTTTTGTTGACAGGCAAACAG |
| | | CACAAGCAGCTGGTACGGACACAACTATTACAGTTAATGTTTTAGCTTGGTTGTACGCTGCTGTTATAAA |
| | | TGGAGACAGGTGGTTTCTCAATCGATTTACCACAACTCTTAATGACTTTAACCTTGTGGCTATGAAGTAC |
| | | AATTATGAACCTCTAACACAAGACCATGTTGACATACTAGGACCTCTTTCTGCTCAAACTGGAATTGCCG |
| | | TTTTAGATATGTGTGCTTCATTAAAAGAATTACTGCAAAATGGTATGAATGGACGTACCATATTGGGTAG |
| | | TGCTTTATTAGAAGATGAATTTACACCTTTTGATGTTGTTAGACAATGCTCAGGTGTTACTTTCCAAAGT |
| | | GCAGTGAAAAGAACAATCAAGGGTACACACCACTGGTTGTTACTCACAATTTTGACTTCACTTTTAGTTT |
| | | TAGTCCAGAGTACTCAATGGTCTTTGTTCTTTTTTTTGTATGAAAATGCCTTTTTACCTTTTGCTATGGG |
| | | TATTATTGCTATGTCTGCTTTTGCAATGATGTTTGTCAAACATAAGCATGCATTTCTCTGTTTGTTTTTG |
| | | TTACCTTCTCTTGCCACTGTAGCTTATTTTAATATGGTCTATATGCCTGCTAGTTGGGTGATGCGTATTA |
| | | TGACATGGTTGGATATGGTTGATACTAGTTTGTCTGGTTTTAAGCTAAAAGACTGTGTTATGTATGCATC |
| | | AGCTGTAGTGTTACTAATCCTTATGACAGCAAGAACTGTGTATGATGATGGTGCTAGGAGAGTGTGGACA |
| | | CTTATGAATGTCTTGACACTCGTTTATAAAGTTTATTATGGTAATGCTTTAGATCAAGCCATTTCCATGT |
| | | GGGCTCTTATAATCTCTGTTACTTCTAACTACTCAGGTGTAGTTACAACTGTCATGTTTTTGGCCAGAGG |
| | | TATTGTTTTTATGTGTGTTGAGTATTGCCCTATTTTCTTCATAACTGGTAATACACTTCAGTGTATAATG |
| | | CTAGTTTATTGTTTCTTAGGCTATTTTTGTACTTGTTACTTTGGCCTCTTTTGTTTACTCAACCGCTACT |
| | | TTAGACTGACTCTTGGTGTTTATGATTACTTAGTTTCTACACAGGAGTTTAGATATATGAATTCACAGGG |
| | | ACTACTCCCACCCAAGAATAGCATAGATGCCTTCAAACTCAACATTAAATTGTTGGGTGTTGGTGGCAAA |
| | | CCTTGTATCAAAGTAGCCACTGTACAGTCTAAAATGTCAGATGTAAAGTGCACATCAGTAGTCTTACTCT |
| | | CAGTTTTGCAACAACTCAGAGTAGAATCATCATCTAAATTGTGGGCTCAATGTGTCCAGTTACACAATGA |
| | | CATTCTCTTAGCTAAAGATACTACTGAAGCCTTTGAAAAAATGGTTTCACTACTTTCTGTTTTGCTTTCC |
| | | ATGCAGGGTGCTGTAGACATAAACAAGCTTTGTGAAGAAATGCTGGACAACAGGGCAACCTTACAAGCTA |
| | | TAGCCTCAGAGTTTAGTTCCCTTCCATCATATGCAGCTTTTGCTACTGCTCAAGAAGCTTATGAGCAGGC |
| | | TGTTGCTAATGGTGATTCTGAAGTTGTTCTTAAAAAGTTGAAGAAGTCTTTGAATGTGGCTAAATCTGAA |
| | | TTTGACCGTGATGCAGCCATGCAACGTAAGTTGGAAAAGATGGCTGATCAAGCTATGACCCAAATGTATA |
| | | AACAGGCTAGATCTGAGGACAAGAGGGCAAAAGTTACTAGTGCTATGCAGACAATGCTTTTCACTATGCT |
| | | TAGAAAGTTGGATAATGATGCACTCAACAACATTATCAACAATGCAAGAGATGGTTGTGTTCCCTTGAAC |
| | | ATAATACCTCTTACAACAGCAGCCAAACTAATGGTTGTCATACCAGACTATAACACATATAAAAATACGT |
| | | GTGATGGTACAACATTTACTTATGCATCAGCATTGTGGGAAATCCAACAGGTTGTAGATGCAGATAGTAA |
| | | AATTGTTCAACTTAGTGAAATTAGTATGGACAATTCACCTAATTTAGCATGGCCTCTTATTGTAACAGCT |
| | | TTAAGGGCCAATTCTGCTGTCAAATTACAGAATAATGAGCTTAGTCCTGTTGCACTACGACAGATGTCTT |
| | | GTGCTGCCGGTACTACACAAACTGCTTGCACTGATGACAATGCGTTAGCTTACTACAACACAACAAAGGG |
| | | AGGTAGGTTTGTACTTGCACTGTTATCCGATTTACAGGATTTGAAATGGGCTAGATTCCCTAAGAGTGAT |
| | | GGAACTGGTACTATCTATACAGAACTGGAACCACCTTGTAGGTTTGTTACAGACACACCTAAAGGTCCTA |
| | | AAGTGAAGTATTTATACTTTATTAAAGGATTAAACAACCTAAATAGAGGTATGGTACTTGGTAGTTTAGC |
| | | TGCCACAGTACGTCTACAAGCTGGTAATGCAACAGAAGTGCCTGCCAATTCAACTGTATTATCTTTCTGT |
| | | GCTTTTGCTGTAGATGCTGCTAAAGCTTACAAAGATTATCTAGCTAGTGGGGGACAACCAATCACTAATT |
| | | GTGTTAAGATGTTGTGTACACACACTGGTACTGGTCAGGCAATAACAGTTACACCGGAAGCCAATATGGA |
| | | TCAAGAATCCTTTGGTGGTGCATCGTGTTGTCTGTACTGCCGTTGCCACATAGATCATCCAAATCCTAAA |
| | | GGATTTTGTGACTTAAAAGGTAAGTATGTACAAATACCTACAACTTGTGCTAATGACCCTGTGGGTTTTA |
| | | CACTTAAAAACACAGTCTGTACCGTCTGCGGTATGTGGAAAGGTTATGGCTGTAGTTGTGATCAACTCCG |
| | | CGAACCCATGCTTCAGTCAGCTGATGCACAATCGTTTTTAAACCGGGTTTGCGGTGTAAGTGCAGCCCGT |
| | | CTTACACCGTGCGGCACAGGCACTAGTACTGATGTCGTATACAGGGCTTTTGACATCTACAATGATAAAG |
| | | TAGCTGGTTTTGCTAAATTCCTAAAAACTAATTGTTGTCGCTTCCAAGAAAAGGATGAAGATGACAATTT |
| | | AATTGATTCTTACTTTGTAGTTAAGAGACACACTTTCTCTAACTACCAACATGAAGAAACAATTTATAAT |
| | | TTACTTAAGGATTGTCCAGCTGTTGCTAAACATGACTTCTTTAAGTTTAGAATAGACGGTGACATGGTAC |
| | | CACATATATCACGTCAACGTCTTACTAAATACACAATGGCAGACCTCGTCTATGCTTTAAGGCATTTTGA |
| | | TGAAGGTAATTGTGACACATTAAAAGAAATACTTGTCACATACAATTGTTGTGATGATGATTATTTCAAT |
| | | AAAAAGGACTGGTATGATTTTGTAGAAAACCCAGATATATTACGCGTATACGCCAACTTAGGTGAACGTG |
| | | TACGCCAAGCTTTGTTAAAAACAGTACAATTCTGTGATGCCATGCGAAATGCTGGTATTGTTGGTGTACT |
| | | GACATTAGATAATCAAGATCTCAATGGTAACTGGTATGATTTCGGTGATTTCATACAAACCACGCCAGGT |
| | | AGTGGAGTTCCTGTTGTAGATTCTTATTATTCATTGTTAATGCCTATATTAACCTTGACCAGGGCTTTAA |
| | | CTGCAGAGTCACATGTTGACACTGACTTAACAAAGCCTTACATTAAGTGGGATTTGTTAAAATATGACTT |
| | | CACGGAAGAGAGGTTAAAACTCTTTGACCGTTATTTTAAATATTGGGATCAGACATACCACCCAAATTGT |
| | | GTTAACTGTTTGGATGACAGATGCATTCTGCATTGTGCAAACTTTAATGTTTTATTCTCTACAGTGTTCC |
| | | CACTTACAAGTTTTGGACCACTAGTGAGAAAAATATTTGTTGATGGTGTTCCATTTGTAGTTTCAACTGG |
| | | ATACCACTTCAGAGAGCTAGGIGTTGTACATAATCAGGATGTAAACTTACATAGCTCTAGACTTAGTTTT |
| | | AAGGAATTACTTGTGTATGCTGCTGACCCTGCTATGCACGCTGCTTCTGGTAATCTATTACTAGATAAAC |
| | | GCACTACGTGCTTTTCAGTAGCTGCACTTACTAACAATGTTGCTTTTCAAACTGTCAAACCCGGTAATTT |
| | | TAACAAAGACTTCTATGACTTTGCTGTGTCTAAGGGTTTCTTTAAGGAAGGAAGTTCTGTTGAATTAAAA |
| | | CACTTCTTCTTTGCTCAGGATGGTAATGCTGCTATCAGCGATTATGACTACTATCGTTATAATCTACCAA |
| | | CAATGTGTGATATCAGACAACTACTATTTGTAGTTGAAGTTGTTGATAAGTACTTTGATTGTTACGATGG |
| | | TGGCTGTATTAATGCTAACCAAGTCATCGTCAACAACCTAGACAAATCAGCTGGTTTTCCATTTAATAAA |
| | | TGGGGTAAGGCTAGACTTTATTATGATTCAATGAGTTATGAGGATCAAGATGCACTTTTCGCATATACAA |
| | | AACGTAATGTCATCCCTACTATAACTCAAATGAATCTTAAGTATGCCATTAGTGCAAAGAATAGAGCTCG |
| | | CACCGTAGCTGGTGTCTCTATCTGTAGTACTATGACCAATAGACAGTTTCATCAAAAATTATTGAAATCA |
| | | ATAGCCGCCACTAGAGGAGCTACTGTAGTAATTGGAACAAGCAAATTCTATGGTGGTTGGCACAACATGT |
| | | TAAAAACTGTTTATAGTGATGTAGAAAACCCTCACCTTATGGGTTGGGATTATCCTAAATGTGATAGAGC |
| | | CATGCCTAACATGCTTAGAATTATGGCCTCACTTGTTCTTGCTCGCAAACATACAACGTGTTGTAGCTTG |
| | | TCACACCGTTTCTATAGATTAGCTAATGAGTGTGCTCAAGTATTGAGTGAAATGGTCATGTGTGGCGGTT |
| | | CACTATATGTTAAACCAGGTGGAACCTCATCAGGAGATGCCACAACTGCTTATGCTAATAGTGTTTTTAA |
| | | CATTTGTCAAGCTGTCACGGCCAATGTTAATGCACTTTTATCTACTGATGGTAACAAAATTGCCGATAAG |
| | | TATGTCCGCAATTTACAACACAGACTTTATGAGTGTCTCTATAGAAATAGAGATGTTGACACAGACTTTG |
| | | TGAATGAGTTTTACGCATATTTGCGTAAACATTTCTCAATGATGATACTCTCTGACGATGCTGTTGTGTG |
| | | TTTCAATAGCACTTATGCATCTCAAGGTCTAGTGGCTAGCATAAAGAACTTTAAGTCAGTTCTTTATTAT |
| | | CAAAACAATGTTTTTATGTCTGAAGCAAAATGTTGGACTGAGACTGACCTTACTAAAGGACCTCATGAAT |
| | | TTTGCTCTCAACATACAATGCTAGTTAAACAGGGTGATGATTATGTGTACCTTCCTTACCCAGATCCATC |
| | | AAGAATCCTAGGGGCCGGCTGTTTTGTAGATGATATCGTAAAAACAGATGGTACACTTATGATTGAACGG |
| | | TTCGTGTCTTTAGCTATAGATGCTTACCCACTTACTAAACATCCTAATCAGGAGTATGCTGATGTCTTTC |
| | | ATTTGTACTTACAATACATAAGAAAGCTACATGATGAGTTAACAGGACACATGTTAGACATGTATTCTGT |
| | | TATGCTTACTAATGATAACACTTCAAGGTATTGGGAACCTGAGTTTTATGAGGCTATGTACACACCGCAT |
| | | ACAGTCTTACAGGCTGTTGGGGCTTGTGTTCTTTGCAATTCACAGACTTCATTAAGATGTGGTGCTTGCA |
| | | TACGTAGACCATTCTTATGTTGTAAATGCTGTTACGACCATGTCATATCAACATCACATAAATTAGTCTT |
| | | GTCTGTTAATCCGTATGTTTGCAATGCTCCAGGTTGTGATGTCACAGATGTGACTCAACTTTACTTAGGA |
| | | GGTATGAGCTATTATTGTAAATCACATAAACCACCCATTAGTTTTCCATTGTGTGCTAATGGACAAGTTT |
| | | TTGGTTTATATAAAAATACATGTGTTGGTAGCGATAATGTTACTGACTTTAATGCAATTGCAACATGTGA |
| | | CTGGACAAATGCTGGTGATTACATTTTAGCTAACACCTGTACTGAAAGACTCAAGCTTTTTGCAGCAGAA |
| | | ACGCTCAAAGCTACTGAGGAGACATTTAAACTGTCTTATGGTATTGCTACTGTACGTGAAGTGCTGTCTG |
| | | ACAGAGAATTACATCTTTCATGGGAAGTTGGTAAACCTAGACCACCACTTAACCGAAATTATGTCTTTAC |
| | | TGGTTATCGTGTAACTAAAAACAGTAAAGTACAAATAGGAGAGTACACCTTTGAAAAAGGTGACTATGGT |
| | | GATGCTGTTGTTTACCGAGGTACAACAACTTACAAATTAAATGTTGGTGATTATTTTGTGCTGACATCAC |
| | | ATACAGTAATGCCATTAAGTGCACCTACACTAGTGCCACAAGAGCACTATGTTAGAATTACTGGCTTATA |
| | | CCCAACACTCAATATCTCAGATGAGTTTTCTAGCAATGTTGCAAATTATCAAAAGGTTGGTATGCAAAAG |
| | | TATTCTACACTCCAGGGACCACCTGGTACTGGTAAGAGTCATTTTGCTATTGGCCTAGCTCTCTACTACC |
| | | CTTCTGCTCGCATAGTGTATACAGCTTGCTCTCATGCCGCTGTTGATGCACTATGTGAGAAGGCATTAAA |
| | | ATATTTGCCTATAGATAAATGTAGTAGAATTATACCTGCACGTGCTCGTGTAGAGTGTTTTGATAAATTC |
| | | AAAGTGAATTCAACATTAGAACAGTATGTCTTTTGTACTGTAAATGCATTGCCTGAGACGACAGCAGATA |
| | | TAGTTGTCTTTGATGAAATTTCAATGGCCACAAATTATGATTTGAGTGTTGTCAATGCCAGATTACGTGC |
| | | TAAGCACTATGTGTACATTGGCGACCCTGCTCAATTACCTGCACCACGCACATTGCTAACTAAGGGCACA |
| | | CTAGAACCAGAATATTTCAATTCAGTGTGTAGACTTATGAAAACTATAGGTCCAGACATGTTCCTCGGAA |
| | | CTTGTCGGCGTTGTCCTGCTGAAATTGTTGACACTGTGAGTGCTTTGGTTTATGATAATAAGCTTAAAGC |
| | | ACATAAAGACAAATCAGCTCAATGCTTTAAAATGTTTTATAAGGGTGTTATCACGCATGATGTTTCATCT |
| | | GCAATTAACAGGCCACAAATAGGCGTGGTAAGAGAATTCCTTACACGTAACCCTGCTTGGAGAAAAGCTG |
| | | TCTTTATTTCACCTTATAATTCACAGAATGCTGTAGCCTCAAAGATTTTGGGACTACCAACTCAAACTGT |
| | | TGATTCATCACAGGGCTCAGAATATGACTATGTCATATTCACTCAAACCACTGAAACAGCTCACTCTTGT |
| | | AATGTAAACAGATTTAATGTTGCTATTACCAGAGCAAAAGTAGGCATACTTTGCATAATGTCTGATAGAG |
| | | ACCTTTATGACAAGTTGCAATTTACAAGTCTTGAAATTCCACGTAGGAATGTGGCAACTTTACAAGCTGA |
| | | AAATGTAACAGGACTCTTTAAAGATTGTAGTAAGGTAATCACTGGGTTACATCCTACACAGGCACCTACA |
| | | CACCTCAGTGTTGACACTAAATTCAAAACTGAAGGTTTATGTGTTGACATACCTGGCATACCTAAGGACA |
| | | TGACCTATAGAAGACTCATCTCTATGATGGGTTTTAAAATGAATTATCAAGTTAATGGTTACCCTAACAT |
| | | GTTTATCACCCGCGAAGAAGCTATAAGACATGTACGTGCATGGATTGGCTTCGATGTCGAGGGGTGTCAT |
| | | GCTACTAGAGAAGCTGTTGGTACCAATTTACCTTTACAGCTAGGTTTTTCTACAGGTGTTAACCTAGTTG |
| | | CTGTACCTACAGGTTATGTTGATACACCTAATAATACAGATTTTTCCAGAGTTAGTGCTAAACCACCGCC |
| | | TGGAGATCAATTTAAACACCTCATACCACTTATGTACAAAGGACTTCCTTGGAATGTAGTGCGTATAAAG |
| | | ATTGTACAAATGTTAAGTGACACACTTAAAAATCTCTCTGACAGAGTCGTATTTGTCTTATGGGCACATG |
| | | GCTTTGAGTTGACATCTATGAAGTATTTTGTGAAAATAGGACCTGAGCGCACCTGTTGTCTATGTGATAG |
| | | ACGTGCCACATGCTTTTCCACTGCTTCAGACACTTATGCCTGTTGGCATCATTCTATTGGATTTGATTAC |
| | | GTCTATAATCCGTTTATGATTGATGTTCAACAATGGGGTTTTACAGGTAACCTACAAAGCAACCATGATC |
| | | TGTATTGTCAAGTCCATGGTAATGCACATGTAGCTAGTTGTGATGCAATCATGACTAGGTGTCTAGCTGT |
| | | CCACGAGTGCTTTGTTAAGCGTGTTGACTGGACTATTGAATATCCTATAATTGGTGATGAACTGAAGATT |
| | | AATGCGGCTTGTAGAAAGGTTCAACACATGGTTGTTAAAGCTGCATTATTAGCAGACAAATTCCCAGTTC |
| | | TTCACGACATTGGTAACCCTAAAGCTATTAAGTGTGTACCTCAAGCTGATGTAGAATGGAAGTTCTATGA |
| | | TGCACAGCCTTGTAGTGACAAAGCTTATAAAATAGAAGAATTATTCTATTCTTATGCCACACATTCTGAC |
| | | AAATTCACAGATGGTGTATGCCTATTTTGGAATTGCAATGTCGATAGATATCCTGCTAATTCCATTGTTT |
| | | GTAGATTTGACACTAGAGTGCTATCTAACCTTAACTTGCCTGGTTGTGATGGTGGCAGTTTGTATGTAAA |
| | | TAAACATGCATTCCACACACCAGCTTTTGATAAAAGTGCTTTTGTTAATTTAAAACAATTACCATTTTTC |
| | | TATTACTCTGACAGTCCATGTGAGTCTCATGGAAAACAAGTAGTGTCAGATATAGATTATGTACCACTAA |
| | | AGTCTGCTACGTGTATAACACGTTGCAATTTAGGTGGTGCTGTCTGTAGACATCATGCTAATGAGTACAG |
| | | ATTGTATCTCGATGCTTATAACATGATGATCTCAGCTGGCTTTAGCTTGTGGGTTTACAAACAATTTGAT |
| | | ACTTATAACCTCTGGAACACTTTTACAAGACTTCAGAGTTTAGAAAATGTGGCTTTTAATGTTGTAAATA |
| | | AGGGACACTTTGATGGACAACAGGGTGAAGTACCAGTTTCTATCATTAATAACACTGTTTACACAAAAGT |
| | | TGATGGTGTTGATGTAGAATTGTTTGAAAATAAAACAACATTACCTGTTAATGTAGCATTTGAGCTTTGG |
| | | GCTAAGCGCAACATTAAACCAGTACCAGAGGTGAAAATACTCAATAATTTGGGTGTGGACATTGCTGCTA |
| | | ATACTGTGATCTGGGACTACAAAAGAGATGCTCCAGCACATATATCTACTATTGGTGTTTGTTCTATGAC |
| | | TGACATAGCCAAGAAACCAACTGAAACGATTTGTGCACCACTCACTGTCTTTTTTGATGGTAGAGTTGAT |
| | | GGTCAAGTAGACTTATTTAGAAATGCCCGTAATGGTGTTCTTATTACAGAAGGTAGTGTTAAAGGTTTAC |
| | | AACCATCTGTAGGTCCCAAACAAGCTAGTCTTAATGGAGTCACATTAATTGGAGAAGCCGTAAAAACACA |
| | | GTTCAATTATTATAAGAAAGTTGATGGTGTTGTCCAACAATTACCTGAAACTTACTTTACTCAGAGTAGA |
| | | AATTTACAAGAATTTAAACCCAGGAGTCAAATGGAAATTGATTTCTTAGAATTAGCTATGGATGAATTCA |
| | | TTGAACGGTATAAATTAGAAGGCTATGCCTTCGAACATATCGTTTATGGAGATTTTAGTCATAGTCAGTT |
| | | AGGTGGTTTACATCTACTGATTGGACTAGCTAAACGTTTTAAGGAATCACCTTTTGAATTAGAAGATTTT |
| | | ATTCCTATGGACAGTACAGTTAAAAACTATTTCATAACAGATGCGCAAACAGGTTCATCTAAGTGTGTGT |
| | | GTTCTGTTATTGATTTATTACTTGATGATTTTGTTGAAATAATAAAATCCCAAGATTTATCTGTAGTTTC |
| | | TAAGGTTGTCAAAGTGACTATTGACTATACAGAAATTTCATTTATGCTTTGGTGTAAAGATGGCCATGTA |
| | | GAAACATTTTACCCAAAATTACAATCTAGTCAAGCGTGGCAACCGGGTGTTGCTATGCCTAATCTTTACA |
| | | AAATGCAAAGAATGCTATTAGAAAAGTGTGACCTTCAAAATTATGGTGATAGTGCAACATTACCTAAAGG |
| | | CATAATGATGAATGTCGCAAAATATACTCAACTGTGTCAATATTTAAACACATTAACATTAGCTGTACCC |
| | | TATAATATGAGAGTTATACATTTTGGTGCTGGTTCTGATAAAGGAGTTGCACCAGGTACAGCTGTTTTAA |
| | | GACAGTGGTTGCCTACGGGTACGCTGCTTGTCGATTCAGATCTTAATGACTTTGTCTCTGATGCAGATTC |
| | | AACTTTGATTGGTGATTGTGCAACTGTACATACAGCTAATAAATGGGATCTCATTATTAGTGATATGTAC |
| | | GACCCTAAGACTAAAAATGTTACAAAAGAAAATGACTCTAAAGAGGGTTTTTTCACTTACATTTGTGGGT |
| | | TTATACAACAAAAGCTAGCTCTTGGAGGTTCCGTGGCTATAAAGATAACAGAACATTCTTGGAATGCTGA |
| | | TCTTTATAAGCTCATGGGACACTTCGCATGGTGGACAGCCTTTGTTACTAATGTGAATGCGTCATCATCT |
| | | GAAGCATTTTTAATTGGATGTAATTATCTTGGCAAACCACGCGAACAAATAGATGGTTATGTCATGCATG |
| | | CAAATTACATATTTTGGAGGAATACAAATCCAATTCAGTTGTCTTCCTATTCTTTATTTGACATGAGTAA |
| | | ATTTCCCCTTAAATTAAGGGGTACTGCTGTTATGTCTTTAAAAGAAGGTCAAATCAATGATATGATTTTA |
| | | TCTCTTCTTAGTAAAGGTAGACTTATAATTAGAGAAAACAACAGAGTTGTTATTTCTAGTGATGTTCTTG |
| | | TTAACAACTAA |
| 4 | Severe | ATGGAGAGCCTTGTCCCTGGTTTCAACGAGAAAACACACGTCCAACTCAGTTTGCCTGTTTTACAGGTTC |
| | acute | GCGACGTGCTCGTACGTGGCTTTGGAGACTCCGTGGAGGAGGTCTTATCAGAGGCACGTCAACATCTTAA |
| | respiratory | AGATGGCACTTGTGGCTTAGTAGAAGTTGAAAAAGGCGTTTTGCCTCAACTTGAACAGCCCTATGTGTTC |
| | syndrome | ATCAAACGTTCGGATGCTCGAACTGCACCTCATGGTCATGTTATGGTTGAGCTGGTAGCAGAACTCGAAG |
| | coronavirus 2 | GCATTCAGTACGGTCGTAGTGGTGAGACACTTGGTGTCCTTGTCCCTCATGTGGGCGAAATACCAGTGGC |
| | isolate | TTACCGCAAGGTTCTTCTTCGTAAGAACGGTAATAAAGGAGCTGGTGGCCATAGTTACGGCGCCGATCTA |
| | SARS- | AAGTCATTTGACTTAGGCGACGAGCTTGGCACTGATCCTTATGAAGATTTTCAAGAAAACTGGAACACTA |
| | CoV- | AACATAGCAGTGGTGTTACCCGTGAACTCATGCGTGAGCTTAACGGAGGGGCATACACTCGCTATGTCGA |
| | 2/human/ | TAACAACTTCTGTGGCCCTGATGGCTACCCTCTTGAGTGCATTAAAGACCTTCTAGCACGTGCTGGTAAA |
| | BRA/LRV- | GCTTCATGCACTTTGTCCGAACAACTGGACTTTATTGACACTAAGAGGGGTGTATACTGCTGCCGTGAAC |
| | SARS.CoV- | ATGAGCATGAAATTGCTTGGTACACGGAACGTTCTGAAAAGAGCTATGAATTGCAGACACCTTTTGAAAT |
| | 2.1/2020, | TAAATTGGCAAAGAAATTTGACACCTTCAATGGGGAATGTCCAAATTTTGTATTTCCCTTAAATTCCATA |
| | complete | ATCAAGACTATTCAACCAAGGGTTGAAAAGAAAAAGCTTGATGGCTTTATGGGTAGAATTCGATCTGTCT |
| | genome | ATCCAGTTGCGTCACCAAATGAATGCAACCAAATGTGCCTTTCAACTCTCATGAAGTGTGATCATTGTGG |
| | | TGAAACTTCATGGCAGACGGGCGATTTTGTTAAAGCCACTTGCGAATTTTGTGGCACTGAGAATTTGACT |
| | | AAAGAAGGTGCCACTACTTGTGGTTACTTACCCCAAAATGCTGTTGTTAAAATTTATTGTCCAGCATGTC |
| | | ACAATTCAGAAGTAGGACCTGAGCATAGTCTTGCCGAATACCATAATGAATCTGGCTTGAAAACCATTCT |
| | | TCGTAAGGGTGGTCGCACTATTGCCTTTGGAGGCTGTGTGTTCTCTTATGTTGGTTGCCATAACAAGTGT |
| | | GCCTATTGGGTTCCACGTGCTAGCGCTAACATAGGTTGTAACCATACAGGTGTTGTTGGAGAAGGTTCCG |
| | | AAGGTCTTAATGACAACCTTCTTGAAATACTCCAAAAAGAGAAAGTCAACATCAATATTGTTGGTGACTT |
| | | TAAACTTAATGAAGAGATCGCCATTATTTTGGCATCTTTTTCTGCTTCCACAAGTGCTTTTGTGGAAACT |
| | | GTGAAAGGTTTGGATTATAAAGCATTCAAACAAATTGTTGAATCCTGTGGTAATTTTAAAGTTACAAAAG |
| | | GAAAAGCTAAAAAAGGTGCCTGGAATATTGGTGAACAGAAATCAATACTGAGTCCTCTTTATGCATTTGC |
| | | ATCAGAGGCTGCTCGTGTTGTACGATCAATTTTCTCCCGCACTCTTGAAACTGCTCAAAATTCTGTGCGT |
| | | GTTTTACAGAAGGCCGCTATAACAATACTAGATGGAATTTCACAGTATTCACTGAGACTCATTGATGCTA |
| | | TGATGTTCACATCTGATTTGGCTACTAACAATCTAGTTGTAATGGCCTACATTACAGGTGGTGTTGTTCA |
| | | GTTGACTTCGCAGTGGCTAACTAACATCTTTGGCACTGTTTATGAAAAACTCAAACCCGTCCTTGATTGG |
| | | CTTGAAGAGAAGTTTAAGGAAGGTGTAGAGTTTCTTAGAGACGGTTGGGAAATTGTTAAATTTATCTCAA |
| | | CCTGTGCTTGTGAAATTGTCGGTGGACAAATTGTCACCTGTGCAAAGGAAATTAAGGAGAGTGTTCAGAC |
| | | ATTCTTTAAGCTTGTAAATAAATTTTTGGCTTTGTGTGCTGACTCTATCATTATTGGTGGAGCTAAACTT |
| | | AAAGCCTTGAATTTAGGTGAAACATTTGTCACGCACTCAAAGGGATTGTACAGAAAGTGTGTTAAATCCA |
| | | GAGAAGAAACTGGCCTACTCATGCCTCTAAAAGCCCCAAAAGAAATTATCTTCTTAGAGGGAGAAACACT |
| | | TCCCACAGAAGTGTTAACAGAGGAAGTTGTCTTGAAAACTGGTGATTTACAACCATTAGAACAACCTACT |
| | | AGTGAAGCTGTTGAAGCTCCATTGGTTGGTACACCAGTTTGTATTAACGGGCTTATGTTGCTCGAAATCA |
| | | AAGACACAGAAAAGTACTGTGCCCTTGCACCTAATATGATGGTAACAAACAATACCTTCACACTCAAAGG |
| | | CGGTGCACCAACAAAGGTTACTTTTGGTGATGACACTGTGATAGAAGTGCAAGGTTACAAGAGTGTGAAT |
| | | ATCACTTTTGAACTTGATGAAAGGATTGATAAAGTACTTAATGAGAAGTGCTCTGCCTATACAGTTGAAC |
| | | TCGGTACAGAAGTAAATGAGTTCGCCTGTGTTGTGGCAGATGCTGTCATAAAAACTTTGCAACCAGTATC |
| | | TGAATTACTTACACCACTGGGCATTGATTTAGATGAGTGGAGTATGGCTACATACTACTTATTTGATGAG |
| | | TCTGGTGAGTTTAAATTGGCTTCACATATGTATTGTTCTTTTTACCCTCCAGATGAGGATGAAGAAGAAG |
| | | GTGATTGTGAAGAAGAAGAGTTTGAGCCATCAACTCAATATGAGTATGGTACTGAAGATGATTACCAAGG |
| | | TAAACCTTTGGAATTTGGTGCCACTTCTGCTGCTCTTCAACCTGAAGAAGAGCAAGAAGAAGATTGGTTA |
| | | GATGATGATAGTCAACAAACTGTTGGTCAACAAGACGGCAGTGAGGACAATCAGACAACTACTATTCAAA |
| | | CAATTGTTGAGGTTCAACCTCAATTAGAGATGGAACTTACACCAGTTGTTCAGACTATTGAAGTGAATAG |
| | | TTTTAGTGGTTATTTAAAACTTACTGACAATGTATACATTAAAAATGCAGACATTGTGGAAGAAGCTAAA |
| | | AAGGTAAAACCAACAGTGGTTGTTAATGCAGCCAATGTTTACCTTAAACATGGAGGAGGTGTTGCAGGAG |
| | | CCTTAAATAAGGCTACTAACAATGCCATGCAAGTTGAATCTGATGATTACATAGCTACTAATGGACCACT |
| | | TAAAGTGGGTGGTAGTTGTGTTTTAAGCGGACACAATCTTGCTAAACACTGTCTTCATGTTGTCGGCCCA |
| | | AATGTTAACAAAGGTGAAGACATTCAACTTCTTAAGAGTGCTTATGAAAATTTTAATCAGCACGAAGTTC |
| | | TACTTGCACCATTATTATCAGCTGGTATTTTTGGTGCTGACCCTATACATTCTTTAAGAGTTTGTGTAGA |
| | | TACTGTTCGCACAAATGTCTACTTAGCTGTCTTTGATAAAAATCTCTATGACAAACTTGTTTCAAGCTTT |
| | | TTGGAAATGAAGAGTGAAAAGCAAGTTGAACAAAAGATCGCTGAGATTCCTAAAGAGGAAGTTAAGCCAT |
| | | TTATAACTGAAAGTAAACCTTCAGTTGAACAGAGAAAACAAGATGATAAGAAAATCAAAGCTTGTGTTGA |
| | | AGAAGTTACAACAACTCTGGAAGAAACTAAGTTCCTCACAGAAAACTTGTTACTTTATATTGACATTAAT |
| | | GGCAATCTTCATCCAGATTCTGCCACTCTTGTTAGTGACATTGACATCACTTTCTTAAAGAAAGATGCTC |
| | | CATATATAGTGGGTGATGTTGTTCAAGAGGGTGTTTTAACTGCTGTGGTTATACCTACTAAAAAGGCTGG |
| | | TGGCACTACTGAAATGCTAGCGAAAGCTTTGAGAAAAGTGCCAACAGACAATTATATAACCACTTACCCG |
| | | GGTCAGGGTTTAAATGGTTACACTGTAGAGGAGGCAAAGACAGTGCTTAAAAAGTGTAAAAGTGCCTTTT |
| | | ACATTCTACCATCTATTATCTCTAATGAGAAGCAAGAAATTCTTGGAACTGTTTCTTGGAATTTGCGAGA |
| | | AATGCTTGCACATGCAGAAGAAACACGCAAATTAATGCCTGTCTGTGTGGAAACTAAAGCCATAGTTTCA |
| | | ACTATACAGCGTAAATATAAGGGTATTAAAATACAAGAGGGTGTGGTTGATTATGGTGCTAGATTTTACT |
| | | TTTACACCAGTAAAACAACTGTAGCGTCACTTATCAACACACTTAACGATCTAAATGAAACTCTTGTTAC |
| | | AATGCCACTTGGCTATGTAACACATGGCTTAAATTTGGAAGAAGCTGCTCGGTATATGAGATCTCTCAAA |
| | | GTGCCAGCTACAGTTTCTGTTTCTTCACCTGATGCTGTTACAGCGTATAATGGTTATCTTACTTCTTCTT |
| | | CTAAAACACCTGAAGAACATTTTATTGAAACCATCTCACTTGCTGGTTCCTATAAAGATTGGTCCTATTC |
| | | TGGACAATCTACACAACTAGGTATAGAATTTCTTAAGAGAGGTGATAAAAGTGTATATTACACTAGTAAT |
| | | CCTACCACATTCCACCTAGATGGTGAAGTTATCACCTTTGACAATCTTAAGACACTTCTTTCTTTGAGAG |
| | | AAGTGAGGACTATTAAGGTGTTTACAACAGTAGACAACATTAACCTCCACACGCAAGTTGTGGACATGTC |
| | | AATGACATATGGACAACAGTTTGGTCCAACTTATTTGGATGGAGCTGATGTTACTAAAATAAAACCTCAT |
| | | AATTCACATGAAGGTAAAACATTTTATGTTTTACCTAATGATGACACTCTACGTGTTGAGGCTTTTGAGT |
| | | ACTACCACACAACTGATCCTAGTTTTCTGGGTAGGTACATGTCAGCATTAAATCACACTAAAAAGTGGAA |
| | | ATACCCACAAGTTAATGGTTTAACTTCTATTAAATGGGCAGATAACAACTGTTATCTTGCCACTGCATTG |
| | | TTAACACTCCAACAAATAGAGTTGAAGTTTAATCCACCTGCTCTACAAGATGCTTATTACAGAGCAAGGG |
| | | CTGGTGAAGCTGCTAACTTTTGTGCACTTATCTTAGCCTACTGTAATAAGACAGTAGGTGAGTTAGGTGA |
| | | TGTTAGAGAAACAATGAGTTACTTGTTTCAACATGCCAATTTAGATTCTTGCAAAAGAGTCTTGAACGTG |
| | | GTGTGTAAAACTTGTGGACAACAGCAGACAACCCTTAAGGGTGTAGAAGCTGTTATGTACATGGGCACAC |
| | | TTTCTTATGAACAATTTAAGAAAGGTGTTCAGATACCTTGTACGTGTGGTAAACAAGCTACAAAATATCT |
| | | AGTACAACAGGAGTCACCTTTTGTTATGATGTCAGCACCACCTGCTCAGTATGAACTTAAGCATGGTACA |
| | | TTTACTTGTGCTAGTGAGTACACTGGTAATTACCAGTGTGGTCACTATAAACATATAACTTCTAAAGAAA |
| | | CTTTGTATTGCATAGACGGTGCTTTACTTACAAAGTCCTCAGAATACAAAGGTCCTATTACGGATGTTTT |
| | | CTACAAAGAAAACAGTTACACAACAACCATAAAACCAGTTACTTATAAATTGGATGGTGTTGTTTGTACA |
| | | GAAATTGACCCTAAGTTGGACAATTATTATAAGAAAGACAATTCTTATTTCACAGAGCAACCAATTGATC |
| | | TTGTACCAAACCAACCATATCCAAACGCAAGCTTCGATAATTTTAAGTTTGTATGTGATAATATCAAATT |
| | | TGCTGATGATTTAAACCAGTTAACTGGTTATAAGAAACCTGCTTCAAGAGAGCTTAAAGTTACATTTTTC |
| | | CCTGACTTAAATGGTGATGTGGTGGCTATTGATTATAAACACTACACACCCTCTTTTAAGAAAGGAGCTA |
| | | AATTGTTACATAAACCTATTGTTTGGCATGTTAACAATGCAACTAATAAAGCCACGTATAAACCAAATAC |
| | | CTGGTGTATACGTTGTCTTTGGAGCACAAAACCAGTTGAAACATCAAATTCGTTTGATGTACTGAAGTCA |
| | | GAGGACGCGCAGGGAATGGATAATCTTGCCTGCGAAGATCTAAAACCAGTCTCTGAAGAAGTAGTGGAAA |
| | | ATCCTACCATACAGAAAGACGTTCTTGAGTGTAATGTGAAAACTACCGAAGTTGTAGGAGACATTATACT |
| | | TAAACCAGCAAATAATAGTTTAAAAATTACAGAAGAGGTTGGCCACACAGATCTAATGGCTGCTTATGTA |
| | | GACAATTCTAGTCTTACTATTAAGAAACCTAATGAATTATCTAGAGTATTAGGTTTGAAAACCCTTGCTA |
| | | CTCATGGTTTAGCTGCTGTTAATAGTGTCCCTTGGGATACTATAGCTAATTATGCTAAGCCTTTTCTTAA |
| | | CAAAGTTGTTAGTACAACTACTAACATAGTTACACGGTGTTTAAACCGTGTTTGTACTAATTATATGCCT |
| | | TATTTCTTTACTTTATTGCTACAATTGTGTACTTTTACTAGAAGTACAAATTCTAGAATTAAAGCATCTA |
| | | TGCCGACTACTATAGCAAAGAATACTGTTAAGAGTGTCGGTAAATTTTGTCTAGAGGCTTCATTTAATTA |
| | | TTTGAAGTCACCTAATTTTTCTAAACTGATAAATATTATAATTTGGTTTTTACTATTAAGTGTTTGCCTA |
| | | GGTTCTTTAATCTACTCAACCGCTGCTTTAGGTGTTTTAATGTCTAATTTAGGCATGCCTTCTTACTGTA |
| | | CTGGTTACAGAGAAGGCTATTTGAACTCTACTAATGTCACTATTGCAACCTACTGTACTGGTTCTATACC |
| | | TTGTAGTGTTTGTCTTAGTGGTTTAGATTCTTTAGACACCTATCCTTCTTTAGAAACTATACAAATTACC |
| | | ATTTCATCTTTTAAATGGGATTTAACTGCTTTTGGCTTAGTTGCAGAGTGGTTTTTGGCATATATTCTTT |
| | | TCACTAGGTTTTTCTATGTACTTGGATTGGCTGCAATCATGCAATTGTTTTTCAGCTATTTTGCAGTACA |
| | | TTTTATTAGTAATTCTTGGCTTATGTGGTTAATAATTAATCTTGTACAAATGGCCCCGATTTCAGCTATG |
| | | GTTAGAATGTACATCTTCTTTGCATCATTTTATTATGTATGGAAAAGTTATGTGCATGTTGTAGACGGTT |
| | | GTAATTCATCAACTTGTATGATGTGTTACAAACGTAATAGAGCAACAAGAGTCGAATGTACAACTATTGT |
| | | TAATGGTGTTAGAAGGTCCTTTTATGTCTATGCTAATGGAGGTAAAGGCTTTTGCAAACTACACAATTGG |
| | | AATTGTGTTAATTGTGATACATTCTGTGCTGGTAGTACATTTATTAGTGATGAAGTTGCGAGAGACTTGT |
| | | CACTACAGTTTAAAAGACCAATAAATCCTACTGACCAGTCTTCTTACATCGTTGATAGTGTTACAGTGAA |
| | | GAATGGTTCCATCCATCTTTACTTTGATAAAGCTGGTCAAAAGACTTATGAAAGACATTCTCTCTCTCAT |
| | | TTTGTTAACTTAGACAACCTGAGAGCTAATAACACTAAAGGTTCATTGCCTATTAATGTTATAGTTTTTG |
| | | ATGGTAAATCAAAATGTGAAGAATCATCTGCAAAATCAGCGTCTGTTTACTACAGTCAGCTTATGTGTCA |
| | | ACCTATACTGTTACTAGATCAGGCATTAGTGTCTGATGTTGGTGATAGTGCGGAAGTTGCAGTTAAAATG |
| | | TTTGATGCTTACGTTAATACGTTTTCATCAACTTTTAACGTACCAATGGAAAAACTCAAAACACTAGTTG |
| | | CAACTGCAGAAGCTGAACTTGCAAAGAATGTGTCCTTAGACAATGTCTTATCTACTTTTATTTCAGCAGC |
| | | TCGGCAAGGGTTTGTTGATTCAGATGTAGAAACTAAAGATGTTGTTGAATGTCTTAAATTGTCACATCAA |
| | | TCTGACATAGAAGTTACTGGCGATAGTTGTAATAACTATATGCTCACCTATAACAAAGTTGAAAACATGA |
| | | CACCCCGTGACCTTGGTGCTTGTATTGACTGTAGTGCGCGTCATATTAATGCGCAGGTAGCAAAAAGTCA |
| | | CAACATTGCTTTGATATGGAACGTTAAAGATTTCATGTCATTGTCTGAACAACTACGAAAACAAATACGT |
| | | AGTGCTGCTAAAAAGAATAACTTACCTTTTAAGTTGACATGTGCAACTACTAGACAAGTTGTTAATGTTG |
| | | TAACAACAAAGATAGCACTTAAGGGTGGTAAAATTGTTAATAATTGGTTGAAGCAGTTAATTAAAGTTAC |
| | | ACTTGTGTTCCTTTTTGTTGCTGCTATTTTCTATTTAATAACACCTGTTCATGTCATGTCTAAACATACT |
| | | GACTTTTCAAGTGAAATCATAGGATACAAGGCTATTGATGGTGGTGTCACTCGTGACATAGCATCTACAG |
| | | ATACTTGTTTTGCTAACAAACATGCTGATTTTGACACATGGTTTAGCCAGCGTGGTGGTAGTTATACTAA |
| | | TGACAAAGCTTGCCCATTGATTGCTGCAGTCATAACAAGAGAAGTGGGTTTTGTCGTGCCTGGTTTGCCT |
| | | GGCACGATATTACGCACAACTAATGGTGACTTTTTGCATTTCTTACCTAGAGTTTTTAGTGCAGTTGGTA |
| | | ACATCTGTTACACACCATCAAAACTTATAGAGTACACTGACTTTGCAACATCAGCTTGTGTTTTGGCTGC |
| | | TGAATGTACAATTTTTAAAGATGCTTCTGGTAAGCCAGTACCATATTGTTATGATACCAATGTACTAGAA |
| | | GGTTCTGTTGCTTATGAAAGTTTACGCCCTGACACACGTTATGTGCTCATGGATGGCTCTATTATTCAAT |
| | | TTCCTAACACCTACCTTGAAGGTTCTGTTAGAGTGGTAACAACTTTTGATTCTGAGTACTGTAGGCACGG |
| | | CACTTGTGAAAGATCAGAAGCTGGTGTTTGTGTATCTACTAGTGGTAGATGGGTACTTAACAATGATTAT |
| | | TACAGATCTTTACCAGGAGTTTTCTGTGGTGTAGATGCTGTAAATTTACTTACTAATATGTTTACACCAC |
| | | TAATTCAACCTATTGGTGCTTTGGACATATCAGCATCTATAGTAGCTGGTGGTATTGTAGCTATCGTAGT |
| | | AACATGCCTTGCCTACTATTTTATGAGGTTTAGAAGAGCTTTTGGTGAATACAGTCATGTAGTTGCCTTT |
| | | AATACTTTACTATTCCTTATGTCATTCACTGTACTCTGTTTAACACCAGTTTACTCATTCTTACCTGGTG |
| | | TTTATTCTGTTATTTACTTGTACTTGACATTTTATCTTACTAATGATGTTTCTTTTTTAGCACATATTCA |
| | | GTGGATGGTTATGTTCACACCTTTAGTACCTTTCTGGATAACAATTGCTTATATCATTTGTATTTCCACA |
| | | AAGCATTTCTATTGGTTCTTTAGTAATTACCTAAAGAGACGTGTAGTCTTTAATGGTGTTTCCTTTAGTA |
| | | CTTTTGAAGAAGCTGCGCTGTGCACCTTTTTGTTAAATAAAGAAATGTATCTAAAGTTGCGTAGTGATGT |
| | | GCTATTACCTCTTACGCAATATAATAGATACTTAGCTCTTTATAATAAGTACAAGTATTTTAGTGGAGCA |
| | | ATGGATACAACTAGCTACAGAGAAGCTGCTTGTTGTCATCTCGCAAAGGCTCTCAATGACTTCAGTAACT |
| | | CAGGTTCTGATGTTCTTTACCAACCACCACAAACCTCTATCACCTCAGCTGTTTTGCAGAGTGGTTTTAG |
| | | AAAAATGGCATTCCCATCTGGTAAAGTTGAGGGTTGTATGGTACAAGTAACTTGTGGTACAACTACACTT |
| | | AACGGTCTTTGGCTTGATGACGTAGTTTACTGTCCAAGACATGTGATCTGCACCTCTGAAGACATGCTTA |
| | | ACCCTAATTATGAAGATTTACTCATTCGTAAGTCTAATCATAATTTCTTGGTACAGGCTGGTAATGTTCA |
| | | ACTCAGGGTTATTGGACATTCTATGCAAAATTGTGTACTTAAGCTTAAGGTTGATACAGCCAATCCTAAG |
| | | ACACCTAAGTATAAGTTTGTTCGCATTCAACCAGGACAGACTTTTTCAGTGTTAGCTTGTTACAATGGTT |
| | | CACCATCTGGTGTTTACCAATGTGCTATGAGGCCCAATTTCACTATTAAGGGTTCATTCCTTAATGGTTC |
| | | ATGTGGTAGTGTTGGTTTTAACATAGATTATGACTGTGTCTCTTTTTGTTACATGCACCATATGGAATTA |
| | | CCAACTGGAGTTCATGCTGGCACAGACTTAGAAGGTAACTTTTATGGACCTTTTGTTGACAGGCAAACAG |
| | | CACAAGCAGCTGGTACGGACACAACTATTACAGTTAATGTTTTAGCTTGGTTGTACGCTGCTGTTATAAA |
| | | TGGAGACAGGTGGTTTCTCAATCGATTTACCACAACTCTTAATGACTTTAACCTTGTGGCTATGAAGTAC |
| | | AATTATGAACCTCTAACACAAGACCATGTTGACATACTAGGACCTCTTTCTGCTCAAACTGGAATTGCCG |
| | | TTTTAGATATGTGTGCTTCATTAAAAGAATTACTGCAAAATGGTATGAATGGACGTACCATATTGGGTAG |
| | | TGCTTTATTAGAAGATGAATTTACACCTTTTGATGTTGTTAGACAATGCTCAGGTGTTACTTTCCAAAGT |
| | | GCAGTGAAAAGAACAATCAAGGGTACACACCACTGGTTGTTACTCACAATTTTGACTTCACTTTTAGTTT |
| | | TAGTCCAGAGTACTCAATGGTCTTTGTTCTTTTTTTTGTATGAAAATGCCTTTTTACCTTTTGCTATGGG |
| | | TATTATTGCTATGTCTGCTTTTGCAATGATGTTTGTCAAACATAAGCATGCATTTCTCTGTTTGTTTTTG |
| | | TTACCTTCTCTTGCCACTGTAGCTTATTTTAATATGGTCTATATGCCTGCTAGTTGGGTGATGCGTATTA |
| | | TGACATGGTTGGATATGGTTGATACTAGTTTGTCTGGTTTTAAGCTAAAAGACTGTGTTATGTATGCATC |
| | | AGCTGTAGTGTTACTAATCCTTATGACAGCAAGAACTGTGTATGATGATGGTGCTAGGAGAGTGTGGACA |
| | | CTTATGAATGTCTTGACACTCGTTTATAAAGTTTATTATGGTAATGCTTTAGATCAAGCCATTTCCATGT |
| | | GGGCTCTTATAATCTCTGTTACTTCTAACTACTCAGGTGTAGTTACAACTGTCATGTTTTTGGCCAGAGG |
| | | TATTGTTTTTATGTGTGTTGAGTATTGCCCTATTTTCTTCATAACTGGTAATACACTTCAGTGTATAATG |
| | | CTAGTTTATTGTTTCTTAGGCTATTTTTGTACTTGTTACTTTGGCCTCTTTTGTTTACTCAACCGCTACT |
| | | TTAGACTGACTCTTGGTGTTTATGATTACTTAGTTTCTACACAGGAGTTTAGATATATGAATTCACAGGG |
| | | ACTACTCCCACCCAAGAATAGCATAGATGCCTTCAAACTCAACATTAAATTGTTGGGTGTTGGTGGCAAA |
| | | CCTTGTATCAAAGTAGCCACTGTACAGTCTAAAATGTCAGATGTAAAGTGCACATCAGTAGTCTTACTCT |
| | | CAGTTTTGCAACAACTCAGAGTAGAATCATCATCTAAATTGTGGGCTCAATGTGTCCAGTTACACAATGA |
| | | CATTCTCTTAGCTAAAGATACTACTGAAGCCTTTGAAAAAATGGTTTCACTACTTTCTGTTTTGCTTTCC |
| | | ATGCAGGGTGCTGTAGACATAAACAAGCTTTGTGAAGAAATGCTGGACAACAGGGCAACCTTACAAGCTA |
| | | TAGCCTCAGAGTTTAGTTCCCTTCCATCATATGCAGCTTTTGCTACTGCTCAAGAAGCTTATGAGCAGGC |
| | | TGTTGCTAATGGTGATTCTGAAGTTGTTCTTAAAAAGTTGAAGAAGTCTTTGAATGTGGCTAAATCTGAA |
| | | TTTGACCGTGATGCAGCCATGCAACGTAAGTTGGAAAAGATGGCTGATCAAGCTATGACCCAAATGTATA |
| | | AACAGGCTAGATCTGAGGACAAGAGGGCAAAAGTTACTAGTGCTATGCAGACAATGCTTTTCACTATGCT |
| | | TAGAAAGTTGGATAATGATGCACTCAACAACATTATCAACAATGCAAGAGATGGTTGTGTTCCCTTGAAC |
| | | ATAATACCTCTTACAACAGCAGCCAAACTAATGGTTGTCATACCAGACTATAACACATATAAAAATACGT |
| | | GTGATGGTACAACATTTACTTATGCATCAGCATTGTGGGAAATCCAACAGGTTGTAGATGCAGATAGTAA |
| | | AATTGTTCAACTTAGTGAAATTAGTATGGACAATTCACCTAATTTAGCATGGCCTCTTATTGTAACAGCT |
| | | TTAAGGGCCAATTCTGCTGTCAAATTACAGAATAATGAGCTTAGTCCTGTTGCACTACGACAGATGTCTT |
| | | GTGCTGCCGGTACTACACAAACTGCTTGCACTGATGACAATGCGTTAGCTTACTACAACACAACAAAGGG |
| | | AGGTAGGTTTGTACTTGCACTGTTATCCGATTTACAGGATTTGAAATGGGCTAGATTCCCTAAGAGTGAT |
| | | GGAACTGGTACTATCTATACAGAACTGGAACCACCTTGTAGGTTTGTTACAGACACACCTAAAGGTCCTA |
| | | AAGTGAAGTATTTATACTTTATTAAAGGATTAAACAACCTAAATAGAGGTATGGTACTTGGTAGTTTAGC |
| | | TGCCACAGTACGTCTACAAGCTGGTAATGCAACAGAAGTGCCTGCCAATTCAACTGTATTATCTTTCTGT |
| | | GCTTTTGCTGTAGATGCTGCTAAAGCTTACAAAGATTATCTAGCTAGTGGGGGACAACCAATCACTAATT |
| | | GTGTTAAGATGTTGTGTACACACACTGGTACTGGTCAGGCAATAACAGTTACACCGGAAGCCAATATGGA |
| | | TCAAGAATCCTTTGGTGGTGCATCGTGTTGTCTGTACTGCCGTTGCCACATAGATCATCCAAATCCTAAA |
| | | GGATTTTGTGACTTAAAAGGTAAGTATGTACAAATACCTACAACTTGTGCTAATGACCCTGTGGGTTTTA |
| | | CACTTAAAAACACAGTCTGTACCGTCTGCGGTATGTGGAAAGGTTATGGCTGTAGTTGTGATCAACTCCG |
| | | CGAACCCATGCTTCAGTCAGCTGATGCACAATCGTTTTTAAACCGGGTTTGCGGTGTAAGTGCAGCCCGT |
| | | CTTACACCGTGCGGCACAGGCACTAGTACTGATGTCGTATACAGGGCTTTTGACATCTACAATGATAAAG |
| | | TAGCTGGTTTTGCTAAATTCCTAAAAACTAATTGTTGTCGCTTCCAAGAAAAGGACGAAGATGACAATTT |
| | | AATTGATTCTTACTTTGTAGTTAAGAGACACACTTTCTCTAACTACCAACATGAAGAAACAATTTATAAT |
| | | TTACTTAAGGATTGTCCAGCTGTTGCTAAACATGACTTCTTTAAGTTTAGAATAGACGGTGACATGGTAC |
| | | CACATATATCACGTCAACGTCTTACTAAATACACAATGGCAGACCTCGTCTATGCTTTAAGGCATTTTGA |
| | | TGAAGGTAATTGTGACACATTAAAAGAAATACTTGTCACATACAATTGTTGTGATGATGATTATTTCAAT |
| | | AAAAAGGACTGGTATGATTTTGTAGAAAACCCAGATATATTACGCGTATACGCCAACTTAGGTGAACGTG |
| | | TACGCCAAGCTTTGTTAAAAACAGTACAATTCTGTGATGCCATGCGAAATGCTGGTATTGTTGGTGTACT |
| | | GACATTAGATAATCAAGATCTCAATGGTAACTGGTATGATTTCGGTGATTTCATACAAACCACGCCAGGT |
| | | AGTGGAGTTCCTGTTGTAGATTCTTATTATTCATTGTTAATGCCTATATTAACCTTGACCAGGGCTTTAA |
| | | CTGCAGAGTCACATGTTGACACTGACTTAACAAAGCCTTACATTAAGTGGGATTTGTTAAAATATGACTT |
| | | CACGGAAGAGAGGTTAAAACTCTTTGACCGTTATTTTAAATATTGGGATCAGACATACCACCCAAATTGT |
| | | GTTAACTGTTTGGATGACAGATGCATTCTGCATTGTGCAAACTTTAATGTTTTATTCTCTACAGTGTTCC |
| | | CACTTACAAGTTTTGGACCACTAGTGAGAAAAATATTTGTTGATGGTGTTCCATTTGTAGTTTCAACTGG |
| | | ATACCACTTCAGAGAGCTAGGTGTTGTACATAATCAGGATGTAAACTTACATAGCTCTAGACTTAGTTTT |
| | | AAGGAATTACTTGTGTATGCTGCTGACCCTGCTATGCACGCTGCTTCTGGTAATCTATTACTAGATAAAC |
| | | GCACTACGTGCTTTTCAGTAGCTGCACTTACTAACAATGTTGCTTTTCAAACTGTCAAACCCGGTAATTT |
| | | TAACAAAGACTTCTATGACTTTGCTGTGTCTAAGGGTTTCTTTAAGGAAGGAAGTTCTGTTGAATTAAAA |
| | | CACTTCTTCTTTGCTCAGGATGGTAATGCTGCTATCAGCGATTATGACTACTATCGTTATAATCTACCAA |
| | | CAATGTGTGATATCAGACAACTACTATTTGTAGTTGAAGTTGTTGATAAGTACTTTGATTGTTACGATGG |
| | | TGGCTGTATTAATGCTAACCAAGTCATCGTCAACAACCTAGACAAATCAGCTGGTTTTCCATTTAATAAA |
| | | TGGGGTAAGGCTAGACTTTATTATGATTCAATGAGTTATGAGGATCAAGATGCACTTTTCGCATATACAA |
| | | AACGTAATGTCATCCCTACTATAACTCAAATGAATCTTAAGTATGCCATTAGTGCAAAGAATAGAGCTCG |
| | | CACCGTAGCTGGTGTCTCTATCTGTAGTACTATGACCAATAGACAGTTTCATCAAAAATTATTGAAATCA |
| | | ATAGCCGCCACTAGAGGAGCTACTGTAGTAATTGGAACAAGCAAATTCTATGGTGGTTGGCACAACATGT |
| | | TAAAAACTGTTTATAGTGATGTAGAAAACCCTCACCTTATGGGTTGGGATTATCCTAAATGTGATAGAGC |
| | | CATGCCTAACATGCTTAGAATTATGGCCTCACTTGTTCTTGCTCGCAAACATACAACGTGTTGTAGCTTG |
| | | TCACACCGTTTCTATAGATTAGCTAATGAGTGTGCTCAAGTATTGAGTGAAATGGTCATGTGTGGCGGTT |
| | | CACTATATGTTAAACCAGGTGGAACCTCATCAGGAGATGCCACAACTGCTTATGCTAATAGTGTTTTTAA |
| | | CATTTGTCAAGCTGTCACGGCCAATGTTAATGCACTTTTATCTACTGATGGTAACAAAATTGCCGATAAG |
| | | TATGTCCGCAATTTACAACACAGACTTTATGAGTGTCTCTATAGAAATAGAGATGTTGACACAGACTTTG |
| | | TGAATGAGTTTTACGCATATTTGCGTAAACATTTCTCAATGATGATACTCTCTGACGATGCTGTTGTGTG |
| | | TTTCAATAGCACTTATGCATCTCAAGGTCTAGTGGCTAGCATAAAGAACTTTAAGTCAGTTCTTTATTAT |
| | | CAAAACAATGTTTTTATGTCTGAAGCAAAATGTTGGACTGAGACTGACCTTACTAAAGGACCTCATGAAT |
| | | TTTGCTCTCAACATACAATGCTAGTTAAACAGGGTGATGATTATGTGTACCTTCCTTACCCAGATCCATC |
| | | AAGAATCCTAGGGGCCGGCTGTTTTGTAGATGATATCGTAAAAACAGATGGTACACTTATGATTGAACGG |
| | | TTCGTGTCTTTAGCTATAGATGCTTACCCACTTACTAAACATCCTAATCAGGAGTATGCTGATGTCTTTC |
| | | ATTTGTACTTACAATACATAAGAAAGCTACATGATGAGTTAACAGGACACATGTTAGACATGTATTCTGT |
| | | TATGCTTACTAATGATAACACTTCAAGGTATTGGGAACCTGAGTTTTATGAGGCTATGTACACACCGCAT |
| | | ACAGTCTTACAGGCTGTTGGGGCTTGTGTTCTTTGCAATTCACAGACTTCATTAAGATGTGGTGCTTGCA |
| | | TACGTAGACCATTCTTATGTTGTAAATGCTGTTACGACCATGTCATATCAACATCACATAAATTAGTCTT |
| | | GTCTGTTAATCCGTATGTTTGCAATGCTCCAGGTTGTGATGTCACAGATGTGACTCAACTTTACTTAGGA |
| | | GGTATGAGCTATTATTGTAAATCACATAAACCACCCATTAGTTTTCCATTGTGTGCTAATGGACAAGTTT |
| | | TTGGTTTATATAAAAATACATGTGTTGGTAGCGATAATGTTACTGACTTTAATGCAATTGCAACATGTGA |
| | | CTGGACAAATGCTGGTGATTACATTTTAGCTAACACCTGTACTGAAAGACTCAAGCTTTTTGCAGCAGAA |
| | | ACGCTCAAAGCTACTGAGGAGACATTTAAACTGTCTTATGGTATTGCTACTGTACGTGAAGTGCTGTCTG |
| | | ACAGAGAATTACATCTTTCATGGGAAGTTGGTAAACCTAGACCACCACTTAACCGAAATTATGTCTTTAC |
| | | TGGTTATCGTGTAACTAAAAACAGTAAAGTACAAATAGGAGAGTACACCTTTGAAAAAGGTGACTATGGT |
| | | GATGCTGTTGTTTACCGAGGTACAACAACTTACAAATTAAATGTTGGTGATTATTTTGTGCTGACATCAC |
| | | ATACAGTAATGCCATTAAGTGCACCTACACTAGTGCCACAAGAGCACTATGTTAGAATTACTGGCTTATA |
| | | CCCAACACTCAATATCTCAGATGAGTTTTCTAGCAATGTTGCAAATTATCAAAAGGTTGGTATGCAAAAG |
| | | TATTCTACACTCCAGGGACCACCTGGTACTGGTAAGAGTCATTTTGCTATTGGCCTAGCTCTCTACTACC |
| | | CTTCTGCTCGCATAGTGTATACAGCTTGCTCTCATGCCGCTGTTGATGCACTATGTGAGAAGGCATTAAA |
| | | ATATTTGCCTATAGATAAATGTAGTAGAATTATACCTGCACGTGCTCGTGTAGAGTGTTTTGATAAATTC |
| | | AAAGTGAATTCAACATTAGAACAGTATGTCTTTTGTACTGTAAATGCATTGCCTGAGACGACAGCAGATA |
| | | TAGTTGTCTTTGATGAAATTTCAATGGCCACAAATTATGATTTGAGTGTTGTCAATGCCAGATTACGTGC |
| | | TAAGCACTATGTGTACATTGGCGACCCTGCTCAATTACCTGCACCACGCACATTGCTAACTAAGGGCACA |
| | | CTAGAACCAGAATATTTCAATTCAGTGTGTAGACTTATGAAAACTATAGGTCCAGACATGTTCCTCGGAA |
| | | CTTGTCGGCGTTGTCCTGCTGAAATTGTTGACACTGTGAGTGCTTTGGTTTATGATAATAAGCTTAAAGC |
| | | ACATAAAGACAAATCAGCTCAATGCTTTAAAATGTTTTATAAGGGTGTTATCACGCATGATGTTTCATCT |
| | | GCAATTAACAGGCCACAAATAGGCGTGGTAAGAGAATTCCTTACACGTAACCCTGCTTGGAGAAAAGCTG |
| | | TCTTTATTTCACCTTATAATTCACAGAATGCTGTAGCCTCAAAGATTTTGGGACTACCAACTCAAACTGT |
| | | TGATTCATCACAGGGCTCAGAATATGACTATGTCATATTCACTCAAACCACTGAAACAGCTCACTCTTGT |
| | | AATGTAAACAGATTTAATGTTGCTATTACCAGAGCAAAAGTAGGCATACTTTGCATAATGTCTGATAGAG |
| | | ACCTTTATGACAAGTTGCAATTTACAAGTCTTGAAATTCCACGTAGGAATGTGGCAACTTTACAAGCTGA |
| | | AAATGTAACAGGACTCTTTAAAGATTGTAGTAAGGTAATCACTGGGTTACATCCTACACAGGCACCTACA |
| | | CACCTCAGTGTTGACACTAAATTCAAAACTGAAGGTTTATGTGTTGACATACCTGGCATACCTAAGGACA |
| | | TGACCTATAGAAGACTCATCTCTATGATGGGTTTTAAAATGAATTATCAAGTTAATGGTTACCCTAACAT |
| | | GTTTATCACCCGCGAAGAAGCTATAAGACATGTACGTGCATGGATTGGCTTCGATGTCGAGGGGTGTCAT |
| | | GCTACTAGAGAAGCTGTTGGTACCAATTTACCTTTACAGCTAGGTTTTTCTACAGGTGTTAACCTAGTTG |
| | | CTGTACCTACAGGTTATGTTGATACACCTAATAATACAGATTTTTCCAGAGTTAGTGCTAAACCACCGCC |
| | | TGGAGATCAATTTAAACACCTCATACCACTTATGTACAAAGGACTTCCTTGGAATGTAGTGCGTATAAAG |
| | | ATTGTACAAATGTTAAGTGACACACTTAAAAATCTCTCTGACAGAGTCGTATTTGTCTTATGGGCACATG |
| | | GCTTTGAGTTGACATCTATGAAGTATTTTGTGAAAATAGGACCTGAGCGCACCTGTTGTCTATGTGATAG |
| | | ACGTGCCACATGCTTTTCCACTGCTTCAGACACTTATGCCTGTTGGCATCATTCTATTGGATTTGATTAC |
| | | GTCTATAATCCGTTTATGATTGATGTTCAACAATGGGGTTTTACAGGTAACCTACAAAGCAACCATGATC |
| | | TGTATTGTCAAGTCCATGGTAATGCACATGTAGCTAGTTGTGATGCAATCATGACTAGGTGTCTAGCTGT |
| | | CCACGAGTGCTTTGTTAAGCGTGTTGACTGGACTATTGAATATCCTATAATTGGTGATGAACTGAAGATT |
| | | AATGCGGCTTGTAGAAAGGTTCAACACATGGTTGTTAAAGCTGCATTATTAGCAGACAAATTCCCAGTTC |
| | | TTCACGACATTGGTAACCCTAAAGCTATTAAGTGTGTACCTCAAGCTGATGTAGAATGGAAGTTCTATGA |
| | | TGCACAGCCTTGTAGTGACAAAGCTTATAAAATAGAAGAATTATTCTATTCTTATGCCACACATTCTGAC |
| | | AAATTCACAGATGGTGTATGCCTATTTTGGAATTGCAATGTCGATAGATATCCTGCTAATTCCATTGTTT |
| | | GTAGATTTGACACTAGAGTGCTATCTAACCTTAACTTGCCTGGTTGTGATGGTGGCAGTTTGTATGTAAA |
| | | TAAACATGCATTCCACACACCAGCTTTTGATAAAAGTGCTTTTGTTAATTTAAAACAATTACCATTTTTC |
| | | TATTACTCTGACAGTCCATGTGAGTCTCATGGAAAACAAGTAGTGTCAGATATAGATTATGTACCACTAA |
| | | AGTCTGCTACGTGTATAACACGTTGCAATTTAGGTGGTGCTGTCTGTAGACATCATGCTAATGAGTACAG |
| | | ATTGTATCTCGATGCTTATAACATGATGATCTCAGCTGGCTTTAGCTTGTGGGTTTACAAACAATTTGAT |
| | | ACTTATAACCTCTGGAACACTTTTACAAGACTTCAGAGTTTAGAAAATGTGGCTTTTAATGTTGTAAATA |
| | | AGGGACACTTTGATGGACAACAGGGTGAAGTACCAGTTTCTATCATTAATAACACTGTTTACACAAAAGT |
| | | TGATGGTGTTGATGTAGAATTGTTTGAAAATAAAACAACATTACCTGTTAATGTAGCATTTGAGCTTTGG |
| | | GCTAAGCGCAACATTAAACCAGTACCAGAGGTGAAAATACTCAATAATTTGGGTGTGGACATTGCTGCTA |
| | | ATACTGTGATCTGGGACTACAAAAGAGATGCTCCAGCACATATATCTACTATTGGTGTTTGTTCTATGAC |
| | | TGACATAGCCAAGAAACCAACTGAAACGATTTGTGCACCACTCACTGTCTTTTTTGATGGTAGAGTTGAT |
| | | GGTCAAGTAGACTTATTTAGAAATGCCCGTAATGGTGTTCTTATTACAGAAGGTAGTGTTAAAGGTTTAC |
| | | AACCATCTGTAGGTCCCAAACAAGCTAGTCTTAATGGAGTCACATTAATTGGAGAAGCCGTAAAAACACA |
| | | GTTCAATTATTATAAGAAAGTTGATGGTGTTGTCCAACAATTACCTGAAACTTACTTTACTCAGAGTAGA |
| | | AATTTACAAGAATTTAAACCCAGGAGTCAAATGGAAATTGATTTCTTAGAATTAGCTATGGATGAATTCA |
| | | TTGAACGGTATAAATTAGAAGGCTATGCCTTCGAACATATCGTTTATGGAGATTTTAGTCATAGTCAGTT |
| | | AGGTGGTTTACATCTACTGATTGGACTAGCTAAACGTTTTAAGGAATCACCTTTTGAATTAGAAGATTTT |
| | | ATTCCTATGGACAGTACAGTTAAAAACTATTTCATAACAGATGCGCAAACAGGTTCATCTAAGTGTGTGT |
| | | GTTCTGTTATTGATTTATTACTTGATGATTTTGTTGAAATAATAAAATCCCAAGATTTATCTGTAGTTTC |
| | | TAAGGTTGTCAAAGTGACTATTGACTATACAGAAATTTCATTTATGCTTTGGTGTAAAGATGGCCATGTA |
| | | GAAACATTTTACCCAAAATTACAATCTAGTCAAGCGTGGCAACCGGGTGTTGCTATGCCTAATCTTTACA |
| | | AAATGCAAAGAATGCTATTAGAAAAGTGTGACCTTCAAAATTATGGTGATAGTGCAACATTACCTAAAGG |
| | | CATAATGATGAATGTCGCAAAATATACTCAACTGTGTCAATATTTAAACACATTAACATTAGCTGTACCC |
| | | TATAATATGAGAGTTATACATTTTGGTGCTGGTTCTGATAAAGGAGTTGCACCAGGTACAGCTGTTTTAA |
| | | GACAGTGGTTGCCTACGGGTACGCTGCTTGTCGATTCAGATCTTAATGACTTTGTCTCTGATGCAGATTC |
| | | AACTTTGATTGGTGATTGTGCAACTGTACATACAGCTAATAAATGGGATCTCATTATTAGTGATATGTAC |
| | | GACCCTAAGACTAAAAATGTTACAAAAGAAAATGACTCTAAAGAGGGTTTTTTCACTTACATTTGTGGGT |
| | | TTATACAACAAAAGCTAGCTCTTGGAGGTTCCGTGGCTATAAAGATAACAGAACATTCTTGGAATGCTGA |
| | | TCTTTATAAGCTCATGGGACACTTCGCATGGTGGACAGCCTTTGTTACTAATGTGAATGCGTCATCATCT |
| | | GAAGCATTTTTAATTGGATGTAATTATCTTGGCAAACCACGCGAACAAATAGATGGTTATGTCATGCATG |
| | | CAAATTACATATTTTGGAGGAATACAAATCCAATTCAGTTGTCTTCCTATTCTTTATTTGACATGAGTAA |
| | | ATTTCCCCTTAAATTAAGGGGTACTGCTGTTATGTCTTTAAAAGAAGGTCAAATCAATGATATGATTTTA |
| | | TCTCTTCTTAGTAAAGGTAGACTTATAATTAGAGAAAACAACAGAGTTGTTATTTCTAGTGATGTTCTTG |
| | | TTAACAACTAA |

In some embodiments, such pathogens are a virus from a plant, an animal, a bacterium, or an archaeon. Additional viruses contemplated herein include, but are not limited to, viruses comprising nucleic acid components of vegetable mosaic viruses (tomato mosaic virus, tobacco mosaic virus, cucumber mosaic virus), and viruses related to common animal diseases, including rabies virus. Similarly contemplated as nucleic acids within the current disclosure are viroids and subviral pathogens such as hepatitis delta RNA, citrus exocortis viroid, columnea latent viroid, pepper chat fruit viroid, potato spindle tuber viroid, tomato chlorotic dwarf viroid, coconut cadang-cadang viroid, and tomato apical stunt viroid, and the like.

In some embodiments, such pathogens are a virus comprising an RNA virus or a DNA virus. In some embodiments, such RNA or DNA virus is single-stranded or double-stranded. In some embodiments, such RNA or DNA virus is a negative-sense or a positive-sense virus.

In some embodiments, such pathogens are a virus comprising single-stranded DNA (ssDNA). Such virus comprising single-stranded DNA is from the family of Anelloviridae, Bacillariodnaviridae, Bidnaviridae, Circoviridae, Geminiviridae, Inoviridae, Microviridae, Nanoviridae, Parvoviridae, and Spiraviridae.

In some embodiments, such pathogens are a virus comprising double-stranded DNA (dsDNA). Such virus comprising double-stranded DNA is from the family of Adenoviridae, Alloherpesviridae, Ampullaviridae, Ascoviridae, Asfaviridae, Baculoviridae, Bicaudaviridae, Clavaviridae, Corticoviridae, Fuselloviridae, Globuloviridae, Guttaviridae, Herpesviridae, Hytrosaviridae, Iridoviridae, Lipothrixviridae, Malacoherpesviridae, Marseilleviridae, Mimiviridae, Myoviridae, Nimaviridae, Pandoraviridae, Papillomaviridae, Phycodnaviridae, Plasmaviridae, Podoviridae, Polydnaviruses, Polyomaviridae, Poxviridae, Rudiviridae, Siphoviridae, Tectiviridae, Sphaerolipoviridae, and the like.

In some embodiments, such pathogens are a virus comprising both ssDNA and dsDNA regions. Such virus comprising both ssDNA and ds DNA regions is from the family of Pleolipoviruses comprising Haloarcula hispanica pleomorphic virus 1, Halogeometricum pleomorphic virus 1, Halorubrum pleomorphic virus 1, Halorubrum pleomorphic virus 2, Halorubrum pleomorphic virus 3, Halorubrum pleomorphic virus 6, and the like.

In some embodiments, such pathogens are a virus comprising double-stranded RNA (dsRNA). Such virus comprising double-stranded RNA is from the family comprising Birnaviridae, Chrysoviridae, Cystoviridae, Endornaviridae, Hypoviridae, Megavirnaviridae, Partitiviridae, Picobirnaviridae, Reoviridae, Rotavirus, Totiviridae, and the like.

In some embodiments, such pathogens are a virus comprising the negative-sense RNA. Such virus comprising negative-sense RNA virus is from the family of Arenaviridae, Bornaviridae, Bunyaviridae, Filoviridae, Nyamiviridae, Ophioviridae, Orthomyxoviridae, Paramyxoviridae, Rhabdoviridae, and the like.

In some embodiments, such pathogens are a virus comprising the positive-sense RNA. Such virus comprising the positive-sense RNA is from the family of Alphaflexiviridae, Alphatetraviridae, Alvernaviridae, Arteriviridae, Astroviridae, Barnaviridae, Betaflexiviridae, Bromoviridae, Caliciviridae, Carmotetraviridae, Closteroviridae, Coronaviridae, Dicistroviridae, Flaviviridae, Gammaflexiviridae, Iflaviridae, Leviviridae, Luteoviridae, Marnaviridae, Mesoniviridae, Narnaviridae, Nodaviridae, Permutotetraviridae, Picornaviridae, Potyviridae, Roniviridae, Secoviridae, Togaviridae, Tombusviridae, Tymoviridae, Virgaviridae, and the like.

### Target Nucleic Acid Molecules

The disclosed methods and systems may be used to detect any of a variety of target nucleic acid molecules (sometimes referred to as “analytes”). Examples include, but are not limited to, DNA molecules or fragments thereof, genomic DNA or fragments thereof, mitochondrial DNA or fragments thereof, chromosomal DNA or fragments thereof, plasmid DNA or fragments thereof, gene sequences or fragments thereof, exon sequences or fragments thereof, intron sequences or fragments thereof, bacterial DNA or fragments thereof, viral DNA or fragments thereof, RNA molecules or fragments thereof, mRNA molecules or fragments thereof, tRNA molecules or fragments thereof, rRNA molecules or fragments thereof, bacterial RNA or fragments thereof, viral RNA or fragments thereof, and the like, or any combination thereof.

### Samples

The disclosed methods and systems may be used to detect target nucleic acid molecules in any of a variety of samples. Non-limiting examples of a sample include, but are not limited to, tissue samples, cell suspension samples, surgical resection samples, biopsy samples, nasopharyngeal swab samples, sputum samples, bronchoalveolar lavage fluid samples, blood samples, urine samples, feces samples, or any combination thereof. In some embodiments, the sample is obtained from soil, sewage, biological tissue, food, a surface of an object in contact with one or more of the preceding samples, or any combination thereof. In some embodiments, multiple samples are obtained at different time points, or at different locations, or both. In such an embodiment, a presence of the target nucleic acid (e.g., derived from a pathogen disclosed herein) is indicative of a spread of infection by the pathogen. In some embodiments, processing of samples may be required for extraction and purification of the target nucleic acid molecules of interest.

### Barcoding Assay Workflow

Padlock (or molecular inversion) probes comprising target nucleic acid-specific recognition sequences, e.g., COVID-19 locus specific sequences, are designed and synthesized. Each padlock probe includes a locus-specific probe barcode positioned in the non-targeting region of the probe molecule. Upon recognition of the target sequence, the padlock probe will hybridize to the target, generating a circularizable intermediate that can then be fully circularized through ligation. Optionally, any remaining unreacted probe molecules or linear sample nucleic acid molecules, e.g., DNA, can be then digested by an exonuclease, leaving a number of circularized probes proportional to the number of target nucleic acid molecules, e.g., viral RNA molecules, where each species of circularized probe in a multiplexed assay is identifiable by the probe barcode inserted therein. Sample-indexed rolling circle amplification (RCA) is then performed (e.g., using amplification primers comprising a unique sample barcode sequence) to generate concatemer molecules comprising multiple copies of the circularized probe sequences.

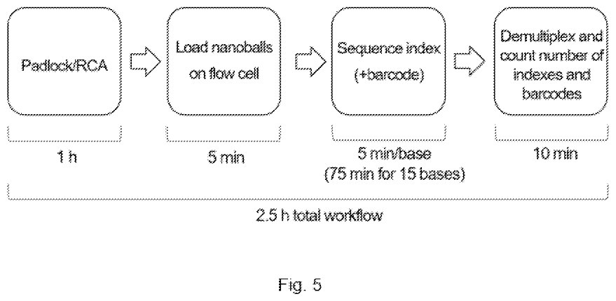

FIG. 5 illustrates an example of a workflow for the disclosed barcoded padlock probe or molecular inversion probe assays. The use of several different probe pools, each identified by a unique probe barcode, allows one to perform multiplexed testing for detection of multiple targets or diseases. An isothermal padlock assay followed by indexed RCA may be executed in less than, for example, 1 hour. The resulting concatemer molecules are then be condensed into nanoballs and loaded into a sequencing flow cell. The barcodes and indexes may then be sequenced using, e.g., 15 sequencing cycles, thereby providing for rapid sequencing data read-out (in, e.g., approximately 75 min). Sample index demultiplexing and probe barcode counting provides a yes/no answer for the presence of the target nucleic acid in a given sample with high precision due to the large number of probe barcodes counted for each sample. Viral titer data is also accessible since the number of probe barcodes counted will be proportional to the number of viral copies that were present in the sample.

The proposed barcoding methods can be implemented for introducing both sample indexes for multiplexed sample processing or unique probe barcodes to identify the specific locus targeted by a given probe, thereby enabling assay multiplexing. Besides sample multiplexing, the disclosed methods enable assays that can target multiple sites within the genome of an infectious disease agent (e.g., the COVID-19 genome), thereby increasing the specificity of the assay (e.g., a COVID-19 assay) and allowing for the identification of multiple strains.

### Sample Processing and DNA Extraction

In some instances, samples may require processing to extract the target nucleic acid molecules of interest. Any of a variety of existing sample processing and nucleic acid extraction techniques may be utilized.

In some embodiments, DNA extraction comprises: (i) collection of the sample (e.g., a swab sample, a cell sample, a blood sample, or tissue sample) from which the DNA is to be extracted; (ii) disruption of cell membranes (e.g., cell lysis) to release DNA and other cytoplasmic components in the presence of a lysis buffer, (iii) treatment of the lysed sample with a concentrated salt solution to precipitate proteins, lipids, and RNA followed by centrifugation to separate out the precipitated proteins, lipids, and RNA; and (iv) purification of DNA from the supernatant to remove detergents, proteins, salts, or other reagents used during the cell membrane lysis step.

Disruption of cell membranes for DNA (or RNA) extraction may be performed using a variety of mechanical shear (e.g., by passing through a French press or fine needle), bead-based disruption, or ultrasonic disruption techniques. The cell lysis step often comprises the use of detergents and surfactants to solubilize lipids the cellular and nuclear membranes. In some instances, the lysis step may further comprise use of proteases to break down protein, or the use of an RNase for digestion of RNA in the sample.

Examples of existing techniques for DNA purification include, but are not limited, to (i) precipitation in ice-cold ethanol or isopropanol followed by centrifugation (precipitation of DNA may be enhanced by increasing ionic strength, e.g., by addition of sodium acetate); (ii) phenol-chloroform extraction followed by centrifugation to separate the aqueous phase containing the nucleic acid from the organic phase containing denatured protein; and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate or through extraction with a phenol-chloroform mixture prior to a DNA precipitation step.

In some instances, DNA may be extracted using any of a variety of commercial DNA extraction and purification kits. Examples include, but are not limited to, the QIAamp (for isolation of genomic DNA from human samples) and DNAeasy kits (for isolation of genomic DNA from animal or plant samples) from Qiagen (Germantown, MD) or the Maxwell® and ReliaPrep™ series of kits from Promega (Madison, WI).

After isolation, the DNA is dissolved in a slightly alkaline buffer, e.g., Tris-EDTA (TE) buffer, or in ultra-pure water. Additional DNA fragmentation, if necessary, may be performed using mechanical fragmentation (e.g., using sonication, needle shear, nebulization, point-sink shearing, or passage through a pressure cell) or enzymatic digestion techniques (e.g., with the use of restriction enzymes or endonucleases).

### Sample Processing and RNA Extraction

An existing RNA extraction procedure comprises: (i) collection of a sample (e.g., a swab sample, a cell sample, a blood sample, or tissue sample) from which the RNA is to be extracted; (ii) optionally, protection and freezing of the sample for later processing, where an RNA stabilization reagent, such as the Invitrogen™ RNAlater™ and Invitrogen™ RNAlater™-ICE RNA stabilization solutions, may be used to stabilize the RNA in the sample for later purification; (iii) RNA extraction using, e.g., organic extraction methods, spin basket formats, magnetic particle methods, and direct lysis methods.

Organic extraction methods are widely used for RNA preparation. The sample is homogenized in, e.g., a phenol-containing solution and then centrifuged to yield three separate phases: a lower organic phase, a middle phase that contains denatured proteins and genomic DNA, and an upper aqueous phase that contains the RNA. The upper aqueous phase is recovered, and RNA is collected by alcohol precipitation and rehydration. Although the organic extraction methods provide for rapid denaturation of nucleases and stabilization of RNA in a scalable format, these methods, comprising the use of chlorinated organic reagents, may be labor-intensive and can be difficult to automate.

Filter-based, spin basket RNA preparation techniques utilize glass fiber, derivatized silica, or ion exchange membranes seated at the bottom of a small plastic basket. Samples are lysed in a buffer that contains RNase inhibitors (e.g., guanidine salts), and nucleic acids are bound to the membrane by passing the lysate through the membrane using centrifugal force or applied vacuum followed by several wash steps. An elution solution is then applied, and the extracted RNA is collected into a tube by centrifugation. Some methods combine the use of organic extraction with the sample collection, washing, and elution steps of a spin basket format. Spin-basket techniques for RNA extraction are convenient and easy to use, amenable to processing of sample in both single-sample and 96-well formats, and relatively easy to automate. Drawbacks include a propensity of the filter material to clog with particulates, the retention of large nucleic acid molecules such as genomic DNA, and fixed binding capacity within a manufactured format.

Magnetic particle extraction methods utilize small (0.5-1 μm diameter) particles that contain a paramagnetic core and surrounding shell that has been modified to bind to molecules of interest. Paramagnetic particles migrate in an applied magnetic field, but they retain minimal magnetic memory once the field is removed. This phenomenon allows the magnetic particles to interact with the molecules of interest in solution based on their surface modification, to be collected rapidly using an external magnetic field, and then to be easily resuspended once the field is removed. Samples are lysed in a solution comprising RNase inhibitors and allowed to bind to the magnetic particles. The magnetic particles and associated RNA may be collected by applying a magnetic field and subjected to several rounds of release, resuspension in wash solutions, and recapture, following which the RNA is released into an elution buffer, and the magnetic particles are removed. One of the advantages of magnetic particle extraction techniques is that the solution-based binding kinetics increase the efficiency of target capture. The magnetic bead format also allows for rapid collection/concentration of sample RNA (or other biomolecules depending on the bead surface; there are a wide variety of surface chemistries available) and is amenable to automation. Potential drawbacks include carry-through of magnetic particles into eluted samples, slow migration of magnetic particles in viscous solutions, and laborious capture/release steps when performed manually.

Direct lysis methods perform sample preparation (not purification) by utilizing lysis buffer formulations that disrupt samples, stabilize nucleic acids, and are compatible with downstream analysis. A sample is mixed with a lysis agent and incubated for a specified time under specified conditions. In some instances, the lysate may be used directly for downstream analysis. In many instances, the samples, e.g., RNA samples, may be purified from stabilized lysates, e.g., using magnetic beads, spin filter baskets, or other existing techniques. By eliminating the need to bind and elute from solid surfaces, direct lysis methods may avoid sample bias and recovery efficiency effects that may occur when using other extraction/purification methods. Directly lysis methods are fast, compatible with small samples, amenable to automation, and provide the highest potential for accurate representation of the distribution of RNA species within the sample. Potential drawbacks of direct lysis methods may include significant dilution of the sample, incompatibility with existing analytical methods such as spectrophotometric measurements of yield, and sample degradation due to residual RNase activity if lysates are not handled properly, and the like.

### Fragmenting Nucleic Acid Molecules

Provided here, in some embodiments, are methods for fragmenting a nucleic acid that has been obtained. In some embodiments, fragmenting comprises at least one of shearing, sonicating, restriction digesting, sequence specific endonuclease treatment, sequence-independent endonuclease treatment and chemical digesting, as well as other shearing approaches. Various shearing options include acoustic shearing, point-sink shearing, and needle shearing. In some steps, the restriction digesting is the intentional sequence specific breaking of nucleic acid molecules. One type of the restriction digesting is an enzyme-based treatment to fragment the double-stranded nucleic acid molecules either by the simultaneous cleavage of both strands, or by generation of nicks on each strand of the double-stranded nucleic acid molecules to produce double-stranded nucleic acid molecules breaks. One type of sonication subject nucleic acid molecules to acoustic cavitation and hydrodynamic shearing by exposure to brief periods of sonication. As one type of shearing, the acoustic shearing transmits high-frequency acoustic energy waves to nucleic acid molecules. As another type of shearing, the point-sink shearing uses a syringe pump to create hydrodynamic shear forces by pushing a nucleic acid library through a small abrupt contraction. As yet another type of shearing, the needle shearing creates shearing forces by passing DNA libraries through small gauge needle. After the fragmenting, some of the double-stranded nucleic acid fragments contain a region of a nucleic acid sequence with at least about 20, 30, 40, 50, 60, 70, 80, 90, 100, 110, 120, 130, 140, 150, 160, 170, 180, 190, 200, 225, 250, 300, 350, 400, 450, 500, 550, 600 bp or more. In some cases, after the fragmenting, some of the double-stranded nucleic acid fragments contain a region of a nucleic acid sequence with less than about 20.

In some embodiments, the fragmenting further comprises end repair, sticky end generation, and overhang generation. One type of the overhang generation comprises 5′ end generation. One type of the overhang generation comprises 3′ end generation. Some of the steps, such as end repair, sticky end generation, or overhang generation are performed in a tube. Some of the steps, such as end repair, sticky end generation, or overhang generation are performed with a solution containing the double-stranded nucleic acid fragments, end repair buffer, and end repair enzyme.

In some embodiments, the fragmenting further comprises end repair, sticky end generation, and overhang generation. One type of the overhang generation comprises 5′ end generation. One type of the overhang generation comprises 3′ end generation. Some of the steps, such as end repair, sticky end generation, or overhang generation are performed in a tube. Some of the steps, such as end repair, sticky end generation, or overhang generation are performed with a solution containing the double-stranded nucleic acid fragments, end repair buffer, and end repair enzyme.

### Immobilizing Fragmented Nucleic Acid Molecules to Surface

Provided herein, in some embodiments, are methods of immobilizing the fragmented nucleic acid molecules to a surface described herein. In some embodiments, the surface is a low non-specific binding surface, such as a hydrophilic surface. In some embodiments, the immobilizing comprises hybridizing a surface-bound capture nucleic acid molecule with at least a portion of the fragmented nucleic acid molecules (serving as a template for the sequencing reaction).

### Capture Nucleic Acid Molecule

In general, at least one layer of one or more layers of low non-specific binding material may comprise functional groups for covalently or non-covalently attaching nucleic acid molecules, e.g., adapter or primer sequences, or the at least one layer may already comprise covalently or non-covalently attached nucleic acid adapter or primer sequences at the time that it is deposited on the support surface. In some instances, the nucleic acid adaptor or primer sequences tethered to the polymer molecules of at least one third layer may be distributed at a plurality of depths throughout the layer.

In some instances, the nucleic acid adapter or primer molecules are covalently coupled to the polymer in solution, that is, prior to coupling or depositing the polymer on the surface. In some instances, the nucleic acid adapter or primer molecules are covalently coupled to the polymer after it has been coupled to or deposited on the surface. In some instances, at least one hydrophilic polymer layer comprises a plurality of covalently attached oligonucleotide adapter or primer molecules. In some instances, at least two, at least three, at least four, or at least five layers of hydrophilic polymer comprise a plurality of covalently attached adapter or primer molecules.

In some instances, the nucleic acid adapter or primer molecules may be coupled to the one or more layers of hydrophilic polymer using any of a variety of conjugation chemistries. For example, the oligonucleotide adapter or primer sequences may comprise moieties that are reactive with amine groups, carboxyl groups, thiol groups, and the like. Examples of amine-reactive conjugation chemistries that may be used include, but are not limited to, reactions involving isothiocyanate, isocyanate, acyl azide, NHS ester, sulfonyl chloride, aldehyde, glyoxal, epoxide, oxirane, carbonate, aryl halide, imidoester, carbodiimide, anhydride, and fluorophenyl ester groups. Examples of carboxyl-reactive conjugation chemistries include, but are not limited to, reactions involving carbodiimide compounds, e.g., water soluble EDC (1-ethyl-3-(3-dimethylaminopropyl)carbodiimide·HCL). Examples of sulfydryl-reactive conjugation chemistries include maleimides, haloacetyls and pyridyl disulfides.

One or more types of nucleic acid molecules may be attached or tethered to the support surface. In some instances, the one or more types of oligonucleotide adapters or primers may comprise spacer sequences, adapter sequences for hybridization to adapter-ligated template library nucleic acid sequences, forward amplification primers, reverse amplification primers, sequencing primers, molecular barcoding sequences, or any combination thereof. In some instances, 1 primer or adapter sequence may be tethered to at least one layer of the surface. In some instances, at least 2, 3, 4, 5, 6, 7, 8, 9, 10, or more than 10 different primer or adapter sequences may be tethered to at least one layer of the surface.

In some instances, the tethered nucleic acid adapter or primer sequences may range in length from about 10 nucleotides to about 100 nucleotides. In some instances, the tethered oligonucleotide adapter or primer sequences may be at least 10, at least 20, at least 30, at least 40, at least 50, at least 60, at least 70, at least 80, at least 90, or at least 100 nucleotides in length. In some instances, the tethered oligonucleotide adapter or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some instances the length of the tethered oligonucleotide adapter or primer sequences may range from about 20 nucleotides to about 80 nucleotides. In an example, the length of the tethered oligonucleotide adapter or primer sequences may have any value within this range, e.g., about 24 nucleotides.

In some instances, the resultant surface density of primers on the low binding support surfaces of the present disclosure may range from about 100 primer molecules per μm² to about 100,000 primer molecules per μm². In some instances, the resultant surface density of primers on the low binding support surfaces of the present disclosure may range from about 1,000 primer molecules per μm² to about 1,000,000 primer molecules per μm². In some instances, the surface density of primers may be at least 1,000, at least 10,000, at least 100,000, or at least 1,000,000 molecules per μm². In some instances, the surface density of primers may be at most 1,000,000, at most 100,000, at most 10,000, or at most 1,000 molecules per μm². Any of the lower and upper values described in this paragraph may be combined to form a range included within the present disclosure, for example, in some instances the surface density of primers may range from about 10,000 molecules per μm² to about 100,000 molecules per μm². In some instances, the surface density of primer molecules may have any value within this range, e.g., about 455,000 molecules per μm². In some instances, the surface density of target library nucleic acid sequences initially hybridized to adapter or primer sequences on the support surface may be less than or equal to that indicated for the surface density of tethered primers. In some instances, the surface density of clonally amplified target library nucleic acid sequences hybridized to adapter or primer sequences on the support surface may span the same range as that indicated for the surface density of tethered primers. In some instances, the surface properties of the capillary or channel lumen coating, including the surface density of tethered oligonucleotide primers, may be adjusted so as to optimize, e.g., solid-phase nucleic acid hybridization specificity and efficiency or solid-phase nucleic acid amplification rate, specificity, and efficiency.

Local densities as listed above do not preclude variation in density across a surface, such that a surface may comprise a region having an oligo density of, for example, 500,000/μm², while also comprising at least a second region having a substantially different local density.

In some instances, the tethered adapter or primer sequences may comprise modifications designed to facilitate the specificity and efficiency of nucleic acid amplification as performed on the low-binding supports. For example, in some instances the primer may comprise polymerase stop points such that the stretch of primer sequence between the surface conjugation point and the modification site is always in single-stranded form and functions as a loading site for 5′ to 3′ helicases in some helicase-dependent isothermal amplification methods. Other examples of primer modifications that may be used to create polymerase stop points include, but are not limited to, an insertion of a PEG chain into the backbone of the primer between two nucleotides towards the 5′ end, insertion of an abasic nucleotide (that is, a nucleotide that has neither a purine nor a pyrimidine base), or a lesion site which can be bypassed by the helicase.

As will be discussed further in the examples below, it may be desirable to vary the surface density of tethered oligonucleotide adapters or primers on the support surface or the spacing of the tethered adapter or primers away from the support surface (e.g., by varying the length of a linker molecule used to tether the adapter or primers to the surface) in order to “tune” the support for optimal performance when using a given amplification method. As noted below, adjusting the surface density of tethered oligonucleotide adapters or primers may impact the level of specific or non-specific amplification observed on the support in a manner that varies according to the amplification method selected. In some instances, the surface density of tethered nucleic acid adapters or primers may be varied by adjusting the ratio of molecular components used to create the support surface. For example, in the case that an nucleic acid primer—PEG conjugate is used to create the outer layer of a low-binding support, the ratio of the oligonucleotide primer—PEG conjugate to a non-conjugated PEG molecule may be varied. The resulting surface density of tethered primer molecules may then be estimated or measured using any of a variety of techniques. Examples include, but are not limited to, the use of radioisotope labeling and counting methods, covalent coupling of a cleavable molecule that comprises an optically-detectable tag (e.g., a fluorescent tag) that may be cleaved from a support surface of defined area, collected in a fixed volume of an appropriate solvent, and then quantified by comparison of fluorescence signals to that for a calibration solution of known optical tag concentration, or using fluorescence imaging techniques provided that care has been taken with the labeling reaction conditions and image acquisition settings to ensure that the fluorescence signals are linearly related to the number of fluorophores on the surface (e.g., that there is no significant self-quenching of the fluorophores on the surface).

In some instances, the resultant surface density of nucleic acid adapters or primers on the low binding support surfaces of the present disclosure may range from about 100 primer molecules per μm² to about 1,000,000 primer molecules per μm². In some instances, the surface density of oligonucleotide adapters or primers may be at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 5,500, at least 6,000, at least 6,500, at least 7,000, at least 7,500, at least 8,000, at least 8,500, at least 9,000, at least 9,500, at least 10,000, at least 15,000, at least 20,000, at least 25,000, at least 30,000, at least 35,000, at least 40,000, at least 45,000, at least 50,000, at least 55,000, at least 60,000, at least 65,000, at least 70,000, at least 75,000, at least 80,000, at least 85,000, at least 90,000, at least 95,000, at least 100,000, at least 150,000, at least 200,000, at least 250,000, at least 300,000, at least 350,000, at least 400,000, at least 450,000, at least 500,000, at least 550,000, at least 600,000, at least 650,000, at least 700,000, at least 750,000, at least 800,000, at least 850,000, at least 900,000, at least 950,000, or at least 1,000,000 molecules per μm². In some instances, the surface density of oligonucleotide adapters or primers may be at most 1,000,000, at most 950,000, at most 900,000, at most 850,000, at most 800,000, at most 750,000, at most 700,000, at most 650,000, at most 600,000, at most 550,000, at most 500,000, at most 450,000, at most 400,000, at most 350,000, at most 300,000, at most 250,000, at most 200,000, at most 150,000, at most 100,000, at most 95,000, at most 90,000, at most 85,000, at most 80,000, at most 75,000, at most 70,000, at most 65,000, at most 60,000, at most 55,000, at most 50,000, at most 45,000, at most 40,000, at most 35,000, at most 30,000, at most 25,000, at most 20,000, at most 15,000, at most 10,000, at most 9,500, at most 9,000, at most 8,500, at most 8,000, at most 7,500, at most 7,000, at most 6,500, at most 6,000, at most 5,500, at most 5,000, at most 4,500, at most 4,000, at most 3,500, at most 3,000, at most 2,500, at most 2,000, at most 1,500, at most 1,000, at most 900, at most 800, at most 700, at most 600, at most 500, at most 400, at most 300, at most 200, or at most 100 molecules per μm². Any of the lower and upper values described in this paragraph may be combined to form a range included within the present disclosure, for example, in some instances the surface density of adapters or primers may range from about 10,000 molecules per μm² to about 100,000 molecules per μm². The surface density of adapter or primer molecules may have any value within this range, e.g., about 3,800 molecules per μm² in some instances, or about 455,000 molecules per μm² in other instances. In some instances, as will be discussed further below, the surface density of template library nucleic acid sequences (e.g., sample DNA molecules) initially hybridized to adapter or primer sequences on the support surface may be less than or equal to that indicated for the surface density of tethered nucleic acid primers. In some instances, as will also be discussed further below, the surface density of clonally-amplified template library nucleic acid sequences hybridized to adapter or primer sequences on the support surface may span the same range or a different range as that indicated for the surface density of tethered oligonucleotide adapters or primers.

In some embodiments, nucleic acids in a library are coupled to a surface (e.g., low non-specific binding surface). In some embodiments, the coupling is performed by way of hybridization between a region of the nucleic acid molecule and a region of a capture molecule coupled to the surface. Unless noted otherwise, hybridization may occur between nucleic acids of any length and the hybridized nucleic acid may take on one or a combination of many structural forms, including, but not limited to: the B-form, the A-form, Z-form, stem loop, pseudoknot, or other hybridization structures formed by base-pairing interactions between two or more single-stranded nucleic acids. In some embodiments, hybridization occurs between two single-stranded nucleic acids of any length. In some embodiments, hybridization occurs between a single-stranded linear nucleic acid and a single-stranded linear nucleic acid. In some embodiments, hybridization occurs between a single-stranded linear nucleic acid and a single-stranded circularized nucleic acid. In some embodiments, hybridization occurs between a single-stranded circularized nucleic acid and a single-stranded circularized nucleic acid. In some embodiments, hybridization occurs between a DNA molecule and a DNA molecule. In some embodiments, hybridization occurs between a DNA molecule and an RNA molecule. In some embodiments, hybridization occurs between an RNA molecule and an RNA molecule. In some embodiments, hybridization occurs between a DNA molecule and a DNA/RNA hybrid molecule. In some embodiments, hybridization occurs between an RNA molecule and a DNA/RNA hybrid molecule. In some embodiments, hybridization occurs between a DNA/RNA hybrid molecule and a DNA/RNA hybrid molecule.

In some embodiments, a nucleic acid molecule of the library is coupled to the surface by hybridization between a nucleic acid sequence of the nucleic acid molecule and one or more capture nucleic acid molecules coupled the surface. In some embodiments, the one or more capture nucleic acid molecules is a splint nucleic acid molecule described herein and facilitates circularization of the nucleic acid molecule on the surface in the presence of a ligating enzyme or catalytically active portion thereof described herein.

In some embodiments, the one or more capture nucleic acid molecules (as referred to here as surface-bound primer) hybridizes to one or more adaptors of the nucleic acid molecule, such as an adaptor containing an index sequence disclosed herein. In some embodiments, the index sequence is any unique sequence of 8 to 10 nucleotides, usable as unique index sequence pairs.

### Hybridization Ofbarcoded Probe Molecules to Target

In some instances, hybridization of the disclosed barcoded padlock probe or molecular inversion probe molecules to target nucleic acid sequences may be performed in samples comprising, e.g., purified, partially purified, or non-purified target nucleic acid molecules. Hybridization may be performed using any of a variety of existing hybridization protocols.

In some instances, the hybridization reaction may comprise the use of a hybridization buffer formulation comprising a pH buffer, an organic solvent, a molecular crowding agent, an additive for controlling melting temperature of double-stranded nucleic acids, an additive that impacts nucleic acid hydration, or any combination thereof.

In some aspects of the present disclosure, hybridization buffer formulations are described which, in combination with the disclosed low non-specific binding supports, provide for improved hybridization rates, hybridization specificity (or stringency), and hybridization efficiency (or yield). As used herein, hybridization specificity is a measure of the ability of tethered adapter sequences, primer sequences, or oligonucleotide sequences in general to correctly hybridize to completely complementary sequences, while hybridization efficiency is a measure of the percentage of total available tethered adapter sequences, primer sequences, or oligonucleotide sequences in general that are hybridized to complementary sequences.

Improved hybridization specificity or efficiency may be achieved through optimization of the hybridization buffer formulation used with the disclosed low-binding surfaces and will be discussed in more detail in the examples below. Examples of hybridization buffer components that may be adjusted to achieve improved performance include, but are not limited to, buffer type, organic solvent mixtures, buffer pH, buffer viscosity, detergents and zwitterionic components, ionic strength (including adjustment of both monovalent and divalent ion concentrations), antioxidants and reducing agents, carbohydrates, BSA, poly(ethylene glycol), dextran sulfate, betaine, other additives, and the like.

In some instances, the hybridization buffer formulation may comprise a pH buffer selected from the group comprising Tris, HEPES, TAPS, Tricine, Bicine, Bis-Tris, NaOH, KOH, TES, EPFS, and MOPS. In some instances, the pH of the hybridization buffer formulation may range from about 3 to about 10. In some instances, the pH may be at least 3, at least 4, at least 5, at least 6, at least 7, at least 8, at least 9, or at least 10. In some instances, the pH may be at most 10, at most 9, at most 8, at most 7, at most 6, at most 5, at most 4, or at most 3. Any of the lower and upper values described in this paragraph may be combined to form a range included within the present disclosure, for example, the pH of the hybridization buffer may range from about 4 to about 8. It is possible that the pH of the hybridization buffer may have any value within this range, e.g., about pH 7.8.

Examples of detergents for use in hybridization buffer formulation include, but are not limited to, zitterionic detergents (e.g., 1-dodecanoyl-sn-glycero-3-phosphocholine, 3-(4-tert-butyl-1-pyridinio)-1-propanesulfonate, 3-(N,N-dimethylmyristylammonio)propanesulfonate, 3-(N,N-dimethylmyristylammonio) propanesulfonate, ASB-C80, C7BzO, CHAPS, CHAPS hydrate, CHAPSO, DDMAB, dimethylethylammoniumpropane sulfonate, N,N-dimethyldodecylamine N-oxide, N-dodecyl-N,N-dimethyl-3-ammonio-1-propanesulfonate, or N-dodecyl-N,N-dimethyl-3-ammonio-1-propanesulfonate) and anionic, cationic, and non-ionic detergents. Examples of nonionic detergents include poly(oxyethylene) ethers and related polymers (e.g. Brij®, TWEEN®, TRITON®, TRITON X-100 and IGEPAL® CA-630), bile salts, and glycosidic detergents.

The use of the disclosed low non-specific binding supports either alone or in combination with optimized buffer formulations may yield relative hybridization rates that range from about 2× to about 20×faster than that for an existing hybridization protocol. In some instances, the relative hybridization rate may be at least 2×, at least 3×, at least 4×, at least 5×, at least 6×, at least 7×, at least 8×, at least 9×, at least 10×, at least 12×, at least 14×, at least 16×, at least 18×, at least 20×, at least 25×, at least 30×, or at least 40× that for an existing hybridization protocol.

In some instances, the use of the disclosed low-binding supports alone or in combination with optimized buffer formulations may yield total hybridization reaction times (that is, the time required to reach 90%, 95%, 98%, or 99% completion of the hybridization reaction) of less than 60 minutes, 50 minutes, 40 minutes, 30 minutes, 20 minutes, 15 minutes, 10 minutes, or 5 minutes for any of these completion metrics.

In some instances, the use of the disclosed low non-specific binding supports alone or in combination with optimized buffer formulations may yield improved hybridization specificity compared to that for an existing hybridization protocol. In some instances, the hybridization specificity that may be achieved is better than 1 base mismatch in 10 hybridization events, 1 base mismatch in 20 hybridization events, 1 base mismatch in 30 hybridization events, 1 base mismatch in 40 hybridization events, 1 base mismatch in 50 hybridization events, 1 base mismatch in 75 hybridization events, 1 base mismatch in 100 hybridization events, 1 base mismatch in 200 hybridization events, 1 base mismatch in 300 hybridization events, 1 base mismatch in 400 hybridization events, 1 base mismatch in 500 hybridization events, 1 base mismatch in 600 hybridization events, 1 base mismatch in 700 hybridization events, 1 base mismatch in 800 hybridization events, 1 base mismatch in 900 hybridization events, 1 base mismatch in 1,000 hybridization events, 1 base mismatch in 2,000 hybridization events, 1 base mismatch in 3,000 hybridization events, 1 base mismatch in 4,000 hybridization events, 1 base mismatch in 5,000 hybridization events, 1 base mismatch in 6,000 hybridization events, 1 base mismatch in 7,000 hybridization events, 1 base mismatch in 8,000 hybridization events, 1 base mismatch in 9,000 hybridization events, or 1 base mismatch in 10,000 hybridization events.

In some instances, the use of the disclosed low-binding supports alone or in combination with optimized buffer formulations may yield improved hybridization efficiency (e.g., the fraction of available oligonucleotide primers on the support surface that are successfully hybridized with target oligonucleotide sequences) compared to that for an existing hybridization protocol. In some instances, the hybridization efficiency that may be achieved is better than 50%, 60%, 70%, 80%, 85%, 90%, 95%, 98%, or 99% for any of the input target oligonucleotide concentrations specified below and in any of the hybridization reaction times specified above. In some instances, e.g., wherein the hybridization efficiency is less than 100%, the resulting surface density of target nucleic acid sequences hybridized to the support surface may be less than the surface density of oligonucleotide adapter or primer sequences on the surface.

In some instances, the hybridization buffer formulation may comprise an organic solvent. Examples of solvents include, but are not limited to, acetonitrile, ethanol, DMF, and methanol, or any combination thereof at varying percentages (>5%). In some instances, the percentage of organic solvent (by volume) included in the hybridization buffer may range from about 1% to about 20%. In some instances, the percentage by volume of organic solvent may be at least 1%, at least 2%, at least 3%, at least 4%, at least 5%, at least 6%, at least 7%, at least 8%, at least 9%, at least 10%, at least 15%, or at least 20%. In some instances, the percentage by volume of organic solvent may be at most 20%, at most 15%, at most 10%, at most 9%, at most 8%, at most 7%, at most 6%, at most 5%, at most 4%, at most 3%, at most 2%, or at most 1%. Any of the lower and upper values described in this paragraph may be combined to form a range included within the present disclosure, for example, the percentage by volume of organic solvent may range from about 4% to about 15%. It is possible that the percentage by volume of organic solvent may have any value within this range, e.g., about 7.5%.

In some instances, the hybridization buffer formulation may comprise a molecular crowding agent selected from the group comprising poly(ethylene glycol) (PEG), dextran, hydroxypropyl methyl cellulose (HPMC), hydroxyethyl methyl cellulose (HEMC), hydroxybutyl methyl cellulose, hydroxypropyl cellulose, methyl cellulose, and hydroxyl methyl cellulose, ovalbumin, hemoglobin, Ficoll, or any combination thereof. In some instances, the percentage of molecular crowding agent included in the hybridization buffer formulation may range from about 1% to about 60%. In some instances, the percentage of molecular crowding agent in the hybridization buffer may be at least 1%, 2%, 3%, 4%, 5%, 10%, 15%, 20%, 25%, 30%, 35%, 40%, 50%, 60%, or higher, by volume based on the total volume of the formulation. In some instances, the percentage of molecular crowding agent in the hybridization buffer may be at most 60%, 50%, 40%, 35%, 30%, 25%, 20%, 15%, 10%, 5%, 4%, 3%, 2%, 1%, or lower, by volume based on the total volume of the formulation. Any of the lower and upper values described in this paragraph may be combined to form a range included within the present disclosure, for example, in some instances the percentage by volume of molecular crowding agent may range from about 5% to about 25%. It is possible that the percentage by volume of molecular crowding agent may have any value within this range, e.g., about 8.5%.

In some instances, the hybridization buffer formulation may comprise an additive for controlling melting temperature. Examples include, but are not limited to, formamide, tetramethyl ammonium chloride (TMAC), or any combination thereof. The amount of the additive for controlling melting temperature of nucleic acid can vary depending on other agents used in the hybridization buffer formulation. In some instances, the percentage of melting temperature additive included in the hybridization buffer formulation may range from about 1% to about 60%. In some instances, the percentage of melting temperature additive in the hybridization buffer may be at least 1%, 2%, 3%, 4%, 5%, 10%, 15%, 20%, 25%, 30%, 35%, 40%, 50%, 60%, or higher, by volume based on the total volume of the formulation. In some instances, the percentage of melting temperature additive in the hybridization buffer may be at most 60%, 50%, 40%, 35%, 30%, 25%, 20%, 15%, 10%, 5%, 4%, 3%, 2%, 1%, or lower, by volume based on the total volume of the formulation. Any of the lower and upper values described in this paragraph may be combined to form a range included within the present disclosure, for example, in some instances the percentage by volume of melting temperature additive may range from about 4% to about 35%. It is possible that the percentage by volume of melting temperature additive may have any value within this range, e.g., about 6.5%.

In some instances, the hybridization buffer formulation may comprise an additive that impacts nucleic acid hydration. Examples include, but are not limited to, betaine, urea, glycine betaine, or any combination thereof. In some instances, the percentage by volume of a hydration additive included in the hybridization buffer formulation may range from about 1% to about 50%. In some instances, the percentage by volume of a hydration additive may be at least 1%, at least 5%, at least 10%, at least 15%, at least 20%, at least 25%, at least 30%, at least 35%, at least 40%, at least 45%), or at least 50%. In some instances, the percentage by volume of a hydration additive may be at most 50%, at most 45%, at most 40%, at most 35%, at most 30%, at most 25%, at most 20%, at most 15%, at most 10%, at most 5%, or at most 1%. Any of the lower and upper values described in this paragraph may be combined to form a range included within the present disclosure, for example, the percentage by volume of a hydration additive may range from about 1% to about 30%. It is possible that the percentage by volume of a melting temperature additive may have any value within this range, e.g., about 6.5%.

### Ligation

In addition to DNA ligase, the ligation of the disclosed barcoded padlock probe or molecular inversion probe molecules to create circularized probe molecule may comprise the use of an optimized ligation buffer. The two adjacent ends of the barcoded padlock probe (after hybridization to the target sequence) or of the molecule inversion probe (after hybridization to the target sequence and gap-filling) are joined together by DNA ligase which catalyzes the formation of a phosphodiester bond between the 3-OH at one end of the probe and the 5-phosphate group of the other end. Factors that affect the rate and yield of the ligation reaction include the nucleic acid concentration, the ligase concentration, the reaction temperature (the optimum temperature for DNA ligase activity is 37° C., but the optimal reaction temperature will also depend on the melting temperature (Tₘ) of the hybridized probe-target sequences), and ligation buffer composition (e.g., the ionic strength and species of cations present). In some instances, the ligation buffer composition may be the same as the hybridization buffer formulations described above or may comprise any of the hybridization buffer components, or combinations thereof, described above.

### Nucleic Acid Amplification

In some instances, the disclosed methods may comprise one or more nucleic acid amplification steps. In some embodiments, such amplification is performed in solution. In some embodiments, such application is performed on the surface. In some embodiments, amplification is performed prior to sequencing the nucleic acid molecules or derivatives thereof. Examples of nucleic acid amplification techniques include, but are not limited to, polymerase chain reaction (PCR), multiple displacement amplification (MDA), transcription-mediated amplification (TMA), nucleic acid sequence-based amplification (NASBA), strand displacement amplification (SDA), real-time SDA, rolling circle amplification, or circle-to-circle amplification.

As used herein, the phrase “nucleic acid surface amplification” (NASA) is used interchangeably with the phrase “solid-phase nucleic acid amplification” (or simply “solid-phase amplification”). In some aspects of the present disclosure, nucleic acid amplification formulations are described which, in combination with the disclosed low non-specific binding supports, provide for improved amplification rates, amplification specificity, and amplification efficiency. As used herein, specific amplification refers to amplification of template library oligonucleotide strands that have been tethered to the solid support either covalently or non-covalently. As used herein, non-specific amplification refers to amplification of primer-dimers or other non-template nucleic acids. As used herein, amplification efficiency is a measure of the percentage of tethered oligonucleotides on the support surface that are successfully amplified during a given amplification cycle or amplification reaction. Nucleic acid amplification performed on surfaces disclosed herein may obtain amplification efficiencies of at least 50%, 60%, 70%, 80%, 90%, 95%, or greater than 95%, such as 98% or 99%.

In some instances, an indexed amplification primer may be used to add a sample barcode to each amplified nucleic acid molecule during amplification of circularized padlock probe or molecular inversion probe molecules for a given sample, thereby allowing pooling of the amplicons from multiple samples prior to performing sequencing. In some instances, the amplification primer may also be used to add an adapter sequence, sequencing primer binding site, an additional primer binding site, or any combination thereof, to amplified product for a given sample.

Any of a variety of thermal cycling or isothermal nucleic acid amplification schemes may be used with the disclosed low non-specific binding supports. Examples of nucleic acid amplification methods that may be utilized with the disclosed low-binding supports include, but are not limited to, polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification, circle-to-circle amplification, helicase-dependent amplification, recombinase-dependent amplification, or single-stranded binding (SSB) protein-dependent amplification.

Often, improvements in amplification rate, amplification specificity, and amplification efficiency may be achieved using the disclosed low non-specific binding supports alone or in combination with formulations of the amplification reaction components. In addition to inclusion of nucleotides, one or more polymerases, helicases, single-stranded binding proteins, etc. (or any combination thereof), the amplification reaction mixture may be adjusted in a variety of ways to achieve improved performance including, but are not limited to, choice of buffer type, buffer pH, organic solvent mixtures, buffer viscosity, detergents and zwitterionic components, ionic strength (including adjustment of both monovalent and divalent ion concentrations), antioxidants and reducing agents, carbohydrates, BSA, poly(ethylene glycol), dextran sulfate, betaine, other additives, and the like.

In some instances, solid-phase amplification may be performed after tethering an amplicon comprising the circularized probe molecules (or re-linearized copies thereof) to a sequencing surface, thereby generating clonal colonies or clusters of the barcode sequences on the surface.

### Isothermal Rolling Circle Amplification (RCA)

In some embodiments, the disclosed methods may comprise the use of rolling circle amplification (RCA) to generate concatemer molecules comprising multiple copies of the circularized probe molecules. RCA is an isothermal nucleic acid amplification technique where the polymerase continuously adds single nucleotides to a primer annealed to a circular template, thereby generating a concatemer molecule comprising single-stranded DNA that contains tens to hundreds of tandem repeats of the nucleic acid sequence (complementary to the circular template). The components required for performing RCA include a DNA polymerase, a polymerase-compatible buffer, a short DNA or RNA primer, a circular DNA template, and deoxynucleotide triphosphates (dNTPs). The polymerases used in RCA are Phi29, Bst, and Vent exo-DNA polymerase for DNA amplification, and T7 RNA polymerase for RNA amplification. Phi29 DNA polymerase is frequently used as it has the best processivity and strand displacement ability. RCA is conducted at constant temperature (e.g., ranging from room temperature to about 37° C.) in both free solution and for solid phase amplification.

There are three steps involved in a DNA RCA reaction: (i) circular template ligation, which can be conducted via template-mediated enzymatic ligation (e.g., T4 DNA ligase) or template-free ligation using special DNA ligases (e.g., CircLigase); (ii) primer-induced single-strand DNA elongation; multiple primers can be hybridized to the same circular template (“multiprimed RCA”), resulting in the initiation of multiple amplification events and producing multiple RCA products (optionally, the conversion of linear RCA product into multiple circles using restriction enzyme digestion followed by template-mediated enzymatic ligation); and (iii) amplification product detection and visualization, e.g., by method of fluorescent detection using a fluorophore-conjugated dNTP, a fluorophore-labeled complementary sequence, or fluorescently-labeled molecular beacons.

In some instances, an indexed amplification primer may be used during RCA to add a sample barcode to each amplified nucleic acid molecule during amplification of circularized padlock probe or molecular inversion probe molecules for a given sample, thereby allowing pooling of the amplicons from multiple samples prior to performing sequencing. In some instances, the amplification primer may also be used to add an adapter sequence, sequencing primer binding site, an additional primer binding site, or any combination thereof, to amplified product for a given sample.

### Identifying a Nucleic Acid Sequence

Disclosed herein, in some embodiments, are methods of identifying a nucleic acid sequence of a pathogen disclosed herein. In some embodiments, the pathogen is a severe respiratory syndrome 2 (SARS-CoV-2). In some embodiments, identifying a nucleic acid sequence comprises sequencing. In some embodiments, identifying a nucleic acid sequence comprises targeted enrichment of a region of a pathogen genome, such as using a panel of nucleic acid probes specific to regions within the pathogen genome. In some embodiments, the entire genome of the pathogen is identified. In some embodiments, in the case of a coronavirus, a region of the genome is identified. In some embodiments, the region encodes a structural protein of the coronavirus comprises the spike glycoprotein, nucleocapsid protein, envelope glycoprotein, or membrane glycoprotein, or a combination thereof.

### Nucleic Acid Sequencing

The disclosed compositions and methods enable extremely high degrees of assay or sample multiplexing due to the large number of unique labels that may be generated using relatively short DNA sequences as barcodes. Furthermore, the relatively short sequencing reads required for implementing the disclosed barcoded padlock probe or molecular inversion probe assays leads to fast turn-round times and lower assay costs. Existing approaches to DNA barcoding rely on a standard sequencing run to identify the barcodes and then map them to a known manifest, such as in spatial transcriptomics applications, synthetic long reads, or Swab Seq. While effective, these approaches can be lengthy since an entire sequencing run, including clustering, has to be completed. They can also be cost-prohibitive unless a very large number of samples is multiplexed to amortize the cost of a sequencing kit.

Although any of a variety of commercial nucleic acid sequencing methods and platforms may be used for sequencing the barcoded RCA amplification product to demultiplex assay and sample data, in some embodiments, “nanoball” sequencing may be employed. Nanoball sequencing is a high throughput sequencing methodology that uses rolling circle replication to amplify short template nucleic acid sequences and generate concatemers which are then condensed to form nanoballs. The nanoballs may subsequently be tethered to a sequencing surface, e.g., an interior surface of a sequencing flow cell and subjected to an iterative series of, e.g., sequencing-by-synthesis reactions to determine the sequence of the short template nucleic acid sequences. Large numbers of nanoballs may be hybridized to adapters on, or otherwise tethered to, a sequencing surface to enable massively parallel sequencing to be performed at lower reagent costs compared to other next generation sequencing techniques.

In some instances, the sequencing of concatemer sequences, or concatemer sequences which have been condensed to form nanoballs, generated using the disclosed compositions and methods may comprise the use of existing sequencing by incorporation (sequencing-by-synthesis^{T}M) chemistries and commercially-available platforms such as those available from Illumina (San Diego, CA). In some instances, the sequencing may comprise the use of single molecule sequencing chemistries and commercially available instruments such as those available from Pacific Biosciences (Menlo Park, CA). In some instances, the sequencing may comprise the use of nanopore sequencing techniques and commercially available instruments such as those available from Oxford Nanopore (Oxford, United Kingdom). In some instances, the disclosed compositions and methods may comprise the use of sequencing by binding techniques and commercially available instruments such as those available from Omniome (Omniome™, San Diego, CA). In some instances, the disclosed sequencing comprises bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore DNA sequencing, shot gun sequencing, RNA sequencing, Enigma sequencing, or any combination thereof.

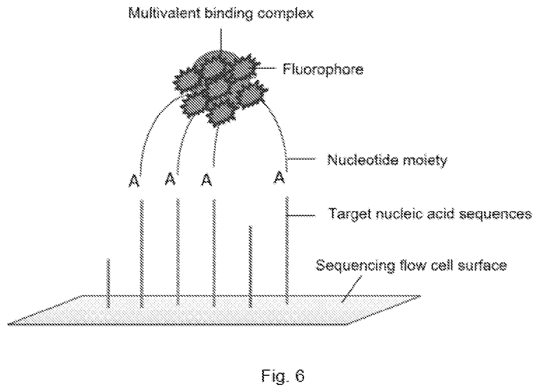

In some instances, the sequencing of concatemer sequences, or concatemer sequences which have been condensed to form nanoballs, generated using the disclosed compositions and methods may comprise the use of novel polymer-nucleotide conjugates (and related compositions) that enable “sequencing-by-trapping” methods as described in co-pending U.S. patent application Ser. No. 16/579,794 and International Patent Application Serial No. PCT/US2020/034409, both of which applications are incorporated herein in their entirety. Briefly, these methods comprise the use of polymer-nucleotide conjugates (or more generally, multivalent binding compositions) comprising a core structure to which a plurality of known nucleotide moieties, nucleotide analog moieties, or other binding elements are attached, and which optionally include a plurality of fluorophores or other detectable tags, are contacted with primed target nucleic acid molecules in the presence of a polymerase under conditions which promote hybridization between two or more nucleotide moieties of the polymer-nucleotide conjugate and two or more copies of the target sequence (e.g., two or more copies within a clonal cluster of replicate target nucleic acid molecules tethered to a surface, or two or more copies of the target nucleic acid sequence in a concatemer tethered to a surface) to form multivalent binding complexes which may be detected, e.g., using fluorescent labels as detectable tags and fluorescence imaging as the read-out for detection of the complex, to determine the identify of a nucleotide in the target nucleic acid sequence. FIG. 6 provides a schematic illustration of a multivalent binding complex formed between a polymer nucleotide conjugate comprising a plurality of nucleotide moieties and fluorophores and a plurality of target nucleic acid sequences tethered to a sequencing flow cell surface. In some instances, the nucleotide moieties attached to the polymer-nucleotide conjugate are not incorporated into the primed target nucleic acid strand. Instead, the multivalent binding complex is disrupted, and a single nucleotide extension reaction is performed prior to repeating the cycle of contacting with another polymer-nucleotide conjugate (or mixture thereof) comprising another or different known nucleotide moieties.

In some instances, multivalent binding compositions may comprise a plurality of nucleotides conjugated to a particle (e.g., a polymer, branched polymer, dendrimer, or equivalent structure) or other core structure. Contacting the multivalent binding composition with a polymerase and multiple copies of a primed target nucleic acid may result in the formation of a ternary complex which may be detected and in turn achieve a more accurate determination of the bases of the target nucleic acid.

Disclosed herein are methods of preparing multivalent binding or incorporation compositions comprising: a) a polymer core; and b) two or more nucleotide, nucleotide analog, nucleoside, or nucleoside analog moieties attached to the polymer core; wherein the length of the linker is dependent on the nucleotide, nucleotide analog, nucleoside, or nucleoside analog moiety that is attached to the polymer core. Also disclosed herein are methods of preparing multivalent binding compositions comprising: a) a mixture of polymer-nucleotide conjugates, wherein each polymer-nucleotide conjugate comprises: i) a polymer core; and ii) two or more nucleotide, nucleotide analog, nucleoside, or nucleoside analog moieties attached to the polymer core, wherein the length of the linker is dependent on the nucleotide, nucleotide analog, nucleoside, or nucleoside analog moiety that is attached to the polymer core; and wherein the mixture comprises polymer-nucleotide conjugates having at least two different types of attached nucleotide, nucleotide analog, nucleoside, or nucleoside analog moiety. In some embodiments, the polymer core comprises a polymer having a plurality of branches and the two or more nucleotide, nucleotide analog, nucleoside, or nucleoside analog moieties are attached to said branches. In some embodiments, polymer has a star, comb, cross-linked, bottle brush, or dendrimer configuration. In some embodiments, the polymer-nucleotide conjugate comprises one or more binding groups selected from the group comprising an avidin, a biotin, an affinity tag, and combinations thereof. In some embodiments, the polymer core comprises a branched polyethylene glycol (PEG) molecule. In some embodiments, the polymer-nucleotide conjugate comprises a blocked nucleotide moiety. In some embodiments, the blocked nucleotide is a 3′-0-azidomethyl nucleotide, a 3′-0-methyl nucleotide, or a 3′-0-alkyl hydroxylamine nucleotide. In some embodiments, the polymer-nucleotide conjugate further comprises one or more fluorescent labels.

Disclosed herein are methods of preparing multivalent binding compositions and analyzing nucleic acid molecules, including in sequencing or other bioassay applications. An increase in binding or incorporation of a nucleotide to an enzyme (e.g., polymerase) or an enzyme complex can be affected by increasing the effective concentration of the nucleotide. The increase can be achieved by increasing the concentration of the nucleotide in free solution, or by increasing the amount of the nucleotide in proximity to the relevant binding or incorporation site. The increase can also be achieved by physically restricting a number of nucleotides into a limited volume thus resulting in a local increase in concentration, and such as structure may thus bind or incorporate to the binding or incorporation site with a higher apparent avidity than can be observed with unconjugated, untethered, or otherwise unrestricted individual nucleotide. One non-limiting mechanism of effecting such restriction is by providing a multivalent binding or incorporation composition in which multiple nucleotides are bound to a particle such as a polymer, a branched polymer, a dendrimer, a micelle, a liposome, a microparticle, a nanoparticle, a quantum dot, or other types of particles.

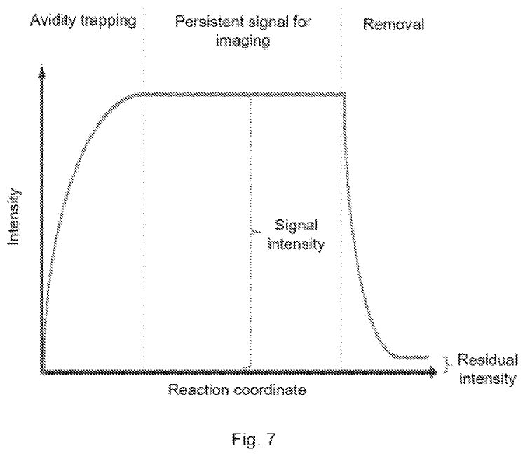

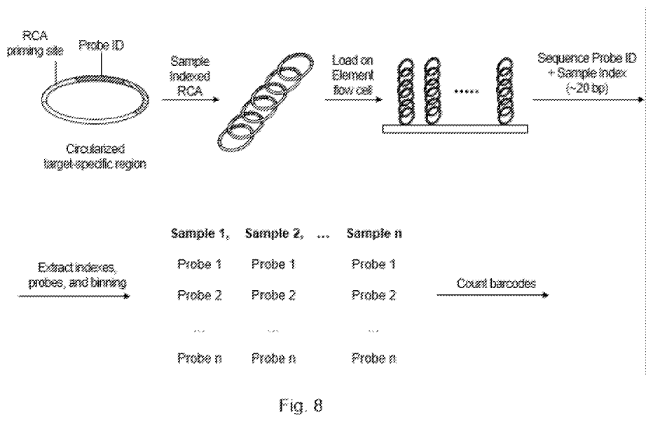

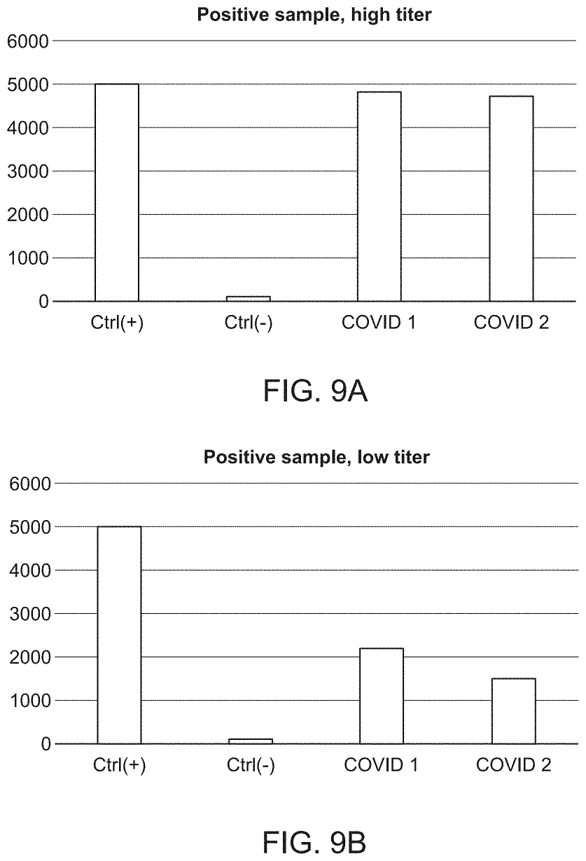

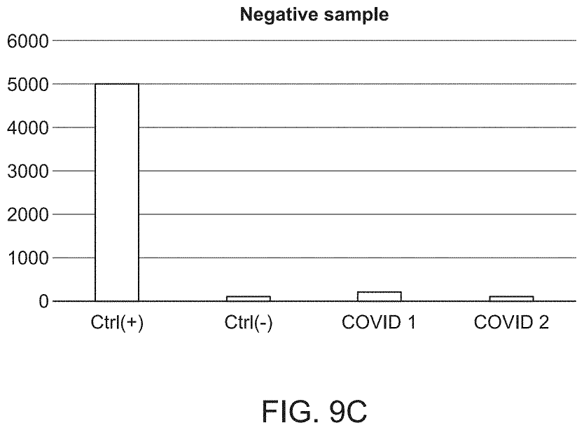

When the multivalent binding composition is used in sequencing reactions (instead of single unconjugated or untethered nucleotides) to form a multivalent binding complex with the polymerase and two or more copies of the target nucleic acid sequence, the effective local concentration of the nucleotide as well as the binding avidity of the complex are increased many-fold, which in turn enhances the persistence time of the complex (as illustrated in FIG. 7), increases signal-to-noise ratios and the differential signal intensity (e.g., the signal intensity for correct base-pairing versus mismatch), enables the use of shorter imaging steps, and improves base-calling accuracy. The multivalent binding composition described herein can include at least one particle-nucleotide conjugate (each particle-nucleotide conjugate comprising multiple copies of a single nucleotide moiety) for interacting with the target nucleic acid. The multivalent composition can also include two, three, or four different particle-nucleotide conjugates, each having a different nucleotide conjugated to the particle.

The present disclosure provides a method of using composition comprising a particle (e.g., a nanoparticle or polymer core), said particle comprising a plurality of enzyme or protein binding or incorporation substrates, wherein the enzyme or protein binding or incorporation substrates bind with one or more enzymes or proteins to form one or more binding or incorporation complexes (e.g., a multivalent binding or incorporation complex), and wherein said binding or incorporation may be monitored or identified by observation of the location, presence, or persistence of the one or more binding or incorporation complexes. In some embodiments, said particle may comprise a polymer, branched polymer, dendrimer, liposome, micelle, nanoparticle, or quantum dot. In some embodiments, said substrate may comprise a nucleotide, a nucleoside, a nucleotide analog, or a nucleoside analog. In some embodiments, the enzyme or protein binding or incorporation substrate may comprise an agent that can bind with a polymerase. In some embodiments, the enzyme or protein may comprise a polymerase. In some embodiments, said observation of the location, presence, or persistence of one or more binding or incorporation complexes may comprise fluorescence detection.

The multivalent binding or incorporation composition can comprise 1, 2, 3, 4, or more types of particle-nucleotide conjugates, wherein each particle-nucleotide conjugate comprises a different type of nucleotide. A first type of the particle-nucleotide conjugate can comprise a nucleotide selected from the group comprising ATP, ADP, AMP, dATP, dADP, and dAMP. A second type of the particle-nucleotide conjugate can comprise a nucleotide selected from the group comprising TTP, TDP, T1\1P, dTTP, dTDP, dTNIP, UTP, UDP, UNIP, dUTP, dUDP, and dUMP. A third type of the particle-nucleotide conjugate can comprise a nucleotide selected from the group comprising CTP, CDP, C1\1P, dCTP, dCDP, and dCMP. A fourth type of the particle-nucleotide conjugate can comprise a nucleotide selected from the group comprising GTP, GDP, G1\1P, dGTP, dGDP, and dGMP. In some embodiments, each particle-nucleotide conjugate comprises a single type of nucleotide respectively corresponding to one or more nucleotide selected from the group comprising ATP, ADP, AMP, dATP, dADP, dAMP TTP, TDP, TMP, dTTP, dTDP, dTMP, UTP, UDP, UMP, dUTP, dUDP, dUMP, CTP, CDP, CMP, dCTP, dCDP, dCMP, GTP, GDP, GMP, dGTP, dGDP, and dGMP.

Each multivalent binding or incorporation composition may further comprise one or more labels corresponding to the particular nucleotide conjugated to each respective conjugate. Non-limiting examples of labels include fluorescent labels (e.g., cyanine dye 3 (Cy3), cyanine dye 3.5 (Cy3.5), cyanine dye 5 (Cy5), and cyanine dye 5.5. (Cy5.5)), colorimetric labels, electrochemical labels (for example, glucose or other reducing sugars, or thiols or other redox active moieties), luminescent labels, chemiluminescent labels, spin labels, radioactive labels, steric labels, affinity tags, or the like.

In some embodiments, the present disclosure provides methods of preparing and using said composition wherein one or more labels comprise a fluorescent label, a FRET donor, or a FRET acceptor. In some embodiments, the present disclosure provides methods of preparing and using said composition wherein the substrate (e.g., nucleotide, nucleotide analog, nucleoside, or nucleoside analog) is attached to the particle through a linker. In some embodiments, the present disclosure provides methods of preparing and using said composition wherein at least one nucleotide or nucleotide analog is a nucleotide that has been modified to inhibit elongation during a polymerase reaction or a sequencing reaction, for example, a nucleotide that lacks a 3′ hydroxyl group; a nucleotide that has been modified to contain a blocking group at the 3′ position; a nucleotide that has been modified with a 3′-O-azido group, a 3′-O-azidomethyl group, a 3′-O-alkyl hydroxylamino group, a 3′-phosphorothioate group, a 3′-O-malonyl group, or a 3′-O-benzyl group; or a nucleotide that has not been modified at the 3′ position.

One non-limiting example of the particle-nucleotide conjugate is a polymer-nucleotide conjugate comprising a polymer core to which a plurality of nucleotide moieties, nucleotide analog moieties, other binding elements, linkers, or detectable labels may be tethered. In some instances, the polymer core may comprise linear or branched polymers. Examples of linear or branched polymers include linear or branched poly(ethylene glycol) (PEG), linear or branched poly(propylene glycol), linear or branched poly(vinyl alcohol), linear or branched polylactic acid, linear or branched poly(glycolic acid), linear or branched polyglycine, linear or branched poly(vinyl acetate), a dextran, or other such polymers, or copolymers incorporating any two or more of the foregoing or incorporating other polymers. In one embodiment, the polymer is a PEG.

In another embodiment, the polymer can have PEG branches.

Polymers may be characterized by a repeating unit incorporating a functional group for derivatization such as an amine, a hydroxyl, a carbonyl, or an allyl group. The polymer can also have one or more pre-derivatized substituents such that one or more particular subunits will incorporate a site of derivatization or a branch site, whether or not other subunits incorporate the same site, substituent, or moiety. A pre-derivatized substituent may comprise or may further comprise, for example, a nucleotide, a nucleoside, a nucleotide analog, a label such as a fluorescent label, radioactive label, or spin label, an interaction moiety, an additional polymer moiety, or the like, or any combination of the foregoing.

In the polymer-nucleotide conjugate, the polymer can have a plurality of branches. The branched polymer can have various configurations, including but are not limited to, stellate (“starburst”) forms, aggregated stellate (“helter skelter”) forms, bottle brush, or dendrimer. The branched polymer can radiate from a central attachment point or central moiety or may incorporate multiple branch points, for example, 2, 3, 4, 5, 6, 7, 8, 9, 10, or more branch points. In some embodiments, each subunit of a polymer may optionally constitute a separate branch point.

The length and size of the branch can differ based on the type of polymer. In some branched polymers, the branch may have a length of between 1 and 1,000 nm, between 1 and 100 nm, between 1 and 200 nm, between 1 and 300 nm, between 1 and 400 nm, between 1 and 500 nm, between 1 and 600 nm, between 1 and 700 nm, between 1 and 800 nm, or between 1 and 900 nm, or more, or having a length falling within or between any of the values disclosed herein.

In some polymer-nucleotide conjugates, the polymer core may have a size corresponding to an apparent molecular weight of 1 kDa, 2 kDa, 3 kDa, 4 kDa, 5 kDa, 10 kDa, 15 kDa, 20 kDa, 30 kDa, 50 kDa, 80 kDa, 100 kDa, or any value within a range defined by any two of the foregoing. The apparent molecular weight of a polymer may be calculated from the known molecular weight of a representative number of subunits, as determined by size exclusion chromatography, as determined by mass spectrometry, or as determined by any other existing methods.

In some branched polymers, the branch may have a size corresponding to an apparent molecular weight of 1 kDa, 2 kDa, 3 kDa, 4 kDa, 5 kDa, 10 kDa, 15 kDa, 20 kDa, 30 kDa, 50 kDa, 80 kDa, 100 kDa, or any value within a range defined by any two of the foregoing. The apparent molecular weight of a polymer may be calculated from the known molecular weight of a representative number of subunits, as determined by size exclusion chromatography, as determined by mass spectrometry, or as determined by any other method as is known in the art. The polymer can have multiple branches. The number of branches in the polymer can be 2, 3, 4, 5, 6, 7, 8, 12, 16, 24, 32, 64, 128 or more, or a number falling within a range defined by any two of these values.

For polymer-nucleotide conjugates comprising a branched polymer of, for example, a branched PEG comprising 4, 8, 16, 32, or 64 branches, the polymer nucleotide conjugate can have nucleotides attached to the ends of the PEG branches, such that each end has attached thereto 0, 1, 2, 3, 4, 5, 6 or more nucleotides. In one non-limiting example, a branched PEG polymer of between 3 and 128 PEG arms may have attached to the ends of the polymer branches one or more nucleotides, such that each end has attached thereto 0, 1, 2, 3, 4, 5, 6 or more nucleotides or nucleotide analogs. In some embodiments, a branched polymer or dendrimer has an even number of arms. In some embodiments, a branched polymer or dendrimer has an odd number of arms.

In some instances, the length of the linker (e.g., a PEG linker) may range from about 1 nm to about 1,000 nm. In some instances, the length of the linker may be at least 1 nm, at least 10 nm, at least 25 nm, at least 50 nm, at least 75 nm, at least 100 nm, at least 200 nm, at least 300 nm, at least 400 nm, at least 500 nm, at least 600 nm, at least 700 nm, at least 800 nm, at least 900 nm, or at least 1,000 nm. In some instances, the length of the linker may range between any two of the values in this paragraph. For example, in some instances, the length of the linker may range from about 75 nm to about 400 nm. It is possible that in some instances, the length of the linker may have any value within the range of values in this paragraph, e.g., 834 nm.

In some instances, the length of the linker is different for different nucleotides (including deoxyribonucleotides and ribonucleotides), nucleotide analogs (including deoxyribonucleotide analogs and ribonucleotide analogs), nucleosides (including deoxyribonucleosides or ribonucleosides), or nucleoside analogs (including deoxyribonucleoside analogs or ribonucleoside analogs). In some instances, one of the nucleotides, nucleotide analogs, nucleosides, or nucleoside analogs comprises, for example, deoxyadenosine, and the length of the linker is between 1 nm and 1,000 nm. In some instances, one of the nucleotides, nucleotide analogs, nucleosides, or nucleoside analogs comprises, for example, deoxyguanosine, and the length of the linker is between 1 nm and 1,000 nm. In some instances, one of the nucleotides, nucleotide analogs, nucleosides, or nucleoside analogs comprises, for example, thymidine, and the length of the linker is between 1 nm and 1,000 nm. In some instances, one of the nucleotides, nucleotide analogs, nucleosides, or nucleoside analogs comprises, for example, deoxyuridine, and the length of the linker is between 1 nm and 1,000 nm. In some instances, one of the nucleotides, nucleotide analogs, nucleosides, or nucleoside analogs comprises, for example, deoxycytidine, and the length of the linker is between 1 nm and 1,000 nm. In some instances, one of the nucleotides, nucleotide analogs, nucleosides, or nucleoside analogs comprises, for example, adenosine, and the length of the linker is between 1 nm and 1,000 nm. In some instances, one of the nucleotides, nucleotide analogs, nucleosides, or nucleoside analogs comprises, for example, guanosine, and the length of the linker is between 1 and 1,000 nm. In some instances, one of the nucleotides, nucleotide analogs, nucleosides, or nucleoside analogs comprises, for example, 5-methyl-uridine, and the length of the linker is between 1 nm and 1,000 nm. In some instances, one of the nucleotides, nucleotide analogs, nucleosides, or nucleoside analogs comprises, for example, uridine, and the length of the linker is between 1 nm and 1,000 nm. In some instances, one of the nucleotides, nucleotide analogs, nucleosides, or nucleoside analogs comprises, for example, cytidine, and the length of the linker is between 1 nm and 1,000 nm.

In the polymer-nucleotide conjugate, each branch or a subset of branches of the polymer may have attached thereto a moiety comprising a nucleotide (e.g., an adenine, a thymine, a uracil, a cytosine, or a guanine residue or a derivative or mimetic thereof), and the moiety is capable of binding or incorporation to a polymerase, reverse transcriptase, or other nucleotide binding or incorporation domain. Optionally, the moiety may be capable of being incorporated into an elongating nucleic acid chain during a polymerase reaction. In some instances, said moiety may be blocked such that it is not capable of being incorporated into an elongating nucleic acid chain during a polymerase reaction. In some other instances, said moiety may be reversibly blocked such that it is not capable of being incorporated into an elongating nucleic acid chain during a polymerase reaction until such block is removed, after which said moiety is then capable of being incorporated into an elongating nucleic acid chain during a polymerase reaction.

The nucleotide can be conjugated to the polymer branch through the 5′ end of the nucleotide. In some instances, the nucleotide may be modified to inhibit or prevent incorporation of the nucleotide into an elongating nucleic acid chain during a polymerase reaction. By way of example, the nucleotide may include a 3′ deoxyribonucleotide, a 3′ azidonucleotide, a 3′-methyl azido nucleotide, or another such nucleotide as is or may be known in the art, to be incapable of being incorporated into an elongating nucleic acid chain during a polymerase reaction. In some embodiments, the nucleotide can include a 3′-O-azido group, a 3′-O-azidomethyl group, a 3′ phosphorothioate group, a 3′-O-malonyl group, a 3′-O-alkyl hydroxylamino group, or a 3′-O-benzyl group. In some embodiments, the nucleotide lacks a 3′ hydroxyl group.

The polymer can further have a binding or incorporation moiety in each branch or a subset of branches. Some examples of the binding or incorporation moiety include, but are not limited to biotin, avidin, strepavidin or the like, polyhistidine domains, complementary paired nucleic acid domains, G-quartet forming nucleic acid domains, calmodulin, maltose-binding protein, cellulase, maltose, sucrose, glutathione-S-transferase, glutathione, O-6-methylguanine-DNA methyltransferase, benzylguanine and derivatives thereof, benzylcysteine and derivatives thereof, an antibody, an epitope, a protein A, a protein G. The binding or incorporation moiety can be any interactive molecules or fragment thereof known in the art to bind to or facilitate interactions between proteins, between proteins and ligands, between proteins and nucleic acids, between nucleic acids, or between small molecule interaction domains or moieties.

Without intending to be bound by any particular theory, it has been observed that multivalent binding compositions disclosed herein associate with polymerase nucleotide complexes in order to form a ternary binding complexes with a rate that is time-dependent, though substantially slower than the rate of association is obtainable by nucleotides in free solution. Thus, the on-rate (Kₒₙ) is substantially and surprisingly slower than the on rate for single nucleotides or nucleotides not attached to multivalent ligand complexes. Importantly, however, the off rate (K_{off}) of the multivalent ligand complex is substantially slower than that observed for nucleotides in free solution. Therefore, the multivalent ligand complexes of the present disclosure provide a surprising and beneficial improvement of the persistence of ternary polymerase-polynucleotide-nucleotide complexes (especially over such complexes that are formed with free nucleotides) allowing, for example, significant improvements in imaging quality for nucleic acid sequencing applications over currently available methods and reagents. Importantly, this property of the multivalent binding compositions disclosed herein renders the formation of visible ternary complexes controllable, such that subsequent visualization, modification, or processing operations may be undertaken essentially without regard to the dissociation of the complex, that is, the complex can be formed, imaged, modified, or used in other ways, and will remain stable until a user carries out an affirmative dissociation operation, such as exposing the complexes to a dissociation buffer.

In some instances, the multivalent binding complexes formed between a multivalent binding composition (at low effective nucleotide concentration) such as a polymer-nucleotide conjugate, a polymerase, and two or more copies of a target nucleic acid sequence may have a persistence time ranging from about 0.1 second to about 600 second under non-destabilizing conditions. In some instances, the persistence time may be at least 0.1 second, at least 1 second, at least 2 seconds, at least 3 second, at least 4 second, at least 5 seconds, at least 6 seconds, at least 7 seconds, at least 8 seconds, at least 9 seconds, at least 10 seconds, at least 20 seconds, at least 30 second, at least 40 second, at least 50 seconds, at least 60 seconds, at least 120 seconds, at least 180 seconds, at least 240 seconds, at least 300 seconds, at least 360 seconds, at least 420 seconds, at least 480 seconds, at least 540 seconds, or at least 600 seconds. In some instances, the persistence time may range between any two of the values specified in this paragraph. For example, in some instances, the persistence time may range from about 10 seconds to about 360 seconds. It is possible that, in some instances, the persistence time may have any value within the range of values specified in this paragraph, e.g., 78 seconds.

In some instances, the aforementioned persistence times may be achieved when using the multivalent binding composition, e.g., a polymer-nucleotide conjugate, for performing sequencing-by-trapping reactions using effective nucleotide concentrations of less than 1,000 nM, less than 500 nM, less than 400 nM, less than 300 nM, less than 200 nM, less than 150 nM, less than 100 nM, less than 90 nM, less than 80 nM, less than 70 nM, less than 60 nM, less than 50 nM, less than 40 nM, less than 30 nM, less than 20 nM, less than 15 nM, less than 10 nM, less than 9 nM, less than 8 nM, less than 7 nM, less than 6 nM, less than 5 nM, less than 4 nM, less than 3 nM, less than 2 nM, or less than 1 nM.

### Polymerase for the Multivalent Binding Complexes

In various embodiments, polymerases for the binding or incorporation interaction describe herein may include any polymerase as is or may be known in the art. Examples of polymerases may include but are not limited to: Klenow DNA polymerase, Thermus aquaticus DNA polymerase I (Taq polymerase), KlenTaq polymerase, and bacteriophage T7 DNA polymerase; human alpha, delta and epsilon DNA polymerases; bacteriophage polymerases such as T4, RB69 and phi29 bacteriophage DNA polymerases, Pyrococcus furiosus DNA polymerase (Pfu polymerase); Bacillus subtilis DNA polymerase III, and E. coli DNA polymerase III alpha and epsilon; 9 degree N polymerase, reverse transcriptases such as HIV type M or O reverse transcriptases, avian myeloblastosis virus reverse transcriptase, or Moloney Murine Leukemia Virus (MMLV) reverse transcriptase, or telomerase. Further non-limiting examples of DNA polymerases can include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including polymerases such as Vent™, Deep Vent™, Pfu, KOD, Pfx, Therminator™, and Tgo polymerases. In some embodiments, the polymerase is a klenow polymerase.

The ternary complex has longer persistence time when the nucleotide on the polymer-nucleotide conjugate is complementary to the target nucleic acid than when a non-complementary nucleotide. The ternary complex also has longer persistence time when the nucleotide on the polymer-nucleotide conjugate is complementary to the target nucleic acid than a complementary nucleotide that is not conjugated or tethered. For example, in some embodiments, said ternary complexes may have a persistence time of less than Is, greater than Is, greater than 2s, greater than 3s, greater than 5s, greater than 10s, greater than 15s, greater than 20s, greater than 30s, greater than 60s, greater than 120s, greater than 360s, greater than 3600s, or more, or for a time lying within a range defined by any two or more of these values.

The persistence time can be measured, for example, by observing the onset or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex.

It has been observed that different ranges of persistence times are achievable with different salts or ions, showing, for example, that complexes formed in the presence of, for example, magnesium ions (Mg²⁺) form more quickly than complexes formed with other ions. It has also been observed that complexes formed in the presence of, for example, strontium ions (Sr²⁺), form readily and dissociate completely or with substantial completeness upon withdrawal of the ion or upon washing with buffer lacking one or more components of the present compositions, such as, e.g., a polymer or one or more nucleotides, or one or more interaction moieties, or a buffer containing, for example, a chelating agent which may cause or accelerate the removal of a divalent cation from the multivalent reagent containing complex. Thus, in some embodiments, a composition of the present disclosure comprises Mg²⁺. In some embodiments, a composition of the present disclosure comprises Ca²⁺. In some embodiments, a composition of the present disclosure comprises Sr²⁺. In some embodiments, a composition of the present disclosure comprises cobalt ions (Co²⁺). In some embodiments, a composition of the present disclosure comprises MgCl₂. In some embodiments, a composition of the present disclosure comprises CaCl₂. In some embodiments, a composition of the present disclosure comprises SrCl₂. In some embodiments, a composition of the present disclosure comprises CoCl₂. In some embodiments, the composition comprises no, or substantially no magnesium. In some embodiments, the composition comprises no, or substantially no calcium. In some embodiments, the methods of the present disclosure provide for the contacting of one or more nucleic acids with one or more of the compositions disclosed herein wherein said composition lacks either one of calcium or magnesium or lacks both calcium and magnesium.

The dissociation of ternary complexes can be controlled by changing the buffer conditions. After the imaging operation, a buffer with increased salt content is used to cause dissociation of the ternary complexes such that labeled polymer-nucleotide conjugates can be washed out, providing a mechanism by which signals can be attenuated or terminated, such as in the transition between one sequencing cycle and the next. This dissociation may be affected, in some embodiments, by washing the complexes with a buffer lacking a metal or cofactor. In some embodiments, a wash buffer may comprise one or more compositions for the purpose of maintaining pH control. In some embodiments, a wash buffer may comprise one or more monovalent cations, such as sodium. In some embodiments, a wash buffer lacks or substantially lacks a divalent cation, for example, having no or substantially no strontium, calcium, magnesium, or manganese. In some embodiments, a wash buffer further comprises a chelating agent, for example, EDTA, EGTA, nitrilotriacetic acid, polyhistidine, imidazole, or the like. In some embodiments, a wash buffer may maintain the pH of the environment at the same level as for the bound complex. In some embodiments, a wash buffer may raise or lower the pH of the environment relative to the level seen for the bound complex. In some embodiments, the pH may be within a range from 2-4, 2-7, 5-8, 7-9, 7-10, or lower than 2, or higher than 10, or a range defined by any two of the values provided herein.

Addition of a particular ion may affect the binding of the polymerase to a primed target nucleic acid, the formation of a ternary complex, the dissociation of a ternary complex, or the incorporation of one or more nucleotides into an elongating nucleic acid such as during a polymerase reaction. In some embodiments, relevant anions may comprise chloride, acetate, gluconate, sulfate, phosphate, or the like. In some embodiments, an ion may be incorporated into the compositions of the present disclosure by the addition of one or more acids, bases, or salts, such as NiCl₂, CoCl₂, MgCl₂, MnCl₂, SrCl₂, CaCl₂, CaSO₄, SrCO₃, BaCl₂ or the like. Representative salts, ions, solutions and conditions may be found in Remington: The Science and Practice of Pharmacy, 20th. Edition, Gennaro, A. R., Ed. (2000), which is hereby incorporated by reference in its entirety, and especially with respect to Chapter 17 and related disclosure of salts, ions, salt solutions, and ionic solutions.

The present disclosure contemplates contacting the multivalent binding or incorporation composition comprising at least one particle-nucleotide conjugate with one or more polymerases. The contacting can be optionally done in the presence of one or more target nucleic acids. In some embodiments, said target nucleic acids are single stranded nucleic acids. In some embodiments, said target nucleic acids are primed single stranded nucleic acids. In some embodiments, said target nucleic acids are double stranded nucleic acids. In some embodiments, said contacting comprises contacting the multivalent binding or incorporation composition with one polymerase. In some embodiments, said contacting comprises the contacting of said composition comprising one or more nucleotides with multiple polymerases. The polymerase can be bound to a single nucleic acid molecule.

The target nucleic acid can refer to a target nucleic acid sample having one or more nucleic acid molecules. In some embodiments, the target nucleic acid can include a plurality of nucleic acid molecules. In some embodiments, the target nucleic acid can include two or more nucleic acid molecules. In some embodiments, the target nucleic acid can include two or more nucleic acid molecules having the same sequences.

The binding between target nucleic acid and multivalent binding composition may be provided in the presence of a polymerase that has been rendered catalytically inactive. In one embodiment, the polymerase may have been rendered catalytically inactive by mutation. In one embodiment, the polymerase may have been rendered catalytically inactive by chemical modification. In some embodiments, the polymerase may have been rendered catalytically inactive by the absence of a substrate, ion, or cofactor. In some embodiments, the polymerase enzyme may have been rendered catalytically inactive by the absence of magnesium ions.

The binding between target nucleic acid and multivalent binding composition occur in the presence of a polymerase wherein the binding solution, reaction solution, or buffer lacks magnesium or manganese. Alternatively, the binding between target nucleic acid and multivalent binding composition occur in the presence of a polymerase wherein the binding solution, reaction solution, or buffer comprises calcium or strontium.

When the catalytically inactive polymerases are used to help a nucleic acid interact with a multivalent binding composition, the interaction between said composition and said polymerase stabilizes a ternary complex so as to render the complex detectable by fluorescence or by other methods as disclosed herein or otherwise known in the art. Unbound polymer-nucleotide conjugates may optionally be washed away prior to detection of the ternary binding complex.

Contacting of one or more nucleic acids with the polymer-nucleotide conjugates disclosed herein in a solution containing either one of calcium or magnesium or containing both calcium and magnesium. Alternatively, the contacting of one or more nucleic acids with the polymer-nucleotide conjugates disclosed herein in a solution lacking either one of calcium or magnesium, or lacking both calcium or magnesium, and in a separate operation, without regard to the order of the operations, adding to the solution one of calcium or magnesium, or both calcium and magnesium. In some embodiments, the contacting of one or more nucleic acids with the polymer-nucleotide conjugates disclosed herein in a solution lacking strontium, and comprises in a separate operation, without regard to the order of the operations, adding to the solution strontium.

### Sequencing Systems

Also disclosed herein are sequencing systems configured to perform the disclosed barcoded padlock probe and molecular inversion probe assays. The disclosed sequencing systems may comprise novel sequencing chemistries, sequencing flow cells, imaging modules, fluid flow controllers or fluid dispensing systems, processors or computer systems, or any combination thereof. Applicant is developing proprietary sequencing chemistries (e.g., “sequencing-by-trapping” chemistries), sequencing flow cells, and sequencing systems that provide high quality nucleic acid sequence data at high throughput and low cost in a compact, modular format. The sequencing platform (and associated consumable kit) will be configured as a highly multiplexed barcode reader that minimizes reagent consumption and assay cost while affording a barcode reading efficiency that is unprecedented in the context of conventional molecular diagnostic testing. The implementation of the disclosed padlock probe or molecular inversion probe assays followed by RCA amplification generates a huge number of data points, where each concatemer generated corresponds to a unique assay replicate. The disclosed sequencing platform and sequencing consumables allow one to discriminate between 100s of millions of these concatemers. The large number of replicates involved will thus yield very accurate assays and will also provide information on viral load since the number of concatemers generated will be proportional to the viral copies initially present in the sample.

### Sequencing Flow Cells

In some instances, one or more interior surfaces of the sequencing flow cells of the disclosed systems may comprise novel low non-specific binding surface chemistries that have been optimized for low background/high foreground fluorescence signals that yield high contrast-to-noise ratio images of fluorescently tagged molecules tethered to a flow cell surface. In some instances, one or more sequencing flow cells may be fixed components of the sequencing system. In some instances, one or more sequencing flow cells may be removable or disposable components of the sequencing system.

In some instances, the sequencing flow cell may be fabricated from off-the-shelf components such as glass capillaries, fused-silica capillaries, or polymer capillaries. Examples of materials include, but are not limited to, glass, fused-silica, silicon, a polymer (e.g., polystyrene (PS), microporous polystyrene (MPPS), poly(methyl methacrylate) (PMMA), polycarbonate (PC), polypropylene (PP), polyethylene (PE), high-density polyethylene (HDPE), cyclic olefin polymers (COP), cyclic olefin copolymers (COC), poly(ethylene terephthalate) (PET)), or any combination thereof. Various flow cell designs constructed of both glass and polymer components are contemplated.

In some instances, the one or more interior surfaces of the sequencing flow cell may comprise one or more layers of a covalently or non-covalently attached low-binding, chemical modification layers, e.g., silane layers, polymer films, and one or more covalently or non-covalently attached primer sequences that may be used for tethering single-stranded target nucleic acid(s) to the support surface. In some instances, the formulation of the surface, e.g., the chemical composition of one or more layers, the coupling chemistry used to cross-link the one or more layers to the support surface or to each other, and the total number of layers, may be varied such that non-specific binding of proteins, nucleic acid molecules, and other hybridization and amplification reaction components to the support surface is minimized or reduced relative to a comparable monolayer. Often, the formulation of the surface may be varied such that non-specific hybridization on the support surface is minimized or reduced relative to a comparable monolayer. The formulation of the surface may be varied such that non-specific amplification on the support surface is minimized or reduced relative to a comparable monolayer or unmodified surface. The formulation of the surface may be varied such that specific amplification rates or yields on the support surface are maximized in those instances where a solid-phase amplification step is incorporated into the assay.

In some instances, the low non-specific binding surfaces may comprise 1, 2, 3, 4, 5, 6, 7, 8, 9, 10, or more than 10 layers of a hydrophilic polymer coating. Examples of polymers include, but are not limited to, poly(ethylene glycol) (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(hydroxyethyl methacrylate) (PHEMA), poly(oligo(ethylene glycol) methyl ether methacrylate) (POEGMA), polyglutamic acid (PGA), poly-lysine, poly-glucoside, streptavidin, and dextran.

In some instances, one or more polymer coating layers may comprise a branched or multibranched polymer. Examples of branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(hydroxylethyl methacrylate) (branched PHEMA), branched poly(oligo(ethylene glycol) methyl ether methacrylate) (branched POEGMA), branched poly(glutamic acid) (branched PGA), branched polylysine, branched polyglucoside, and dextran.

In some instances, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches. Molecules often exhibit a ‘power of 2’ number of branches, such as 2, 4, 8, 16, 32, 64, or 128 branches.

In some instances, the linear, branched, or multi-branched polymers used to create one or more layers of any of the low non-specific binding surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 Da.

In some instances, 1, 2, 3, 4, or more than 4 polymer coating layers of the low non-specific binding surfaces may comprise a plurality of tethered oligonucleotide primer or adapter sequences attached or tethered thereto. One or more types of oligonucleotide primer or adapter sequences may be attached one or more polymer coating layers on the surface. In some instances, the one or more types of oligonucleotide adapters or primers may comprise spacer sequences, adapter sequences for hybridization to adapter-ligated template library nucleic acid sequences, forward amplification primers, reverse amplification primers, sequencing primers, or molecular barcoding sequences, or any combination thereof. In some instances, 1 primer or adapter sequence may be tethered to at least one layer of the surface. In some instances, at least 2, 3, 4, 5, 6, 7, 8, 9, 10, or more than 10 different primer or adapter sequences may be tethered to at least one layer of the surface.

In some instances, the tethered oligonucleotide adapter or primer sequences may range in length from about 10 nucleotides to about 100 nucleotides. In some instances, the tethered oligonucleotide adapter or primer sequences may be at least 10, at least 20, at least 30, at least 40, at least 50, at least 60, at least 70, at least 80, at least 90, or at least 100 nucleotides in length. In some instances, the tethered oligonucleotide adapter or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some instances the length of the tethered oligonucleotide adapter or primer sequences may range from about 20 nucleotides to about 80 nucleotides. It is possible that the length of the tethered oligonucleotide adapter or primer sequences may have any value within this range, e.g., about 24 nucleotides.

In some instances, the effective surface density of oligonucleotide adapter or primer sequences on the low non-specific binding surfaces may range from about 100 molecules per μm² to about 100,000 molecules per μm². In some instances, the effective surface density of oligonucleotide adapter or primer sequences may range from about 1,000 molecules per μm² to about 1,000,000 molecules per μm². In some instances, the effective surface density of oligonucleotide adapter or primer sequences may be at least 100, at least 1,000, at least 10,000, at least 100,000, or at least 1,000,000 molecules per μm². In some instances, the effective surface density of oligonucleotide adapter or primer sequences may be at most 1,000,000, at most 100,000, at most 10,000, at most 1,000 molecules, or at most 100 molecules per μm². Any of the lower and upper values described in this paragraph may be combined to form a range included within the present disclosure, for example, in some instances the effective surface density of oligonucleotide adapter or primer sequences may range from about 10,000 molecules per μm² to about 100,000 molecules per μm². It is possible that the surface density of primer molecules may have any value within this range, e.g., about 455,000 molecules per μm². In some instances, the effective surface density of target nucleic acid sequences (e.g., concatemer or nanoball sequences) initially hybridized to the primer or adapter on the surface may be less than or equal to that indicated for the effective surface density of oligonucleotide primer or adapters. In some instances, the surface density of hybridized concatemer or nanoball sequences, or of clonally-amplified target nucleic acid sequences hybridized to primer or adapter sequences on the surface may span the same range as that indicated for the effective surface density of the oligonucleotide primer or adapter sequences. The local surface densities as listed above do not preclude variation in surface density across a surface, such that a surface may comprise a region having an oligonucleotide primer or adapter sequence surface density of, for example, 50,000 molecules per μm², while also comprising at least a second region having a substantially different local surface density.

In some instances, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some instances, a static contact angle may be determined. In some instances, an advancing or receding contact angle may be determined. In some instances, the water contact angle for the hydrophilic, low-binding support surfaced disclosed herein may range from about 0 degrees to about 50 degrees. In some instances, the water contact angle for the hydrophilic, low-binding support surfaced disclosed herein may be less than 50 degrees, less than 40 degrees, less than 30 degrees, less than 25 degrees, less than 20 degrees, less than 18 degrees, less than 16 degrees, less than 14 degrees, less than 12 degrees, less than 10 degrees, less than 8 degrees, less than 6 degrees, less than 4 degrees, less than 2 degrees, or less than 1 degree. In some cases, the contact angle is no more than 40 degrees. It is possible that a given hydrophilic, low-binding support surface of the present disclosure may exhibit a water contact angle having a value of anywhere within the range of 0 degrees to 50 degrees.

### Fluorescence Imaging of Support Surfaces

The disclosed solid-phase nucleic acid amplification reaction formulations and low-binding supports may be used in any of a variety of nucleic acid analysis applications, e.g., nucleic acid base discrimination, nucleic acid base classification, nucleic acid base calling, nucleic acid detection applications, nucleic acid sequencing applications, and nucleic acid-based (genetic and genomic) diagnostic applications. In many of these applications, fluorescence imaging techniques may be used to monitor hybridization, amplification, or sequencing reactions performed on the low non-specific binding supports.

Fluorescence imaging may be performed using any of a variety of fluorophores, fluorescence imaging techniques, and fluorescence imaging instruments. Examples of fluorescence dyes that may be used (e.g., by conjugation to nucleotides, oligonucleotides, or proteins) include, but are not limited to, fluorescein, rhodamine, coumarin, cyanine, and derivatives thereof, including the cyanine derivatives Cyanine dye-3 (Cy3), Cyanine dye-5 (Cy5), Cyanine dye-7 (Cy7), etc. Examples of fluorescence imaging techniques that may be used include, but are not limited to, wide-field fluorescence microscopy fluorescence microscopy imaging, fluorescence confocal imaging, two-photon fluorescence, and the like. Examples of fluorescence imaging instruments that may be used include, but are not limited to, fluorescence microscopes equipped with an image sensor or camera, wide-field fluorescence microscopy, confocal fluorescence microscopes, two-photon fluorescence microscopes, or custom instruments that comprise a selection of light sources, lenses, mirrors, prisms, dichroic reflectors, apertures, and image sensors or cameras, etc. A non-limiting example of a fluorescence microscope equipped for acquiring images of the disclosed low-binding support surfaces and clonally-amplified colonies (or clusters) of target nucleic acid sequences hybridized thereon is the Olympus IX83 inverted fluorescence microscope equipped with) 20×, 0.75 NA, a 532 nm light source, a bandpass and dichroic mirror filter set optimized for 532 nm long-pass excitation and Cy3 fluorescence emission filter, a Semrock 532 nm dichroic reflector, and a camera (Andor sCMOS, Zyla 4.2) where the excitation light intensity is adjusted to avoid signal saturation. Often, the support surface may be immersed in a buffer (e.g., 25 mM ACES, pH 7.4 buffer) while the image is acquired.

In some instances, the low non-specific binding surfaces exhibit reduced non-specific binding of proteins, nucleic acids, and other components of the hybridization or amplification formulations used for tethering target nucleic acid sequences (e.g., concatemer or nanoball sequences) to the surface or for performing solid-phase nucleic acid amplification. The degree of non-specific binding exhibited by a given support surface may be assessed either qualitatively or quantitatively. For example, in some instances, exposure of the surface to fluorescent dyes (e.g., Cy3, Cy5, etc.), fluorescently-labeled nucleotides, fluorescently-labeled oligonucleotides, or fluorescently-labeled proteins (e.g. polymerases) under a standardized set of conditions followed by a specified rinse protocol and fluorescence imaging may be used as a qualitative or quantitative tool for comparison of non-specific binding on surfaces comprising different surface formulations—provided that care has been taken to ensure that the fluorescence imaging is performed under conditions where fluorescence signal is linearly related (or related in a predictable manner) to the number of fluorophores on the support surface (e.g., under conditions where signal saturation or self-quenching of the fluorophore is not an issue) and calibration standards are used. In some instances, other existing techniques, for example, radioisotope labeling and counting methods may be used for quantitative assessment of the degree to which non-specific binding is exhibited by different surface formulations. In some instances, the low non-specific-binding surfaces of the present disclosure may exhibit non-specific protein binding (or non-specific binding of other specified molecules, e.g., Cy3 dye) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². It is possible that a given surface may exhibit non-specific binding falling anywhere within this range, for example, of less than 86 molecules per μm².

In some instances, the performance of nucleic acid hybridization or amplification reactions using the disclosed low non-specific binding surfaces may be assessed using fluorescence imaging techniques, where the contrast-to-noise ratio (CNR) of the images provides a key metric in assessing, e.g., amplification specificity or non-specific binding on the support. CNR is commonly defined as: CNR=(Signal−Background)/Noise. The background term is commonly taken to be the signal measured for the interstitial regions surrounding a particular feature (e.g., a diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times may be minimized). At high CNR the imaging time required to reach accurate discrimination (and thus accurate base-calling in the case of sequencing applications) can be drastically reduced even with moderate improvements in CNR.

In most ensemble-based sequencing approaches, the background term is measured as the signal associated with ‘interstitial’ regions. In addition to “interstitial” background (Binter), “intrastitial” background (Bᵢₙₜᵣₐ) exists within the region occupied by, e.g., an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bar background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In next generation sequencing (NGS) applications, this background signal in the current field-of-view (FOV) is averaged over time and subtracted. The signal arising from individual DNA colonies (e.g., (S)−Bᵢₙₜₑᵣ in the FOV) yields a discernable feature that can be classified. In some instances, the Bᵢₙₜᵣₐ can contribute a confounding fluorescence signal that is not specific to the target of interest but is present in the same ROI thus making it far more difficult to average and subtract. A more accurate calculation of CNR is thus provided by the formula CNR=(Signal−Background)/Noise, where Background=Bᵢₙₜₑᵣ+Bᵢₙₜᵣₐ.

In some instances, as a result of the extremely low non-specific binding (low background signal) and dense, tightly packed nanoball sequences (or clonally-amplified target nucleic acid clusters) that are achievable on the disclosed surfaces, fluorescence images of said surfaces may exhibit improvements in CNR by a factor of 2, 5, 10, 100, or 1000-fold over those achieved using conventional support surfaces. In some instances, fluorescence images of one or more interior surfaces of the sequencing flow cells disclosed herein, when used in nucleic acid hybridization or amplification applications to create clusters of hybridized or clonally-amplified nucleic acid molecules (e.g., that have been directly or indirectly labeled with a fluorophore), or when used to perform sequencing of the disclosed barcoded padlock probe and molecular inversion probe assays) may exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250 when the image is acquired under a defined set of conditions, e.g., when the nucleic acid molecules or complementary sequences thereof are labeled with a Cy3 fluorophore, and when the fluorescence image is acquired using an Olympus IX83 inverted fluorescence microscope equipped with a total internal reflectance fluorescence (TIRF) 100×, 1.5 NA objective, a 100 W Hg lamp, a 532 nm long-pass excitation filter, a Semrock 532 nm dichroic reflector, and an Olympus EM-CCD camera under non-signal saturating conditions while the surface is immersed in a 25 mM ACES, pH 7.4 buffer.

In some embodiments, sequencing methods utilizing the compositions and methods disclosed herein may incorporate a detection method enabling base calling to reveal the sequence of the target nucleic acid. In some embodiments, these detection methods may include any method for nucleic acid detection or nucleic acid sequencing. In some embodiments, the systems described herein are used to perform the base calling procedure. In some embodiments, said detection methods may include, for example, one or more of fluorescence detection, colorimetric detection, luminescence (such as chemiluminescence of bioluminescence) detection, interferometric detection, resonance-based detection such as Raman detection, spin resonance-based detection, NMR-based detection, and the like, and other methods such as electrical detection, for example, capacitance-based detection, impedance based detection, or electrochemical detection, such as detection of electrons generated by or within a chemical reaction, or combinations of electrical, such as, e.g., impedance measurements, with other, e.g., optical measurements.

It may be advantageous to provide the multivalent binding compositions in combination with other elements such as to provide optimized signals, for example to provide identification of a nucleotide at a particular position in a nucleic acid sequence. In some embodiments, the compositions disclosed herein are provided in combination with a surface providing low background binding or low levels of protein binding, especially a hydrophilic or polymer coated surface. Representative surfaces may be found, for example, in U.S. patent application Ser. No. 16/363,842, the contents of which are hereby incorporated by reference in their entirety.

### Imaging Modules

In some instances, the disclosed systems may comprise one or more imaging modules, where an imaging module comprises, e.g., one or more light sources (e.g., lasers, laser diodes, arc lamps, tungsten-halogen lamps, etc.), one or more optical components (e.g., lenses, mirrors, prisms, optical filters, colored glass filters, narrowband interference filters, broadband interference filters, dichroic reflectors, diffraction gratings, apertures, optical fibers, or optical waveguides and the like), and one or more image sensors (e.g., charge-coupled device (CCD) sensors or cameras, complementary metal-oxide-semiconductor (CMOS) image sensors or cameras, or negative-channel metal-oxide semiconductor (NMOS) image sensors or cameras) configured for imaging one or more interior surfaces of a sequencing flow cell or detection of binding of the disclosed multivalent binding compositions to target (or template) nucleic acid sequences tethered to a surface on the interior of a sequencing flow cell.

### Fluid Flow Controllers or Fluid Dispensing Systems

In some instances, the system may further comprise one or more fluid flow controllers or fluid dispensing modules configured to sequentially and iteratively contact template nucleic acid sequences hybridized to adapter or primer sequences on the interior surface(s) of the flow cell (or otherwise tethered thereto) with the disclosed multivalent binding compositions or reagents. In some instances, said contacting may be performed within one or more flow cells. In some instances, said one or more flow cells may be fixed components of the system. In some instances, said one or more flow cells may be removable or disposable components of the system.

### Computer Control Systems.

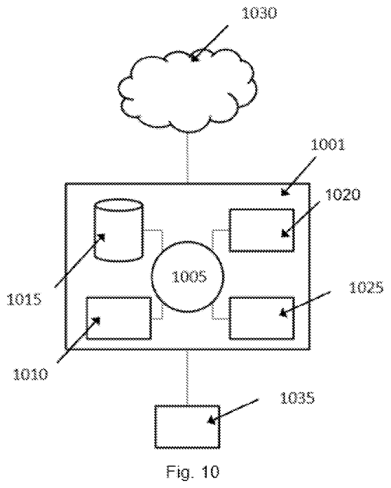

The present disclosure provides computer systems that are programmed or otherwise configured to implement methods provided herein, for example, methods for nucleic sequencing, storing reference nucleic acid sequences, conducting sequence analysis or comparing sample and reference nucleic acid sequences as described herein. An example of such a computer system is shown in FIG. 10. The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet or extranet, or an intranet or extranet that is in communication with the Internet. The network 1030 in some cases is a telecommunication or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. Examples of operations performed by the CPU 1005 can include fetch, decode, execute, and writeback.

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems provided herein, such as the computer system 1001, can be embodied in programming. Various aspects of the technology may be thought of as “products” or “articles of manufacture” in the form of machine (or processor) executable code or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code or data. Many of these forms of computer readable media may be involved in carrying one or more sequences of one or more instructions to a processor for execution. The computer system 1001 can include or be in communication with an electronic display 1035 that comprises a user interface (UI) for providing, for example, an output or readout of a nucleic acid sequencing instrument coupled to the computer system 1001. Such readout can include a nucleic acid sequencing readout, such as a sequence of nucleic acid bases that comprise a given nucleic acid sample. The UI may also be used to display the results of an analysis making use of such readout. Examples of UI's include, without limitation, a graphical user interface (GUI) and web-based user interface. The electronic display 1035 can be a computer monitor, or a capacitive or resistive touchscreen.

### Processors and Computer Systems

One or more processors may be employed to implement the systems for nucleic acid sequencing or other nucleic acid detection and analysis methods disclosed herein. The one or more processors may comprise a hardware processor such as a central processing unit (CPU), a graphic processing unit (GPU), a general-purpose processing unit, or computing platform. The one or more processors may be comprised of any of a variety of integrated circuits (e.g., application specific integrated circuits (ASICs) designed specifically for implementing deep learning network architectures, or field-programmable gate arrays (FPGAs) to accelerate compute time, etc., or to facilitate deployment), microprocessors, emerging next-generation microprocessor designs (e.g., memristor-based processors), logic devices and the like. Although the disclosure is described with reference to a processor, other types of integrated circuits and logic devices may also be applicable. The processor may have any data operation capability. For example, the processor may perform 512 bit, 256 bit, 128 bit, 64 bit, 32 bit, or 16 bit data operations. The one or more processors may be single core or multi core processors, or a plurality of processors configured for parallel processing.

The one or more processors or computers used to implement the disclosed methods may be part of a larger computer system or may be operatively coupled to a computer network (a “network”) with the aid of a communication interface to facilitate transmission of and sharing of data. The network may be a local area network, an intranet or extranet, an intranet or extranet that is in communication with the Internet, or the Internet. The network in some cases is a telecommunication or data network. The network may include one or more computer servers, which in some cases enables distributed computing, such as cloud computing. The network, in some cases with the aid of the computer system, may implement a peer-to-peer network, which may enable devices coupled to the computer system to behave as a client or a server.

The computer system may also include memory or memory locations (e.g., random-access memory, read-only memory, flash memory, Intel® Optane™ technology), electronic storage units (e.g., hard disks), communication interfaces (e.g., network adapters) for communicating with one or more other systems, and peripheral devices, such as cache, other memory, data storage or electronic display adapters. The memory, storage units, interfaces and peripheral devices may be in communication with the one or more processors, e.g., a CPU, through a communication bus, e.g., as is found on a motherboard. The storage unit(s) may be data storage unit(s) (or data repositories) for storing data.

The one or more processors, e.g., a CPU, execute a sequence of machine-readable instructions, which are embodied in a program (or software). The instructions are stored in a memory location. The instructions are directed to the CPU, which subsequently program or otherwise configure the CPU to implement the methods of the present disclosure. Examples of operations performed by the CPU include fetch, decode, execute, and write back. The CPU may be part of a circuit, such as an integrated circuit. One or more other components of the system may be included in the circuit. In some cases, the circuit is an application specific integrated circuit (ASIC).

The storage unit stores files, such as drivers, libraries and saved programs. The storage unit stores user data, e.g., user-specified preferences and user-specified programs. The computer system in some cases may include one or more additional data storage units that are external to the computer system, such as located on a remote server that is in communication with the computer system through an intranet or the Internet.

Some aspects of the methods and systems provided herein may be implemented by way of machine (e.g., processor) executable code stored in an electronic storage location of the computer system, for example, in the memory or electronic storage unit. The machine-executable or machine-readable code may be provided in the form of software. During use, the code is executed by the one or more processors. In some cases, the code is retrieved from the storage unit and stored in the memory for ready access by the one or more processors. In some situations, the electronic storage unit is precluded, and machine-executable instructions are stored in memory. The code may be pre-compiled and configured for use with a machine having one or more processors adapted to execute the code or may be compiled at run time. The code may be supplied in a programming language that is selected to enable the code to execute in a pre-compiled or as-compiled fashion.

Various aspects of the technology may be thought of as “products” or “articles of manufacture”, e.g., “computer program or software products”, often in the form of machine—(or processor—) executable code or associated data that is stored in a type of machine readable medium, where the executable code comprises a plurality of instructions for controlling a computer or computer system in performing one or more of the methods disclosed herein. Machine-executable code may be stored in an optical storage unit comprising an optically readable medium such as an optical disc, CD-ROM, DVD, or Blu-Ray disc. Machine-executable code may be stored in an electronic storage unit, such as memory (e.g., read-only memory, random-access memory, flash memory) or on a hard disk. “Storage” type media include any or all of the tangible memory of the computers, processors or the like, or associated modules thereof, such as various semiconductor memory chips, optical drives, tape drives, disk drives and the like, which may provide non-transitory storage at any time for the software that encodes the methods and algorithms disclosed herein.

All or a portion of the software code may at times be communicated via the Internet or various other telecommunication networks. Such communications, for example, enable loading of the software from one computer or processor into another, for example, from a management server or host computer into the computer platform of an application server. Thus, other types of media that are used to convey the software encoded instructions include optical, electrical and electromagnetic waves, such as those used across physical interfaces between local devices, through wired and optical landline networks, and over various atmospheric links. The physical elements that carry such waves, such as wired or wireless links, optical links, or the like, are also considered media that convey the software encoded instructions for performing the methods disclosed herein. As used herein, unless restricted to non-transitory, tangible “storage” media, terms such as computer or machine “readable medium” refer to any medium that participates in providing instructions to a processor for execution.

The computer system often includes, or may be in communication with, an electronic display for providing, for example, images captured by a machine vision system. The display is often also capable of providing a user interface (UI). Examples of UI's include but are not limited to graphical user interfaces (GUIs), web-based user interfaces, and the like.

### System Control Software

In some instances, the disclosed systems may comprise a computer (or processor) and computer-readable media that includes code for providing a user interface as well as manual, semi-automated, or fully-automated control of all system functions, e.g. control of a fluid flow controller or fluid dispensing system (or sub-system), a temperature control system (or subsystem), an imaging system (or sub-system), etc. In some instances, the system computer or processor may be an integrated component of the instrument system (e.g. a microprocessor or mother board embedded within the instrument). In some instances, the system computer or processor may be a stand-alone module, for example, a personal computer or laptop computer. Examples of fluid flow control functions that may be provided by the instrument control software include, but are not limited to, volumetric fluid flow rates, fluid flow velocities, the timing and duration for sample and reagent additions, rinse steps, and the like. Examples of temperature control functions that may be provided by the instrument control software include, but are not limited to, specifying temperature set point(s) and control of the timing, duration, and ramp rates for temperature changes. Examples of imaging system control functions that may be provided by the instrument control software include, but are not limited to, autofocus capability, control of illumination or excitation light exposure times and intensities, control of image acquisition rate, exposure time, data storage options, and the like.

### Image Processing Software

In some instances of the disclosed systems, the system may further comprise computer-readable media that includes code for providing image processing and analysis capability. Examples of image processing and analysis capability that may be provided by the software include, but are not limited to, manual, semi-automated, or fully-automated image exposure adjustment (e.g. white balance, contrast adjustment, signal-averaging and other noise reduction capability, etc.), manual, semi-automated, or fully-automated edge detection and object identification (e.g., for identifying clusters of amplified template nucleic acid molecules on a substrate surface), manual, semi-automated, or fully-automated signal intensity measurements or thresholding in one or more detection channels (e.g., one or more fluorescence emission channels), manual, semi-automated, or fully-automated statistical analysis (e.g., for comparison of signal intensities to a reference value for base-calling purposes).

In some instances, the system software may provide integrated real-time image analysis and instrument control, so that sample loading, reagent addition, rinse, or imaging/base-calling steps may be prolonged, modified, or repeated until, e.g., optimal base-calling results are achieved. Any of a variety of existing image processing and analysis algorithms may be used to implement real-time or post-processing image analysis capability. Examples include, but are not limited to, the Canny edge detection method, the Canny-Deriche edge detection method, first-order gradient edge detection methods (e.g. the Sobel operator), second order differential edge detection methods, phase congruency (phase coherence) edge detection methods, other image segmentation algorithms (e.g. intensity thresholding, intensity clustering methods, intensity histogram-based methods, etc.), feature and pattern recognition algorithms (e.g. the generalized Hough transform for detecting arbitrary shapes, the circular Hough transform, etc.), and mathematical analysis algorithms (e.g. Fourier transform, fast Fourier transform, wavelet analysis, auto-correlation, etc.), or combinations thereof.

In some instances, the system control and image processing/analysis software may be written as separate software modules. In some instances, the system control and image processing/analysis software may be incorporated into an integrated software package.

### Data Analysis Software

In some instances of the disclosed systems, the system may further comprise computer-readable media that includes code for performing data analysis, e.g., software for decoding of probe barcodes, sample demultiplexing, binning of probe barcode sequences detected for a given sample barcode, counting of barcode sequencing, etc. In some instances, the data analysis software may further comprise data analytics (e.g., statistical analysis) and data display capabilities. In some instances, the data analysis software may comprise tools for performing a preliminary assessment of assay specificity or for determining other assay performance quality metrics.

### Kits for Detecting Pathogenic Nucleic Acids

Disclosed herein are kits. In some instances, the kits of the present disclosure may comprise one or more sets of barcoded padlock probes or molecular inversion probes, one or more sets of sample-indexed amplification primers, assay buffers and reagents required to perform sample purification, nucleic acid extraction, hybridization, ligation, and amplification (including RCA), and sequencing (including any combination of the multivalent binding compositions disclosed herein), one or more sequencing flow cells, or any combination thereof.

Disclosed herein, in some embodiments, are kits for preparing a nucleic acid sequencing library using the compositions, methods, or systems disclosed herein. In some embodiments, the kits comprise compositions described herein, such as reagents and substrates for detecting a presence of a target nucleic acid sequence in one or more samples of a plurality of samples.

The kit disclosed herein comprise enzymes, nucleic acids, nucleotides, supports with functionalized surfaces, a polymer-nucleotide composition, a buffer system, or instructions. In some embodiments, the kit disclosed herein may comprise a solution comprising nucleic acid molecules extracted from a sample of the plurality of samples with a linear nucleic acid probe molecule under conditions that promote hybridization of complementary sequences. In some embodiments, the linear nucleic acid probe molecule comprises a target-specific 5′ region that is complementary to a first region of the target nucleic acid sequence, an amplification primer binding region, a probe barcode sequence, and a target-specific 2′ region that is complementary to a second region of the target nucleic acid sequence. In some embodiments, the linear nucleic acid probe molecule comprises a target-specific 5′ region that is complementary to a first region of the target nucleic acid sequence, an amplification primer binding region, a sample barcode sequence, a probe barcode sequence, and a target-specific 2′ region that is complementary to a second region of the target nucleic acid sequence. In some embodiments, the sample barcode sequence is unique for each sample in the plurality of samples. In some embodiments, the probe barcode sequence is unique for each pair of target-specific 5′ and target-specific 3′regions. In some embodiments, the first region of the target nucleic acid sequence and the second region of the target nucleic acid sequence are contiguous sequences in the target nucleic acid molecule. In some embodiments, the amplification primer is complementary to the amplification primer binding region. In some embodiments, the enzymes may be ligating enzymes, proteases, transposases, any one of enzymes described herein and combination thereof. In some embodiments, the nucleic acids may be oligonucleotides, splint oligonucleotides, any oligonucleotides or nucleic acids described herein, or any combinations thereof. In some embodiments, nucleotides may comprise nucleotides with blocking moieties. In some embodiments, nucleotides may comprise polymer-nucleotide conjugates. In some embodiments, nucleotides may comprise detection moieties. In some embodiments, supports with functionalized surfaces may comprise a plastic, metal, glass, or any combinations thereof for the support. In some embodiments, supports with functionalized surfaces may comprise hydrophilic, hydrophobic, polymeric, primed, or any combinations thereof for the functionalization.

In some embodiments, the instructions may comprise a description for a method of circularizing single stranded nucleic acid, single stranded DNA, single stranded RNA, double-stranded nucleic acid, double-stranded DNA, double-stranded RNA, or any nucleic acid described herein and combinations thereof. In some embodiments, the instructions may further comprise a description for a method of attaching nucleic acid adapters or primers before circularization, simultaneously with circularization, or after circularization. In some embodiments, the instructions may further comprise a description for processing the genetic material from a biological source. In some embodiments, the instructions may comprise a description for detecting nucleic acid sequences. In some embodiments, the instructions may comprise a description for planning multiple stages, each stage employing one of the methods described herein. For example, one embodiment of such a description may describe the operations comprising a) incubating a solution comprising nucleic acid molecules extracted from a sample of the plurality of samples with a linear nucleic acid probe molecule under conditions that promote hybridization of complementary sequences; b) subjecting the solution to conditions for performing a ligation reaction to create circularized nucleic acid probe molecules from hybridized linear nucleic acid probe molecules; c) subjecting the solution to conditions for amplifying the circularized linear nucleic acid probe molecules using an amplification primer that is complementary to the amplification primer binding region, thereby creating an amplified product for the sample; d) pooling an amplified product, or derivative thereof, for each sample of the plurality of samples; and e) detecting the presence of one or more sample barcode sequences in the pooled amplified product, or a derivative thereof, thereby detecting the presence of the target nucleic acid in one or more samples of the plurality of samples. In some embodiments, the detecting in e) comprises sequencing.

Disclosed herein, in some embodiments, are kits for performing nucleic acid sequencing using the compositions, methods, or systems disclosed herein. In some embodiments, the kits comprise compositions described herein, such as reagents and substrates for performing nucleic acid sequencing using the compositions, methods, or systems disclosed herein.

In some embodiments, the polymer-nucleotide composition may comprise a polymer core and a plurality of nucleotide moieties coupled thereto. In some embodiments, the surface may comprise the primed nucleic acid sequence couple thereto and a hydrophilic polymer layer. In some embodiments, the hydrophilic polymer layer comprises a polymer comprising a polymer elected from the coup comprising poly(ethylene glycol) (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), poly(glutamic acid) (PGA), poly-lysine, poly-glucoside, streptavidin, or dextran, or a combination thereof. In some embodiments, the surface may comprise one or more interior surfaces of a flow cell. In some embodiments, the kit may further comprise at least two types of the nucleotide-polymer conjugate. In some embodiments, the kit may further comprise at least three types of the nucleotide-polymer conjugate. In some embodiments, the kit may further comprise at least four types of the nucleotide-polymer conjugate. In some embodiments, the kit may further comprise a plurality of types of the nucleotide-polymer conjugates, and wherein each of the plurality of the types of comprises a nucleotide moiety having a distinct nucleobase. In some embodiments, the kit may further comprise a plurality of types of the nucleotide-polymer conjugates, and wherein each of the plurality of the types of comprises a nucleotide moiety having a distinct nucleobase.

In some embodiments, the kit may further comprise a plurality of types of the nucleotide-polymer conjugate, and wherein each of the plurality of the types comprises a distinct detectable label coupled to the polymer core. In some embodiments, the detectable label comprises a fluorescent label. In some embodiments, the polymer core may comprise a polymer selected from the coup comprising poly(ethylene glycol) (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), poly(glutamic acid) (PGA), poly-lysine, poly-glucoside, streptavidin, and dextran.

In some embodiments, the kit may further comprise one or more unlabeled nucleotides comprising a blocking group at a 3′ position of a sugar of the one or more unlabeled nucleotide. In some embodiments, the blocking group may comprise a 3′-O-methyl nucleotide, or a 3′-O-alkyl hydroxylamine nucleotide, 3′-O-azidomethyl nucleotide, 3′-phosphorothioate group, a 3′-O-malonyl group, a 3′-O-benzyl group, a 3′-O-amino group, or a derivative thereof. In some embodiments, the kit may comprise a buffer system comprising strontium ions, magnesium ions, calcium ions, or any combination thereof.

In some embodiments, the kit may comprise instructions comprising a description for identifying a nucleotide in a primed nucleic acid sequence that is derived from a sample of a subject having or suspected of having a disease of a condition caused by SARS-CoV-2 virus or a variant thereof by a) incubating a solution comprising nucleic acid molecules extracted from a sample of the plurality of samples with a linear nucleic acid probe molecule under conditions that promote hybridization of complementary sequences, wherein: i) the linear nucleic acid probe molecule comprises a target-specific 5′ region that is complementary to a first region of the target nucleic acid sequence, an amplification primer binding region, a probe barcode sequence, and a target-specific 3′ region that is complementary to a second region of the target nucleic acid sequence; ii) the probe barcode sequence is unique for each pair of target-specific 5′ and target-specific 3′ regions; and iii) the first region of the target nucleic acid sequence and the second region of the target nucleic acid sequence are contiguous sequences in the target nucleic acid molecule; b) subjecting the solution to conditions for performing a ligation reaction to create circularized nucleic acid probe molecules from hybridized linear nucleic acid probe molecules; c) subjecting the solution to conditions for amplifying the circularized linear nucleic acid probe molecules using an amplification primer that is complementary to the amplification primer binding region, thereby creating an amplified product for the sample; d) pooling an amplified product, or derivative thereof, for each sample of the plurality of samples; and e) detecting the presence of one or more sample barcode sequences in the pooled amplified product, or a derivative thereof, thereby detecting the presence of the target nucleic acid in one or more samples of the plurality of samples. In some embodiments, the kit may comprise instructions comprising a description for identifying a nucleotide in a primed nucleic acid sequence that is derived from a sample of a subject having or suspected of having a disease of a condition caused by SARS-CoV-2 virus or a variant thereof by a) incubating a solution comprising nucleic acid molecules extracted from a sample of the plurality of samples with a linear nucleic acid probe molecule under conditions that promote hybridization of complementary sequences, wherein: i) the linear nucleic acid probe molecule comprises a target-specific 5′ region that is complementary to a first region of the target nucleic acid sequence, an amplification primer binding region, a sample barcode sequence, a probe barcode sequence, and a target-specific 3′ region that is complementary to a second region of the target nucleic acid sequence; ii) the sample barcode sequence is unique for each sample in the plurality of samples; iii) the probe barcode sequence is unique for each pair of target-specific 5′ and target-specific 3′ regions; and iv) the first region of the target nucleic acid sequence and the second region of the target nucleic acid sequence are contiguous sequences in the target nucleic acid molecule; b) subjecting the solution to conditions for performing a ligation reaction to create circularized nucleic acid probe molecules from hybridized linear nucleic acid probe molecules; c) subjecting the solution to conditions for amplifying the circularized linear nucleic acid probe molecules using an amplification primer that is complementary to the amplification primer binding region, thereby creating an amplified product for the sample; d) pooling an amplified product, or derivative thereof, for each sample of the plurality of samples; and e) detecting the presence of one or more sample barcode sequences in the pooled amplified product, or a derivative thereof, thereby detecting the presence of the target nucleic acid in one or more samples of the plurality of samples. In some embodiments, the detecting in e) comprises sequencing. In some embodiments, the target nucleic acid molecules comprise RNA molecules. In some embodiments, the target nucleic acid molecules comprise viral and nucleic acid molecules. In some embodiments, the viral RNA molecules comprise COVID-19 RNA molecules. In some embodiments, the target-specific 5′ and target specific 3′ regions of one or more linear nucleic acid probe molecules comprise sequences that are complementary to the COVID-19 S gene or fragments thereof, the COVID-19 Orflab gene or fragments thereof, the COVID-19 N gene or fragments thereof, or any combination thereof. In some embodiments, the target-specific 5′ and target specific 3′ regions of one or more linear nucleic acid probe molecules comprise sequences that are complementary to the Ca-Y132H sequence. In some embodiments, the plurality of samples comprise nasopharyngeal swab samples, sputum samples, bronchoalveolar lavage fluid samples, blood samples, urine samples, feces samples, or any combination thereof.

### Assay & Sequencing System Performance

To illustrate the projected performance of the disclosed methods and systems, assume a configuration where a single instrument processes 384 samples per run, with a run performed every 2 hours. Assuming a system duty cycle (or uptime) of 80% and 24/7 operation, a single instrument will perform 10 runs per day, corresponding to the processing of 3,840 samples per day or 1.4 M samples per instrument per year. Adopting a 1,536-sample-format effectively quadruples this throughput to 5.6 M samples per instrument per year, providing an order of magnitude greater sample processing throughput than any currently existing platform. The sequencing kit usage will be minimal due to the basic sequencing requirements of short barcode sequences, e.g., 15 bases, and the cost per sample will be further reduced by spreading the kit cost across all samples. The result is an assay that can be profitably commercialized for less than $10 a sample.

In some instances, the use of multivalent binding compositions for sequencing-by-trapping effectively shortens the sequencing time. In some instances, the sequencing reaction cycle comprising the contacting, detecting, and incorporating steps may be performed in a total time ranging from about 5 minutes to about 60 minutes. In some instances, the sequencing reaction cycle time may be at least 5 minutes, at least 10 minutes, at least 20 minutes, at least 30 minutes, at least 40 minutes, at least 50 minutes, or at least 60 minutes. In some instances, the sequencing reaction cycle time may be at most 60 minutes, at most 50 minutes, at most 40 minutes, at most 30 minutes, at most 20 minutes, at most 10 minutes, or at most 5 minutes. Any of the lower and upper values described in this paragraph may be combined to form a range included within the present disclosure, for example, in some instances the sequencing reaction time per cycle may range from about 10 minutes to about 30 minutes. It is possible that the sequencing reaction cycle time may have any value within this range, e.g., about 16 minutes.

In some instances, the disclosed multivalent binding compositions and methods for nucleic acid sequencing will provide an average base-calling accuracy of at least 80%, at least 85%, at least 90%, at least 92%, at least 94%, at least 96%, at least 98%, at least 99%, at least 99.5%, at least 99.8%, or at least 99.9% correct over the course of a sequencing run. In some instances, the disclosed multivalent binding compositions and methods for nucleic acid sequencing will provide an average base-calling accuracy of at least 80%, at least 85%, at least 90%, at least 92%, at least 94%, at least 96%, at least 98%, at least 99%, at least 99.5%, at least 99.8%, or at least 99.9% correct per every 1,000 bases, 10,0000 bases, 25,000 bases, 50,000 bases, 75,000 bases, or 100,000 bases called.

In some instance, the use of multivalent binding compositions for sequencing provides more accurate base readout. In some instances, the disclosed compositions and methods for nucleic acid sequencing may provide an average Q-score for base-calling accuracy over a sequencing run that ranges from about 20 to about 50. In some instances, the average Q-score is at least 20, at least 25, at least 30, at least 35, at least 40, at least 45, or at least 50. It is possible that the average Q-score may have any value within this range, e.g., about 32.

In some instances, the disclosed multivalent binding compositions and methods for nucleic acid sequencing may provide a Q-score of greater than 30 for at least 50%, at least 60%, at least 70%, at least 80%, at least 85%, at least 90%, at least 95%, at least 98%, or at least 99% of the terminal (or N+1) nucleotides identified. In some instances, the disclosed compositions and methods for nucleic acid sequencing may provide a Q-score of greater than 35 for at least 50%, at least 60%, at least 70%, at least 80%, at least 85%, at least 90%, at least 95%, at least 98%, or at least 99% of the terminal (or N+1) nucleotides identified. In some instances, the disclosed compositions and methods for nucleic acid sequencing may provide a Q-score of greater than 40 for at least 50%, at least 60%, at least 70%, at least 80%, at least 85%, at least 90%, at least 95%, at least 98%, or at least 99% of the terminal (or N+1) nucleotides identified. In some instances, the disclosed compositions and methods for nucleic acid sequencing may provide a Q-score of greater than 45 for at least 50%, at least 60%, at least 70%, at least 80%, at least 85%, at least 90%, at least 95%, at least 98%, or at least 99% of the terminal (or N+1) nucleotides identified. In some instances, the disclosed compositions and methods for nucleic acid sequencing may provide a Q-score of greater than 50 for at least 50%, at least 60%, at least 70%, at least 80%, at least 85%, at least 90%, at least 95%, at least 98%, or at least 99% of the terminal (or N+1) nucleotides identified.

In some instances, the number of samples processed or sequenced in parallel (on a single instrument) may range from about 8 to about 1,536 samples per run. In some instances, the number of samples processed or sequenced per run may be at least 8, at least 12, at least 24, at least 48, at least 96, at least 192, at least 384, at least 768, or at least 1,536. In some instances, the number of samples processed or sequenced per run may be at most 1,536, at most 768, at most 384, at most 192, at most 96, at most 48, at most 24, at most 12, or at most 8. Any of the lower and upper values described in this paragraph may be combined to form a range included within the present disclosure, for example, in some instances the number of samples processed or sequenced per run may range from about 96 to about 1,536. It is possible that the number of samples processed or sequenced per run may have any value within this range, e.g., about 100.

In general, the number of sequencing cycles required for assay read-out will depend on the length of the probe or sample barcodes used. In some instances, the number of sequencing cycles required may range from about 3 to about 30. In some instances, the number of sequencing cycles may be at least 3, at least 4, at least 5, at least 6, at least 7, at least 8, at least 9, at least 10, at least 15, at least 20, at least 25, or at least 30. In some instances, the number of sequencing cycles required may be at most 30, at most 25, at most 20, at most 15, at most 10, at most 9, at most 8, at most 7, at most 6, at most 5, at most 4, or at most 3. Any of the lower and upper values described in this paragraph may be combined to form a range included within the present disclosure, for example, in some instances the number of sequencing cycles require may range from about 6 to about 20. It is possible that the number of sequencing cycles required may have any value within this range, e.g., about 16.

In some instances, the assay sensitivity (or true positive rate) achieved by the disclosed methods and systems may range from about 90% to about 100%. In some instances, the assay sensitivity may be at least 90%, at least 91%, at least 92%, at least 93%, at least 94%, at least 95%, at least 96%, at least 97%, at least 98%, at least 99%, or 100%. In some instances, the assay sensitivity may be at most 100%, at most 99%, at most 98%, at most 97%, at most 96%, at most 95%, at most 94%, at most 93%, at most 92%, at most 91%, or at most 90%. Any of the lower and upper values described in this paragraph may be combined to form a range included within the present disclosure, for example, in some instances the assay sensitivity may range from about 92% to about 98%. It is possible that the assay sensitivity may have any value within this range, e.g., about 95.6%.

In some instances, the assay specificity (or true negative rate) achieved by the disclosed methods and systems may range from about 90% to about 100%. In some instances, the assay specificity may be at least 90%, at least 91%, at least 92%, at least 93%, at least 94%, at least 95%, at least 96%, at least 97%, at least 98%, at least 99%, or 100%. In some instances, the assay specificity may be at most 100%, at most 99%, at most 98%, at most 97%, at most 96%, at most 95%, at most 94%, at most 93%, at most 92%, at most 91%, or at most 90%. Any of the lower and upper values described in this paragraph may be combined to form a range included within the present disclosure, for example, in some instances the assay specificity may range from about 94% to about 99%. It is possible that the assay sensitivity may have any value within this range, e.g., about 97.2%.

In some instances, the assay limit-of-detection (LoD) achieved by the disclosed methods and systems may range from about 1 target nucleic acid sequence per μL to about 20 target nucleic acid sequence copies per μL. In some instances, the limit-of-detection may be at least 1, at least 2, at least 3, at least 4, at least 5, at least 10, at least 15, or at least 20 target nucleic acid sequence copies per μL. In some instances, the limit-of-detection may be at most 20, at most 15, at most 10, at most 5, at most 4, at most 3, at most 2, or at most 1 target nucleic acid sequence copy per μL. Any of the lower and upper values described in this paragraph may be combined to form a range included within the present disclosure, for example, in some instances the limit of detection may range from about 3 to about 15 target nucleic acid sequence copies per μL. It is possible that the limit of detection may have any value within this range, e.g., about 9 target nucleic acid sequence copies per μL.

In some instances, the disclosed methods and systems may achieve a sample processing throughput ranging from about 10 to about 1,000 samples per hour. In some instances, the sample processing throughput may be at least 10, at least 20, at least 30, at least 40, at least 50, at least 100, at least 200, at least 300, at least 400, at least 500, at least 600, at least 700, at least 800, at least 900, or at least 1,000 samples per hour. In some instances, the sample processing throughput may be at most 1,000, at most 900, at most 800, at most 700, at most 600, at most 500, at most 400, at most 300, at most 200, at most 100, at most 50, at most 40, at most 30, at most 20, or at most 10 samples per hour. Any of the lower and upper values described in this paragraph may be combined to form a range included within the present disclosure, for example, in some instances the sample processing throughput may range from about 50 to about 500 samples per hour. It is possible that the sample processing throughput may have any value within this range, e.g., about 465 samples per hour.

In some instances, the sample-to-answer time achieved using the disclosed methods and systems may range from about 30 minutes to about 4 hours. In some instances, the sample-to-answer time may be at least 30 minutes, at least 1 hour, at least 1.5 hours, at least 2 hours, at least 2.5 hours, at least 3 hours, at least 3.5 hours, or at least 4 hours. In some instances, the sample-to-answer time may be at most 4 hours, at most 3.5 hours, at most 3 hours, at most 2.5 hours, at most 2 hours, at most 1.5 hours, at most 1 hour, or at most 30 minutes. Any of the lower and upper values described in this paragraph may be combined to form a range included within the present disclosure, for example, in some instances the sample-to-answer time may range from about 1 hour to about 3.5 hours. It is possible that the sample-to-answer time may have any value within this range, e.g., about 2 hours and twenty minutes.

In some instances, the testing cost per sample achieved using the disclosed methods and systems may range from about $1 to about $15 per sample. In some instances, the cost per sample may be at least $1, at least $2, at least $3, at least $4, at least $5, at least $6, at least $7, at least $8, at least $9, at least $10, at least $11, at least $12, at least $13, at least $14, or at least $15. In some instances, the cost per sample may be at most $15, at most $14, at most $13, at most $12, at most $11, at most $10, at most $9, at most $8, at most $7, at most $6, at most $5, at most $4, at most $3, at most $2, or at most $1. Any of the lower and upper values described in this paragraph may be combined to form a range included within the present disclosure, for example, in some instances the cost per sample may range from about $2 to about $12. It is possible that the cost per sample may have any value within this range, e.g., about $9.55.

### Applications

Described herein are methods to analyze a large number of different nucleic acid sequences from e.g., amplified nucleic acid arrays in flow cells or from an array of immobilized nucleic acids. The methods described herein can also be useful in, e.g., sequencing for comparative genomics, tracking gene expression, micro RNA sequence analysis, epigenomics, and aptamer and phage display library characterization, and other sequencing applications. The methods herein comprise various combinations of optical, mechanical, fluidic, thermal, electrical, and computing devices/aspects. The advantages conferred by the methods comprising the flow cell devices, cartridges, and systems include, but are not limited to: (i) reduced device and system manufacturing complexity and cost, (ii) significantly lower consumable costs (e.g., as compared to those for currently available nucleic acid sequencing systems), (iii) compatibility with flow cell surface functionalization methods, (iv) flexible flow control when combined with microfluidic components, e.g., syringe pumps and diaphragm valves, etc., and (v) flexible system throughput.

### Diagnosis or Prognosis of a Pathogen-Associated Disease

Disclosed herein, in some embodiments, are systems, kits and methods for diagnosing or prognosing a disease or condition associated with or caused by an infection by a pathogen disclosed herein based, as least in part, on the identification of a nucleic acid sequence from a pathogen described herein. For example, the systems, kits, and methods described herein can be used to diagnose or prognose a disease or condition caused by an infection by a virus, such as, severe respiratory syndrome 2 (SARS-CoV-2) virus or a variant thereof.

In some embodiments, the subject shows a sign or a symptom comprising fever, chills, cough, shortness of breath or difficulty breathing, fatigue, persistent pain or pressure in the chest, inability to wake or stay awake, pale-, gray-, or blue-colored skins, lips or nail beds, muscle or body aches, headache, loss of taste or smell, sore throat, congestion or runny nose, nausea, vomiting, or diarrhea, or any combination thereof.

In some embodiments, the methods described herein comprise: (a) providing a biological sample obtained from a subject suspected of having a disease or a condition associated with an infection by a pathogen; (b) sequencing genetic information derived from the biological sample; (c) identifying a nucleic acid sequence derived from the pathogen from the genetic information; and (d) diagnosing the subject with the disease or the condition associated with the infection by the pathogen.

In some embodiments, the biological sample is obtained from a subject described herein. In some embodiments, the subject is a mammal, such as a mouse, rat, guinea pig, rabbit, non-human primate, or farm animal. In some embodiments, the subject is human. In some embodiments, the subject shows a symptom related to a disease or condition disclosed herein (e.g., fever, chills, cough, shortness of breath or difficulty breathing, fatigue, persistent pain or pressure in the chest, inability to wake or stay awake, pale, gray-, or blue-colored skins, lips or nail beds, muscle or body aches, headache, loss of taste or smell, sore throat, congestion or runny nose, nausea, vomiting, or diarrhea, petechiae, or any combination thereof). In some embodiments, the subject is at least 10 years of age. In some embodiments, the subject is at least 55 years of age. In some embodiments, the subject is between 0-10, 11-19, 20-39, 40-59, 60-75, or 76-100 years old. In some embodiments, the subject has a precondition that impacts a disease prognosis described herein. In some embodiments, the precondition comprises obesity, diabetes, a blood clotting disorder, a concurrent respiratory condition (e.g., bronchitis or pneumonia), a cancer, an immunodeficiency disorder or condition (including therapeutic or medically-induced immunodeficiency, such as following a transplant or cancer therapy), or any combination thereof.

In some embodiments, the biological sample comprises blood, serum, plasma, sweat, hair, tears, urine, feces, mucus, including nasal, lung, gastric, or urogenital mucus, cerebrospinal fluid, lymphatic fluid, saliva, or any other biological sample disclosed herein. In some embodiments, the biological sample is obtained from the subject directly or indirectly.

In some embodiments, the sequencing of genetic information is performed using the methods, kits, or system described herein. In a non-limiting example, the genetic information may be sequenced by (a) bringing a primed nucleic acid sequence derived from the biological sample obtained from the subject into contact with a polymerizing enzyme and one or more nucleotide moieties under conditions sufficient to form a binding complex between the polymerizing enzyme, the one or more nucleotide moieties, and a nucleotide of the primed nucleic acid sequence without incorporation of the one or more nucleotide moieties into the primed nucleic acid sequence, wherein the subject having or suspected of having a disease or a condition caused by a pathogen disclosed herein; and (b) detecting said binding complex to identify said nucleotide in said primed nucleic acid sequence. In some embodiments, the pathogen is severe respiratory syndrome 2 (SARS-CoV-2) virus or a variant thereof.

In some embodiments, diagnosing the subject comprises diagnosing the subject with a disease or condition caused by an infection by the pathogen. In some embodiments, the disease or the condition is caused by Coronavirus Disease 2019 (COVID-19). In some embodiments, the diagnosis comprises diagnosing a severity of disease, such as, by quantifying a relative amount or durability of the pathogen in the biological sample. In some embodiments, a stage of infection may be predicted by the methods of system described herein.

### Pathogen Tracing

The methods, systems and kits described herein are useful for detecting a novel pathogenic infection or minimizing further spread of the infection based, at least in part, on the identification of a nucleic acid sequence of a pathogen disclosed herein. There is an urgent and unmet need of tracing the spread of pathogenic infections, particular those pathogens with undetected transmission, such as the SARS-CoV-2 virus. The duration and severity of each phase of a SARS-CoV-2 infection depends, at least in part, on how quickly the infection is contained, which is particularly challenging given that a significant number of people infected with SARS-CoV-2 do not show symptoms.

In some embodiment, the methods, systems, and kits described herein may be used to monitor the emergence or spread of an infection caused by a pathogen disclosed herein (e.g., SARS-CoV-2) within a geographical space. In some embodiments, the geographical space comprises a village or a town. In some embodiments, the geographical space comprises a rural area or an urban area. In some embodiment, the geographical space comprises a city, a country, a state, or a country.

In some embodiments, the methods described herein comprise: (a) providing a plurality of biological samples obtained from a plurality of subjects; (b) sequencing genetic information derived from the plurality of biological samples; (c) identifying a nucleic acid sequence derived from a pathogen from the genetic information; and (d) associating the presence of the nucleic acid sequence with the emergence or spread of an infection caused by the pathogen.

In some embodiments, the plurality of biological samples is obtained from a plurality of subjects described herein. In some embodiments, the plurality of subjects is a mammal, such as a mouse, rat, guinea pig, rabbit, non-human primate, or farm animal. In some embodiments, the plurality of subjects is human. In some embodiments, the plurality of subjects shows a symptom related to a disease or condition disclosed herein (e.g., fever, chills, cough, shortness of breath or difficulty breathing, fatigue, persistent pain or pressure in the chest, inability to wake or stay awake, pale-, gray-, or blue-colored skins, lips or nail beds, muscle or body aches, headache, loss of taste or smell, sore throat, congestion or runny nose, nausea, vomiting, or diarrhea, petechiae, or any combination thereof). In some embodiments, the subject is at least 10 years of age. In some embodiments, the subject is at least 55 years of age. In some embodiments, the subject is between 0-10, 11-19, 20-39, 40-59, 60-75, or 76-100 years old. In some embodiments, the subject has a precondition that impacts a disease prognosis described herein. In some embodiments, the precondition comprises obesity, diabetes, a blood clotting disorder, a concurrent respiratory condition (e.g., bronchitis or pneumonia), a cancer, an immunodeficiency disorder or condition (including therapeutic or medically-induced immunodeficiency, such as following a transplant or cancer therapy), or any combination thereof.

In some embodiments, the biological sample comprises blood, serum, plasma, sweat, hair, tears, urine, feces, mucus, including nasal, lung, gastric, or urogenital mucus, cerebrospinal fluid, lymphatic fluid, saliva, or any other biological sample disclosed herein. In some embodiments, the biological sample is obtained from the subject directly or indirectly.

In some embodiments, the associating in (d) comprises guiding the subject to self-isolate or contact medical professionals, such as a medical doctor. The medical professionals may further perform a PCR test for confirmation of the infection cause by the pathogens, if the methods show a positive test result. A negative test, if the subject does not have symptoms disclosed herein, makes it very unlikely that the subject is infected. However, the subject needs to continue to follow standard prevention strategies.

In some embodiments, detecting a pathogenic infection and minimizing further spread of the infection comprises diagnosing the subject with a disease or condition caused by an infection by the pathogen. In some embodiments, the disease or the condition is caused by Coronavirus Disease 2019 (COVID-19). In some embodiments, the detecting a pathogenic infection and minimizing further spread of the infection comprises diagnosing a severity of disease, such as, by quantifying a relative amount or durability of the pathogen in the biological sample. In some embodiments, a stage of infection may be predicted by the methods of system described herein.

### Numbered Embodiments

Embodiment 1. A method for detecting a presence of a target nucleic acid sequence in one or more samples of a plurality of samples, the method comprising: a) incubating a solution comprising nucleic acid molecules extracted from a sample of the plurality of samples with a linear nucleic acid probe molecule under conditions that promote hybridization of complementary sequences, wherein: i) the linear nucleic acid probe molecule comprises a target-specific 5′ region that is complementary to a first region of the target nucleic acid sequence, an amplification primer binding region, a probe barcode sequence, and a target-specific 3′ region that is complementary to a second region of the target nucleic acid sequence; ii) the probe barcode sequence is unique for each pair of target-specific 5′ and target-specific 3′ regions; and iii) the first region of the target nucleic acid sequence and the second region of the target nucleic acid sequence are contiguous sequences in the target nucleic acid molecule; b) subjecting the solution to conditions sufficient for performing a ligation reaction to create circularized nucleic acid probe molecules from hybridized linear nucleic acid probe molecules; c) subjecting the solution to conditions sufficient for amplifying the circularized linear nucleic acid probe molecules using an amplification primer that is complementary to the amplification primer binding region and comprises a sample barcode that is unique for a sample in the plurality of samples, thereby creating an amplified product for the sample; d) pooling an amplified product, or a derivative thereof, for each sample of the plurality of samples; and e) detecting the presence of one or more sample barcode sequences in the pooled amplified product, or a derivative thereof, thereby detecting the presence of the target nucleic acid in one or more samples of the plurality of samples.

Embodiment 2. A method for detecting a presence of a target nucleic acid sequence in one or more samples of a plurality of samples, the method comprising: a) incubating a solution comprising nucleic acid molecules extracted from a sample of the plurality of samples with a linear nucleic acid probe molecule under conditions that promote hybridization of complementary sequences, wherein: i) the linear nucleic acid probe molecule comprises a target-specific 5′ region that is complementary to a first region of the target nucleic acid sequence, an amplification primer binding region, a sample barcode sequence, a probe barcode sequence, and a target-specific 3′ region that is complementary to a second region of the target nucleic acid sequence; ii) the sample barcode sequence is unique for each sample in the plurality of samples; iii) the probe barcode sequence is unique for each pair of target-specific 5′ and target-specific 3′ regions; and iv) the first region of the target nucleic acid sequence and the second region of the target nucleic acid sequence are contiguous sequences in the target nucleic acid molecule; b) subjecting the solution to conditions sufficient for performing a ligation reaction to create circularized nucleic acid probe molecules from hybridized; c) subjecting the solution to conditions sufficient for amplifying the circularized linear nucleic acid probe molecules using an amplification primer that is complementary to the amplification primer binding region, thereby creating an amplified product for the sample; d) pooling an amplified product, or derivative thereof, for each sample of the plurality of samples; and e) detecting the presence of one or more sample barcode sequences in the pooled amplified product, or a derivative thereof, thereby detecting the presence of the target nucleic acid in one or more samples of the plurality of samples.

Embodiment 3. The method of embodiment 1 or embodiment 2, wherein the detecting in (e) comprises sequencing. Embodiment 4. The method of any one of embodiments 1 to 3, wherein two or more different linear nucleic acid probe molecules are incubated with the target nucleic acid molecules in (a), and each of the two or more different linear nucleic acid probe comprise a different pair of target-specific 5′ and target-specific 3′ regions. Embodiment 5. The method or any one of embodiments 1 to 4, further comprising determining a copy number for one or more unique probe barcodes for each unique sample barcode in the pooled amplified product, or a derivative thereof, thereby determining a number of target nucleic acid molecules present in each sample of the plurality of samples. Embodiment 6. The method of any one of embodiments 1 to 5, further comprising digesting the target nucleic acid molecules extracted from a sample with an exonuclease following the ligation in (b). Embodiment 7. The method of any one of embodiments 1 to 6, wherein the target nucleic acid molecules comprise RNA molecules. Embodiment 8. The method of any one of embodiments 1 to 7, wherein the target nucleic acid molecules comprise viral nucleic acid molecules. Embodiment 9. The method of any one of embodiments 1 to 8, wherein the target nucleic acid molecules comprise viral RNA molecules. Embodiment 10. The method of embodiment 9, wherein the viral RNA molecules comprise Covid-19 RNA molecules. Embodiment 11. The method of embodiment 9 or embodiment 10, wherein the target-specific 5′ and target-specific 3′ regions of one or more linear nucleic acid probe molecules comprise sequences that are complementary to the Covid-19 S gene or fragments thereof, the Covid-19 Orflab gene or fragments thereof, the Covid-19 N gene or fragments thereof, or any combination thereof. Embodiment 12. The method of embodiment 9 or embodiment 10, wherein the target-specific 5′ and target-specific 3′ regions of one or more linear nucleic acid probe molecules comprise sequences that are complementary to the Ca-Y132H sequence. Embodiment 13. The method of any one of embodiments 1 to 12, wherein the plurality of samples comprises nasopharyngeal swab samples, sputum samples, bronchoalveolar lavage fluid samples, blood samples, urine samples, feces samples, or any combination thereof. Embodiment 14. The method of any one of embodiments 1 to 13, wherein the target-specific 5′ and target-specific 3′ regions of one or more linear nucleic acid probe molecules comprise molecular inversion probes, and the ligation reaction performed in (b) further comprises a gap-filling step. Embodiment 15. The method of any one of embodiments 1 to 14, wherein the sample barcode sequence ranges from about 10 to about 12 nucleotides in length. Embodiment 16. The method of any one of embodiments 1 to 15, wherein the probe barcode sequence ranges from about 6 to about 10 nucleotides in length. Embodiment 17. The method of any once of embodiments 1 to 16, wherein the sample barcode sequence and the probe barcode sequence collectively range from about 16 to about 22 nucleotides in length in total. Embodiment 18. The method of any one of embodiments 1 to 17, wherein the length of a barcode sequence is chosen to maintain a Hamming distance of at least 2 to provide for correction of sequencing errors. Embodiment 19. The method of any one of embodiments 1 to 18, wherein the length of a barcode sequence is chosen to maintain a Hamming distance of at least 5, thereby enabling detection and correction of up to 2 sequencing errors. Embodiment 20. The method of any one of embodiments 1 to 19, wherein the length of a barcode sequence is chosen to maintain a Hamming distance of at least 7, thereby enabling detection and correction of up to 3 sequencing errors. Embodiment 21. The method of any one of embodiments 1 to 20, wherein the amplification in (c) is performed using rolling circle amplification (RCA) to generate concatemers comprising multiple copies of the circularized nucleic acid probe molecules for each sample in the plurality of samples. Embodiment 22. The method of embodiment 21, wherein the detecting in (e) comprises sequencing, and the sequencing comprises hybridizing the concatemers to a surface-bound adapter sequences within a sequencing flow cell and condensing them into individually addressable nanoball sequences. Embodiment 23. The method of embodiment 22, wherein the surface-bound adapter sequences within the sequencing flow cell are bound to a low non-specific binding surface comprising at least one hydrophilic polymer layer. Embodiment 24. The method of embodiment 23, wherein the individually addressable nanoball sequences are tethered to the low non-specific binding surface at a surface density of greater than 1,000 nanoball sequences per mm². Embodiment 25. The method of embodiment 24, wherein the nanoball sequences are labeled with a fluorophore. Embodiment 26. The method of embodiment 25, wherein the fluorophore is cyanine dye-3 (Cy 3), and a fluorescence image of the surface within the sequencing flow cell exhibits a contrast-to-noise ratio (CNR) of greater than 20 when the fluorescence image is acquired using an inverted fluorescence microscope equipped with a 20×objective, NA=0.75, dichroic mirror optimized for 532 nm light, a bandpass filter optimized for Cyanine dye-3 emission, and a camera, under non-signal saturating conditions while the surface is immersed in 25 mM ACES, pH 7.4 buffer. Embodiment 27. The method of any one of embodiments 3 to 26, wherein the sequencing comprises: i) priming nanoball sequences tethered to a surface within a sequencing flow cell with two or more copies of a sequencing primer and a polymerase; ii) contacting the primed nanoball sequences with a polymer-nucleotide conjugate comprising two or more copies of a nucleotide moiety under conditions that promote hybridization of complementary nucleotide bases to form multivalent binding complexes between the polymer-nucleotide conjugate and two or more primed nanoball sequences, or between the polymer-nucleotide conjugate and two or more identical sequences within a single primed “nanoball” sequence; iii) detecting the multivalent binding complexes on the surface within the sequencing flow cell, thereby determining the identity of a nucleotide within a sample barcode sequence or probe barcode sequence of the nanoball sequences; and iv) repeating steps (ii) to (iii) to determine the sample barcode and probe barcode sequences of the nanoball sequences. Embodiment 28. The method of embodiment 27, wherein the two or more nucleotide moieties of the polymer-nucleotide conjugate are not incorporated during the contacting or detecting steps. Embodiment 29. The method of any one of embodiments 1 to 28, wherein the detecting in (e) comprises sequencing sample barcode sequences and probe barcode sequences that collectively comprise a total of 30 or fewer base calls. Embodiment 30. The method of any one of embodiments 1 to 29, wherein the detecting in (e) comprises sequencing sample barcode sequences and probe barcode sequences that collectively comprise a total of 20 or fewer base calls. Embodiment 31. The method of any one of embodiments 1 to 30, wherein a total time required to extract target nucleic acid molecules from a sample, perform the method, and detect the presence of the target nucleic acid in the sample is less than 4 hours. Embodiment 32. The method of any one of embodiments 1 to 31, wherein a total time required to extract target nucleic acid molecules from a sample, perform the method, and detect the presence of the target nucleic acid in the sample is less than 3 hours. Embodiment 33. The method of any one of embodiments 1 to 32, wherein steps (a) through (c) are performed in parallel, and the plurality of samples comprises at least 96 samples per experimental run. Embodiment 34. The method of any one of embodiments 1 to 33, wherein steps (a) through (c) are performed in parallel, and the plurality of samples comprises at least 384 samples per experimental run. Embodiment 35. The method of any one of embodiments 1 to 34, wherein steps (a) through (c) are performed in parallel, and the plurality of samples comprises at least 1,536 samples per experimental run. Embodiment 36. The method of any one of embodiments 1 to 35, wherein the number of unique sample barcodes is at least 1,000. Embodiment 37. The method of any one of embodiments 1 to 36, wherein the number of unique sample barcodes is at least 5,000. Embodiment 38. The method of any one of embodiments 1 to 37, wherein the number of unique sample barcodes is at least 10,000.

### Further Embodiments

1. A method for detecting a presence of a nucleic acid sequence derived from Severe Acute Respiratory Syndrome (SARS)-coronavirus (CoV) in a sample, comprising:
(a) contacting said nucleic acid sequence or a derivative thereof with a nucleic acid probe molecule comprising a distal end and a proximal end under conditions sufficient to couple said distal end of said nucleic acid probe molecule and said proximal end of said nucleic acid probe molecule to couple to said nucleic acid sequence, thereby forming a circular nucleic acid probe molecule; and
(b) identifying a nucleic acid sequence of said circular nucleic acid probe molecule, thereby detecting the presence of said nucleic acid sequence derived from said SARS-CoV in said sample.
2. The method of embodiment 1, wherein said circular nucleic acid probe comprises a gap in a nucleic acid sequence thereof.
3. The method of embodiment 2, further comprising contacting said nucleic acid probe molecule with a polymerizing enzyme under conditions sufficient to perform an extension reaction, thereby filling said gap with a copy of a portion of said nucleic acid sequence derived from said SARS-CoV.
4. The method of embodiment 3, wherein said nucleic acid sequence of said circular nucleic acid probe molecule that is identified in (b) comprises said copy of said portion of said nucleic acid sequence derived from said SARS-CoV.
5. The method of embodiment 3, further comprising contacting said nucleic acid probe molecule with a ligating enzyme under conditions sufficient to ligate said distal end of said nucleic acid probe molecule to said proximal end of said nucleic acid probe molecule following said extension reaction.
6. The method of embodiment 2, wherein said gap comprises between 1 and 200 contiguous nucleotides in length.
7. The method of embodiment 1, further comprising contacting said nucleic acid probe molecule with a ligating enzyme under conditions sufficient to ligate said distal end of said nucleic acid probe molecule to said proximal end of said nucleic acid probe molecule, thereby forming said circular nucleic acid probe molecule.
8. The method of embodiment 1, wherein said nucleic acid probe molecule is linear when unhybridized.
9. The method of embodiment 1, wherein said nucleic acid sequence of said circular nucleic acid probe molecule that is identified in (b) comprises a barcode sequence that uniquely identifies said presence of said nucleic acid sequence derived from said SARS-CoV when it is identified.
10. The method of embodiment 1, further comprising:
(c) repeating (a) to (b) to identify a plurality of said nucleic acid sequence of a plurality of said circular nucleic acid probe molecule in said sample; and
(d) counting a number of times each nucleic acid sequence of said plurality of said nucleic acid sequence is identified in (c).
11. The method of embodiment 10, further comprising determining a copy number of said nucleic acid sequence derived from said SARS-CoV in said sample, wherein said copy number of said nucleic acid sequence derived from said SARS-CoV in said sample is proportional to said number of said times each nucleic acid sequence is counted in (d).
12. The method of embodiment 1, further comprising multiplexing said method comprising:
(c) repeating (a) to (b) to identify a plurality of nucleic acid sequences of a plurality of said circular nucleic acid probe molecule in said sample, wherein a first subset of said plurality of said circular nucleic acid probe molecule is different than a second subset of said plurality of said circular nucleic acid molecule; and
(d) counting a number of times a first nucleic acid sequence of said first subset and a second nucleic acid sequence of said second subset are identified in (c).
13. The method of embodiment 12, further comprising determining a copy number of said SARS-CoV in said sample, wherein said copy number of said SARS-CoV in said sample is proportional to said number of said times said first nucleic acid sequence or said second nucleic acid sequence is counted in (d).
14. The method of embodiment 12, wherein said first subset of said plurality of said circular nucleic acid probe molecule is different than said second subset of said plurality of said circular nucleic acid molecule in that:
(i) said first subset comprises a different barcode than said second subset;
(ii) said first subset comprises a different distal end or proximal end than said second subset; or
(iii) a combination of (i) and (ii).
15. The method of embodiment 1, further comprising detecting a presence of a second nucleic acid sequence derived from a pathogen other than said SARS-CoV in said sample, comprising:
(c) contacting said second nucleic acid sequence in said sample derived from said pathogen with a second nucleic acid probe molecule under conditions sufficient to couple said second nucleic acid sequence with said second nucleic acid probe molecule, thereby forming a second circular nucleic acid probe molecule; and
(d) identifying a nucleic acid sequence of said second circular nucleic acid probe molecule, thereby detecting said presence of said second nucleic acid sequence derived from said pathogen in said sample.
16. The method of embodiment 1, further comprising detecting a presence of a second nucleic acid sequence derived from said SARS-CoV in a second sample, comprising:
(c) contacting said second nucleic acid sequence in said second sample derived from said SARS-CoV with a second nucleic acid probe molecule under conditions sufficient to couple said second nucleic acid sequence with said second nucleic acid probe molecule, thereby forming a second circular nucleic acid probe molecule; and
(d) identifying a nucleic acid sequence of said second circular nucleic acid probe molecule, thereby detecting said presence of said second nucleic acid sequence derived from said SARS-CoV in said second sample.
17. The method of embodiment 16, wherein said second sample is obtained from a different source than said sample.
18. The method of embodiment 16, further comprising tracing a pathogenic infection by said SARS-CoV comprising comparing a location or a time of collection of said sample with a location or a time of collection of said second sample.
19. The method of any one of embodiments 1 to 18, wherein said sample is obtained from a source comprising:
   (i) soil;
   (ii) sewage;
   (iii) biological tissue;
   (iv) food;
   (v) a surface of an object in contact with one or more of (i) to (iv); or
   (vi) any combination of (i) to (v).
20. The method of any one of embodiments 1 to 19, wherein said SARS-CoV comprises SARS-CoV-2, or a variant thereof.
21. The method of embodiment 20, wherein said SARS-CoV-2 or variant thereof is encoded by a sequence comprising at least about 99% sequence identity to SEQ ID NO: 1.
22. The method of embodiment 20, wherein said SARS-CoV-2 or variant thereof is encoded by a sequence comprising any one of SEQ ID NO: 1-4.
23. A system for nucleic acid processing:
a nucleic acid probe molecule comprising (i) a proximal end comprising a first nucleic acid sequence that is complementary to a first portion of a nucleic acid sequence derived from Severe Acute Respiratory Syndrome (SARS)-coronavirus (CoV), and (ii) a distal end comprising a second nucleic acid sequence that is complementary to a second portion of said nucleic acid sequence derived from SARS-CoV; and one or more computer processors that are individually or collectively programmed to perform a method comprising:
   (a) contacting said nucleic acid probe molecule with said nucleic acid sequence derived from SARS-CoV under conditions sufficient to cause (i) said proximal end of said nucleic acid probe molecule to couple with said first portion of said nucleic acid sequence derived from SARS-CoV, and (ii) said distal end of said nucleic acid probe molecule to couple with said second portion of said nucleic acid sequence derived from SARS-CoV, thereby forming a circular nucleic acid probe molecule;
   (b) identifying a nucleic acid sequence of said circular nucleic acid probe molecule, thereby detecting the presence of said nucleic acid sequence derived from said SARS-CoV.
24. The system of embodiment 23, further comprising a substrate having a surface comprising a polymer layer coupled thereto, wherein said nucleic acid probe molecule is coupled to said polymer layer.
25. The system of embodiment 24, wherein said surface comprises two or more interior surfaces of a flow cell.
26. The system of embodiment 24, wherein said polymer layer comprises a hydrophilic polymer.
27. The system of embodiment 26, wherein said hydrophilic polymer comprises polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, or dextran, or any combination thereof.
28. The system of embodiment 23, further comprising a ligating enzyme or catalytically-active fragment thereof configured to ligate said proximal end of said nucleic acid probe molecule and said distal end of said nucleic acid probe molecule to form said circular nucleic acid probe molecule.
29. The system of embodiment 23, wherein said circular nucleic acid probe molecule comprises a gap in a nucleic acid sequence thereof.
30. The system of embodiment 29, further comprising a polymerizing enzyme configured to perform an extension reaction of said circular nucleic acid probe molecule, thereby filling said gap with a copy of a third portion of said nucleic acid sequence derived from said SARS-CoV.
31. The system of embodiment 30, wherein said nucleic acid sequence of said circular nucleic acid probe molecule that is identified in (b) comprises said third portion of said nucleic acid sequence derived from said SARS-CoV.
32. The system of embodiment 29, wherein said gap comprises between 1 and 200 contiguous nucleotides in length.
33. The system of embodiment 23, wherein said nucleic acid probe molecule is linear when unhybridized.
34. The system of embodiment 23, wherein said nucleic acid sequence of said circular nucleic acid probe molecule that is identified in (b) comprises a barcode sequence that uniquely identifies said presence of said nucleic acid sequence derived from said SARS-CoV when it is identified.
35. The system of embodiment 23, wherein said method further comprises:
   (c) repeating (a) to (b) to identify a plurality of said nucleic acid sequence of said circular nucleic acid probe molecule in said sample; and
   (d) counting a number of times each nucleic acid sequence of said plurality of said nucleic acid sequence of said circular nucleic acid probe molecule is identified in (c).
36. The system of embodiment 35, wherein said method further comprises determining a copy number of said SARS-CoV in said sample, wherein said copy number of said SARS-CoV in said sample is proportional to said number of said times each nucleic acid sequence is counted in (d).
37. The system of embodiment 23, further comprising a plurality of said circular nucleic acid probe molecule comprising a first subset of said plurality of said circular nucleic acid probe molecule, and a second subset of said plurality of said circular nucleic acid probe molecule, wherein said first subset is different than said second subset.
38. The system of embodiment 37, wherein said method is a multiplexed method, further comprising:
   (c) repeating (a) to (b) to identify a plurality of nucleic acid sequences of a plurality of said plurality of said circular nucleic acid probe molecule in said sample; and
   (d) counting a number of times a first nucleic acid sequence of said first subset and a second nucleic acid sequence of said second subset are identified in (c).
39. The system of embodiment 38, wherein said method further comprises determining a copy number of said nucleic acid sequence derived from said SARS-CoV in said sample, wherein said copy number of said nucleic acid sequence derived from said SARS-CoV in said sample is proportional to said number of said times said first nucleic acid sequence or said second nucleic acid sequence is counted in (d).
40. The system of embodiment 38, wherein said first subset of said plurality of said circular nucleic acid probe molecule is different than said second subset of said plurality of said circular nucleic acid molecule in that:
   (i) said first subset comprises a different barcode than said second subset;
   (ii) said first subset comprises a different distal end or proximal end than said second subset; or
   (iii) a combination of (i) and (ii).
41. The system of embodiment 23, further comprising a second nucleic acid probe molecule, wherein said second nucleic acid probe molecule configured to couple to a nucleic acid sequence derived from a pathogen other than said SARS-CoV.
42. The system of embodiment 41, wherein said method further comprises detecting a presence of said nucleic acid sequence derived from a pathogen other than said SARS-CoV in said sample, comprising:
   (c) contacting said second nucleic acid sequence in said sample with said second nucleic acid probe molecule under conditions sufficient to couple said second nucleic acid sequence with said second nucleic acid probe molecule, thereby forming a second circular nucleic acid probe molecule; and
   (d) identifying a nucleic acid sequence of said second circular nucleic acid probe molecule, thereby detecting said presence of said second nucleic acid sequence derived from said pathogen in said sample.
43. The system of embodiment 23, wherein said method further comprises detecting a presence of a second nucleic acid sequence derived from said SARS-CoV in a second sample, comprising:
   (c) contacting said second nucleic acid sequence in said second sample derived from said SARS-CoV with a second nucleic acid probe molecule under conditions sufficient to couple said second nucleic acid sequence with said second nucleic acid probe molecule, thereby forming a second circular nucleic acid probe molecule; and
   (b) identifying a nucleic acid sequence of said second circular nucleic acid probe molecule, thereby detecting said presence of said second nucleic acid sequence derived from said SARS-CoV in said second sample.
44. The system of embodiment 43, wherein said second sample is obtained from a different source than said sample.
45. The system of embodiment 43, wherein said method further comprises tracing a pathogenic infection by said SARS-CoV comprising comparing a location or time of collection of said sample with a location or time of collection of said second sample.
46. The system of any one of embodiments 23 to 45, wherein said sample is obtained from a source comprising:
   (i) soil;
   (ii) sewage;
   (iii) biological tissue;
   (iv) food;
   (v) a surface of an object in contact with one or more of (i) to (iv); or
   (vi) any combination of (i) to (v).
47. The system of any one of embodiments 23 to 46, wherein said SARS-CoV comprises SARS-CoV-2, or a variant thereof.
48. The system of embodiment 47, wherein said SARS-CoV-2 or variant thereof is encoded by a sequence comprising at least about 99% sequence identity to SEQ ID NO: 1.
49. The system of embodiment 47, wherein said SARS-CoV-2 or variant thereof is encoded by a sequence comprising any one of SEQ ID NO: 1-4.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the phrase ‘at least one of’ in the context of a series encompasses lists including a single member of the series, two members of the series, up to and including all members of the series, alone or in some cases in combination with unlisted components.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, unrecited additives, components, integers, elements or method steps.

As used herein, the term ‘about’ a number refers to that number plus or minus 10% of that number. The term ‘about’ when used in the context of a range refers to that range minus 10% of its lowest value and plus 10% of its greatest value.

As used herein, “nucleic acid” (also referred to as a “polynucleotide”, “oligonucleotide”, ribonucleic acid (RNA), or deoxyribonucleic acid (DNA)) is a linear polymer of two or more nucleotides joined by covalent internucleosidic linkages, or variants or functional fragments thereof. In naturally occurring examples of nucleic acids, the internucleoside linkage is a phosphodiester bond. However, other examples optionally comprise other internucleoside linkages, such as phosphorothiolate linkages and may or may not comprise a phosphate group. Nucleic acids include double- and single-stranded DNA, as well as double- and single-stranded RNA, DNA/RNA hybrids, peptide-nucleic acids (PNAs), hybrids between PNAs and DNA or RNA, and may also include other types of nucleic acid modifications.

As used herein, a “nucleotide” refers to a nucleotide, nucleoside, or analog thereof. The nucleotide refers to both naturally occurring and chemically modified nucleotides and can include but are not limited to a nucleoside, a ribonucleotide, a deoxyribonucleotide, a protein-nucleic acid residue, or derivatives. Examples of the nucleotide includes an adenine, a thymine, a uracil, a cytosine, a guanine, or residue thereof; a deoxyadenine, a deoxythymine, a deoxyuracil, a deoxycytosine, a deoxyguanine, or residue thereof; a adenine PNA, a thymine PNA, a uracil PNA, a cytosine PNA, a guanine PNA, or residue or equivalents thereof, an N- or C-glycoside of a purine or pyrimidine base (e.g., a deoxyribonucleoside containing 2-deoxy-D-ribose or ribonucleoside containing D-ribose).

The term “barcode” as used herein refers to a natural or synthetic nucleic acid sequence comprised by a polynucleotide allowing for unambiguous identification of the polynucleotide and other sequences comprised by the polynucleotide having said barcode sequence. The number of different barcode sequences theoretically possible can be directly dependent on the length of the barcode sequence; e.g., if a DNA barcode with randomly assembled adenine, thymidine, guanosine and cytidine nucleotides can be used, the theoretical maximal number of barcode sequences possible can be 1,048,576 for a length of ten nucleotides, and can be 1,073,741,824 for a length of fifteen nucleotides.

As used herein, the term “isothermal” refers to a condition in which the temperature remains substantially constant. A temperature that is “substantially constant” may deviate (e.g., increase or decrease) over a period of time by no more than 0.25 degrees, 0.50 degrees, 0.75 degrees, or 1.0 degrees.

The terms “anneal” or “hybridize,” are used herein interchangeably to refer to the ability of two nucleic acid molecules to combine together. In some cases, the “combining” refers to Watson-Crick base pairing between the bases in each of the two nucleic acid molecules.

As used herein, the terms, “isolate” and “purify,” are used interchangeably herein unless specified otherwise.

As used herein, the terms “DNA hybridization” and “nucleic acid hybridization” are used interchangeably and are intended to cover any type of nucleic acid hybridization, e.g., DNA hybridization, RNA hybridization, unless otherwise specified. Hybridization may occur through Watson-Crick base pairing, Hoogsteen pairing, G-loop pairing, or any mechanism for the specific or ordered noncovalent interaction of bases within two or more nucleic acid strands. “Hybridization” may comprise interactions between segments of a single molecule, two molecules, or more than two molecules of a nucleic acid.

As used herein, “hybridization specificity” refers to a measure of the ability of nucleic acid molecules (e.g., adapter sequences, primer sequences, or oligonucleotide sequences) to correctly hybridize to a region of a target nucleic acid molecule with a nucleic acid sequence that is completely complementary to the nucleic acid molecule.

As used herein, the term “hybridization stringency” refer to a percentage of nucleotide bases within at least a portion of a nucleic acid sequence undergoing a hybridization (e.g., a hybridization region) reaction that is complementary through standard Watson-Crick base pairing. In a non-limiting example, a hybridization stringency of 80% means that a stable duplex can be formed in which 80% of the hybridization region undergoes Watson-Crick base pairing. A higher hybridization stringency means a higher degree of Watson-Crick base pairing is required in a given hybridization reaction in order to form a stable duplex.

As used herein, “hybridization sensitivity” refers to a concentration range of sample (or target) nucleic molecules in which hybridization occurs with high specificity. In some cases, as little as 50 picomolar concentration of sample nucleic acid molecules in which hybridization with high specify is achieved with the methods, compositions, systems and kits described herein. In some cases, the range is between about 1 nanomolar to about 50 picomolar concentrations of sample nucleic acid molecules.

As used herein, “hybridization efficiency” refers to a measure of the percentage of total available nucleic acid molecules (e.g., adapter sequences, primer sequences, or oligonucleotide sequences) that are hybridized to the region of the target nucleic acid molecule with the nucleic acid sequence that is completely complementary to the nucleic acid molecule.

“Complementary”, as used herein, refers to the topological compatibility or matching together of interacting surfaces of a ligand molecule and its receptor. Thus, the receptor and its ligand can be described as complementary, and furthermore, the contact surface characteristics are complementary to each other.

“Branched polymer”, as used herein, refers to a polymer having a plurality of functional groups that help conjugate a biologically active molecule such as a nucleotide, and the functional group can be either on the side chain of the polymer or directly attaches to a central core or central backbone of the polymer. The branched polymer can have linear backbone with one or more functional groups coming off the backbone for conjugation. The branched polymer can also be a polymer having one or more sidechains, wherein the side chain has a site for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

“Polymerase”, as used herein, refers to an enzyme that contains a nucleotide binding moiety and helps formation of a binding complex between a target nucleic acid and a complementary nucleotide. The polymerase can have one or more activities including, but not limited to, base analog detection activities, DNA polymerization activity, reverse transcriptase activity, DNA binding or incorporation, strand displacement activity, and nucleotide binding or incorporation and recognition. The polymerase can include catalytically inactive polymerase, catalytically active polymerase, reverse transcriptase, and other enzymes containing a nucleotide binding or incorporation moiety.

“Persistence time”, as used herein, refers to the length of time that a binding complex, which is formed between the target nucleic acid, a polymerase, a conjugated or unconjugated nucleotide, remains stable without any binding component dissociates from the binding complex. The persistence time is indicative of the stability of the binding complex and strength of the binding interactions. Persistence time can be measured by observing the onset or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One non-limiting example of label is a fluorescent label.

In some embodiments, the methods and compositions of the present disclosure comprise a label, such as a fluorescent label or a fluorophore. In some embodiments, the label is a fluorophore. Fluorescent moieties which may serve as fluorescent labels or fluorophores include, but are not limited to, fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and comprise two indolenin, benzo-indolium, pyridium, thiozolium, or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((lE,3E)-5-((E)-1-(6-((2,5-dioxopyrrolidin-1-yl)oxy)-6-oxohexyl)-3,3-dimethyl-5-indolin-2-ylidene)penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene)penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(i-ethyl-1,3-dihydro-2H-indol-2-ylidene)hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene)hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

An “organic solvent,” as used herein refers to a solvent or solvent system comprising carbon-based or carbon-containing substance capable of dissolving or dispersing other substances. An organic solvent may be miscible or immiscible with water.

The term “support” includes any solid or semisolid article on which reagents such as nucleic acids can be immobilized. Nucleic acids may be immobilized on the solid support by any method including but not limited to physical adsorption, by ionic or covalent bond formation, or combinations thereof. A solid support may include a polymeric, a glass, or a metallic material. Examples of solid supports include a membrane, a planar surface, a microtiter plate, a bead, a filter, a test strip, a slide, a cover slip, and a test tube, means any solid phase material upon which an oligomer is synthesized, attached, ligated or otherwise immobilized. A support may comprise a “resin”, “phase”, “surface,” “substrate,” “coating,” or “support.” A support may comprise organic polymers such as polystyrene, polyethylene, polypropylene, polyfluoroethylene, polyethyleneoxy, and polyacrylamide, as well as copolymers and grafts thereof. A support may also be inorganic, such as glass, silica, controlled-pore-glass (CPG), or reverse-phase silica. The configuration of a support may be in the form of beads, spheres, particles, granules, a gel, or a surface. Surfaces may be planar, substantially planar, or non-planar. Supports may be porous or non-porous and may have swelling or non-swelling characteristics. A support can be shaped to comprise one or more wells, depressions or other containers, vessels, features or locations. A plurality of supports may be configured in an array at various locations. A support may be addressable (e.g., for robotic delivery of reagents), or by detection methods including scanning by laser illumination and confocal or deflective light gathering. An amplification support (e.g., a bead) can be placed within or on another support (e.g., within a well of a second support).

As used herein, a “detectable label” refers to any molecule that aids in the detection of another biomolecule. Examples include, but are not limited to, chromophores, fluorophores, quantum dots, upconverting phosphors, luminescent or chemiluminescent molecules, radioisotopes, magnetic nanoparticles, mass tags, and the like. In some instances, a preferred label may comprise a fluorophore.

As used herein, fluorescence is “specific” if it arises from fluorophores that are annealed or otherwise tethered to the surface, such as through a nucleic acid having a region of reverse complementarity to a corresponding segment of an oligo on the surface and annealed to said corresponding segment. This fluorescence is contrasted with fluorescence arising from fluorophores not tethered to the surface through such an annealing process, or in some cases to background florescence of the surface.

As used herein, the term “detection channel” refers to an optical path (and/or the optical components therein) within an optical system that is configured to deliver an optical signal arising from a sample to a detector. In some instances, a detection channel may be configured for performing spectroscopic measurements, e.g., monitoring a fluorescence signal or other optical signal using a detector such as a photomultiplier. In some instances, a “detection channel” may be an “imaging channel”, i.e., an optical path (and/or the optical components therein) within an optical system that is configured to capture and deliver an image to an image sensor.

As used herein, the phrases “imaging module”, “imaging unit”, “imaging system”, “optical imaging module”, “optical imaging unit”, and “optical imaging system” are used interchangeably, and may comprise components or sub-systems of a larger system that may also include, e.g., fluidics modules, temperature control modules, translation stages, robotic fluid dispensing and/or microplate handling, processor or computers, instrument control software, data analysis and display software, etc.

As used herein, the term “excitation wavelength” refers to the wavelength of light used to excite a fluorescent indicator (e.g., a fluorophore or dye molecule) and generate fluorescence. Although the excitation wavelength is typically specified as a single wavelength, e.g., 620 nm, it may refer to a wavelength range or excitation filter bandpass that is centered on the specified wavelength. For example, in some instances, light of the specified excitation wavelength comprises light of the specified wavelength ±2 nm, ±5 nm, ±10 nm, ±20 nm, 40 nm, ±80 nm, or more. In some instances, the excitation wavelength used may or may not coincide with the absorption peak maximum of the fluorescent indicator.

As used herein, the term “emission wavelength” refers to the wavelength of light emitted by a fluorescent indicator (e.g., a fluorophore or dye molecule) upon excitation by light of an appropriate wavelength. Although the emission wavelength is typically specified as a single wavelength, e.g., 670 nm, this specification may refer to a wavelength range or emission filter bandpass that is centered on the specified wavelength. In some instances, light of the specified emission wavelength comprises light of the specified wavelength ±2 nm, ±5 nm, ±10 nm, ±20 nm, ±40 nm, ±80 nm, or more. In some instances, the emission wavelength used may or may not coincide with the emission peak maximum of the fluorescent indicator.

### EXAMPLES

These examples are provided for illustrative purposes only and not to limit the scope of the claims provided herein.

### Example 1—Hydrophilic Substrates

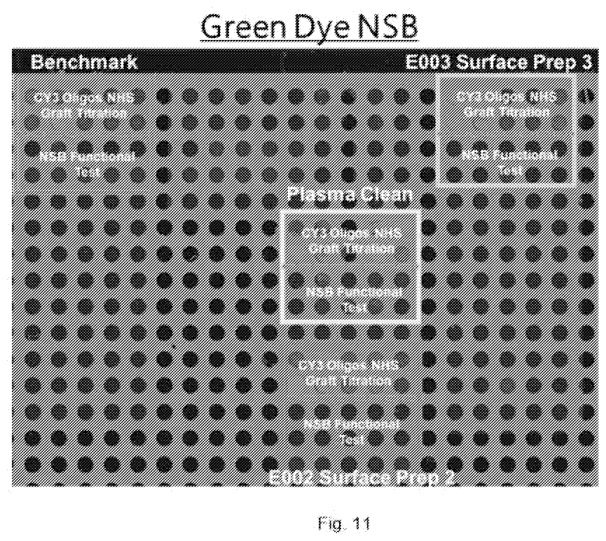

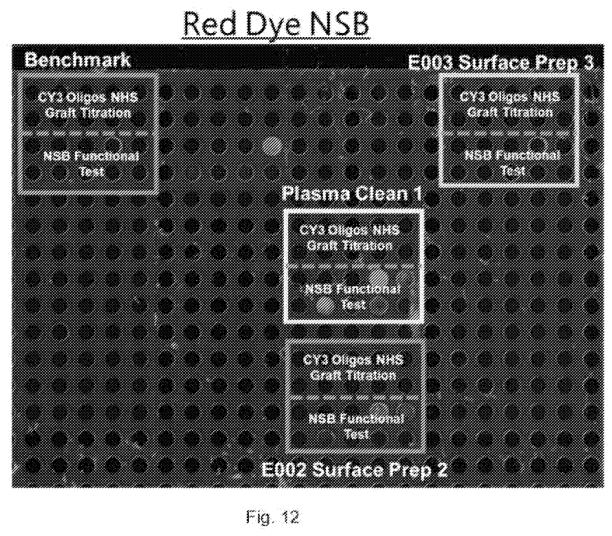

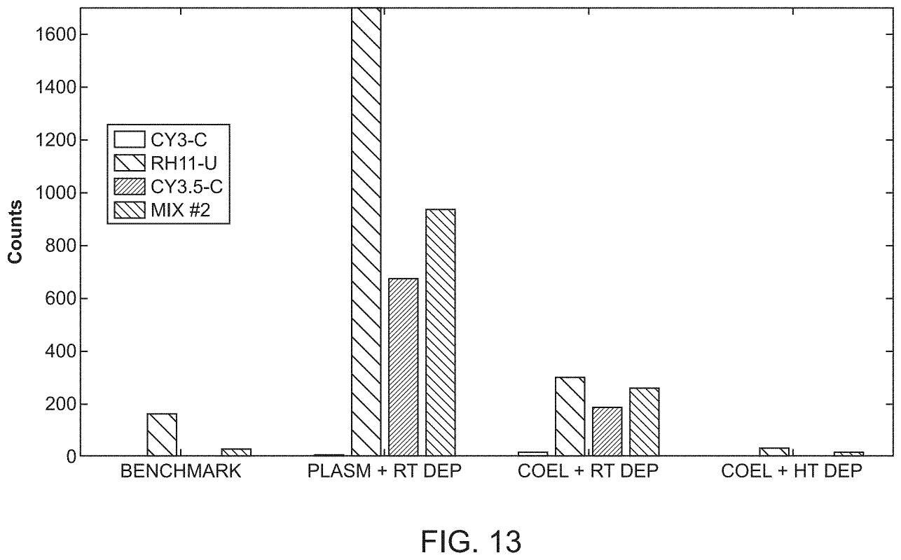

It is possible to generate low NSB/low background substrate surfaces for performing solid-phase nucleic acid amplification and sequencing chemistries that provide significantly improved nucleic acid amplification, such that the signal-to-background ratios can be tuned to meet the needs of a specific sequencing application. FIG. 11 provides an example of image data from a study to determine the relative levels of non-specific binding of a green fluorescent dye to glass substrate surfaces treated according to different surface modification protocols. FIG. 12 provides an example of image data from a study to determine the relative levels of non-specific binding of a red fluorescent dye to glass substrate surfaces treated according to different surface modification protocols. FIG. 13 provides an example of oligonucleotide primer grafting data for substrate surfaces treated according to different surface modification protocols.

### Example 2—Method for Preparing a Multi-Layer PEG Surface with NHS Ester-Amine Chemistry

A glass slide is cleaned by 2M KOH treatment of 30 minutes at room temperature, washed, and then surface silanol groups are activated using an oxygen plasma. Silane-PEG2K-amine (Nanocs, Inc., New York, NY) is applied at a concentration of 0.5% in ethanol solution. After a 2-hour coating reaction, the slide was washed thoroughly with ethanol and water. 100 uM of8-arm PEG NHS (MW=10K, Creative PEGWorks, Inc., Durham, NC) was introduced at room temperature for 20 minute in a solvent composition that can include 5, 10, 20, 30, 40, 50, 60, 70, 80 or 90 percent organic solvent and 5, 10, 20, 30, 40, 50, 60, 70, 80 or 90 percent low ionic strength buffer. The resulting surface was washed and reacted with 20 μM multiarm PEG amine (MW=10K, Creative PEGWorks, Inc., Durham, NC) for 2 hours. The resulting amine-PEG surface was then reacted with a mixture of multiarm PEG-NHS and amine-labeled oligonucleotide primer at varying concentrations. This process can be repeated to generate additional PEG layers on the surface.

### Example 3—Modified Rolling Circle Multiple Displacement Amplification (Modified RCA-MDA)

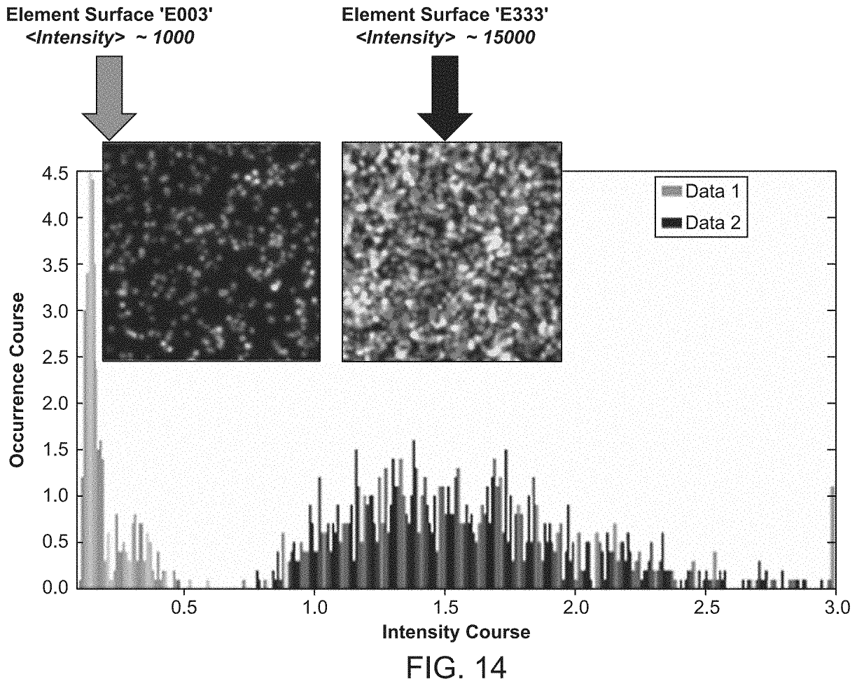

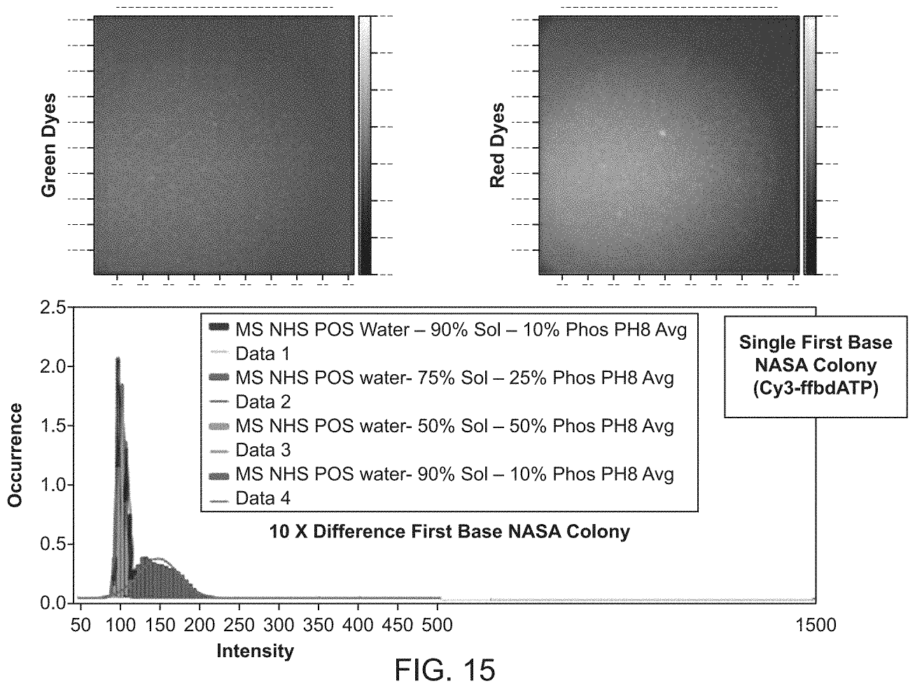

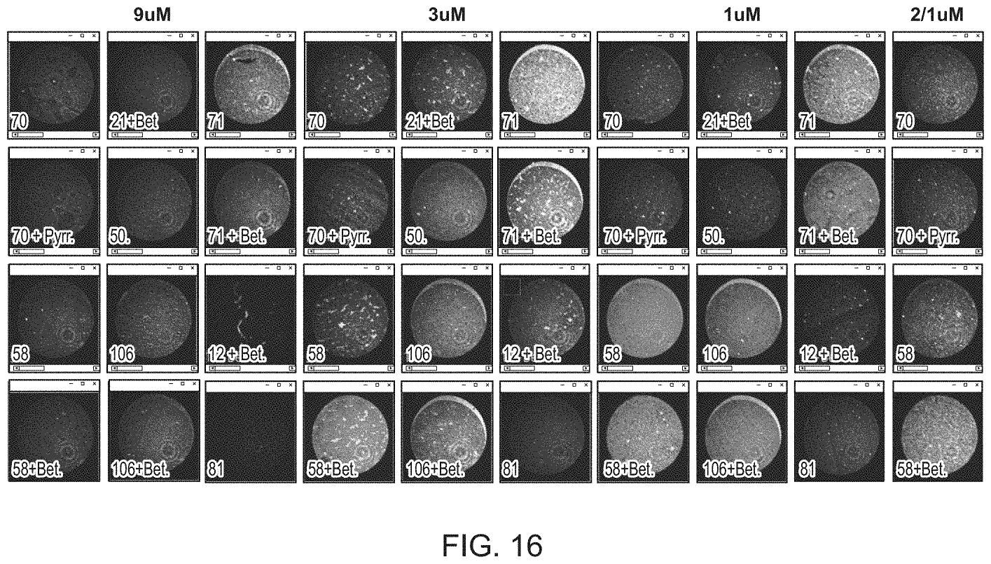

Copy number in the RCA-MDA colonies is determined by the primer surface density, which dictates how frequently and successfully the initial concatemers or displaced concatemers are hybridized with the forward and the reverse primers. Increased primer density on low binding surfaces has proven to generate higher amplification copy numbers in these clusters (FIG. 14). It is possible to increase the copy number or specific amplification and decrease the non-specific amplification on low binding surfaces, using one or a combination of the following methods: (i) specific copy number may be increased by increasing the efficiency of primer template hybridizations through formulation changes (FIG. 16), (ii) specific copy number may be increased by increasing the primer density on low binding substrates (FIG. 15 and FIG. 14), (iii) non-specific amplification of primer dimers or chimeric DNA generation may be decreased by using the additives described above, (iv) amplification incubation temperatures may be increased using thermostable enzymes combined with formulation changes as previously described to reduce the non-specific amplification, and (v) primer compositions that comprise non-self-hybridizing primer sequences may be used in combination with additives or increased amplification incubation temperatures to decrease non-specific primer dimer amplification.

### Example 4—Calculation of CNR on Cluster Data

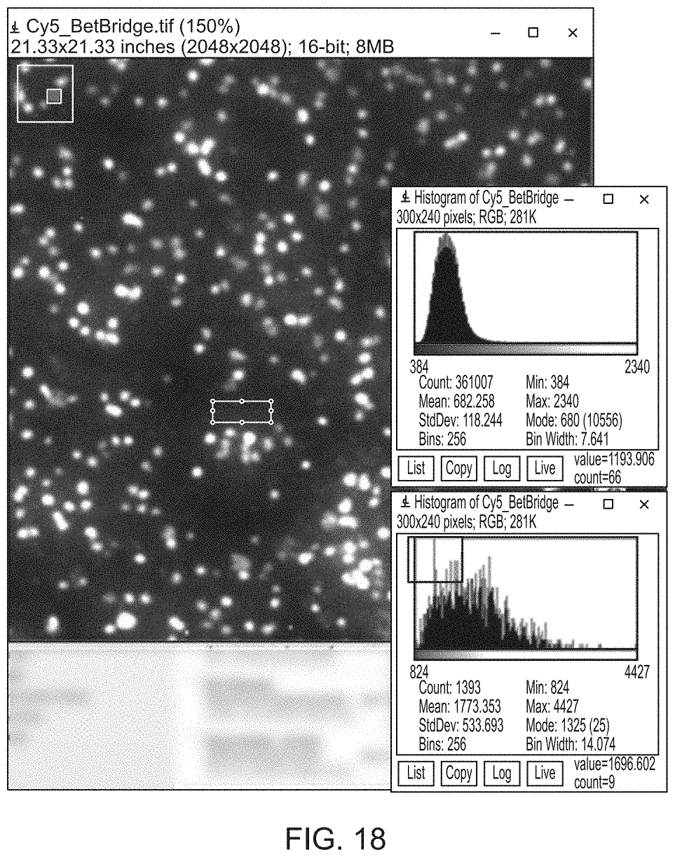

FIGS. 18-20 provide examples of raw image data and intensity data histograms used to calculate CNR for difference combinations of nucleic acid amplification methodology and the low-binding supports described here. In each of these examples, the upper histogram is the background pixel intensity histogram, the lower histogram is the foreground spot intensity histogram, and a portion of the original image is also included.

Surface densities for each of these experiments were estimated to be approximately 100K primers/μm². Primer surface density was estimated using the following methodology: (i) a fluorescence titration curve was prepared using a GE Typhoon (GE Healthcare Lifesciences, Pittsburgh, PA) and a capillary flow cell of known area (40 mm²), height (0.5 mm), and volume (200 μl) containing known concentrations of Cy3-dCTP, (ii) the primers grafted to the low-binding support were hybridized to Cy3-labeled complementary oligonucleotides using a conventional hybridization protocol (3× saline sodium citrate (SSC) at 37 degrees C. or at room temperature (RT); hybridization conditions may be characterized for completeness), fluorescence intensity for the resulting signal on the surface was measured using the same GE Typhoon instrument used to generate the calibration curve, (iii) and the number of primer molecules tethered per unit area of surface was calculated based on a comparison of the measured surface signal to the calibration curve.

DNA library sequences were then hybridized to the tethered primers. The hybridization protocols used for the library hybridization step can vary depending on surface properties, but controlled library input is required to create resolvable DNA amplified colonies.

DNA amplification was performed for this example using the following protocols: (i) bridge amplification at 28 cycles with primer density of approximately 1K primers/um², (ii) bridge amplification @ 28 cycles with higher primer density >5K primers/um², and (iii) rolling circle amplification (RCA) for 90 minutes with primer density of approximately 2-4 K primers/um².

Post amplification, the amplified DNA was hybridized with a complementary “sequencing” primer and a sequencing reaction mix comprising a Cy3-labeled dNTP was added (“first base” assay) to determine the first base CNR for each of the respective methodologies. Following first base incorporation, the sequencing reaction mixture was exchanged with buffer, imaging was performed using the same GE Typhoon instrument, and CNR was calculated on the resulting images.

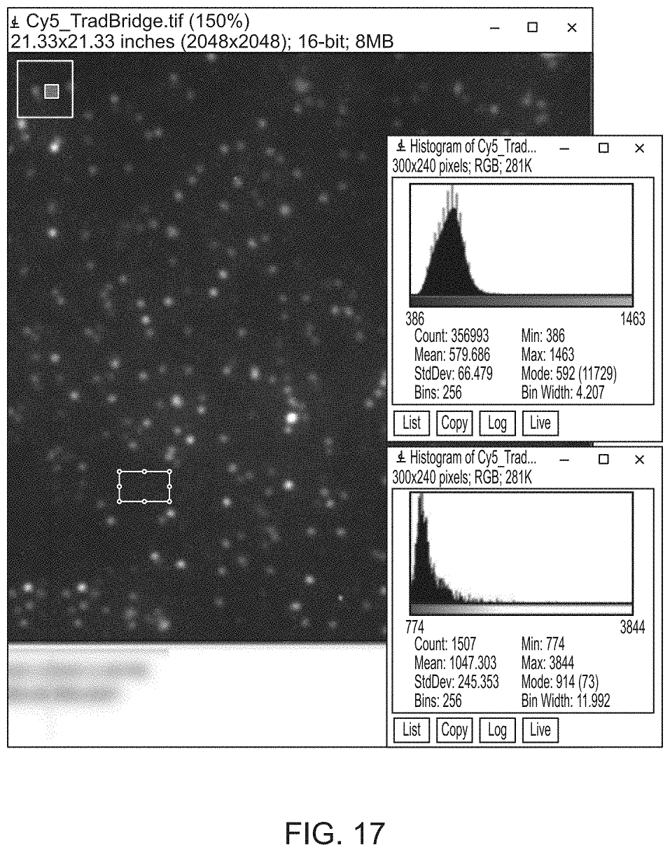

FIG. 17 provides an example of fluorescence image and intensity data for a low-binding support of the present disclosure on which solid-phase nucleic acid amplification was performed using bridge amplification @ 28 cycles with primer density of approximately 2K primers/um² to create clonally-amplified clusters of a template oligonucleotide sequence. In this example, the background intensity was 592 counts (with a standard deviation of 66.5 counts), the foreground intensity was 1047.3 counts, and the calculated CNR=(1047.3 −592)/66.5=455.3/66.5=6.8. The estimated non-specific noise=(592 −100)/(1047 −100)=52%.

FIG. 18 provides a second example of fluorescence image and intensity data for a low-binding support of the present disclosure on which solid-phase nucleic acid amplification was performed using bridge amplification @ 28 cycles with higher primer density >5K primers/um² to create clonally-amplified clusters of a template oligonucleotide sequence. In this example, the background intensity was 680 counts (with a standard deviation of 118.2 counts), the foreground intensity was 1773 counts, and the calculated CNR=(1773-680)/118.2=1093/118.2=9.2. The estimated non-specific noise=(680-100)/(1773-100)=35%.

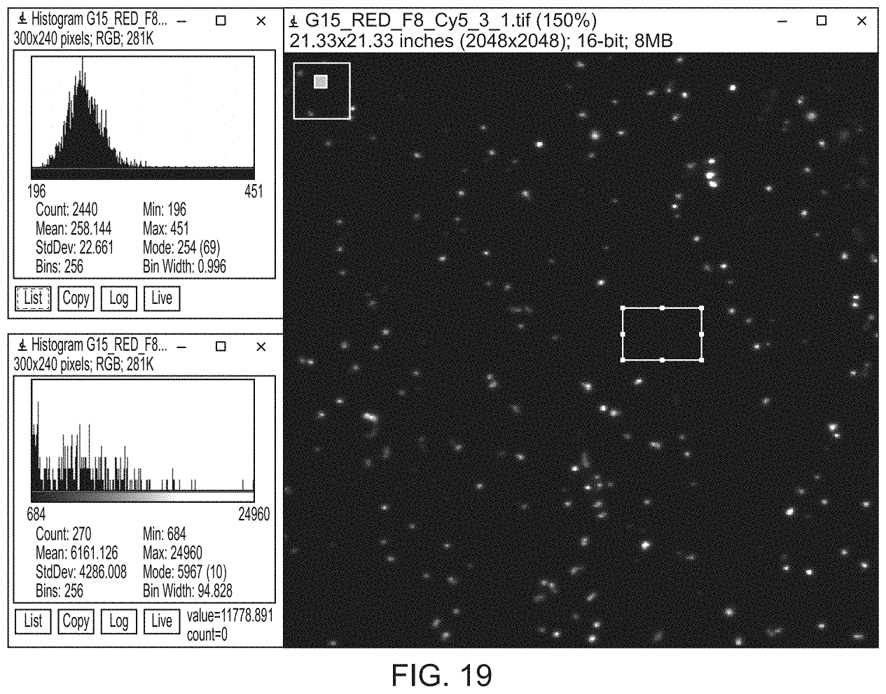

FIG. 19 provides an example of fluorescence image and intensity data for a low-binding support of the present disclosure on which solid-phase nucleic acid amplification was performed using rolling circle amplification (RCA) for 90 minutes with primer density of approximately 100 K primers/um² to create clonally-amplified clusters of a template oligonucleotide sequence. In these examples, the background intensity was 254 counts (with a standard deviation of 22.7 counts), the foreground intensity was 6161 counts, and the calculated CNR=(6161-254)/22.7=5907/22.7=260. Note the dramatic improvement in CNR achieved through the use of this combination of low-binding surface and amplification protocol. The estimated non-specific noise=(254-100)/(6161-100)=3%.

### Example 5—DNA Hybridization on Low Non-Specific Binding Surface

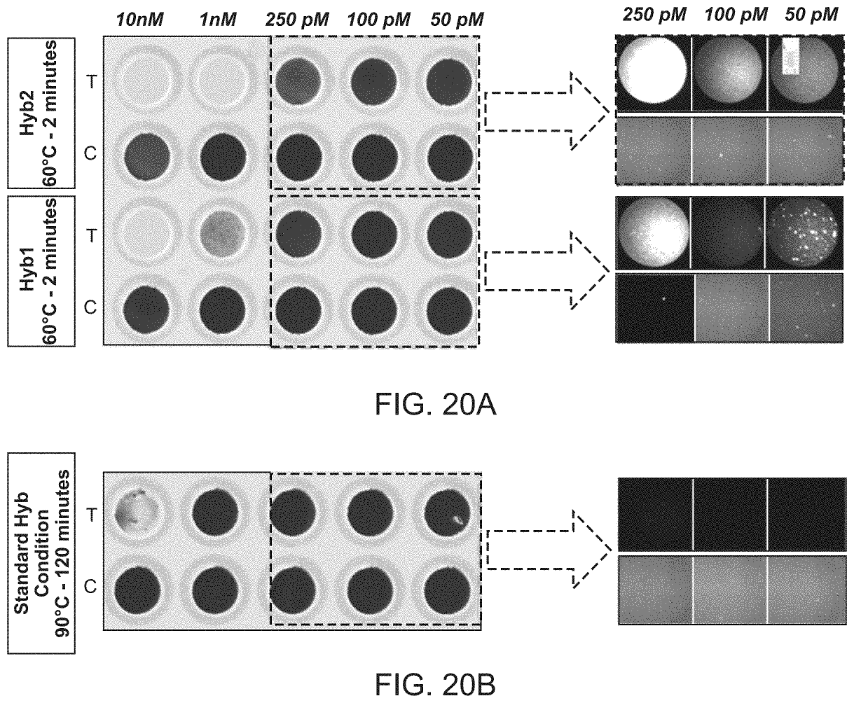

FIGS. 20A and 20B provide examples of the optimized hybridization achieved on low binding surface using the disclosed hybridization method (FIG. 20A) with reduced concentrations of hybridization reporter probe and shortened hybridization times, as compared to the results achieved using a traditional hybridization protocol on the same low binding surface (FIG. 20B).

FIG. 20A shows hybridization reactions on the low binding surface according to the embodiments described herein. The rows provide two test hybridization conditions, hybridization condition 1 (“Hyb 1”) and hybridization condition 2 (“Hyb 2”). Hyb 1 refers to the hybridization buffer composition C10 from Table 2. Hyb 2 refers to the hybridization buffer composition D18 from Table 2. A hybridization reporter probe (complementary oligonucleotide sequences labeled with a Cy™3 fluorophore at the 5′ end) at concentrations reported in FIG. 20A (10 nM, 1 nM, 250 pM, 100 pM, and 50 pM) were hybridized in the buffer compositions at 60 degrees Celsius for 2 minutes.

TABLE 2
Buffer compositions tested for hybridizing target nucleic acid with surface bound nucleic acid

| Graft | | | | | | | | | | | | | |
|---|---|---|---|---|---|---|---|---|---|---|---|---|---|
| concentration | | 1 uM | | | | | | | | 5.1 uM | | 46 uM | |
| | 9 | 10 | 11 | 12 | 13 | 14 | 15 | 16 | 17 | 18 | 19 | 20 | 21 |
| B | Cracked | 75% | 75% | 2x | 25% | Std | 30% | Std | 50% | Std | Std | Std | Std |
| | | ACN | ACN | SSC | ACN | buf. | PEG | | ACN | | | | |
| | | + | + | | + | + | | | + | | | | |
| | | MES | Phos | | 2x SSC | 5% | | | 50% | | | | |
| | | | | | + | PEG | | | Std | | | | |
| | | | | | 10% | + | | | buf. | | | | |
| | | | | | PEG | 30% | | | | | | | |
| | | | | | | Form. | | | | | | | |
| C | 1 uM | 50% | 50% | 4x | 25% | Std | 20% | Std | Std | Tris | Tris | Std | Std |
| | 31- | ACN | ACN | SSC | ACN | buf. | PEG | + | + | + | + | buff | buff |
| | NH2—Cy3 | + | + | | + | + | + | 2 | 2 | 1x SSC | 1x SSC | + | + |
| | | MES | Tris | | MES | 10% | 2x SSC | | | | | 5% | 5% |
| | | | | | + | PEG | | | | | | PEG | PEG |
| | | | | | 20% | + | | | | | | + | + |
| | | | | | PEG | 5% | | | | | | 30% | 30% |
| | | | | | + | Form. | | | | | | Form. | Form. |
| | | | | | 10% | | | | | | | | |
| | | | | | Form. | | | | | | | | |
| D | 1 uM | 25% | 25% | 10x | 50% | Std | 10% | Std | Std | 25% | 25% | Std | Std |
| | 31- | ACN | ACN | SSC | EtOH | buf. | PEG | + | + | ACN | ACN | buff | buff |
| | NH2—Cy3 | + | + | | + | + | + | 4 | 4 | + | + | + | + |
| | | MES | Tris | | 2x SSC | 10% | 2x SSC | | | MES | MES | 10% | 10% |
| | | + | + | | | PEG | + | | | + | + | PEG | PEG |
| | | 2x SSC | 2x SSC | | | + | 5% | | | 20% | 20% | + | + |
| | | | | | | 10% | Form. | | | PEG | PEG | 5% | 5% |
| | | | | | | Form. | | | | + | + | Form. | Form. |
| | | | | | | | | | | 10% | 10% | | |
| | | | | | | | | | | Form. | Form. | | |
| E | 1 uM | MES | Tris | 20x | 50% | Std | 5% | Std | Std | Std | Std | 10% | 10% |
| | 31- | + | + | SSC | EtOH | buf. | Form. | + | + | buf. | buf. | PEG | PEG |
| | NH2—Cy3 | 1x SSC | 1x SSC | | + | + | + | 6 | 6 | + | + | + | + |
| | | | | | 2x SSC | 20% | 2x SSC | | | 20% | 20% | 2x | 2x |
| | | | | | + | PEG | | | | PEG | PEG | SSC | SSC |
| | | | | | 10% | + | | | | + | + | + | + |
| | | | | | PEG | 10% | | | | 10% | 10% | 5% | 5% |
| | | | | | | Form. | | | | Form. | Form. | Form. | Form. |
| F | 10 nM | 10 nM | 10 nM | 10x | Std | Std | 10% | Std | Std | Std | Std | 10% | 10% |
| | 31- | 31- | 31- | SSC | | buf. | Form. | + | + | buf. | buf | Form. | Form. |
| | NH2—Cy3 | NH2—Cy3 | NH2—Cy3 | + | | + | + | 8 | 8 | + | + | + | + |
| | | | | 10% | | 10% | 2x SSC | | | 10% | 10% | 2x | 2x |
| | | | | Form. | | Form. | | | | Form. | Form. | SSC | SSC |

FIG. 20B shows hybridization reactions on the low binding surface according to a standard hybridization protocol with standard hybridization conditions (“Standard Hyb Conditions”). A standard hybridization buffer of 2×-5× saline-sodium citrate (SSC) was used with same hybridization reporter probe above at the same concentrations above, as shown in FIG. 20A. The standard hybridization reaction was performed at 90 degrees Celsius with a slow cool process (2 hours) to reach 37 degrees Celsius.

For each hybridization reaction provided in FIG. 20A and FIG. 20B, the top row for each hybridization reaction is test (“T”), which is the complementary oligos (e.g., CY3′™-5′-ACCCTGAAAGTACGTGCATTACATG-3′ (SEQ ID NO: 5)), and the bottom row for each hybridization reach is a control (“C”), which is a noncomplementary (e.g., CY3™-5′-ATGTCTATTACGTCACACTATTATG-3′(SEQ ID NO: 6)).

The surfaces used for all testing conditions were ultra-low non-specific binding surfaces having a level of non-specific Cy3 dye absorption corresponding to less than or equal to about 0.25 molecules/μm². In this example, the low non-specific binding surfaces used were glass substrates that were functionalized with Silane-PEG-5K-COOH (Nanocs Inc.). Following completion of the hybridization reactions, wells were washed with 50 mM Tris pH 8.0; 50 mM NaCl.

Images were obtained acquired using an inverted microscope (Olympus IX83) equipped with 100×TIRF objective, NA=1.4 (Olympus), dichroic mirror optimized for 532 nm light (Semrock, Di03-R532-t1-25x36), a bandpass filter optimized for Cy3 emission, (Semrock, FF01-562/40-25), and a camera (sCMOS, Andor Zyla) under non-signal saturating conditions for 1 s, (Laser Quantum, Gem 532, <1 W/cm² at the sample) while sample is immersed a buffer (25 mM ACES, pH 7.4 buffer). Images were collected as described above and results shown in FIG. 20A (optimized) and FIG. 20B (standard).

A significant signal was observed from the reaction with 250 picomolar (pM) in both Hyb 1 and Hyb 2 hybridization reactions (FIG. 20A), as compared with the negative control. In contrast, no signal was observed from the reaction with 250 pM in the Standard Hyb conditions, as compared with the negative control. The same result was observed for lower input concentrations (e.g., 100 pM, 50 pM) of the hybridization reporter probe. FIG. 20A shows more than 200-fold decrease in input DNA (labeled oligo) required for specific DNA capture on low non-specific binding surfaces tested, a 50×decrease in hybridization times, and a reduction in the hybridization temperatures by half, as compared with standard hybridization methods and reagents on the same low non-specific binding substrates (FIG. 20B).

### Example 6—Detection of Ternary Complex

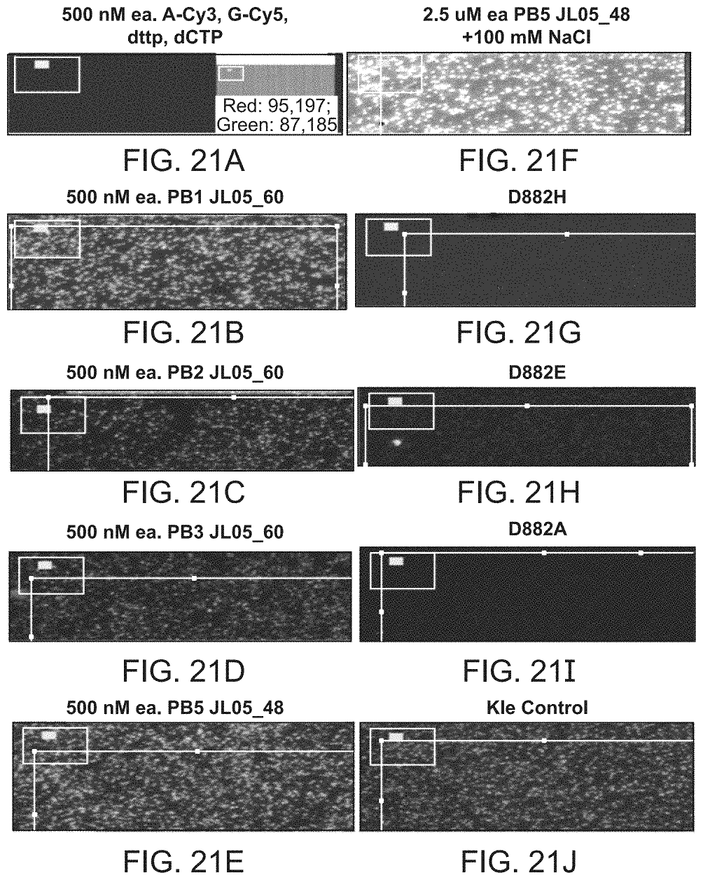

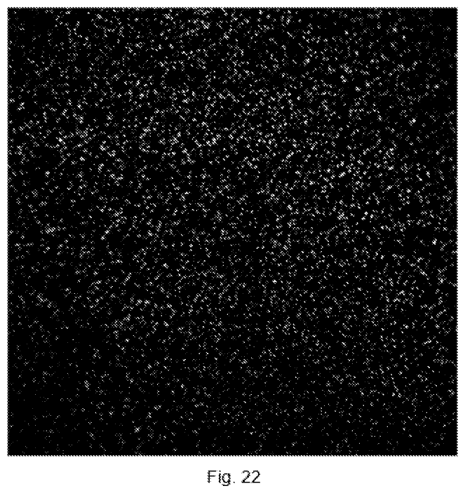

Binding reactions using the multivalent binding composition having PEG polymer-nucleotide conjugates were analyzed to detect possible formation of ternary binding complex, and the fluorescence images of the various steps are illustrated in FIG. 21. In FIG. 21A, red and green fluorescent images post exposure of DNA rolling circle application (RCA) templates (G and A first base) to 500 nM base labeled nucleotides (A-Cy3 and G-Cy5) in exposure buffer containing 20 nM Klenow polymerase and 2.5 mM Sr⁺². Multivalent PEG-substrate compositions were prepared using varying ratios of 4-armed PEG-amine (4ArmPEG-NH), biotin-PEG-amine (Biotin-PEG-NH), and nucleotide (Nuc) as follows: Samples PB1 and PB5, 4ArmPEG-NH: Biotin-PEG-NH: Nuc=0.25:1:0.5; Sample PB2, 4ArmPEG-NH: Biotin-PEG-NH: Nuc=0.125:0.5:0.25; Sample PB3, 4ArmPEG-NH: Biotin-PEG-NH: Nuc=0.25:1:0.5. Images were collected after washing with imaging buffer with the same composition as the exposure buffer but containing no nucleotides or polymerase.

Contrast was scaled to maximize visualization of the dimmest signals, but no signals persisted following washing with imaging buffer (FIG. 21A, inset). In FIGS. 21B-21E, the fluorescence images showing multivalent PEG-nucleotide (base-labeled) ligands at 500 nM after mixing in the exposure buffer and imaging in the imaging buffer as above (FIG. 21B: PB1; FIG. 21C: PB2; FIG. 21D: PB3; FIG. 21E: PB5). FIG. 21F: fluorescence image showing multivalent PEG-nucleotide (base-labeled) ligand PB5 at 2.5 uM after mixing in the exposure buffer and imaging in the imaging buffer as above. In FIGS. 21G-21I, the fluorescence images showing further base discrimination by exposure of multivalent ligands to inactive mutants of Klenow polymerase (FIG. 21G: D882H; FIG. 21H: D882E; FIG. 21I: D882A, and the wild type Klenow (control) enzyme is shown in FIG. 21J).

### Example 7—Sequencing of Target Nucleic Acid Molecules Using Ternary Complexes

Four known templates were amplified using RCA methods on a low binding substrate. Successive cycles were exposed to exposure buffer containing 20 nM Klenow polymerase and 2.5 mM Sr⁺² and washed with imaging buffer and imaged. After imaging, the substrates were washed with wash buffer (EDTA and high salt) and blocked nucleotides were added to proceed to the next base. The cycle was repeated for 5 cycles. Spots were detected using standard imaging processing and spot detection and the sequences were called using a two-color green and red scheme (G-Cy3 and A-Cy5) to identify the templates being cycled.

### Example 8—Coating Flow Cell Surfaces with a Hydrophilic Polymer Coating

Glass flow cell devices were coated by washing prepared glass channels with KOH, followed by rinsing with ethanol and then silanization for 30 minutes at 65° C. Fluid channel surfaces were activated with EDC-NHS for 30 min., followed by grafting of oligonucleotide primers by incubation of the activated surface with 5 pm primer for 20 min. and then passivation with 30 pm of an amino-terminated polyethylene glycol (PEG-NH2).

### Example 9—Imaging of Nucleic Acid Clusters in a Capillary Flow Cell

Nucleic acid clusters were established within a capillary and subjected to fluorescence imaging. A flow device having a capillary tube was used for the test. An example of the resulting cluster images is presented in FIG. 22, which demonstrated that nucleic acid clusters formed by amplification within the lumen of a capillary flow cell device as disclosed herein can be reliably formed and visualized.

### Prophetic Example—Multiplexed COVID-19 Assay

Hydrophilic surface: A glass slide is cleaned by 2M KOH treatment of 30 min at room temperature, washed and then surface silanol groups are activated using an oxygen plasma. Silane-PEG2K-amine (Nanocs, Inc., New York, NY) is applied at a concentration of 0.5% in ethanol solution. After 2 h of coating reaction, the slide is washed thoroughly with ethanol and water. 100 pM of 8-arm PEG NHS (MW=10K, Creative PEGWorks, Inc., Durham, NC) is introduced at room temperature for 20 min in a solvent composition that includes 5, 10, 20, 30, 40, 50, 60, 70, 80 or 90% organic solvent and 5, 10, 20, 30, 40, 50, 60, 70, 80 or 90% low ionic strength buffer. The resulting surface is washed and reacted with 20 pM multi-arm PEG amine (MW=10K, Creative PEGWorks, Inc., Durham, NC) for 2 h. The resulting amine-PEG surface is then reacted with a mixture of multi-arm PEG-NHS and amine-labeled oligonucleotide primer at varying concentrations. This process is repeated to generate additional PEG layers on the surface. In this example, the hydrophilic surface exhibits a contrast-to-noise ratio of at least about 10, as measured according to Example 4 described herein.

Probe design: Four padlock probes are designed to target conserved regions on the SARS-CoV-2 (COVID-19) viral genome (2 probes), a spike in control (1 probe), and a negative control (1 probe) designed to have some complementarity to the viral genome, but containing a mismatch at the 3′ end to prevent ligation. The padlock probes hybridize to the target sequences via the 5′ and 3′ ends. Within the non-complementary region of the probe, there are RCA priming sites, the probe barcode, and any additional random sequence needed to facilitate circularization.

Target sequences are derived from the Centers for Disease Control and Prevention (CDC)-recommended COVID-19 loci and through bioinformatic assessment of both conserved and variable regions of the COVID-19 genome. Additional positive (spike in) controls and negative controls are also designed and included in the assay. Use of a plurality of barcoded padlock probes can be implemented to target multiple COVID-19 loci and can be identified through the associated probe barcode, permitting an assessment of the presence/absence of a COVID-19 target in a given sample and also providing information on a specific strain. For instance, barcoded padlock probes can be designed to target conserved regions for high-level presence/absence determination and variable regions to evaluate the presence or absence of a specific COVID-19 strain. The flexibility of creating the barcoded padlock probe pool, combined with the large data output accessible through the use of a sequencing platform for readout, allows the target probe panel to be constantly updated to include new mutant strains and continuously improve the precision of the assay.

The specificity of probe hybridization to the target are tested using similar target sequences as controls. Limits-of-detection (LoD) are also determined by monitoring ligation with decreasing numbers of target sequence copies present. For this phase of assay development, simple techniques, such as gel electrophoresis, are used to assess circularization and identify the most appropriate oligonucleotide probe set for this assay. It is important to note that, the assay can also be configured as a molecular inversion probe (MIP) assay, where the ligation event is replaced with a gap-fill ligation event, paving the way for a highly multiplexed genotyping assay executable directly on the disclosed sequencing platform.

Assay workflow: The circularized probes are the input for the sequencing-based readout at the heart of the disclosed methods. Following hybridization of the probes to viral RNA sequences in an individual sample, if present, and ligation, any remaining unreacted probe molecules or target nucleic acid may optionally be digested using an exonuclease and removed from the system. A sample index is added to all probes in a well (one sample per well) during the RCA step. The circularized padlock probes are RCA amplified using sample-indexed primers to generate concatemers that are fully compatible with the sequencing platform. In this example, the concatemers are loaded on the sequencing flow cell and become immobilized to the interior surface of the flow cell by hybridizing to the oligonucleotide primers covalently attached to the interior surface as described above. In other examples, the target nucleic acid (prior to RCA amplification) is immobilized to the interior surface of a the flow cell by hybridization to the oligonucleotide primers covalently attached to the interior surface of the flow cell, and a linear probe anneals to the target nucleic acid, followed by ligation, and optional digestion of the unreacted probes or target nucleic acid on the interior surface of the flow cell. In such an example, circularization of the probe, followed by amplification (e.g., RCA), are performed on the interior surface of the flow cell to form the concatemers compatible for sequencing. In either case, a few cycles of sequencing provide the sequence data required for probe barcode decoding as well as demultiplexing of the sample index. Secondary analysis is used to bin all probe barcode sequences belonging to a specific sample (including those for positive and negative controls), and the relative number of the virus-specific probe barcodes and those for the positive and negative controls counted for a given sample provides a determination of the presence/absence and titer of the viral load for the sample.

The advantages of this COVID-19 padlock probe+RCA assay system using sequencing for the read-out include, but are not limited to, (i) the barcoded padlock probe molecules target viral RNA directly without requiring transcription into cDNA; (ii) the assay is isothermal and rapid; (iii) multiple rounds of RCA, monomerization, and RCA can be repeated to increase assay sensitivity (other methods are also available to increase the sensitivity of this assay); and (iv) sample index sequences can be introduced during the RCA step using primers comprising sample-specific barcodes (although in some instances each padlock probe molecule can also include a sample barcode, in practice it is more versatile to introduce the sample index during RCA).

Sample indexing: Several sample indexing approaches for the generation of concatemers are evaluated for their impact on workflow, ability to impart additional flexibility into the assay design, and compatibility with the sequencing platform. Optimization of the RCA reaction conditions to maximize assay sensitivity is also underway. The formation of concatemers can be qualitatively evaluated through simple staining with either target oligos containing fluorophores or using dyes. Concatemer condensation to generate nanoballs is fully compatible with the existing sequencing platform, therefore, quantitative assessment of RCA and concatemer formation cab be executed using existing imaging system upon capture within sequencing flow cells. Sequencing is conducted to decode the locus-specific ID (e.g., probe barcode) and demultiplex the sample index. Depending on the indexing strategy adopted, it can require two separate priming events. Because the probe barcode and sample index are designed to offer a high degree of difference between barcode sequences, probe decoding and sample demultiplexing are accurate even at elevated sequencing error rates, thereby, allowing a focus on the speed of decoding and demultiplexing while still preserving barcode classification accuracy. The data generated from these sequencing runs are initially evaluated qualitatively but will eventually become the data input for the data analysis pipeline described below.

Sequencing: The concatemers generated by sample-indexed RCA are immobilized to an interior surface of a sequencing flow cell, where they are condensed into individually addressable nanoballs. Each nanoball contains multiple copies of both sample index and probe ID, both of which can be rapidly sequenced with about 15 cycles of sequencing, resulting in a very fast demultiplexing and locus ID determination (<2h). Since the number of nanoballs is proportional to the number of viral copies, counting the index sequences and probe IDs results in a precise assessment of the titer as it addresses 10s or even 100s of thousands of reads for each assay. In this example, the sequencing reaction comprises priming the concatemers, bringing the primed concatemers (serving as a template) into contact with labeled nucleotide moieties (e.g., conjugated to a polymer core to form a polymer-nucleotide conjugate) in the presence of a polymerizing enzyme under conditions sufficient to cause a nucleotide binding reaction between the labeled nucleotide moieties and the concatemers such that the labeled nucleotide moieties are not incorporated into the growing primers annealed to the concatemers. A binding complex formed between the labeled nucleotide moieties and the primed concatemer occurs when the labeled nucleotide moiety and the next nucleotide to be sequenced in the primed concatemer template base-pair. In some cases, the binding complex is a ternary complex described herein comprising the labeled nucleotide moiety, the primed concatemer and the polymerizing enzyme. The binding complex is detected for each subsequent nucleotide in the primed concatemer template. In the case of the polymer-nucleotide conjugate shown, for example, in FIG. 6, multiple primed concatemers may bind to a single polymer-nucleotide conjugate to form a multivalent binding complex.

The available data output is very large in the disclosed platform designed for genomic application, and, in principle, it is also possible to accommodate a very large number of samples. However, the ideal configuration is for medium multiplexing (384 to 1536 samples per run) since this lends itself better to decentralization and more approachable sample batch sizes. Because of the limited amount of sequencing required, it is expected for a run to be complete in less than 2 h and available for less than $10 per sample. Further, decentralization of testing combined with the throughput of a single instrument, exceeding a few million samples/year, will allow for the deployment of a cloud-based data analysis infrastructure for the real-time monitoring of pandemic evolution.

Probe barcode and sample index Identification: After sequencing is complete, the sequenced sample index and probe barcode is matched to the set of known indices and probe barcode. In most cases, the sequence is a perfect match to one of the expected sequences. When this is not the case, the Hamming distance between the sequence and the known barcode sequences is computed. If the sequence is within a sufficiently small Hamming distance of an expected sequence, then the match is assigned. Otherwise, the sequencing read is discarded. The fraction of assigned reads out of the total amount of reads is tracked for quality control purposes, generating a number of quality metrics that is regularly tracked. If both the sample index sequence and the probe barcode sequence are matched, then the read is retained for downstream data interpretation.

Data interpretation: For each sample, a quality control step verifies that the number of probe barcodes for the positive control is within a specified range, and the number of probe barcodes for the negative control is below some specified threshold value. These values are empirically defined through controlled experiments conducted on known samples comprising known viral RNA copy numbers. The number of viral copies in the sample correlates with the ratio of the virus-specific probes and the positive control. An estimate of the number of viral copies is made from each of the virus-specific probes and then averaged if the two estimates are comparable (else the test is considered failed). To assess the extent to which the assay is quantitative, spike-in controls at different concentrations is used. The disclosed sequencing platform generates hundreds of millions of reads (or individual assays) in a cost-effective manner. Therefore, LoD, viral load, and accuracy are tuned by increasing the density of the reads on the flow cell. A positive sample yields a number of counts above a threshold for both positive control and COVID specific sites. The count number is proportional to the amount of RNA copies present in the originating sample, with fewer copies resulting in lower count number. A negative sample shows counts for the positive control.

The number of sequencing reads required per sample to meet a target assay sensitivity is determined by performing trial sequencing runs. For example, the current CDC standard for COVID-19 is 10 RNA copies/uL for >95% true positives identified in replicate studies. Studies are performed to determine actual assay sensitivity, precision, false-positive rate, false-negative rate, and other quality metrics. Preliminary assay validation is performed by comparison to a gold-standard method such as RT-PCR.

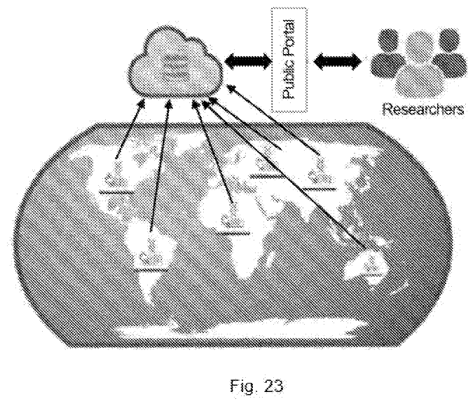

Data aggregation and pandemic monitoring: Participating laboratories have the option to automatically transfer anonymized results to a centralized, cloud-based database that can be used to monitor the progression of the pandemic and to identify potential new hotspots. The data transferred are de-identified, and aggregate statistics based on general location and sample collection dates are publicly accessible. A portal is developed that allows researchers to query and visualize the aggregate statistics. A schematic representation of a cloud-based approach to global pandemic monitoring is illustrated in FIG. 23. While the strength of this approach lies in the opportunity to decentralize testing, it also has appeal for centralized testing service providers that a very large sample processing capacity is immediately accessible, and the automation and logistical infrastructure to handle these large numbers of samples is already in place.

Key attributes that drive the commercial success of this assay are rapid turn-around time (comparable to that of current PCR assays) and ease of use. The approach has the potential for providing an unprecedented level of performance along all assay dimensions of low cost per sample, assay precision, sample throughput, and setup cost. Expected sample-to-answer times are less than 3 hours with minimal hands on time required. Optimization of the workflow and sequencing platform performance accelerates both the assay and the readout components of the method. Tuning of the sequencing platform performance is required since, for existing genomics application, speed of sequencing is valuable in the context of a very low error rate. For this application, in which pre-defined probe barcode and sample index sequences are read, error rate is less important, and sequencing speed at rates above those for an existing system can be acceptable.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. A method for nucleic acid detection, said method comprising:
(a) contacting a nucleic acid sequence obtained from a sample with a nucleic acid probe molecule comprising a distal end and a proximal end under conditions sufficient to couple said distal end of said nucleic acid probe molecule and said proximal end of said nucleic acid probe molecule to said nucleic acid sequence, thereby forming a circular nucleic acid probe molecule; and
(b) detecting a presence of said nucleic acid sequence by identifying a sequence of said circular nucleic acid probe molecule, wherein said detecting comprises performing a nucleotide binding reaction in the presence of a polymerizing enzyme between (i) said circular nucleic acid probe molecule or a derivative thereof and (ii) a nucleotide moiety comprising a detectable label, wherein said nucleotide binding reaction is performed in the absence of incorporation of said nucleotide moiety into said circular nucleic acid probe molecule or derivative thereof.

2. The method of claim 1, wherein said circular nucleic acid probe molecule comprises a gap in a sequence thereof.

3. The method of claim 2, further comprising contacting said nucleic acid probe molecule with a polymerizing enzyme under conditions sufficient to perform an extension reaction, thereby filling said gap with a copy of a portion of said nucleic acid sequence.

4. The method of claim 3, wherein said sequence of said circular nucleic acid probe molecule that is identified in (b) comprises said portion of said nucleic acid sequence.

5. The method of claim 3, further comprising contacting said nucleic acid probe molecule with a ligating enzyme under conditions sufficient to ligate said distal end of said nucleic acid probe molecule to said proximal end of said nucleic acid probe molecule following said extension reaction.

6. The method of claim 2, wherein said gap comprises between 1 and 200 contiguous nucleotides in length.

7. The method of claim 1, further comprising contacting said nucleic acid probe molecule with a ligating enzyme under conditions sufficient to ligate said distal end of said nucleic acid probe molecule to said proximal end of said nucleic acid probe molecule, thereby forming said circular nucleic acid probe molecule.

8. The method of claim 1, wherein said nucleic acid probe molecule is linear when unhybridized.

9. The method of claim 1, wherein said nucleic acid sequence of said circular nucleic acid probe molecule that is identified in (b) comprises a barcode sequence that uniquely identifies said presence of said nucleic acid sequence when it is identified.

10. The method of claim 1, further comprising:
(c) repeating (a) to (b) to identify a plurality of said nucleic acid sequence of a plurality of said circular nucleic acid probe molecule in a sample; and
(d) counting a number of times each said nucleic acid sequence of said plurality of said nucleic acid sequence is identified in (c).

11. The method of claim 10, further comprising determining a copy number of said nucleic acid sequence in said sample, wherein said copy number of said nucleic acid sequence in said sample is proportional to said number of said times said each said nucleic acid sequence is counted in (d).

12. The method of claim 1, further comprising multiplexing said method comprising:
(c) repeating (a) to (b) to identify a plurality of said nucleic acid sequence of a plurality of said circular nucleic acid probe molecule in said sample, wherein a first subset of said plurality of said circular nucleic acid probe molecule is different from a second subset of said plurality of said circular nucleic acid molecule; and
(d) counting a number of times a first nucleic acid sequence of said first subset and a second nucleic acid sequence of said second subset are identified in (c).

13. The method of claim 12, wherein said first subset of said plurality of said circular nucleic acid probe molecule is different from said second subset of said plurality of said circular nucleic acid molecule in that:
(i) said first subset comprises a different barcode sequence from said second subset;
(ii) said first subset comprises a different distal end or proximal end from said second subset; or
(iii) a combination of (i) and (ii).

14. The method of claim 1, further comprising detecting a presence of a second nucleic acid sequence in said sample, comprising:
(c) contacting said second nucleic acid sequence in said sample with a second nucleic acid probe molecule under conditions sufficient to couple said second nucleic acid sequence with said second nucleic acid probe molecule, thereby forming a second circular nucleic acid probe molecule; and
(d) bringing said second circular nucleic acid probe molecule or derivative thereof in contact with (i) a second polymerizing enzyme and (ii) a second nucleotide moiety comprising a second detectable label under conditions sufficient to cause a second nucleotide binding reaction to occur between said second circular nucleic acid probe molecule or derivative thereof and said second nucleotide moiety in the absence of incorporation of said second nucleotide moiety into said second circular nucleic acid probe molecule or derivative thereof, wherein said second nucleic acid sequence is different from said nucleic acid sequence detected in (b).

15. The method of claim 1, further comprising amplifying said circular nucleic acid probe molecule to produce said derivative thereof.

16. The method of claim 15, wherein said amplifying comprises performing rolling circle amplification.

17. The method of claim 1, wherein said nucleotide moiety is coupled to a polymer core in a polymer-nucleotide composition, forming a polymer-nucleotide conjugate.

18. The method of claim 17, wherein said detectable label is coupled to said polymer core of said polymer-nucleotide composition.

19. The method of claim 1, wherein said nucleotide binding reaction comprises two or more binding events between two or more of said nucleotide moiety and two or more copies of said nucleic acid sequence.

20. The method of claim 1, wherein said detectable label comprises a fluorescent label.

21. The method of claim 1, further comprising detecting a presence of a second nucleic acid sequence derived from a second sample, comprising:
(c) contacting said second nucleic acid sequence in said second sample with a second nucleic acid probe molecule under conditions sufficient to couple said second nucleic acid sequence with said second nucleic acid probe molecule, thereby forming a second circular nucleic acid probe molecule; and
(d) bringing said second circular nucleic acid probe molecule or derivative thereof in contact with (i) a second polymerizing enzyme and (ii) a second nucleotide moiety comprising a second detectable label under conditions sufficient to cause a second nucleotide binding reaction to occur between said second circular nucleic acid probe molecule or derivative thereof and said second nucleotide moiety in the absence of incorporation of said second nucleotide moiety into said second circular nucleic acid probe molecule or derivative thereof, wherein said second nucleic acid sequence is different from said nucleic acid sequence detected in (b), thereby detecting said presence of said second nucleic acid sequence in said second sample.

22. The method of claim 21, wherein said second sample is obtained from a different source from said sample.

23. The method of claim 21, further comprising tracing a pathogenic infection by a pathogenic source of said nucleic acid sequence and said second nucleic acid sequence, wherein said tracing comprises comparing a first location or a first time of collection of said sample with a second location or a second time of collection of said second sample.

24. The method of any one of claims 1-23, wherein said sample is obtained from a source comprising:
(i) soil;
(ii) sewage;
(iii) biological tissue;
(iv) food;
(v) a surface of an object in contact with one or more of (i) to (iv); or
(vi) any combination of (i) to (v).

25. A system for nucleic acid detection, said system comprising:
one or more computer processors that are individually or collectively programmed to implement a method comprising:
(a) contacting a nucleic acid sequence with a nucleic acid probe molecule under conditions sufficient to cause (i) a proximal end of said nucleic acid probe molecule to couple with a first portion of said nucleic acid sequence, and (ii) a distal end of said nucleic acid probe molecule to couple with a second portion of said nucleic acid sequence, thereby forming a circular nucleic acid probe molecule; and
(b) bringing said circular nucleic acid probe molecule or a derivative thereof in contact with (i) a polymerizing enzyme and (ii) a nucleotide moiety comprising a detectable label under conditions sufficient to cause a nucleotide binding reaction to occur between said circular nucleic acid probe molecule or derivative thereof and said nucleotide moiety in the absence of incorporation of said nucleotide moiety into said circular nucleic acid probe molecule or derivative thereof.

26. The system of claim 25, further comprising said nucleic acid probe molecule, wherein said nucleic acid probe molecule comprises (i) said proximal end comprising a first nucleic acid sequence that is complementary to said first portion of said nucleic acid sequence, and (ii) said distal end comprising a second nucleic acid sequence that is complementary to said second portion of said nucleic acid sequence.

27. The system of claim 25, further comprising a substrate having a surface comprising a polymer layer coupled thereto, wherein said circular nucleic acid probe molecule is coupled to said polymer layer.

28. The system of claim 27, wherein said polymer layer comprises a hydrophilic polymer.

29. The system of claim 28, wherein said hydrophilic polymer comprises poly(ethylene glycol) (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, dextran, or any combination thereof.

30. The system of claim 27, wherein said surface comprises two or more interior surfaces of a flow cell.

31. The system of claim 25, further comprising a ligating enzyme or catalytically-active fragment thereof configured to ligate said proximal end of said nucleic acid probe molecule and said distal end of said nucleic acid probe molecule to form said circular nucleic acid probe molecule.

32. The system of claim 25, wherein said circular nucleic acid probe molecule comprises a gap in a nucleic acid sequence thereof.

33. The system of claim 32, further comprising a polymerizing enzyme configured to perform an extension reaction of said circular nucleic acid probe molecule, thereby filling said gap.

34. The system of claim 33, wherein said gap is filled with a copy of a third portion of said nucleic acid sequence.

35. The system of claim 32, wherein said gap comprises between 1 and 200 contiguous nucleotides in length.

36. The system of claim 25, wherein said nucleic acid probe molecule is linear when unhybridized.

37. The system of claim 25, wherein said method further comprises repeating (a) and (b) to identify a sequence of said circular nucleic acid probe molecule or derivative thereof, wherein said sequence comprises a barcode sequence that uniquely identifies said sequence.

38. The system of claim 25, wherein said method further comprises:
(c) repeating (a) to (b) to identify a plurality of said nucleic acid sequence of a plurality of said circular nucleic acid probe molecule in said sample; and
(d) counting a number of times each sequence of said plurality of said sequence of said plurality of said circular nucleic acid probe molecule is identified in (c).

39. The system of claim 25, further comprising a plurality of said circular nucleic acid probe molecule comprising a first subset of said plurality of said circular nucleic acid probe molecule and a second subset of said plurality of said circular nucleic acid probe molecule, wherein said first subset is different from said second subset.

40. The system of claim 39, wherein said method further comprises:
(c) repeating (a) to (b) to identify a plurality of said nucleic acid sequence of a plurality of said circular nucleic acid probe molecule in said sample; and
(d) counting a number of times a first sequence of said first subset and a second sequence of said second subset are identified in (c).

41. The system of claim 39 or claim 40, wherein said first subset of said plurality of said circular nucleic acid probe molecule is different from said second subset of said plurality of said circular nucleic acid probe molecule in that:
(i) said first subset comprises a different barcode sequence from said second subset;
(ii) said first subset comprises a different distal end or proximal end from said second subset; or
(iii) a combination of (i) and (ii).

42. The system of claim 25, further comprising a second nucleic acid probe molecule, wherein said second nucleic acid probe molecule is configured to couple to a second nucleic acid sequence that is different from said nucleic acid sequence.

43. The system of claim 42, wherein said method further comprises detecting a presence of said second nucleic acid in said sample, comprising:
(c) contacting said second nucleic acid sequence in said sample with said second nucleic acid probe molecule under conditions sufficient to couple said second nucleic acid sequence with said second nucleic acid probe molecule, thereby forming a second circular nucleic acid probe molecule; and
(b) bringing said second circular nucleic acid probe molecule or derivative thereof in contact with (i) a second polymerizing enzyme and (ii) a second nucleotide moiety comprising a second detectable label under conditions sufficient to cause a second nucleotide binding reaction to occur between said second circular nucleic acid probe molecule or derivative thereof and said second nucleotide moiety in the absence of incorporation of said second nucleotide moiety into said second circular nucleic acid probe molecule or derivative thereof.

44. The system of claim 25, wherein said nucleotide moiety is coupled to a polymer core in a polymer-nucleotide composition.

45. The system of claim 44, wherein said detectable label is coupled to said polymer core in said polymer-nucleotide composition, forming a polymer-nucleotide conjugate.

46. The system of claim 25, wherein said nucleotide binding reaction comprises two or more binding events between two or more of said nucleotide moiety and two or more copies of said nucleic acid sequence.

47. The system of claim 25, wherein said detectable label comprises a fluorescent label.

48. The system of claim 25, wherein said nucleic acid sequence is obtained from a sample comprising:
(i) soil;
(ii) sewage;
(iii) biological tissue;
(iv) food;
(v) a surface of an object in contact with one or more of (i) to (iv); or
(vi) any combination of (i) to (v).
